(19) United States
(12) Patent Application Publication
(10) Pub. No.: US 2023/0173093 A1
(43) Pub. Date: Jun. 8, 2023

(54) CHARGE VARIANT LINKERS

(71) Applicant: Seagen Inc., Bothell, WA (US)

(72) Inventors: Christopher Scott NEUMANN, Bothell, WA (US); Joshua HUNTER, Bothell, WA (US)

(21) Appl. No.: 17/917,531

(22) PCT Filed: Apr. 9, 2021
(86) PCT No.: PCT/US2021/026718

§ 371 Date: Oct. 6, 2022

Related U.S. Application Data
(60) Provisional application No. 63/016,219, filed on Apr. 27, 2020.
(60) Provisional application No. 63/008,551, filed on Apr. 10, 2020.

(51) Int. Cl. A61K 47/68; A61K 47/54; A61K 47/60; A61P 35/00

(52) U.S. Cl. CPC A61K 47/6889; A61K 47/545; A61K 47/60; A61K 47/6817; A61K 47/6809; A61K 47/6849; A61P 35/00

(57) ABSTRACT

The present disclosure provides, inter alia, ADCs with charge variant chemical linkers useful in treating various diseases such as cancer and autoimmune disorders.

### BACKGROUND

Antibody-drug conjugates (ADCs) combine the tumor targeting specificity of monoclonal antibodies with the potent cell-killing activity of cytotoxic warheads. There has been a surge of interest in designing new ADC formats due in part to the recent clinical success of ADCs, which includes the approvals of brentuximab vedotin (ADCETRIS©) in relapsed Hodgkin lymphoma and anaplastic large-cell lymphoma, and ado-trastuzumab mertansine (KADCYLA©) in HER2-positive metastatic breast cancer.

The absolute quantity of delivered drug is limited, in part, by the level of antigen expression, the internalization rate of the ADC, and the number of molecules of drug conjugated to the antibody (the drug-antibody ratio or “DAR”). These restrictions contribute to the observation that highly potent cytotoxic molecules are typically used for the construction of active ADCs, because payloads of more modest potency tend to show more limited activity. One route to increasing the amount of drug delivered to cells is to increase the DAR of the conjugate; however, this approach often leads to a reduced half-life and reduced in vivo efficacy. The fast clearance of many such higher-loaded ADCs is often attributed to poor biophysical properties, but specific identification of these properties is lacking. Recent developments in higher loaded conjugates, such as those with hydrophobic drugs leading to ADC aggregation, have depended on hydrophilic polymer-based systems having heterogenous structure and drug loading to avoid aggregation and related issues.

### SUMMARY

Some embodiments provide an antibody-drug conjugate (ADC) compound of Formula (I):

Ab-{(S*-L¹)-[(M)ₓ-(L²-D)_{y}]}ₚ (I)

wherein:

Ab is an antibody;

each S* is a sulfur atom from a cysteine residue of the antibody, an ϵ-nitrogen atom from a lysine residue of the antibody, or a triazole moiety, and

each L¹ is a first linker optionally substituted with a PEG Unit ranging from PEG2 to PEG72;

wherein S*-L¹ is selected from the group consisting of formulae A-K:

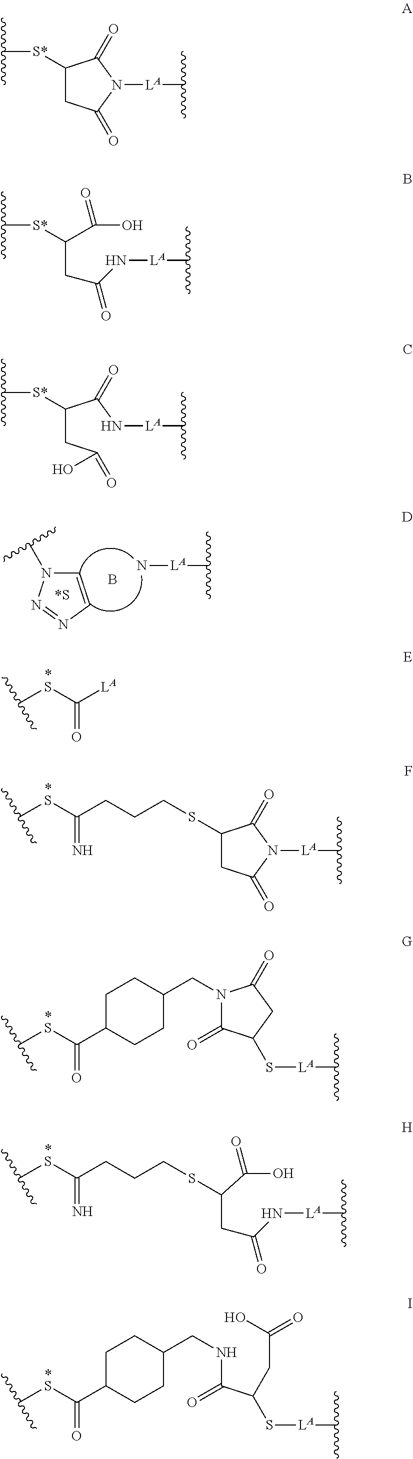

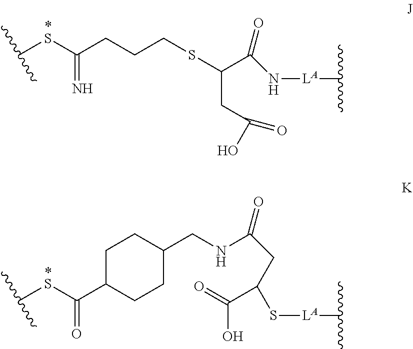

wherein:

each L^{A} is a C₁₋₁₀ alkylene optionally substituted with 1-3 independently selected R^{a}, or a 2-24 membered heteroalkylene optionally substituted with 1-3 independently selected R^{b};

each Ring B is an 8-12 membered heterocyclyl optionally substituted with 1-3 independently selected R^{c}, and further optionally fused to 1-2 rings each independently selected from the group consisting of C₆₋₁₀ aryl and 5-6 membered heteroaryl;

each R^{a}, R^{b}, and R^{c} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d}R^{e}, —C(O)NR^{d}R^{e}, —C(O)(C₁₋₆ alkyl), —(C₁₋₆ alkylene)-NR^{d}R^{e}, and —C(O)O(C₁₋₆ alkyl);

each R^{d} and R^{e} are independently hydrogen or C₁₋₃ alkyl; or R^{d} and R^{e} together with the nitrogen atom to which both are attached form a 5-6 membered heterocyclyl;

L² is an optional second linker optionally substituted with a PEG Unit selected from PEG2 to PEG20;

each M is a multiplexer;

subscript x is 0, 1, 2, 3, or 4;

subscript y is 2^{x};

each D is a Drug Unit;

wherein L¹ and each (M)ₓ-(D)_{y} when L² is absent, or each (M)ₓ-(L²-D)_{y} when L² is present, have a net zero charge at physiological pH;

subscript p is an integer ranging from 2 to 10; and

the ratio of D to Ab is 8:1 to 64:1.

Some embodiments provide a composition comprising an ADC as describe herein, or a pharmaceutically acceptable salt thereof.

Some embodiments provide a method of treating cancer in a subject in need thereof, comprising administering to the subject a therapeutically effective amount an ADC as describe herein, or a pharmaceutically acceptable salt thereof, or a composition comprising an ADC as describe herein, or a pharmaceutically acceptable salt thereof, as described herein.

Some embodiments provide a method of treating an autoimmune disorder in a subject in need thereof, comprising administering to the subject a therapeutically effective amount an ADC as describe herein, or a pharmaceutically acceptable salt thereof, or a composition comprising an ADC as describe herein, or a pharmaceutically acceptable salt thereof, as described herein.

### BRIEF DESCRIPTION OF THE DRAWINGS

FIG. 1 provides the HIC chromatogram (at 280 nm) of hAC10ec and its conjugates with MC1 or MC3 (DAR=10, 20, or 38.5).

FIG. 2 schematically depicts sequential reactions of MC2 and N-ethyl maleimide onto cysteine residues of an antibody. An antibody (cAC10) having a L0=23152 was reacted with MC2 to form an antibody-duplexer compound (expected mass: 23,476; observed mass: 23,475). The disulfide bond of the MC2 duplexer of the antibody-duplexer compound was then reduced with TCEP, followed by reaction of the reduced antibody-duplexer compound with N-ethylmaleimide (NEM) (2 equivalents) to form an antibody-duplexer-NEM compound (expected mass 23,723; observed mass 23,725).

FIG. 3 provides the size exclusion chromatogram of auristatin ADCs (DAR=16). FIG. 3A provides the size exclusion chromatogram of the ADC cAC10-MC2(8)-MC4(16) (retention time: about 6.6 minutes). FIG. 3B provides the size exclusion chromatogram of the ADC cAC10-MC2(8)-MC5(16) (retention time: about 6.6 minutes).

FIG. 4A provides the PLRP chromatogram of reduced cAC10 antibody that has undergone sequential reactions with MC2 and MC4 (retention time of light chain: about 1.29 minutes; retention time of heavy chain: about 1.97 minutes). FIG. 4B provides the mass spectrum of antibody (cAC10) light chain from the intact antibody that has undergone reaction with one unit of MC2 (expected: 25,737; observed 25,737). FIG. 4C provides the mass spectrum of antibody (cAC10) light chain from the intact antibody attached to MC2(1)-MC4(2) (expected: 28,072; observed 28,072). FIG. 4D provides the mass spectrum of antibody (cAC10) heavy chain from the intact antibody attached to MC2(3)-MC4(6) (expected: 63,364; observed: 63,364).

FIG. 5A provides the PLRP chromatogram of reduced cAC10 antibody that has undergone sequential reactions with MC2 and MC5 (retention time of light chain: about 0.33 minutes; retention time of heavy chain: about 1.0 minutes. FIG. 5B provides the mass spectrum of the antibody (cAC10) light chain to MC2(1)-MC5(2) (expected: 26,244; observed: 26,244). FIG. 5C provides the mass spectrum data of the antibody (cAC10) heavy chain attached to MC2(3)-MC5(6) (expected: 57,880; observed: 57,879).

FIG. 6 schematically depicts an exemplary method for the preparation of ADCs comprising one or more multiplexer moieties. In that method an individual antibody is reduced and reacted with MC2. In a monoclonal antibody with engineered two cysteine residues (ECmAb), having 10 total Cys residues (eight native and two engineered), the thiol group of each cysteine is reacted with a MC2 unit. Each MC2 unit (after disulfide reduction) is then reacted with two additional MC2 units. Conjugation of L²-D moieties to the terminal MC2 units upon reduction of their disulfide bonds forms ADCs with DAR=40. Those ADCs have the general formula of Ab-MC2(10)-MC2(20)-(L²-D)(40).

FIG. 7 provides the HIC chromatogram of hAC10 conjugates with MC1 or MC3 having different DARs (DAR=0, 10, 20, and 38.5).

FIG. 8 provides the in vitro cytotoxicity of cAc10ec-MC1 ADCs having different DARs (DAR=10, 20, and 38.5) to Hodgkin's Lymphoma cell line L540cy.

FIG. 9 provides the rat pharmacokinetic data of DAR16 conjugates of a non-binding IgG1 antibody with conjugation to a NAMPT inhibitor, with each conjugate having different charges in the L²-D moieties. ADCs with L²-D=MC9 (neutral) or MC8 (zwitterionic) are compared with those having L²-D=MC7 (negatively charged) and MC10 (positively charged).

FIG. 10 provides the efficacy of cAC10 or non-binding IgG1 conjugates with an NAMPT inhibitor, which have the general formula of cAC10-MC6(8)-(L²-D)(16) or IgG1-MC6(8)-(L²-D)(16), respectively, in an in vivo xenograft model with L540cy cells, wherein L²-D is MC7, MC8, MC9, or MC10.

FIG. 11 provides the efficacy of Ab3(ec)-MC6(10)-MC9(20) and Ab3(ec)-MC7(10) ADCs on KG1-22 cells in an in vivo xenograft model using both antibody- and drug-normalized dosing (mean tumor data).

### DETAILED DESCRIPTION

It is expected that ADCs with linkers having a net charge would have superior biophysical properties due to their greater hydrophilicity. In contrast, it has been unexpectedly found that having a net charge on the linker in a higher-loaded ADC can have a profound negative effect on its biophysical properties. For example, ADCs with drug-linkers having a net zero charge outperform comparator ADCs in which the linkers have a net positive change or a net negative charge.

Accordingly, provided herein are ADCs of Formula (I) having charge-variant linkers and a range of drug-antibody ratios (DARs), including ADCs with high DARs (e.g., DAR>8). Traditional high DAR ADCs exhibit reduced potency and/or require heterogenous polymer-based systems to avoid aggregation (and concomitant loss of potency). In some embodiments, the ADCs described herein exhibit more favorable biophysical properties as compared to that typically observed with traditional high-load ADCs. In some embodiments, the ADCs described herein have more favorable biophysical properties as compared to high DAR ADCs with a linker having a net charge. In some embodiments, the ADCs described herein have improved in vivo efficacy as compared to high DAR ADCs with a linker having a net charge. The in vivo efficacy of ADCs largely depends on their pharmacokinetics and the potency of its payload. ADCs of Formula (I) have charge-variant linkers such that the drug-linker moieties of the ADC are zwitterionic or neutral (i.e., have a net zero charge) at physiological pH. In some embodiments, ADCs of Formula (I) exhibit extended half-lives relative to traditional high-load ADCs or comparator ADC with drug-linker moieties that have a net positive or negative charge. This approach can enable tuning of an ADC's half-life, and the use of less potent compounds (e.g., less cytotoxic compounds) as the Drug Unit of the ADC, which typically requires a higher DAR compared to those with conjugation to more cytotoxic compounds, in order to exhibit the required efficacy for treating cancer.

### Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a linker” includes reference to one or more such linkers, and reference to “the cell” includes reference to a plurality of such cells.

The term “about” when referring to a number or a numerical range means that the number or numerical range referred to is an approximation, for example, within experimental variability and/or statistical experimental error, and thus the number or numerical range may vary up to ±10% of the stated number or numerical range. In reference to an ADC composition comprising a distribution of ADCs as described herein, the average number of conjugated Drug Units to an antibody in the composition can be an integer or a non-integer, particularly when the antibody is to be partially loaded. Thus, the term “about” recited prior to an average drug loading value is intended to capture the expected variations in drug loading within an ADC composition.

The term “inhibit” or “inhibition of” means to reduce by a measurable amount, or to prevent entirely (e.g., 100% inhibition).

The term “therapeutically effective amount” refers to an amount of an ADC, or a salt thereof (as described herein), that is effective to treat a disease or disorder in a mammal. In the case of cancer, the therapeutically effective amount of the ADC provides one or more of the following biological effects: reduction of the number of cancer cells; reduction of tumor size; inhibition of cancer cell infiltration into peripheral organs; inhibition of tumor metastasis; inhibition, to some extent, of tumor growth; and/or relief, to some extent, of one or more of the symptoms associated with the cancer. For cancer therapy, efficacy, in some aspects, is measured by assessing the time to disease progression (TTP) and/or determining the response rate (RR).

Unless otherwise indicated or implied by context, the term “substantial” or “substantially” refers to a majority, i.e. >50% of a population, of a mixture, or a sample, typically more than 50%, 55%, 60%, 65%, 70%, 75%, 80%, 85%, 90%, 91%, 92%, 93%, 94%, 95%, 96%, 97%, 98%, or 99%.

The terms “intracellularly cleaved” and “intracellular cleavage” refer to a metabolic process or reaction occurring inside a cell, in which the cellular machinery acts on the ADC or a fragment thereof, to intracellularly release free drug from the ADC, or other degradant products thereof. The moieties resulting from that metabolic process or reaction are thus intracellular metabolites.

The term “cytotoxic activity” refers to a cell-killing effect of a drug or ADC or an intracellular metabolite of an ADC. Cytotoxic activity is typically expressed by an IC₅₀ value, which is the concentration (molar or mass) per unit volume at which half the cells survive exposure to a cytotoxic agent.

The term “cytostatic activity” refers to an anti-proliferative effect other than cell killing of a cytostatic agent, or an ADC having a cytostatic agent as its Drug Unit (D) or an intracellular metabolite thereof wherein the metabolite is a cytostatic agent.

The term “cytotoxic agent” as used herein refers to a substance that has cytotoxic activity, as defined herein. The term is intended to include chemotherapeutic agents, and toxins such as small molecule toxins or enzymatically active toxins of bacterial, fungal, plant or animal origin, including synthetic analogs and derivatives thereof.

The term “cytostatic agent” as used herein refers to a substance that has cytostatic activity as defined herein. Cytostatic agents include, for example, enzyme inhibitors.

The terms “cancer” and “cancerous” refer to or describe the physiological condition or disorder in mammals that is typically characterized by unregulated cell growth. A “tumor” comprises multiple cancerous cells.

An “autoimmune disorder” herein is a disease or disorder arising from and directed against a subject's own tissues or proteins.

“Subject” as used herein refers to an individual to which an ADC, as described herein, is administered. Examples of a “subject” include, but are not limited to, a mammal such as a human, rat, mouse, guinea pig, non-human primate, pig, goat, cow, horse, dog, cat, bird and fowl. Typically, a subject is a rat, mouse, dog, non-human primate, or human. In some aspects, the subject is a human.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment.

In the context of cancer, the term “treating” includes any or all of: inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disorder, the term “treating” includes any or all of: inhibiting replication of cells associated with an autoimmune disorder state including, but not limited to, cells that produce an autoimmune antibody, lessening the autoimmune-antibody burden and ameliorating one or more symptoms of an autoimmune disorder.

The term “salt,” as used herein, refers to organic or inorganic salts of a compound, such as a Drug Unit (D), a linker such as those described herein, or an ADC. In some aspects, the compound contains at least one amino group, and accordingly, acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion or other counterion. The counterion may be any organic or inorganic moiety that stabilizes the charge on the parent compound. Furthermore, a salt has one or more than one charged atom in its structure. In instances where there are multiple charged atoms as part of the salt multiple counter ions are sometimes present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Ztrich:Wiley-VCH/VHCA, 2002, the list for which is specifically incorporated by reference herein.

The term “alkyl” refers to a straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoromethylene (—CF₂—), tetrafluoroethylene (—CF₂CF₂—), and the like.

The term “heteroalkyl” refers to a stable straight or branched chain hydrocarbon that is fully or partially saturated having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “heteroalkylene” refers to a bivalent unsubstituted straight or branched group derived from heteroalkyl (as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂— —C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N⁺(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “haloalkyl” refers to a straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, difluoromethyl, trifluoromethyl, 2,2,2-trifluoroethyl, and 1-chloroisopropyl.

The term “haloalkoxy” refers to a haloalkyl group, as defined herein, which is attached to a molecule via an oxygen atom. For example, haloalkoxy groups include, but are not limited to difluoromethoxy, trifluoromethoxy, 2,2,2-trifluoroethoxy, and 1,1,1-trifluoro2-methylpropoxy.

The term “aryl” refers to a monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocyclyl” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2, or 3) oxo groups and the sulfur and nitrogen atoms may also be present in their oxidized forms. Such rings include but are not limited to azetidinyl, tetrahydrofuranyl, and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl), or aryls. The rings of a multiple condensed ring system can be connected to each other via fused, spiro, or bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including carbon atoms and heteroatoms (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated 71 system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

As used herein, the term “free drug” refers to a biologically active species that is not covalently attached to an antibody. Accordingly, free drug refers to a compound as it exists immediately upon cleavage from the ADC. The release mechanism can be via a cleavable linker in the ADC, or via intracellular conversion or metabolism of the ADC. In some aspects, the free drug will be protonated and/or may exist as a charged moiety. The free drug is a pharmacologically active species which is capable of exerting the desired biological effect. In some embodiments, the pharamacologically active species is the parent drug alone. In some embodiments, the pharamacologically active species is the parent drug bonded to a component or vestige of the ADC (e.g., a component of the linker, succinimide, hydrolyzed succinimide, and/or antibody that has not undergone subsequent intracellular metabolism).

Exemplary free drug compounds have cytotoxic, cytostatic, immunosuppressive, immunostimulatory, or immunomodulatory drug. In some embodiments, D is a tubulin disrupting agent, DNA minor groove binder, DNA damaging agent or DNA replication inhibitor.

As used herein, the term “Drug Unit” refers to the free drug that is conjugated to an antibody in an ADC, as described herein.

As used herein, the term “hydrophilic drug” refers to a Drug Unit or free drug, as defined herein, having a log P value of 1.0 or less. Exemplary hydrophilic drugs include, but are not limited to antifolates, nucleosides and NAMPT inhibitors.

As used herein, “net zero charge” refers to a compound, or specific part of a compound, that has no net charge at physiological pH. For example, in the compounds of Formula (I) described herein, the L² and/or L¹-[(M)ₓ-(D)_{y}] parts of Formula (I) can have a net zero charge. Compounds, or parts of a compound, having a net zero charge includes those with two or more charged species, wherein the sum of the two or more charges is zero (such as a zwitterionic compound).

“Physiological pH,” as used herein, refers to a pH of about 7.3 to about 7.5, or a pH of 7.3 to 7.5.

Antibody-Drug Conjugates (ADCs) and Intermediates Thereof

First generation ADCs contained highly toxic payloads traditionally used for cancer chemotherapy, such as doxorubicin, microtubule inhibitors, and DNA-damaging agents. See Diamantis and Banerji, Br. J. Cancer, Vol. 114, pp. 362-367 (2016). Those early ADCs were highly toxic and generally had poor physiochemical properties, with only an estimated 1-2% of the payload reaching the targeted cells. See Beck, et al., Nat. Rev. Drug Discov., Vol. 16, pp. 315-337 (2017). Second generation ADCs, such as ado-trastuzumab emtansine (Kadcyla®) also provide cytotoxic payloads and include improved linkers facilitating release of the payload at or near the target cells. Despite these improvements, complex issues still remain in the design of ADCs.

The linker between the antibody and the payload controls the release, and thus the delivery, of the drug to the target. See Gerber, et al., Nat. Prod. Rep., Vol. 30, pp. 625-639 (2013). Premature drug release can cause severe off-target toxicities by killing healthy cells. Indeed, the linker must be stable enough to survive until binding of the antibody to the target, but labile enough for drug release (whether through direct enzymatic action, or a combination of enzymatic cleavage and hydrolysis). However, linkers may also effect the solubility, aggregation, and clearance of ADCs, thus influencing their distribution. See Jain, et al., Pharm. Res., Vol. 32, pp. 3526-3540 (2015). These issues contribute to the high interpatient variability and distribution patterns observed with many ADCs, impeding administration of the correct dose. See Krop, et al., Breast Cancer Res., Vol. 18, p. 34 (2016).

Moreover, a higher DAR generally leads to greater in vitro potency, but typically at the cost of poorer pharmacokinetic properties in vivo. See Hamblett, et al., Clin. Cancer Res., Vol. 10, pp. 7063-7070 (2004); see also, Sun, et al., Bioconj. Chem., Vol. 28, pp. 1371-1381 (2017). Indeed, when otherwise identical ADCs were prepared with DARs of 2, 4, and 8, the clearance of the ADCs increased at the DAR increased. See, e.g., Hamblett, et al. (2004), supra.

The present application is based, in part, on the surprising finding that modulation of the charge of the linker between the antibody and the drug can have a dramatic impact on the pharmacokinetic properties of the ADC. In particular, linkers that are uncharged, or have a net zero charge (e.g., zwitterionic linkers) provide access to ADCs with a range of DARs. In some embodiments, the ADCs provided herein exhibit in vitro potency as well as improved pharmacokinetic properties.

Some embodiments provide an antibody drug conjugate (ADC) compound of Formula (I):

Ab-{(S*-L¹)-[(M)ₓ-(L²-D)_{y}]}ₚ (I)

wherein Ab is an antibody;

each S* is a sulfur atom from a cysteine residue of the antibody, an ϵ-nitrogen atom from a lysine residue of the antibody, or a triazole moiety, and

each L¹ is a first linker optionally substituted with a PEG Unit ranging from PEG2 to PEG72,

wherein S*-L¹ is selected from the group consisting of formulae A-K:

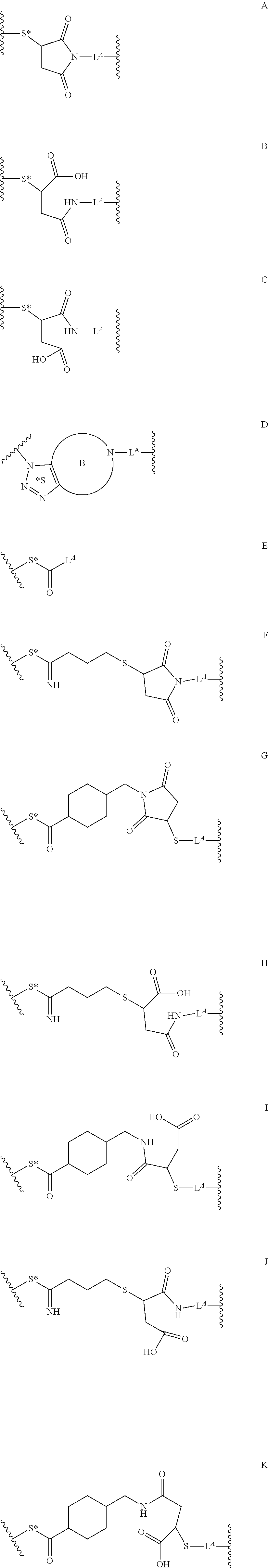

wherein:

each L^{A} is a C₁₋₁₀ alkylene optionally substituted with 1-3 independently selected R^{a}, or a 2-24 membered heteroalkylene optionally substituted with 1-3 independently selected R^{b};

each Ring B is an 8-12 membered heterocyclyl optionally substituted with 1-3 independently selected R^{c}, and further optionally fused to 1-2 rings each independently selected from the group consisting of C₆₋₁₀ aryl and 5-6 membered heteroaryl;

each R^{a}, R^{b}, and R^{c} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d}R^{e}, —(C₁₋₆ alkylene)-NR^{d}R^{e}, —C(O)NR^{d}R^{e}, —C(O)(C₁₋₆ alkyl), and —C(O)O(C₁₋₆ alkyl);

each R^{d} and R^{e} are independently hydrogen or C₁₋₃ alkyl; or R^{d} and R^{e} together with the nitrogen atom to which both are attached form a 5-6 membered heterocyclyl;

L² is an optional second linker optionally substituted with a PEG Unit ranging from PEG2 to PEG72;

each M is a multiplexer;

subscript x is 0, 1, 2, 3, or 4;

subscript y is 2^{x};

each D is a Drug Unit;

wherein each L²-D has a net zero charge at physiological pH; or wherein L¹ and each (M)ₓ-(D)_{y}, when L² is absent or each (M)ₓ-(L²-D)_{y}, when L² is present has a net zero charge at physiological pH;

subscript p is an integer ranging from 2 to 10; and

wherein the ratio of D to Ab is 8:1 to 64:1

In some embodiments, each S* is a sulfur atom from a cysteine residue of the antibody. In some embodiments, the cysteine residue is a native cysteine residue, an engineered cysteine residue, or a combination thereof. In some embodiments, each cysteine residue is from a reduced interchain disulfide bond. In some embodiments, each cysteine residue is an engineered cysteine residue. In some embodiments, each cysteine residue is a native cysteine residue. In some embodiments, one or more S* is a sulfur atom from an engineered cysteine residue; and any remaining S* is a sulfur atom from a native cysteine residue. In some embodiments, 1, 2, 3, or 4 S* is a sulfur atom from an engineered cysteine residue; and any remaining S* is a sulfur atom from a native cysteine residue.

In some embodiments, each S* is an ϵ-nitrogen atom from a lysine residue of the antibody. In some embodiments, the lysine residue is a native lysine residue, an engineered lysine residue, or a combination thereof. In some embodiments, each lysine residue is an engineered lysine residue. In some embodiments, each lysine residue is a native lysine residue. In some embodiments, one or more S* is an ϵ-nitrogen atom from an engineered lysine residue; and any remaining S* is an ϵ-nitrogen atom from a native lysine residue. In some embodiments, 1, 2, 3, or 4 S* is an ϵ-nitrogen atom from an engineered lysine residue; and any remaining S* is an ϵ-nitrogen atom from a native lysine residue.

In some embodiments, each S* is a triazole moiety. In some embodiments, when S* is a triazole moiety, that triazole moiety is formed through an azide-alkyne polar cycloaddition reaction (“click chemistry”) between an azide group and an alkyne group, as described herein. Methods to incorporate the azide or the alkyne precursors for cycloaddition that results in S* being a triazole moiety is by modifying one or more amino acid residues of the antibody.

In some embodiments, L¹ terminates in a component having a sufficiently strained alkyne functional group that is reactive towards a modified antibody bearing a suitable azide functional group. A dipolar cycloaddition between these two functional groups results in a triazole. In some embodiments, Diels-Alder type chemistry (4+2 cycloaddition, inverse electron demand) is used for the covalent attachment of an L¹ having a terminal 1,2,4,5-tetrazine to a modified antibody bearing a suitable trans cyclooctene functional group. For illustration, general depictions of the Click and Diels-Alder (4+2 cycloaddition) reactions are shown in a) and b) respectively. One of skill in the art will appreciate that a variety of modifications are possible, including, but not limited to, varying the substitution patterns of the reactive components, switching the portion (Ab or L¹) to which each reactive component is attached.

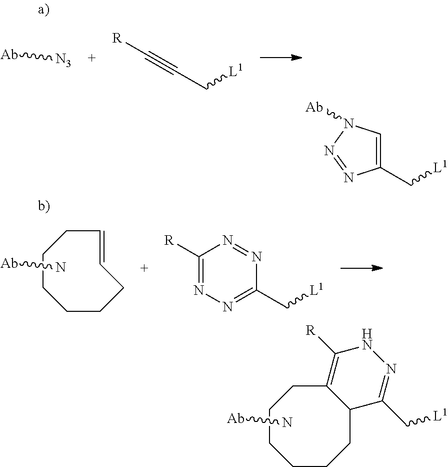

In some embodiments, S*-L¹ has formula A:

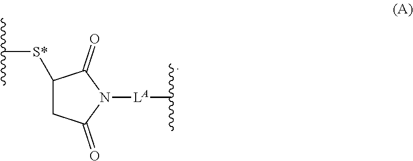

In some embodiments, S*-L¹ has formula B:

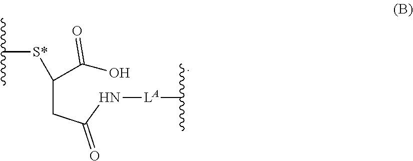

In some embodiments, S*-L¹ has formula C:

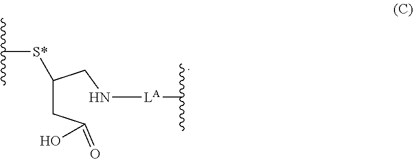

In some embodiments, S*-L¹ has formula D:

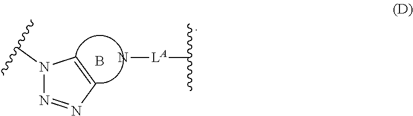

In some embodiments, S*-L¹ has formula E:

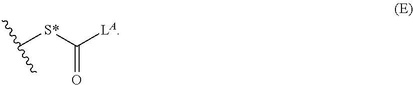

In some embodiments, S*-L¹ has formula F:

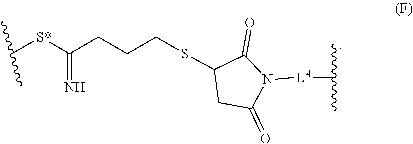

In some embodiments, S*-L¹ has formula G:

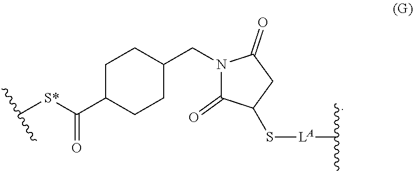

In some embodiments, S*-L¹ has formula H:

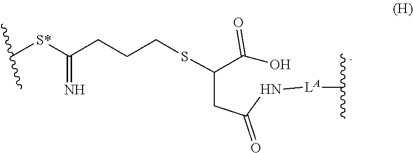

In some embodiments, S*-L¹ has formula I:

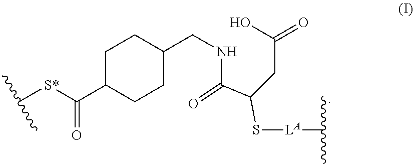

In some embodiments, S*-L¹ has formula J:

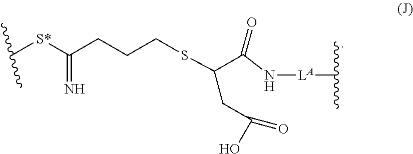

In some embodiments, S*-L¹ has formula K:

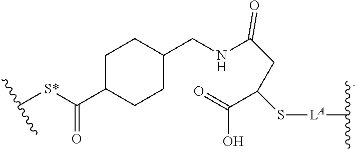

In some embodiments, when each S* is an ϵ-nitrogen atom from a lysine residue of the antibody, S*-L¹ is selected from the group consisting of formulae E1-K1:

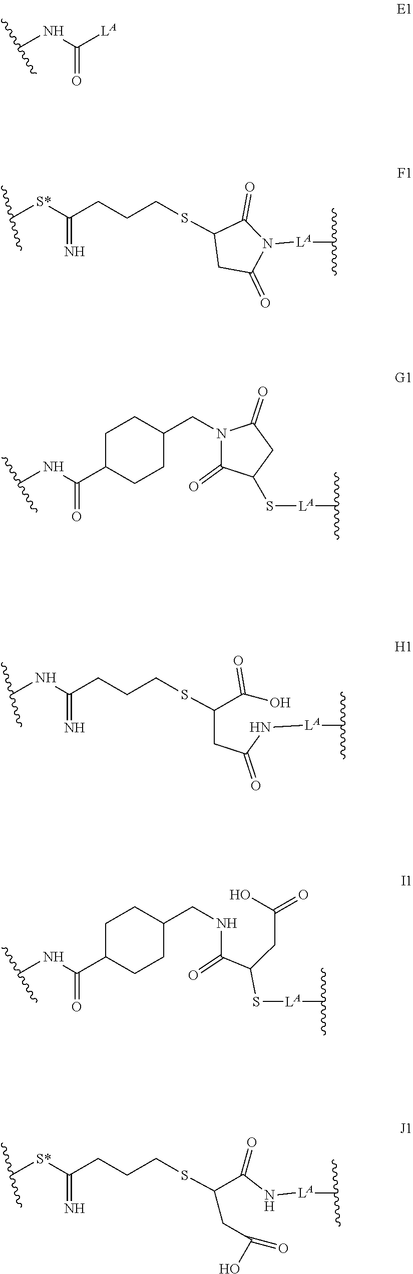

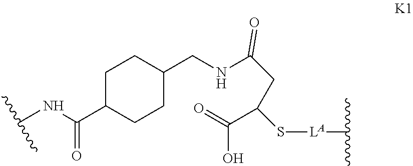

In some embodiments, L¹ is unsubstituted. In some embodiments, L¹ is substituted with a PEG Unit ranging from PEG2 to PEG72, for example, PEG2, PEG4, PEG6, PEG8, PEG10, PEG12, PEG16, PEG20, PEG 24, PEG36, or PEG72.

In some embodiments, L^{A} is C₁₋₁₀ alkylene optionally substituted with 1-3 independently selected R^{a}. In some embodiments, L^{A} is C₁₋₈ alkylene optionally substituted with 1-3 independently selected R^{a}. In some embodiments, L^{A} is C₁₋₆ alkylene optionally substituted with 1-3 independently selected R^{a}. In some embodiments, L^{A} is C₁₋₄ alkylene optionally substituted with 1-3 independently selected R.

In some embodiments, L^{A} is unsubstituted. In some embodiments, L^{A} is substituted with one R^{a}. In some embodiments, L^{A} is substituted with two R^{a}. In some embodiments, L^{A} is substituted with three R^{a}.

In some embodiments, L^{A}, together with its 0, 1, 2, or 3 R^{a}, is uncharged at physiological pH. In some embodiments, L^{A}, together with its 0, 1, 2, or 3 R^{a}, is charged neutral at physiological pH. In some embodiments, L^{A} is substituted with 2 R^{a}; wherein one R^{a} is positively charged and the other R^{a} is negatively charged.

In some embodiments, each R^{a} is selected from the group consisting of: C₁₋₆ alkoxy, halogen, —OH, —(C₁₋₆ alkylene)-NR^{d}R^{e}, —C(O)NR^{d}R^{e} and —C(O)(C₁₋₆ alkyl). In some embodiments, one of R^{a} is NR^{d}R^{e}, and the remaining R^{a} is not —NR^{d}R^{e}. In some embodiments, one of R^{a} is —(C₁₋₆ alkylene)-NR^{d}R^{e}, and the remaining R^{a} is not —(C₁₋₆ alkylene)-NR^{d}R^{e}. In some embodiments, one of R^{a} is NR^{d}R^{e}, and the remaining R^{a} is selected from the group consisting of: C₁₋₆ alkoxy, halogen, —OH, —C(O)NR^{d}R^{e} and —C(O)(C₁₋₆ alkyl). In some embodiments, one of R^{a} is —(C₁₋₆ alkylene)-NR^{d}R^{e}, and the remaining R^{a} is selected from the group consisting of: C₁₋₆ alkoxy, halogen, —OH, —C(O)NR^{d}R^{e} and —C(O)(C₁₋₆ alkyl).

In some embodiments, L^{A} is

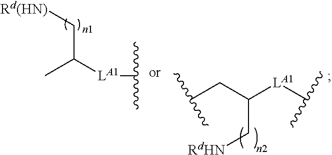

wherein L^{A1} is a bond or a C₁₋₅ alkylene optionally substituted with R^{a}; subscript n1 is 1-4; and subscript n2 is 0-4. In some embodiments, subscript n1 is 1. In some embodiments, subscript n1 is 2. In some embodiments, subscript n1 is 3. In some embodiments, subscript n1 is 4. In some embodiments, subscript n2 is 0. In some embodiments, subscript n2 is 1. In some embodiments, subscript n2 is 2. In some embodiments, subscript n2 is 3. In some embodiments, subscript n2 is 4.

In some embodiments, L^{A1} is a bond. In some embodiments, L^{A1} is a C₁₋₅ alkylene. In some embodiments, L^{A1} is unsubstituted. In some embodiments, L^{A1} is substituted with one R^{a}.

In some embodiments, L^{A} is

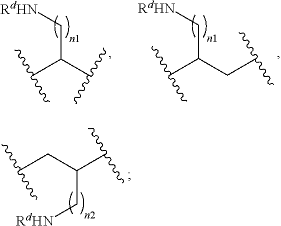

wherein subscript n1 is 1 or 2; and subscript n2 is 0, 1, or 2. In some embodiments, subscript n1 is 1. In some embodiments, subscript n1 is 2. In some embodiments, subscript n2 is 0. In some embodiments, subscript n2 is 1. In some embodiments, subscript n2 is 2. In some embodiments, subscript n1 is 1 and subscript n2 is 0. In some embodiments, subscript n1 is 1 and subscript n2 is 1. In some embodiments, subscript n1 is 1 and subscript n2 is 2. In some embodiments, subscript n1 is 2 and subscript n2 is 0. In some embodiments, subscript n1 is 2, and subscript n2 is 1. In some embodiments, subscript n1 is 2 and subscript n2 is 2.

In some embodiments, L^{A} is an unsubstituted C₁₋₁₀ alkylene, such as methylene, ethylene, propylene, n-butylene, sec-butylene, pentylene, or hexylene.

In some embodiments, L^{A} is a 2-24 membered heteroalkylene optionally substituted with 1-3 independently selected R^{b}, and optionally further substituted with a PEG Unit ranging from PEG2 to PEG24. In some embodiments, L^{A} is 2-12 membered heteroalkylene optionally substituted with 1-3 independently selected R^{b}, and optionally further substituted with a PEG Unit ranging from PEG2 to PEG24. In some embodiments, L^{A} is a 2-24 membered heteroalkylene having no charged heteroatoms at physiological pH optionally substituted with 1-3 independently selected R^{b}, and optionally further substituted with a PEG Unit ranging from PEG2 to PEG24. In some embodiments, L^{A} is unsubstituted. In some embodiments, R^{b} is not —NR^{d}R^{e} in formula A and formula D. In some embodiments, only one of R^{b} is —NR^{d}R^{e} in formula B and formula C.

In some embodiments, when L^{A} is substituted by a PEG Unit, the heteroalkylene of L^{A} is the site of substitution by the PEG Unit.

In some embodiments, L^{A} is substituted with 1-3 independently selected R^{b}, as described herein. In some embodiments, L^{A} is substituted with one R^{b}, as described herein. In some embodiments, L^{A} is substituted with two independently selected R^{b}, as described herein. In some embodiments, L^{A} is substituted with three independently selected R^{b}, as described herein.

In some embodiments, L^{A} is substituted with 1 R^{b} that is a PEG Unit ranging from PEG2 to PEG24.

In some embodiments, L^{A} is substituted with 1-3 independently selected R^{b} as described herein, one of which is a PEG Unit ranging from PEG8 to PEG24.

In some embodiments, each R^{b} is selected from the group consisting of: C₁₋₆ alkoxy, halogen, —OH, —(C₁₋₆ alkylene)-NR^{d}R^{e}, —C(O)NR^{d}R^{e} and —C(O)(C₁₋₆ alkyl). In some embodiments, one of R^{b} is NR^{d}R^{e}, and the remaining R^{b} is not —NR^{d}R^{e}. In some embodiments, one of R^{b} is —(C₁₋₆ alkylene)-NR^{d}R^{e}, and the remaining R^{b} is not —(C₁₋₆ alkylene)-NR^{d}R^{e}. In some embodiments, one of R^{b} is NR^{d}R^{e}, and the remaining R^{b} is selected from the group consisting of: C₁₋₆ alkoxy, halogen, —OH, —C(O)NR^{d}R^{e} and —C(O)(C₁₋₆ alkyl). In some embodiments, one of R^{b} is —(C₁₋₆ alkylene)-NR^{d}R^{e}, and the remaining R^{b} is selected from the group consisting of: C₁₋₆ alkoxy, halogen, —OH, —C(O)NR^{d}R^{e} and —C(O)(C₁₋₆ alkyl).

In some embodiments, L^{A} is

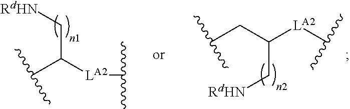

wherein L^{A2} is a 2-19 membered heteroalkylene optionally substituted with 1 R^{b}; subscript n1 is 1-4; subscript n2 is 0-3; and L^{A2} is further optionally substituted with a PEG Unit ranging from PEG2 to PEG24. In some embodiments, R^{d} is hydrogen. In some embodiments, R^{d} is C₁₋₃ alkyl. In some embodiments, R^{d} is methyl.

In some embodiments, L^{A} is

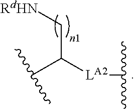

In some embodiments, L^{A} is

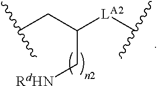

In some embodiments, L^{A2} is a 2-12 membered heteroalkylene optionally substituted with R^{a} and further optionally substituted with a PEG Unit ranging from PEG2 to PEG24. In some embodiments, subscript n1 is 1. In some embodiments, subscript n1 is 2. In some embodiments, subscript n1 is 3. In some embodiments, subscript n1 is 4. In some embodiments, subscript n2 is 0. In some embodiments, subscript n2 is 1. In some embodiments, subscript n2 is 2. In some embodiments, subscript n2 is 3.

In some embodiments, L^{A2} is unsubstituted. In some embodiments, L^{A2} is substituted with 1 R^{a}, as described herein. In some embodiments, L^{A2} is substituted with a PEG Unit ranging from PEG8 to PEG24. In some embodiments, L^{A2} is substituted with 1 R^{a}, as described herein with a PEG Unit ranging from PEG8 to PEG24. In some embodiments, L^{A} is a C₁-C₁₀ alkylene substituted with —(CH₂)NH₂ or —(CH₂CH₂)NH₂. In some embodiments, L^{A} is a C₁-C₆ alkylene substituted with —(CH₂)NH₂ or —(CH₂CH₂)NH₂. In some embodiments, L^{A} is a C₁-C₁₀ alkylene substituted with oxo (C═O); and with one of —(CH₂)NH₂ and —(CH₂CH₂)NH₂. In some embodiments, L^{A} is a C₁-C₆ alkylene substituted with oxo (C═O); and with one of —(CH₂)NH₂ and —(CH₂CH₂)NH₂. In some embodiments, L^{A} is a 2-24 membered heteroalkylene substituted with —(CH₂)NH₂ or —(CH₂CH₂)NH₂. In some embodiments, L^{A} is a 4-12 membered heteroalkylene substituted with —(CH₂)NH₂ or —(CH₂CH₂)NH₂.

In some embodiments, L^{A} is

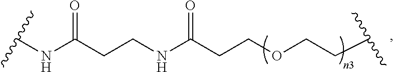

wherein subscript n3 is 1-5. In some embodiments, subscript n3 is 1. In some embodiments, subscript n3 is 2. In some embodiments, subscript n3 is 3. In some embodiments, subscript n3 is 4. In some embodiments, subscript n3 is 5.

In some embodiments, each R^{a} is independently selected from the group consisting of: C₁₋₆ alkyl, C₁₋₆ haloalkyl, C₁₋₆ alkoxy, C₁₋₆ haloalkoxy, halogen, —OH, ═O, —C(O)NR^{d}R^{e}, —C(O)(C₁₋₆ alkyl), —(C₁₋₆ alkylene)-NR^{d}R^{e}, and —C(O)O(C₁₋₆ alkyl). In some embodiments, one of R^{a} is —NR^{d}R^{e} and the other R^{a} are independently selected from the group consisting of: C₁₋₆ alkyl, C₁₋₆ alkoxy, halogen, —OH, ═O, —C(O)(C₁₋₆ alkyl), and —C(O)O(C₁₋₆ alkyl).

In some embodiments, one of R^{a} is C₁₋₆ haloalkyl. In some embodiments, one of R^{a} is C₁₋₆ alkoxy. In some embodiments, one of R^{a} is C₁₋₆ haloalkoxy. In some embodiments, one of R^{a} is halogen. In some embodiments, one of R^{a} is —OH. In some embodiments, one of R^{a} is ═O. In some embodiments, one of R^{a} is C(O)NR^{d}R^{e}. In some embodiments, one of R^{a} is —C(O)(C₁₋₆ alkyl). In some embodiments, one of R^{a} is —C(O)O(C₁₋₆ alkyl). In some embodiments, one R^{a} is —NR^{d}R^{e}. In some embodiments, one R^{a} is —(C₁₋₆ alkylene)-NR^{d}R^{e}.

In some embodiments, each R^{b} is independently selected from the group consisting of: C₁₋₆ alkyl, C₁₋₆ haloalkyl, C₁₋₆ alkoxy, C₁₋₆ haloalkoxy, halogen, —OH, ═O, —C(O)NR^{d}R^{e}, —C(O)(C₁₋₆ alkyl), —(C₁₋₆ alkylene)-NR^{d}R^{e}, and —C(O)O(C₁₋₆ alkyl). In some embodiments, one R^{b} is NR^{d}R^{e} and the other R^{b} are independently selected from the group consisting of: C₁₋₆ alkyl, C₁₋₆ haloalkyl, C₁₋₆ alkoxy, C₁₋₆ haloalkoxy, halogen, —OH, ═O, —C(O)NR^{d}R^{e}, —C(O)(C₁₋₆ alkyl), and —C(O)O(C₁₋₆ alkyl). In some embodiments, one of R^{b} is C₁₋₆ haloalkyl. In some embodiments, one of R^{b} is C₁₋₆ alkoxy. In some embodiments, one of R^{b} is C₁₋₆ haloalkoxy. In some embodiments, one of R^{b} is halogen. In some embodiments, one of R^{b} is —OH. In some embodiments, one of R^{b} is ═O. In some embodiments, one of R^{b} is C(O)NR^{d}R^{e}. In some embodiments, one of R^{b} is —C(O)(C₁₋₆ alkyl). In some embodiments, one of R^{b} is —C(O)O(C₁₋₆ alkyl). In some embodiments, one R^{b} is —NR^{d}R^{e}. In some embodiments, one R^{b} is —(C₁₋₆ alkylene)-NR^{d}R^{e}.

In some embodiments of formulae A and D, the 2-24 membered heteroalkylene is optionally substituted with 1-2 independently selected R^{b} that are uncharged at physiological pH. In some embodiments of formulae A and D, the 2-24 membered heteroalkylene is optionally substituted with 2 R^{b}; wherein one R^{b} is positively charged and the other R^{b} is negatively charged.

In some embodiments, R^{d} and R^{e} are independently selected from hydrogen and C₁-C₃ alkyl. In some embodiments, R^{d} and R^{e} are the same. In some embodiments, R^{d} and R^{e} are different. In some embodiments, one of R^{d} and R^{e} is hydrogen and the other of R^{d} and R^{e} is C₁-C₃ alkyl. In some embodiments, R^{d} and R are both hydrogen. In some embodiments, R^{d} and R are independently C₁-C₃ alkyl. In some embodiments, R^{d} and R^{e} are both methyl. In some embodiments, R^{d} and R^{e} together with the nitrogen atom to which both are attached form a 5-6 membered heterocyclyl.

In some embodiments, the heteroalkylene group of any of formulae A-K is uncharged at physiological pH.

In some embodiments, Ring B is an unfused 8-12 membered heterocyclyl. In some embodiments, Ring B is an unfused 8-10 membered heterocyclyl. In some embodiments, Ring B is an unfused 8 membered heterocyclyl ring. In some embodiments, Ring B contains one carbon-carbon double bond and one nitrogen atom in the ring. In some embodiments, Ring B is (Z)-1,2,3,4,7,8-hexahydroazocine.

In some embodiments, Ring B is an 8-12 membered heterocyclyl fused to a C₆₋₁₀ aryl or 5-6 membered heteroaryl ring. In some embodiments, Ring B is an 8-12 membered heterocyclyl fused to two C₆₋₁₀ aryl rings or two 5-6 membered heteroaryl rings. In some embodiments, Ring B is an 8-10 membered heterocyclyl fused to a C₆₋₁₀ aryl or 5-6 membered heteroaryl ring. In some embodiments, Ring B is an 8-10 membered heterocyclyl fused to two C₆₋₁₀ aryl rings or two 5-6 membered heteroaryl ring rings. In some embodiments, Ring B is fused to one or two C₆₋₁₀ aryl rings. In some embodiments, Ring B is fused to one or two 5-6 membered heteroaryl rings. In some embodiments, Ring B is an 8-12 membered heterocyclyl fused to one or two phenyl rings. In some embodiments, Ring B is an 8-10 membered heterocyclyl fused to one or two phenyl rings. In some embodiments, Ring B is an 8 membered heterocyclyl fused to one or two phenyl rings. In some embodiments, Ring B has one nitrogen atom in the ring. In some embodiments, Ring B has no charged ring heteroatoms at physiological pH.

In some embodiments, Ring B is unsubstituted. In some embodiments, Ring B is substituted with 1-3 independently selected R^{c}. In some embodiments, Ring B is substituted with one R^{c}. In some embodiments, Ring B is substituted with two independently selected R^{c}. In some embodiments, Ring B is substituted with three independently selected R^{c}.

In some embodiments, Ring B is uncharged at physiological pH.

In some embodiments, each R^{c} is independently selected from the group consisting of: C₁₋₆ alkyl, C₁₋₆ haloalkyl, C₁₋₆ alkoxy, C₁₋₆ haloalkoxy, halogen, —OH, ═O, —C(O)NR^{d}R^{e}, —C(O)(C₁₋₆ alkyl), —C(O)O(C₁₋₆ alkyl). In some embodiments, each R^{c} is C₁₋₆ alkyl. In some embodiments, one or two of R^{c} is C₁₋₆ haloalkyl. In some embodiments, 1-3 R^{c} are independently a C₁₋₆ alkoxy. In some embodiments, one of R^{c} is C₁₋₆ haloalkoxy. In some embodiments, each R^{c} is independently a halogen. In some embodiments, 1-3 R^{c} is —OH. In some embodiments, one of R^{c} is ═O. In some embodiments, one of R^{c} is C(O)NR^{d}R^{e}. In some embodiments, one of R^{c} is —C(O)(C₁₋₆ alkyl). In some embodiments, one of R^{c} is —C(O)O(C₁₋₆ alkyl).

In some embodiments, each R^{a}, R^{b} and R^{c} are independently selected from the group consisting of: C₁₋₆ alkyl, C₁₋₆ haloalkoxy, C₁₋₆ alkoxy, halogen, —OH, —NR^{d}R^{e}, —(C₁₋₆ alkylene)-NR^{d}R^{e}, —C(O)NR^{d}R^{e} and —C(O)(C₁₋₆ alkyl). In some embodiments, each R^{a}, R^{b} and R^{c} are independently selected from the group consisting of: C₁₋₆ alkyl, C₁₋₆ alkoxy, halogen, —(C₁₋₆ alkylene)-NR^{d}R^{e}, —OH, and —NR^{d}R^{e}. In some embodiments, none of R^{a}, R^{b} and R^{c} are present in formulae A and D as —(C₁₋₆ alkylene)-NR^{d}R^{e} or —NR^{d}R^{e} (e.g., so that L¹ remains uncharged at physiological pH). In some embodiments, R^{a} or R^{b} is —NR^{d}R^{e} in formulae B and C (e.g., so that the carboxylic acid in deprotonated form and —NR^{d}R^{e} is in protonated form at physiological pH). In some embodiments, R^{a} or R^{b} is —(C₁₋₆ alkylene)-NR^{d}R^{e} in formulae B and C (e.g., so that the carboxylic acid in deprotonated form and —(C₁₋₆ alkylene)-NR^{d}R^{e} is in protonated form at physiological pH).

In some embodiments, Ring B is:

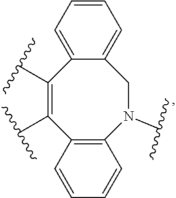 [SMILES: */C1=C(\*)c2ccccc2N(*)Cc2ccccc21]

In some embodiments, S*-L¹ is selected from the group consisting of formulae A1, A2, A3, B1, B2, B3, C1, C2 and C3:

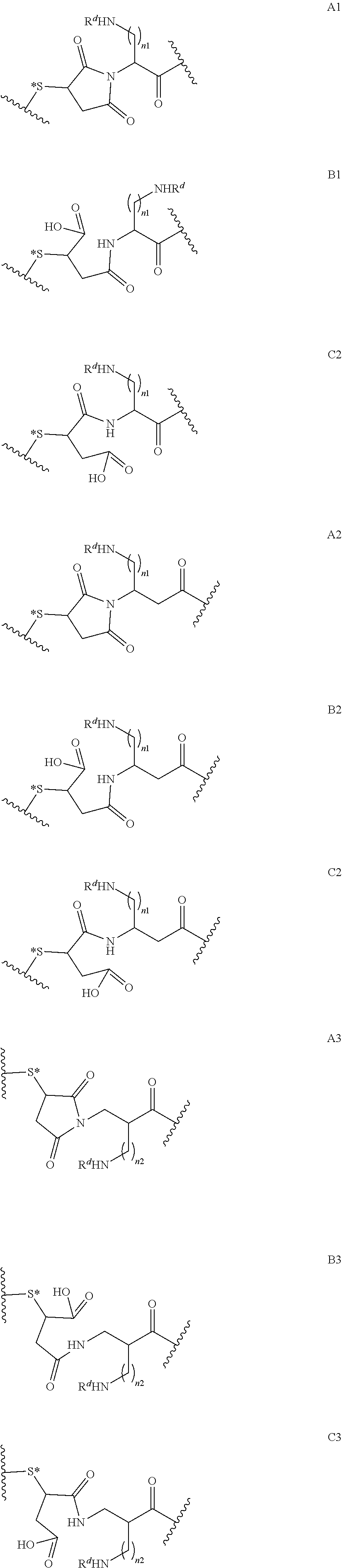

wherein R^{d} is hydrogen or C₁₋₃ alkyl and subscript n1 is 1 or 2; subscript n2 is 0, 1 or 2.

In some embodiments, S*-L¹ is

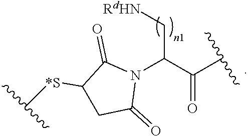

In some embodiments, S*-L¹ is

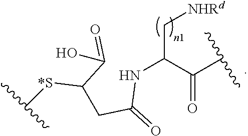

In some embodiments, S*-L¹ is

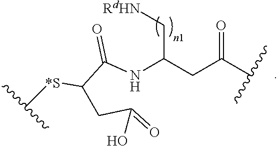

In some embodiments, S*-L¹ is

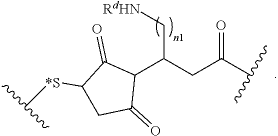

In some embodiments, S*-L¹ is

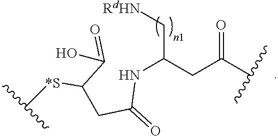

In some embodiments, S*-L¹ is

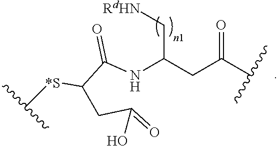

In some embodiments, S*-L¹ is

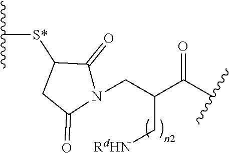

In some embodiments, S*-L¹ is

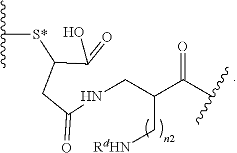

In some embodiments, S*-L¹ is

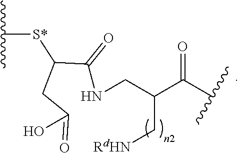

In some embodiments of S*-L¹, subscript n1 is 1 or 2 or subscript n2 is 0, 1, or 2; and S* is a sulfur atom from a cysteine residue of the antibody. In some embodiments, subscript n1 is 1. In some embodiments, subscript n2 is 1. In some embodiments, subscript n2 is 2. In some embodiments, subscript n1 is 2.

In some embodiments, S*-L¹ is

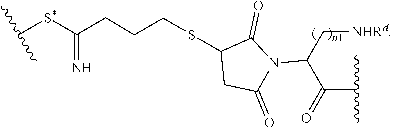

In some embodiments, S*-L¹ is

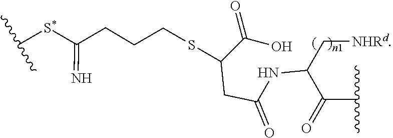

In some embodiments, S*-L¹ is

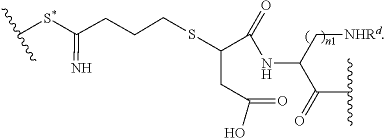

In some embodiments, S*-L¹ is

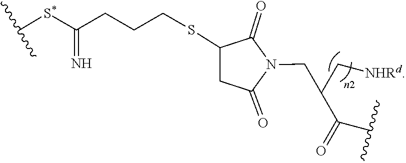

In some embodiments, S*-L¹ is

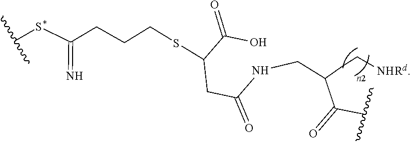

In some embodiments, S*-L¹ is

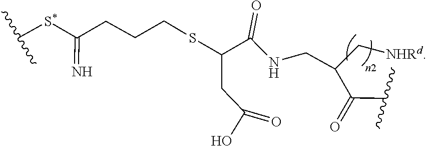

In some embodiments, S*-L¹ is

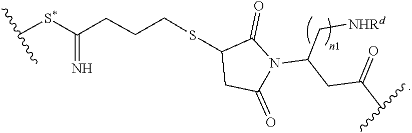

In some embodiments, S*-L¹ is

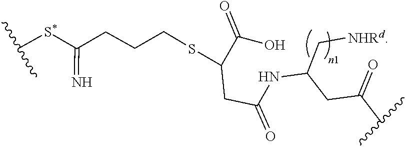

In some embodiments, S*L is

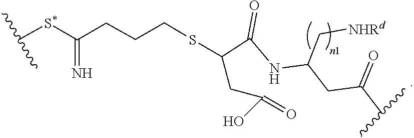

In some embodiments of S*-L¹, subscript n1 is 1 or 2 or subscript n2 is 0, 1, or 2; and S* is an ϵ-nitrogen atom from a lysine residue of the antibody. In some embodiments, subscript n1 is 1. In some embodiments, subscript n2 is 1. In some embodiments, subscript n2 is 2. In some embodiments, subscript n1 is 2.

In some embodiments, R^{d} is hydrogen or C₁₋₃ alkyl. In some embodiments, R^{d} is hydrogen. In some embodiments, R^{d} is C₁₋₃ alkyl. In some embodiments, R^{d} is methyl.

In some embodiments, *S-L¹ is:

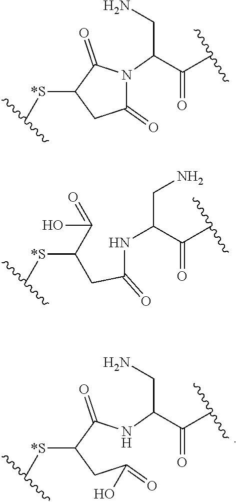

In some embodiments, *S-L¹ is

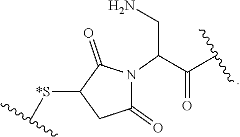

In some embodiments, *S-L¹ is

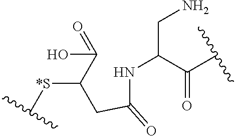

In some embodiments, *S-L¹ is

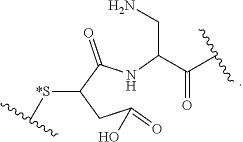

In some embodiments, S*-L¹ is:

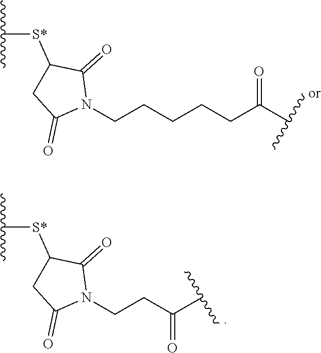

In some embodiments, S*-L¹ is:

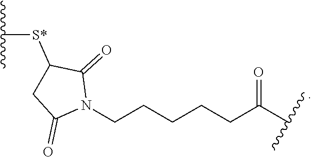

In some embodiments, S*-L¹ is:

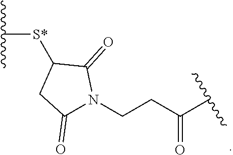

In some embodiments, S*-L¹ is:

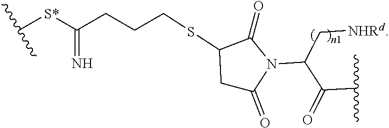

In some embodiments, S*-L¹ is:

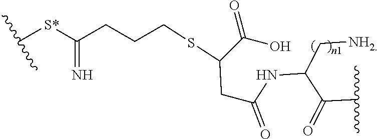

In some embodiments, S*-L¹ is:

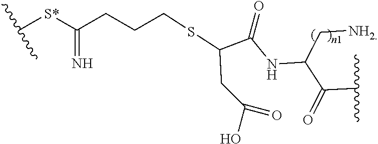

In some embodiments, S*-L¹ is:

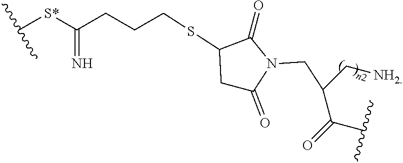

In some embodiments, S*-L¹ is:

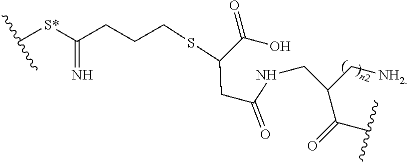

In some embodiments, S*-L¹ is:

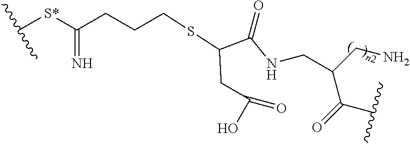

In some embodiments, S*-L¹ is:

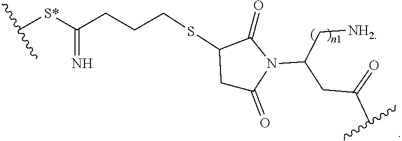

In some embodiments, S*-L¹ is:

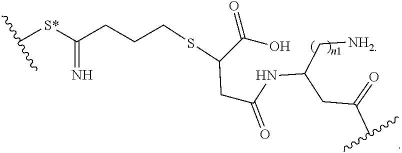

In some embodiments, S*-L¹ is:

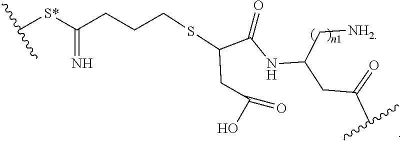

In some embodiments, S*-L¹ is:

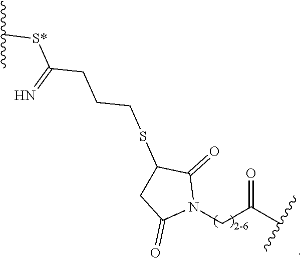

In some embodiments, S*-L¹ is:

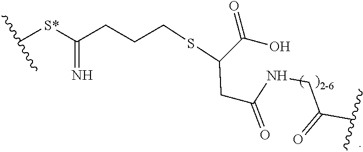

In some embodiments, S*-L¹ is:

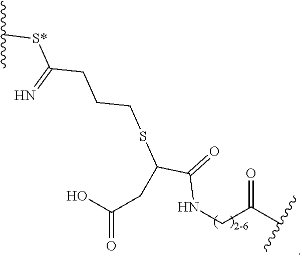

In some embodiments, *S-L¹ is selected from the group consisting of:

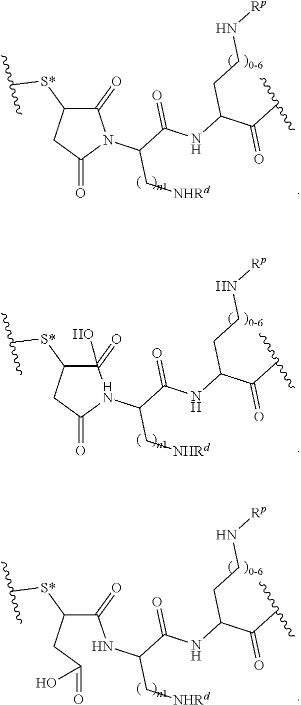

In some embodiments, *S-L¹ is

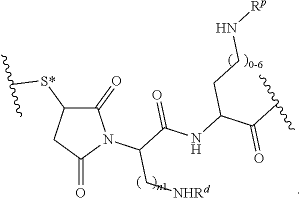

In some embodiments, *S-L¹ is

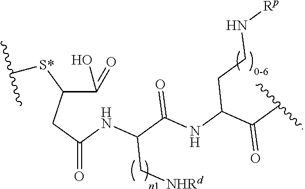

In some embodiments, *S-L¹ is

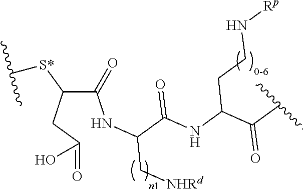

In some embodiments, *S-L¹ is

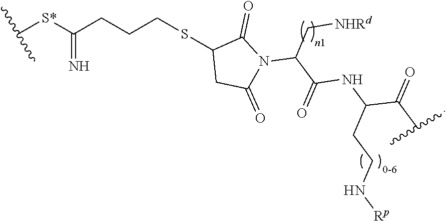

In some embodiments, *S-L¹ is

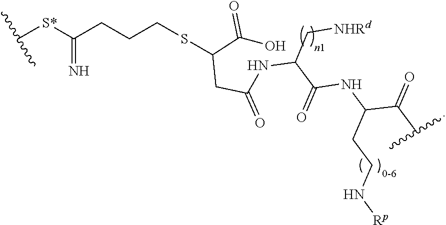

In some embodiments, *S-L¹ is

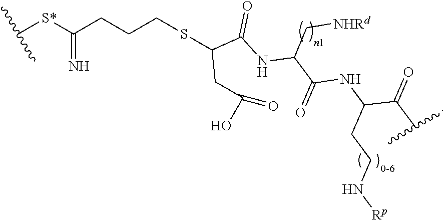

In some embodiments, *S-L¹ comprises R^{P}, wherein R^{P} is attached to the nitrogen atom through a functional group that retains that atom in uncharged form under physiological conditions, such as functional groups comprised of —C(═O)—, in which the carbonyl carbon atom is bonded to that nitrogen atom. In some embodiments, *S-L¹ comprises R^{P}, wherein R is attached to the nitrogen atom via an amide linkage.

In some embodiments, S* is a sulfur atom from a cysteine residue of the antibody. In some embodiments, S* is an ϵ-nitrogen atom from a lysine residue from the antibody.

In some embodiments, R^{P} is —C(═O)—(C₁₋₃ alkylene)-, or is a PEG Unit ranging from PEG2 to PEG72. In some embodiments, R^{P} is —C(═O)—(C₁₋₃ alkylene)-, or is a PEG Unit ranging from PEG8 to PEG24 or PEG12 to PEG36, that is covalently attached to the nitrogen atom through the carbon atom a carbonyl functional group of the PEG Unit. In some embodiments, the ethylene glycol chain of the PEG Unit is connected to the nitrogen atom through a —C(═O)—(C₁₋₃ alkylene)-group.

In some embodiments, *S-L¹ is:

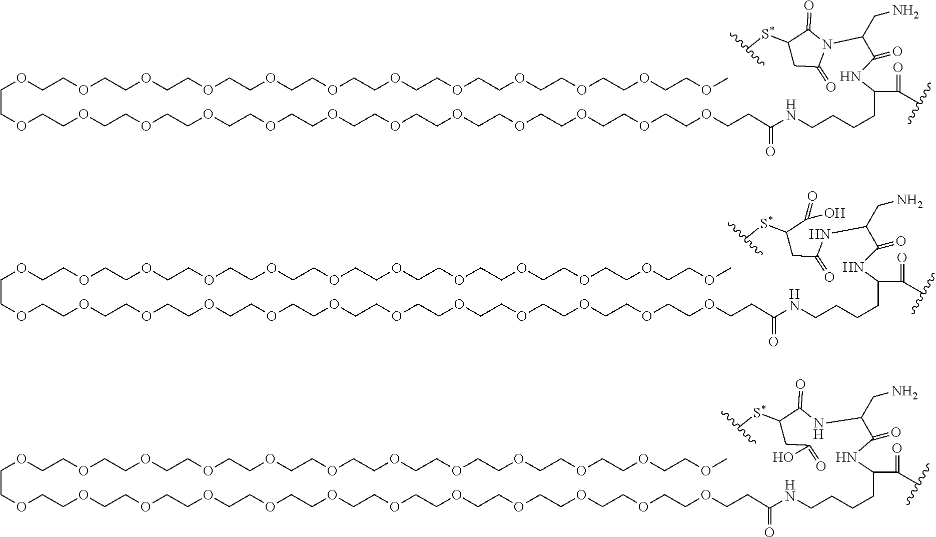

In some embodiments, S* is a triazole moiety.

In some embodiments, *S-L¹ is:

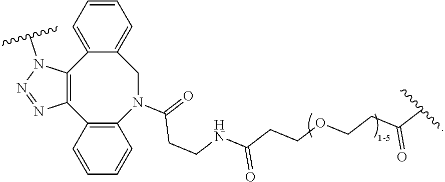

In some embodiments, subscript x is 0. In some embodiments, subscript x is 1, 2, 3, or 4. In some embodiments, subscript x is 1. In some embodiments, subscript x is 2. In some embodiments, subscript x is 3. In some embodiments, subscript x is 4.

The multiplexer (M) in the ADCs described herein serves as a branching component (e.g., a trifunctional linking group). For example, when subscript x=1, the initial multiplexer provides both covalent attachment to the first linker (L¹) as well as covalent attachments to two second linker (L²) groups, when present. As another example, when subscript x=2, the initial multiplexer provides a covalent attachment to L¹ as well as covalent attachments to two subsequent multiplexer (M) groups, each of which is covalently attached to two L² groups, when present. In some embodiments, the multiplexer comprises a single functional group, such as a single tertiary amine, providing covalent attachment to L¹ as well as covalent attachment to two L² groups (when present). In some embodiments, the multiplexer comprises two or three functional groups that provides covalent attachments to L¹ and two L² groups (when present). For example, in some embodiments, a first function group such as a thiol, a hydroxyl, an amine, or another nucleophilic group provide covalent attachment to L¹, while a covalent attachment to either or both of the L² groups (when present) is provided by a second functional group such as a thiol, a hydroxy, an amine, or another nucleophilic group. In embodiments, where the multiplexer comprises two or more functional groups for covalent attachment to L¹ and each L², the two or more functional groups are linked by a C₁-s alkylene or 2-8 membered heteroalkylene. In some embodiments, either or both L² are present.

In some embodiments, the multiplexer is represented by the structure:

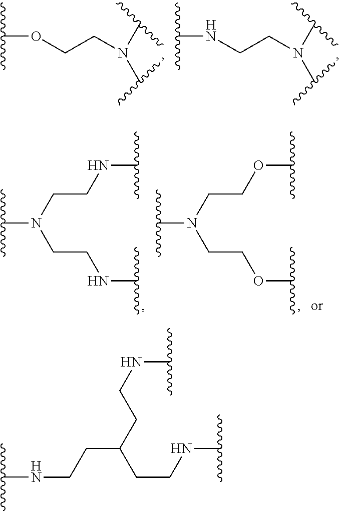

wherein, the wavy lines to the right indicate covalent attachments to two L² groups, and the wavy line to the left indicates covalent attachment to L¹. In some embodiments, the covalent attachments to the nitrogen atoms render those nitrogen atoms uncharged at physiological pH.

In some embodiments, the multiplexer is a thiol multiplexer, where the thiol multiplexer is covalently attached at a single site (shown as ‘a’), is ring closed or ring opened to form two thiols (b) which serve as two sites for further attachments (as in ‘c’) of a linker or drug-linker moiety. Examples of thiol multiplexers include, but are not limited to, the structures shown below.

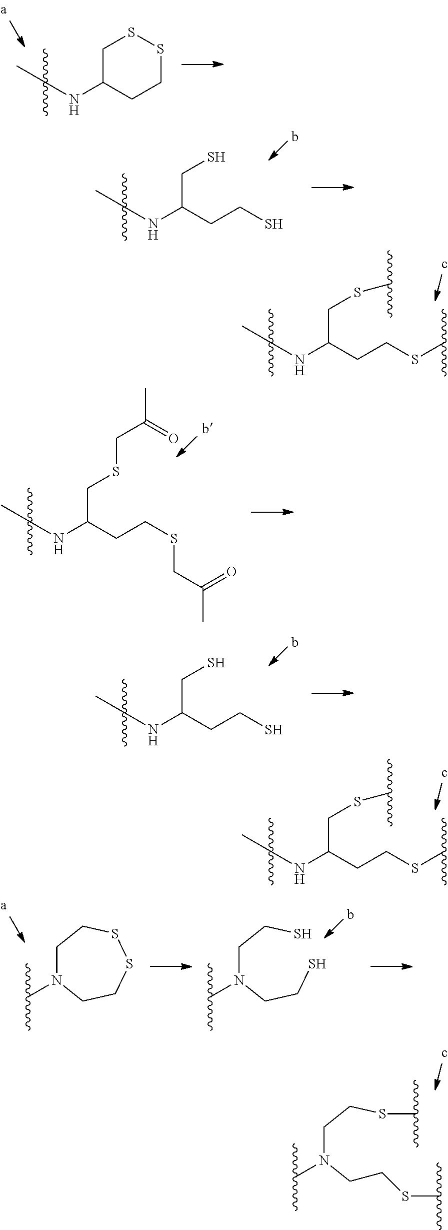

In some embodiments, the wavy line adjacent to the nitrogen atom represents the site of covalent attachment to the ADCs through a functional group that is uncharged at physiological pH. In some embodiments, the functional group comprises —C(═O)—, wherein the carbon atom is bonded to the nitrogen atom adjacent to the wavy line (i.e., at the “a” position noted above).

In some embodiments, the thiol multiplexer is based on a commercially available component having a five-, six-, seven- or eight-membered carbocyclic ring in which two adjacent ring vertices are replaced by sulfur-forming 1,2-dithiolanes, 1,2-dithianes, 1,2-dithiepanes and 1,2-dithiocanes. The five- and six-membered rings will generally have a functional group external to the ring that is suitable for the synthetic chemistries described herein. In some embodiments, the larger seven- and eight-membered rings have an exocyclic functional group that is suitable for the synthetic chemistries described herein, and in other embodiments another ring vertex is replaced with, for example, a nitrogen (amine) which sometimes serves as a functional group in the linking chemistries provided.

Further examples of thiol multiplexers (in disulfide form) include:

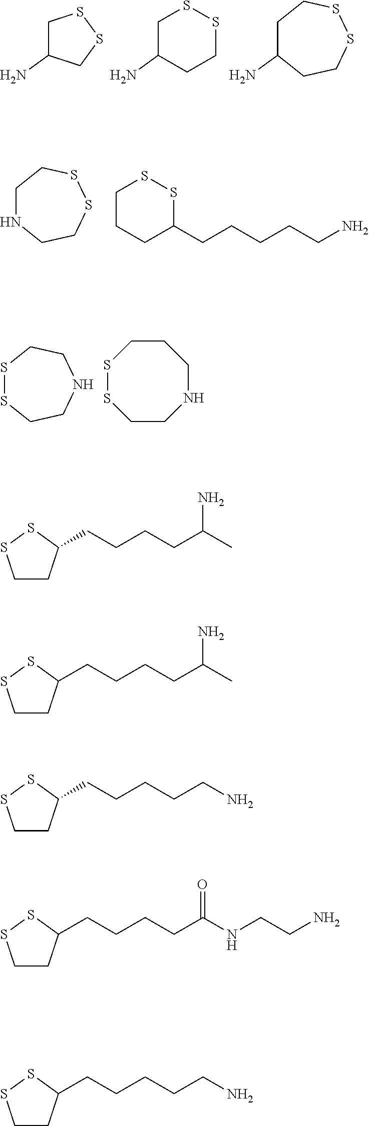

The functional groups present in the above thiol multiplexers in disulfide form are all nucleophilic groups; however, a person of skill in the art will recognize that the choice of the nucleophilic group for covalent attachment of L¹, L², or subsequent multiplexer groups can be changed without departing from the scope of the current disclosure.

Other non-limiting examples of thiol multiplexers in disulfide form include the following:

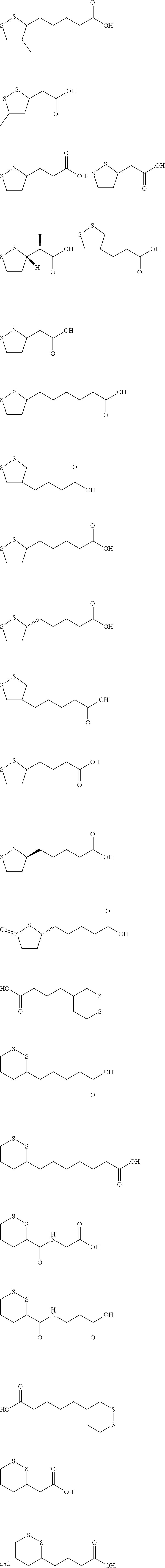

The carboxylic acid groups present in certain thiol multiplexers, as described herein, can be activated for covalent attachment of a nucleophilic group to L¹, L², or subsequent multiplexer groups; however, a person of skill in the art will recognize that the choice of nucleophilic group for that subsequent covalent attachment can be changed without departing from the scope of the current disclosure. Thus, it is apparent that the choice of nucleophilic group or electrophilic group depends on the chemical identity of the functional group providing covalent attachment to the multiplexer in L¹ and L².

In some embodiments, M has the structure of formula M^{a}:

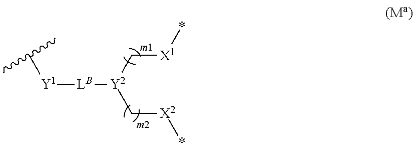

wherein the wavy line represents the covalent attachment of M^{a} to L¹;

each * represents the covalent attachment of M^{a} to -L²-D;

Y¹ is selected from the group consisting of: a bond, —S—, —O—, and —NH—;

Y² is selected from the group consisting of: —CH— and —N—;

L^{B} is absent or a C₁₋₆ alkylene that is optionally interrupted with a group selected from the group consisting of: —O—, —C(═O)NH—, —NHC(═O)—, —C(═O)O—, —O(C═O)—, —NH—, and —N(C₁₋₃ alkyl)-;

X¹ and X² are each independently —S—, —O—, or —NH—; and

subscripts m1 and m2 are each independently 1-4.

In some embodiments, a bond to a nitrogen atom of M when Y¹ is —NH— or Y², X¹ or X² is —N— is through a functional group that retains that atom in uncharged form at physiological pH and includes functional groups comprised of —C(═O)—, in which the carbonyl carbon atom is bonded to that nitrogen atom. In some embodiments, a bond to a nitrogen atom of M when Y¹ is —NH— or Y², X¹ or X² is —N— is via an amide linkage.

In some embodiments, Y¹ is a bond. In some embodiments, Y¹ is —S—. In some embodiments, Y¹ is —O—. In some embodiments, Y¹ is —NH—. In some embodiments, Y² is —CH—. In some embodiments, Y² is —N—. In some embodiments, X¹ and X² are both —NH—.

In some embodiments, L^{B} is present or absent, Y¹ is a bond, and Y² is —CH—. In some embodiments, L^{B} is present or absent, Y¹ is a bond, and Y² is —N—. In some embodiments, L^{B} is present or absent, Y¹ is —S—, and Y² is —CH—. In some embodiments, L^{B} is present, Y¹ is —S—, and Y² is —N—. In some embodiments, L^{B} is present or absent, Y¹ is —O—, and Y² is —CH—. In some embodiments, L^{B} is present, Y¹ is —O—, and Y² is —N—. In some embodiments, L^{B} is present or absent, Y¹ is —NH—, and Y² is —CH—. In some embodiments, L^{B} is present, Y¹ is —NH—, and Y² is —N—.

In some embodiments, X¹ is —S—. In some embodiments, X¹ is —O—. In some embodiments, X¹ is —NH—. In some embodiments, X² is —S—. In some embodiments, X² is —O—. In some embodiments, X² is —NH—. In some embodiments, X¹ and X² are the same. In some embodiments, X¹ and X² are different.

In some embodiments, subscript m1 is 1. In some embodiments, subscript m1 is 2. In some embodiments, subscript m1 is 3. In some embodiments, subscript m1 is 4. In some embodiments, subscript m2 is 1. In some embodiments, subscript m2 is 2. In some embodiments, subscript m2 is 3. In some embodiments, subscript m2 is 4. In some embodiments, subscripts m1 and m2 are equal. In some embodiments, subscripts m1 and m2 are equal and range from 2-4. In some embodiments, subscripts m1 and m2 are each 2.

In some embodiments, Y¹ is —NH—; L^{B} is present; Y² is CH; and X¹ and X² are each —S—. In some embodiments, Y¹ is a bond; L^{B} is absent; Y² is N; and X¹ and X² are each —S—. In some embodiments, Y¹ is a bond; L^{B} is absent; Y² is —N—; and X¹ and X² are each —NH—.

In some embodiments, L^{B} is absent. In some embodiments, when L^{B} is present, L^{B} is a C₁₋₆ alkylene that is optionally interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, when L^{B} is present, L^{B} is a C₁₋₆ alkylene that is optionally interrupted with —NH— or —N(C₁₋₃ alkyl)-. In some embodiments, M^{a} is interrupted with a functional group capable of deprotonation at physiological pH so that the net charge of M^{a} remains zero when so interrupted. In some embodiments, L^{B} is a C₁₋₆ alkylene, a C₁₋₄ alkylene, or a C₁₋₂ alkylene. In some embodiments, L^{B} is a C₁₋₆ alkylene that is interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, L^{B} is a C₁₋₆ alkylene that is interrupted with —NH— or —N(C₁₋₃ alkyl)-, wherein L^{B} is connected via a functional group capable of deprotonation at physiological pH so that the net charge of L^{B} is zero. In some embodiments, the C₁₋₆ alkylene of L^{B} is interrupted with —O—. In some embodiments, the C₁₋₆ alkylene of L^{B} is interrupted with —NH—. In some embodiments, L^{B} is interrupted with —N(C₁₋₃ alkyl)-. In some embodiments, the C₁₋₆ alkylene of L^{B} is interrupted with —C(═O)NH—. In some embodiments, the C₁₋₆ alkylene of L^{B} is interrupted with —NHC(═O)—. In some embodiments, the C₁₋₆ alkylene of L^{B} is interrupted with —C(═O)O—. In some embodiments, the C₁₋₆ alkylene of L^{B} is interrupted with —O(C═O)—.

In some embodiments, M is selected from the group consisting of:

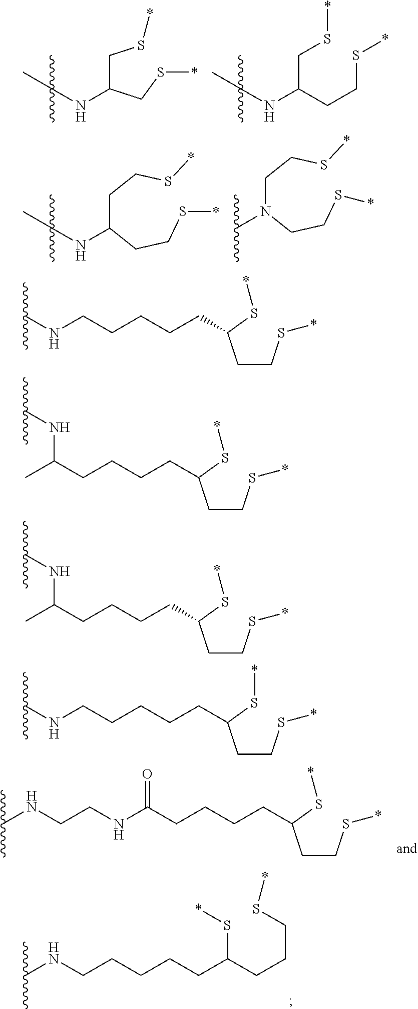

wherein the wavy line represents the covalent attachment of M to L; and

wherein each * represents the covalent attachment of M to -(L²-D).

In some embodiments, M is

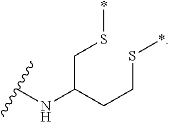

In some embodiments, M is

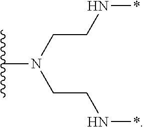

The wavy line(s) to nitrogen atom(s) in the multiplexers disclosed herein represent site(s) of covalent attachment(s) within Formula (I) through a functional group that retains these atoms in uncharged form at physiological pH and includes functional groups comprised of —C(═O)—, in which the carbonyl carbon atom is bonded to that nitrogen atom.

In some embodiments, prior to the attachment of L¹ to Ab, and M to L² (or D, when L² is absent), L¹-M comprises

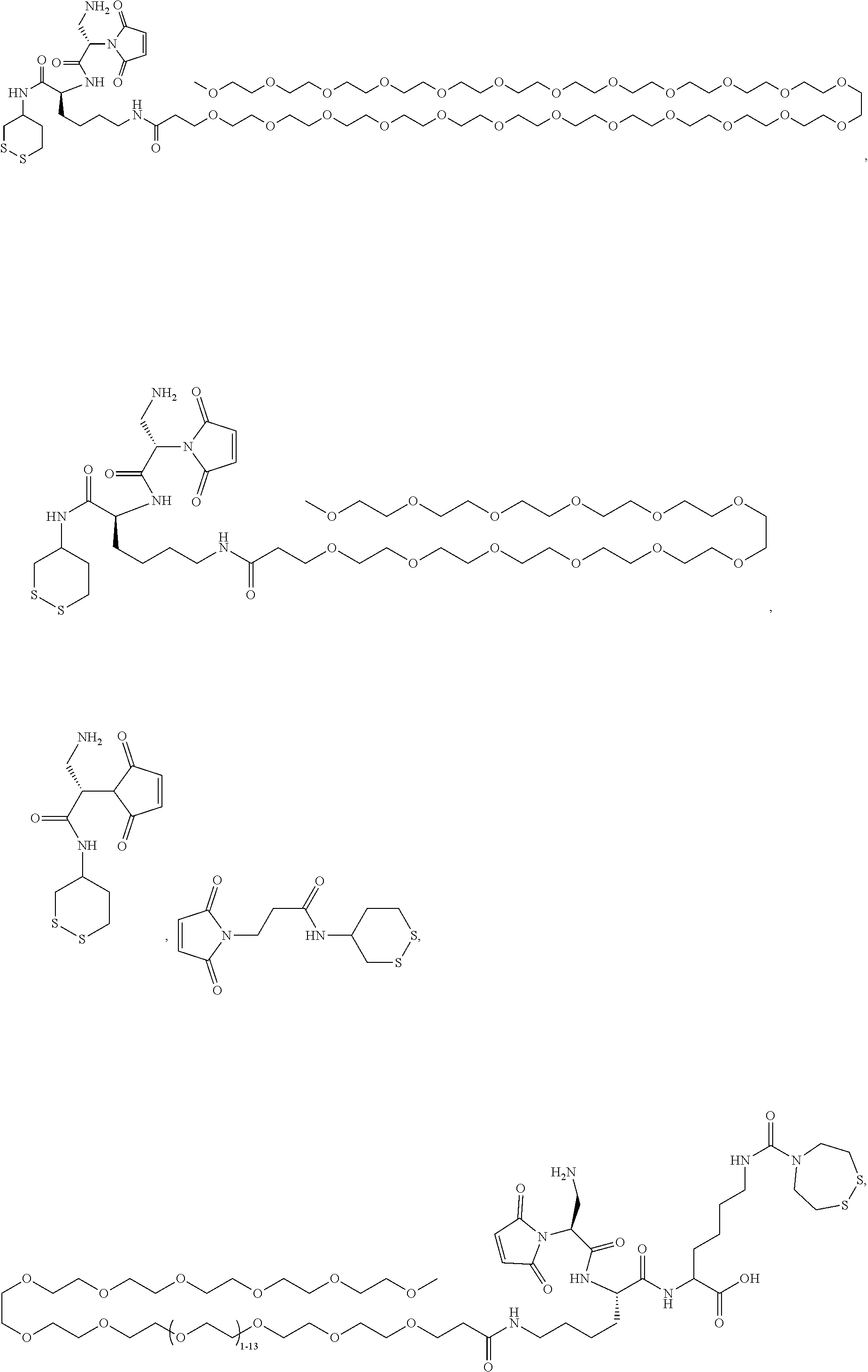

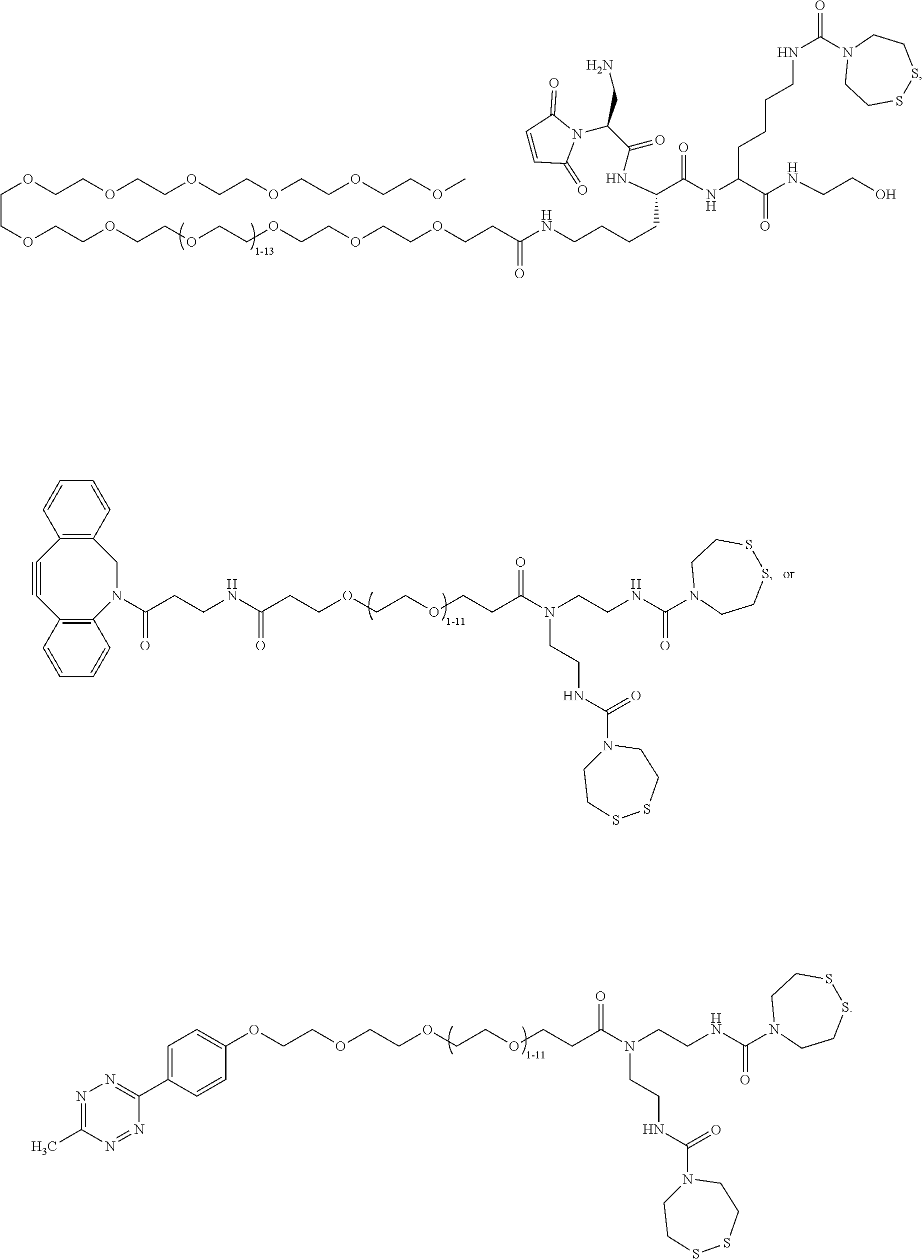

In some embodiments, subscript x is 2-4; and

(M)ₓ is -M¹-(M²)ₓ₋₁, wherein M¹ and each M² are independently selected multiplexers, as described herein. In some embodiments, subscript x is 2; and (M)ₓ is -M¹-M². In some embodiments, subscript x is 3; and (M)ₓ is -M¹-(M²)₂.

In some embodiments, M¹ has the structure of formula M^{1a}.

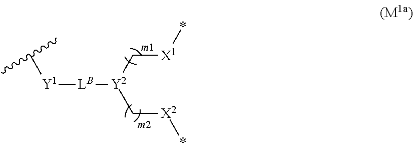

wherein the wavy line represents covalent attachment of M^{1a} to L¹;

each * represents covalent attachment of M^{1a} to M² or M^{2a} as defined herein;

Y¹ is selected from the group consisting of: a bond, —S—, —O—, and —NH—;

Y² is selected from the group consisting of: —CH— and —N—;

L^{B} is absent or a C₁₋₆ alkylene that is optionally interrupted with a group selected from the group consisting of: —O—, —C(═O)NH—, —NHC(═O)—, —C(═O)O—, —O(C═O)—, —NH—, and —N(C₁₋₃ alkyl)-;

X¹ and X² are each independently —S—, —O—, or —NH—; and

m1 and m2 are each independently 1-4.

In some embodiments, a bond to a nitrogen atom of M^{1a} when Y¹, X¹ or X² is —NH— or Y² is —N—, is through a functional group that retains that atom in uncharged form under physiological conditions and includes functional groups comprised of —C(═O)—, in which the carbonyl carbon atom is bonded to that nitrogen atom. In some embodiments, a bond to a nitrogen atom of M^{1a} when Y¹, X¹ or X² is —NH— or Y² is —N—, is via an amide linkage.

In some embodiments, Y¹ is a bond. In some embodiments, Y¹ is —S—. In some embodiments, Y¹ is —O—. In some embodiments, Y¹ is —NH—. In some embodiments, Y² is —CH—. In some embodiments, Y² is —N—. X¹ and X² are each independently —S—, —O—, or —NH—. In some embodiments, X¹ and X² are both —NH—.

In some embodiments, L^{B} is present or absent, Y¹ is a bond, and Y² is —CH—. In some embodiments, L^{B} is present or absent, Y¹ is a bond, and Y² is —N—. In some embodiments, L^{B} is present or absent, Y¹ is —S—, and Y² is —CH—. In some embodiments, L^{B} is present, Y¹ is —S—, and Y² is —N—. In some embodiments, L^{B} is present or absent, Y¹ is —O—, and Y² is —CH—. In some embodiments, L^{B} is present, Y¹ is —O—, and Y² is —N—. In some embodiments, L^{B} is present or absent, Y¹ is —NH—, and Y² is —CH—. In some embodiments, L^{B} is present, Y¹ is —NH—, and Y² is —N—.

In some embodiments, X¹ is —S—. In some embodiments, X¹ is —O—. In some embodiments, X¹ is —NH—. In some embodiments, X² is —S—. In some embodiments, X² is —O—. In some embodiments, X² is —NH—. In some embodiments, X¹ and X² are the same. In some embodiments, X¹ and X² are different.

In some embodiments, subscript m1 is 1. In some embodiments, subscript m1 is 2. In some embodiments, subscript m1 is 3. In some embodiments, subscript m1 is 4. In some embodiments, subscript m2 is 1. In some embodiments, subscript m2 is 2. In some embodiments, subscript m2 is 3. In some embodiments, subscript m2 is 4. In some embodiments, subscripts m1 and m2 are equal and range from 2-4. In some embodiments, subscripts m1 and m2 are each 2.

In some embodiments, Y¹ is —NH—; L^{B} is present; Y² is CH; and X¹ and X² are each —S—. In some embodiments, Y¹ is a bond; L^{B} is absent; Y² is —N—; and X¹ and X² are each —S—. In some embodiments, Y¹ is a bond; L^{B} is absent; Y² is —N—; and X¹ and X² are each —NH—.

In some embodiments, L^{B} is absent. In some embodiments, when L^{B} is present, L^{B} is a C₁₋₆ alkylene that is optionally interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, M^{1a} is interrupted by a functional group capable of deprotonation at physiological pH so that the net charge of M^{a} remains zero when so interrupted. In some embodiments, L^{B} is a C₁₋₆ alkylene, a C₁₋₄ alkylene, or a C₁₋₂ alkylene. In some embodiments, L^{B} is a C₁₋₆ alkylene that is interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, L^{B} is a C₁₋₆ alkylene that is interrupted with —NH— or —N(C₁₋₃ alkyl)-, wherein L^{B} is connected via a functional group capable of deprotonation at physiological pH so that the net charge of L^{B} is zero. In some embodiments, L^{B} is interrupted with —O—. In some embodiments, L^{B} is interrupted with —NH—. In some embodiments, L^{B} is interrupted with —N(C₁₋₃ alkyl)-. In some embodiments, L^{B} is interrupted with —C(═O)NH—. In some embodiments, L^{B} is interrupted with —NHC(═O)—. In some embodiments, L^{B} is interrupted with —C(═O)O—. In some embodiments, L^{B} is interrupted with —O(C═O)—.

In some embodiments, M¹ is selected from the group consisting of:

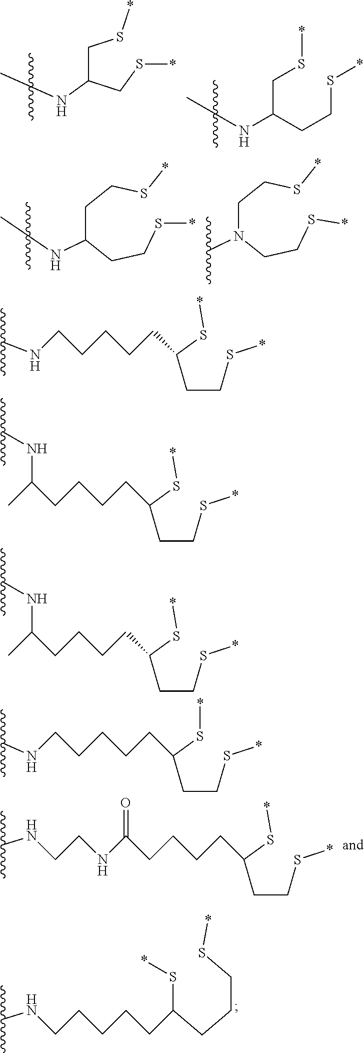

wherein the wavy line represents the covalent attachment of M¹ to L¹; and

wherein each * represents the covalent attachment of M¹ to M².

In some embodiments, M¹ is

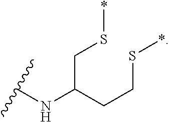

In some embodiments, M¹ is

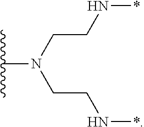

In some embodiments of M¹, each site of covalent attachment from a nitrogen atom of M¹ within Formula (I) is through a functional group that retains the nitrogen atom in uncharged form at physiological pH and includes functional groups comprised of —C(═O)—, in which the carbonyl carbon atom is bonded to that nitrogen atom.

In some embodiments, each M² independently has the structure of M^{2a}:

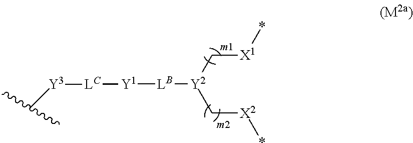

wherein the wavy line represents covalent attachment of M^{2a} to M¹/M^{1a} or to another M²/M^{2a};

each * represents the covalent attachment of M^{2a} to L²-D or another M²/M^{2a};

Y¹ is a bond, —S—, —O—, or —NH—;

Y² is —CH— or —N—;

Y³ is an optional group that provides covalent attachment of M¹/M^{1a} to the L^{C} (when present) or to Y¹ (when L^{C} is absent) of M^{2a};

L^{B} is absent or a C₁₋₆ alkylene that is optionally interrupted with a group selected from the group consisting of: —O—, —C(═O)NH—, —NHC(═O)—, —C(═O)O—, —O(C═O)—, —NH—, and —N(C₁₋₃ alkyl)-;

X¹ and X² are each independently —S—, —O—, or —NH—;

L^{C} is a C₁₋₁₀ alkylene or a C₂₋₁₀ heteroalkylene either of which is optionally substituted with 1-3 substituents each independently selected from —NR^{d}R^{e}, —(C₁₋₆ alkylene)-NR^{d}R^{e}, —CO₂H and oxo; and

subscripts m1 and m2 are each independently 1-4.

In some embodiments, when subscript x is 2 (i.e., there are two multiplexers, M¹/M^{1a} and M²/M^{2a}, the wavy line represents the covalent attachment of M²/M^{2a} to M¹/M^{1a}. In some embodiments, when subscript x is 3 (i.e., there are three multiplexers), the wavy bond either represents the covalent attachment of M²/M^{2a} to M¹/M^{1a} or the covalent attachment of the first M²/M^{2a} to the second M²/M^{2a}.

In some embodiments of M^{2a} Y¹ is a bond. In some embodiments of M^{2a} Y¹ is —S—. In some embodiments of M^{2a} Y¹ is —O—. In some embodiments of M^{2a} Y¹ is —NH—. In some embodiments of M^{2a} Y² is —CH—. In some embodiments, Y² is —N—. In some embodiments, when M^{2a} is charged at physiological pH, then M^{2a} has a net even number of excess positive or negative charges. In some embodiments, when M^{2a} is charged at physiological pH, then M^{2a} has a net odd number of excess positive or negative charges.

In some embodiments, L^{B} is present or absent, Y¹ is a bond, and Y² is —CH—. In some embodiments, L^{B} is present or absent, Y¹ is a bond, and Y² is —N—. In some embodiments, L^{B} is present or absent, Y¹ is —S—, and Y² is —CH—. In some embodiments, L^{B} is present, Y¹ is —S—, and Y² is —N—. In some embodiments, L^{B} is present or absent, Y¹ is —O—, and Y² is —CH—. In some embodiments, L^{B} is present, Y¹ is —O—, and Y² is —N—. In some embodiments, L^{B} is present or absent, Y¹ is —NH—, and Y² is —CH—. In some embodiments, L^{B} is present, Y¹ is —NH—, and Y² is —N—.

In some embodiments, X¹ is —S—. In some embodiments, X¹ is —O—. In some embodiments of M^{2a} X¹ is —NH—. In some embodiments of M^{2a} X² is —S—. In some embodiments of M^{2a} X² is —O—. In some embodiments of M^{2a} X² is —NH—. In some embodiments of M^{2a} X¹ and X² are the same. In some embodiments of M^{2a} X¹ and X² are different.

In some embodiments, subscript m1 is 1. In some embodiments, subscript m1 is 2. In some embodiments, m1 is 3. In some embodiments, subscript m1 is 4. In some embodiments, m2 is 1. In some embodiments, subscript m2 is 2. In some embodiments, subscript m2 is 3. In some embodiments, subscript m2 is 4.

In some embodiments, L^{B} is absent. In some embodiments, L^{B} is a C₁₋₆ alkylene that is interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, L^{B} is a C₁₋₆ alkylene that is interrupted with —NH— or —N(C₁₋₃ alkyl)-, wherein L^{B} is connected via a functional group capable of deprotonation at physiological pH so that the net charge of L^{B} is zero. In some embodiments of M^{2a} L^{B} is present as a C₁₋₆ alkylene, a C₁₋₄ alkylene, or a C₁₋₂ alkylene. In some embodiments, L^{B} is a C₁₋₆ alkylene that is interrupted with a group selected from the group consisting of: —O—, —C(═O)NH—, —NHC(═O)—, —C(═O)O—, —O(C═O)—, —NH—, and —N(C₁₋₃ alkyl)-. In some embodiments, L^{B} is a C₁₋₆ alkylene that is interrupted with —NH— or —N(C₁₋₃ alkyl)-, wherein L^{B} is connected via a functional group capable of deprotonation at physiological pH so that the net charge of L^{B} is zero. In some embodiments, the C₁₋₆ alkylene of L^{B} is interrupted with —O—. In some embodiments, the C₁₋₆ alkylene of L^{B} is interrupted with —NH—. In some embodiments, the C₁₋₆ alkylene of L^{B} is interrupted with —N(C₁₋₃ alkyl)-. In some embodiments, the C₁₋₆ alkylene of L^{B} is interrupted with —C(═O)NH—. In some embodiments, L^{B} is interrupted with —NHC(═O)—. In some embodiments, the C₁₋₆ alkylene of L^{B} is interrupted with —C(═O)O—. In some embodiments, the C₁₋₆ alkylene of L^{B} is interrupted with —O(C═O)—.

In some embodiments, L^{C} is a C₁₋₁₀ alkylene or a C₂₋₁₀ heteroalkylene, each substituted with —(C₁₋₆ alkylene)-NR^{d}R^{e}. In some embodiments, L^{C} is a C₁₋₁₀ alkylene or a C₂₋₁₀ heteroalkylene, each substituted with —(C₁₋₃ alkylene)-NR^{d}R^{e}. In some embodiments, R^{d} and R^{e} are both hydrogen.

In some embodiments, Y³ is present as a carbonyl group (—C(═O—)), a succinimide, or a hydrolyzed succinimide.

In some embodiments, Y³ is —C(═O)—. In some embodiments, Y³ is a succinimide. In some embodiments, Y³ is a hydrolyzed succinimide.

In some embodiments, Y³ is selected from the group consisting of:

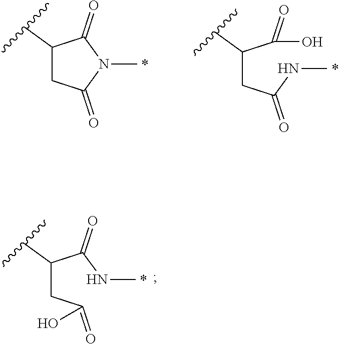

wherein * represents covalent attachment to L^{C}; and the wavy line represents covalent attachment to M¹/M^{1a} or another M²/M^{2a}.

In some embodiments, Y³-L^{C} is selected from the group consisting of:

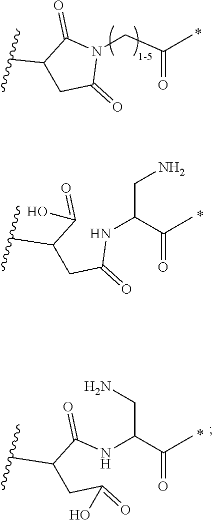

wherein * represents covalent attachment to Y¹; and the wavy line represents covalent attachment to M¹ or another M².

In some embodiments, Y³-L^{C} is selected from the group consisting of:

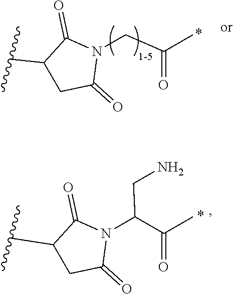

wherein the amino group is protected by an acid-labile protecting group. Exemplary acid-labile protecting groups include, but are not limited to t-butyloxycarbonyl (Boc), triphenylmethyl (trityl), and benzylidene.

In some embodiments, Y¹ is a bond; L^{B} is absent; Y² is —N—; and X¹ and X² are each —NH—. In some embodiments, a bond to a nitrogen atom of M^{2a} when Y¹, X¹ or X² is —NH— or Y² is —N— is through a functional group that retains that atom in uncharged form at physiological pH and includes functional groups comprised of —C(═O)—, in which the carbonyl carbon atom is bonded to that nitrogen atom. In some embodiments, a bond to a nitrogen atom of M^{2a} when Y¹, X¹ or X² is —NH— or Y² is —N— is via an amide linkage.

In some embodiments, M² is selected from the group consisting of:

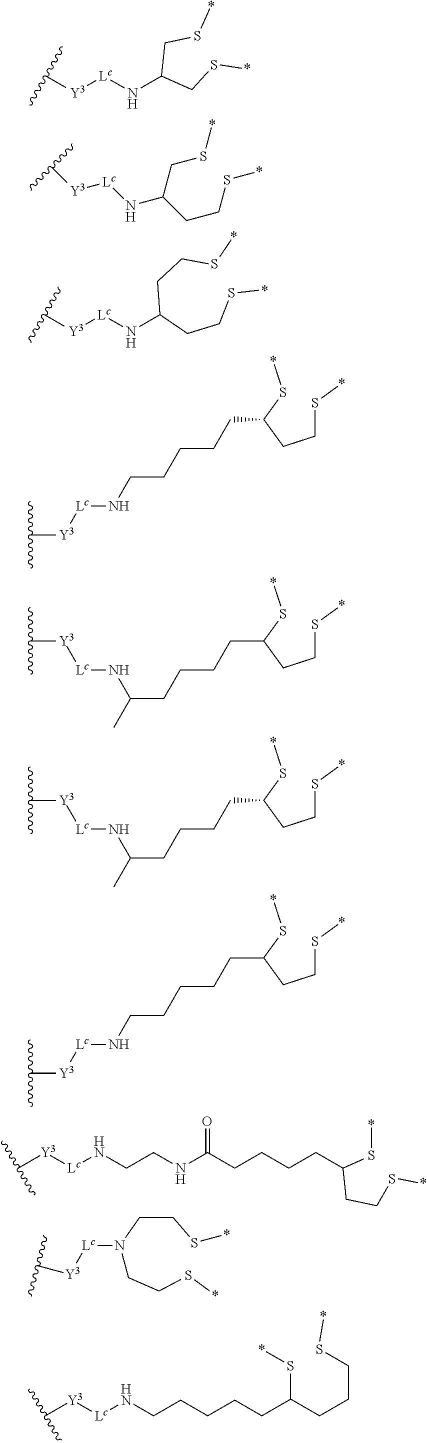

wherein each * represents the covalent attachment to L²-D or another M²/M^{2a} and the wavy bond presents the covalent attachment to M¹/M^{1a} or another M²/M^{2a}. For example, when L² is absent, each * represents a covalent attachment to D. When subscript x is 2 (i.e., there are two multiplexers, M¹/M^{1a} and M²/M^{2a}), the wavy bond represents a covalent attachment to M¹/M^{1a}.

In some embodiments, M² is selected from the group consisting of:

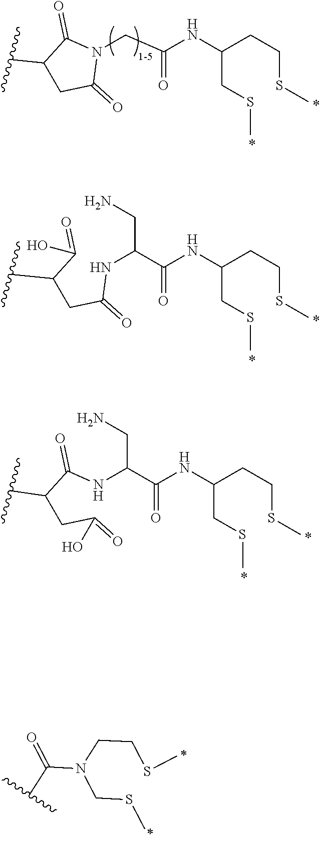

and in some embodiments, M² is selected from the group consisting of:

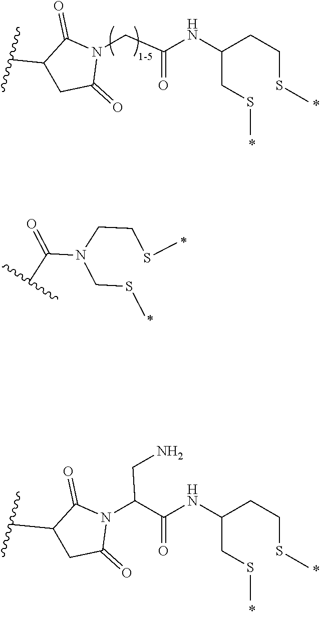

wherein the nitrogen atom of the —CH₂NH₂ moiety is protected by an acid-labile protecting group; and

wherein each * represents covalent attachment to L²-D or another M²/M^{2a}; and the wavy bond presents the covalent attachment to M¹/M^{1a} or another M²/M^{2a}. For example, when L² is absent, each * represents a covalent attachment to D. When subscript x is 2 (i.e., there are two multiplexers, M¹/M^{1a} and M²/M^{2a}, the wavy bond represents a covalent attachment to M¹/M^{1a}.

In some embodiments, subscript x is 2; and (M)ₓ is:

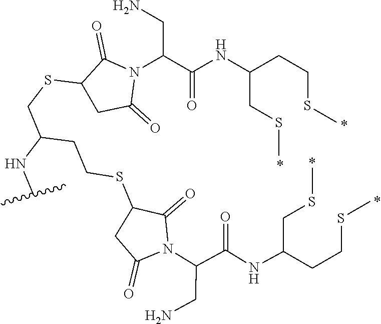 [SMILES: *NC(CCSC1CC(=O)N(C(CN)C(=O)NC(CCSC)CSC)C1=O)CSC1CC(=O)N(C(CN)C(=O)NC(CCSC)CSC)C1=O]

wherein each * represents the covalent attachment to L²-D; the wavy line represents the covalent attachment to L¹; and each succinimide ring is optionally hydrolyzed. When L² is absent, each * represents a covalent attachment to D.

In some embodiments, when (M)ₓ comprises —CH₂NH₂, the nitrogen atoms of that moiety is protonated and the succinimide ring is in hydrolyzed form at physiological pH. In some embodiments, (M)ₓ comprises —CH₂NH₂. In some embodiments, (M)ₓ comprises —CH₂NPG¹PG², wherein PG¹ is an acid-labile nitrogen protecting group and PG² is hydrogen; or PG¹ and PG² together form an acid-labile nitrogen protecting group. In some embodiments, one succinimide ring is hydrolyzed and the other succinimide ring is not hydrolyzed.

In some embodiments, subscript x is 3; and (M)ₓ is:

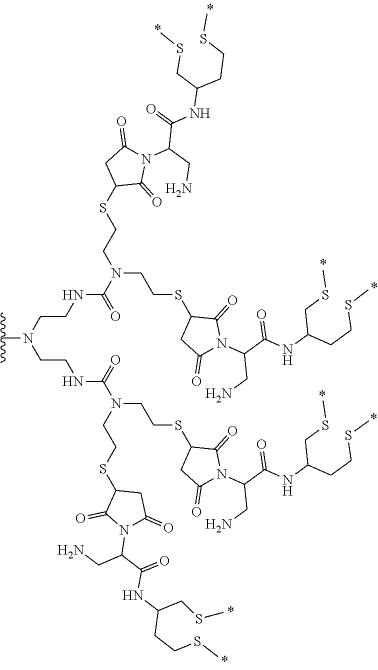 [SMILES: *N(CCNC(=O)N(CCSC1CC(=O)N(C(CN)C(=O)NC(CCSC)CSC)C1=O)CCSC1CC(=O)N(C(CN)C(=O)NC(CCSC)CSC)C1=O)CCNC(=O)N(CCSC1CC(=O)N(C(CN)C(=O)NC(CCSC)CSC)C1=O)CCSC1CC(=O)N(C(CN)C(=O)NC(CCSC)CSC)C1=O]

wherein each * represents covalent attachment to L²-D; and each succinimide ring is optionally hydrolyzed as previously described for Mₓ in which subscript x is 2. When L² is absent, each * represents covalent attachment to D.

In some embodiments, each M of (M)ₓ that comprises —CH₂NH₂ and a succinimide ring, has its succinimide ring in hydrolyzed form. In some embodiments, none of the succinimide rings are in hydrolyzed form. For example, when Mₓ is present, in which each M comprises a succinimide ring and a —CH₂NH₂ moiety having its nitrogen atom protected by an acid-labile protecting group. In some embodiments, one succinimide ring is hydrolyzed and the other succinimide rings are not hydrolyzed. In some embodiments, two succinimide rings are hydrolyzed and the other succinimide rings are not hydrolyzed. In some embodiments, three of the succinimide ring are hydrolyzed and the other succinimide ring is not hydrolyzed.

In some embodiments, x is 0 and the multiplexer (M) is absent.

In some embodiments, L² has the formula -(Q)_{q}-(A)ₐ-(W)_{w}—(Y)_{y}, wherein:

Q is a succinimide or hydrolyzed succinimide;

subscript q is 0 or 1;

A is a C₂₋₂₀ alkylene optionally substituted with 1-3 R^{a1}; or a 2 to 40 membered heteroalkylene optionally substituted with 1-3 R^{b1};

each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);

each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);

each R^{d1} and R^{c1} are independently hydrogen or C₁₋₃ alkyl;

subscript a is 0 or 1;

W is a Peptide Cleavable Unit having from 1-12 amino acids, or W is a Glucuronide Unit having the structure:

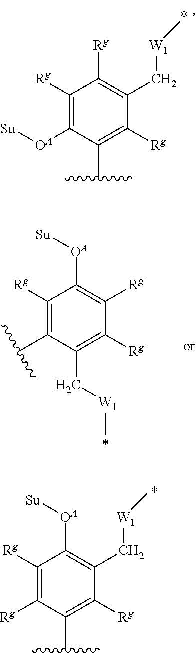

wherein Su is a Sugar moiety;

—O^{A}— represents the oxygen atom of a glycosidic bond;

each R^{g} is independently H, halogen, —CN, or —NO₂;

subscript w is 0 or 1;

W¹ is selected from the group consisting of: —O—, —NH—, —N(C₁₋₆ alkyl)-, —[N(C₁₋₆ alkyl)₂]⁺- and —OC(═O)—;

the wavy line represents covalent attachment to A, Q, or L¹; and

the * represents covalent attachment to Y or D;

subscript w is 0 or 1;

subscript y is 0 or 1;

Y is a self-immolative or non-self-immolative moiety; and

wherein each of L²-D has a net zero charge at physiological pH.

A “sugar moiety” as used herein, refers to a monovalent monosaccharide group, for example, a pyranose or a furanose. A sugar moiety may comprise a hemiacetal or a carboxylic acid (from oxidation of the pendant —CH₂OH group). In some embodiments, the sugar moiety is in the β-D conformation. In some embodiments, the sugar moiety is a glucose, glucuronic acid, or mannose group.

In some embodiments, L² has a net zero charge at physiological pH. In some embodiments, D has a net zero charge at physiological pH. In some embodiments, L² is uncharged at physiological pH. In some embodiments, D is uncharged at physiological pH. In some embodiments, D is charged neutral at physiological pH.

In some embodiments, —O^{A}— represents the oxygen atom of a glycosidic bond. In some embodiments, the glycosidic bond provides a β-glucuronidase or a α-mannosidase-cleavage site. In some embodiments, the β-glucuronidase or a α-mannosidase-cleavage site is cleavable by human lysosomal β-glucuronidase or by human lysosomal α-mannosidase.

In some embodiments, subscript q is 0. In some embodiments, subscript q is 1.

In some embodiments, Q is a succinimide. In some embodiments, Q is a hydrolyzed succinimide. It will be understood that a hydrolyzed succinimide may exist in two regioisomeric form(s). Those forms are exemplified below for Q as a succinimide, wherein the structures representing the regioisomers from that hydrolysis are formula Q′ and Q″; wherein wavy line a indicates the point of covalent attachment to the antibody, and wavy line b indicates the point of covalent attachment to A.

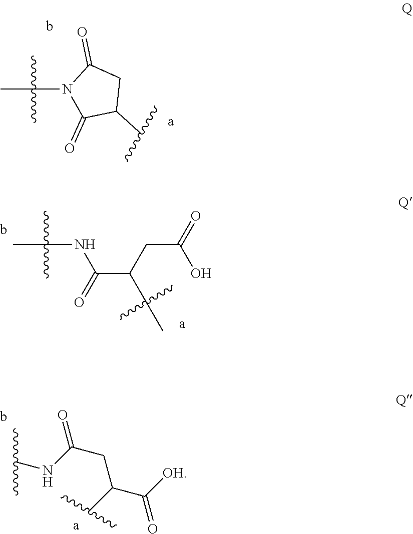 [SMILES: *C1CC(=O)N(*)C1=O ; *NC(=O)C(*)CC(=O)O ; *NC(=O)CC(*)C(=O)O]

In some embodiments, Q′ is

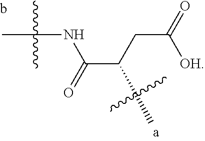 [SMILES: *NC(=O)[C@@H](C)CC(=O)O]

In some embodiments, Q′ is

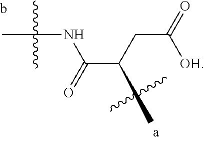 [SMILES: *NC(=O)[C@H](C)CC(=O)O]

In some embodiments, Q″ is

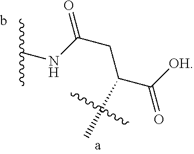 [SMILES: *NC(=O)C[C@@H](C)C(=O)O]

In some embodiments, Q″ is

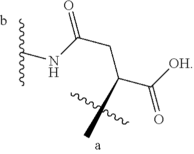 [SMILES: *NC(=O)C[C@H](C)C(=O)O]

In some embodiments, subscript a is 1. In some embodiments, subscript x≥1; and subscript a is 1. In some embodiments, subscript a is 0.

In some embodiments, subscript q is 0 and subscript a is 0.

In some embodiments, A is a C₂₋₂₀ alkylene optionally substituted with 1-3 R^{a1}. In some embodiments, A is a C₂₋₁₀ alkylene optionally substituted with 1-3 R^{a1}. In some embodiments, A is a C₄₋₁₀ alkylene optionally substituted with 1-3 R^{a1}. In some embodiments, A is a C₂₋₂₀ alkylene substituted with one R^{a1}. In some embodiments, A is a C₂₋₁₀ alkylene substituted with one R^{a1}. In some embodiments, A is a C₂₋₁₀ alkylene substituted with one R^{a1}.

In some embodiments, each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(═O)NR^{d1}R^{e1}, —C(═O)(C₁₋₆ alkyl), and —C(═O)O(C₁₋₆ alkyl). In some embodiments, each R^{a1} is C₁₋₆ alkyl. In some embodiments, each R^{a1} is C₁₋₆ haloalkyl. In some embodiments, each R^{a1} is C₁₋₆ alkoxy. In some embodiments, each R^{a1} is C₁₋₆ haloalkoxy. In some embodiments, each R^{a1} is halogen. In some embodiments, each R^{a1} is —OH. In some embodiments, each R^{a1} is ═O. In some embodiments, each R^{a1} is —NR^{d1}R^{e1}. In some embodiments, each R^{a1} is —(C₁₋₆ alkylene)-NR^{d1}R^{e1}. In some embodiments, each R^{a1} is —C(═O)NR^{d1}R^{e1}. In some embodiments, each R^{a1} is —C(═O)(C₁₋₆ alkyl). In some embodiments, each R^{a1} is —C(═O)O(C₁₋₆ alkyl). In some embodiments, one R^{a1} is —NR^{d1}R^{e1}. In some embodiments, one R^{a1} is —(C₁₋₆ alkylene)-NR^{d1}R^{e1}. In some embodiments, one R^{a1} is —(C₁₋₂ alkylene)-NR^{d1}R^{e1}. In some embodiments, A is a C₂₋₂₀ alkylene substituted with 1 or 2 R^{a1}, each of which is ═O.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a C₂₋₂₀ alkylene. In some embodiments, A is a C₂₋₁₀ alkylene. In some embodiments, A is a C₂₋₁₀ alkylene. In some embodiments, A is a C₂₋₆ alkylene. In some embodiments, A is a C₄₋₁₀ alkylene.

In some embodiments, A is a 2 to 40 membered heteroalkylene optionally substituted with 1-3 R^{b1}. In some embodiments, A is a 2 to 20 membered heteroalkylene optionally substituted with 1-3 R^{b1}. In some embodiments, A is a 2 to 12 membered heteroalkylene optionally substituted with 1-3 R^{b1}. In some embodiments, A is a 4 to 12 membered heteroalkylene optionally substituted with 1-3 R^{b1}. In some embodiments, A is a 4 to 8 membered heteroalkylene optionally substituted with 1-3 R^{b1}. In some embodiments, A is a 2 to 40 membered heteroalkylene substituted with one R^{b1}. In some embodiments, A is a 2 to 20 membered heteroalkylene substituted with one R^{b1}. In some embodiments, A is a 2 to 12 membered heteroalkylene substituted with one R^{b1}. In some embodiments, A is a 4 to 12 membered heteroalkylene substituted with one R^{b1}. In some embodiments, A is a 4 to 8 membered heteroalkylene substituted with one R^{b1}.

In some embodiments, each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}—, —C(═O)NR^{d1}R^{e1}, —C(═O)(C₁₋₆ alkyl), and —C(═O)O(C₁₋₆ alkyl). In some embodiments, each R^{b1} is C₁₋₆ alkyl. In some embodiments, each R^{b1} is C₁₋₆ haloalkyl. In some embodiments, each R^{b1} is C₁₋₆ alkoxy. In some embodiments, each R^{b1} is C₁₋₆ haloalkoxy. In some embodiments, each R^{b1} is halogen. In some embodiments, each R^{b1} is —OH. In some embodiments, each R^{b1} is —NR^{d1}R^{e1}. In some embodiments, each R^{b1} is —(C₁₋₆ alkylene)-NR^{d1}R^{e1}. In some embodiments, each R^{b1} is C(═O)NR^{d1}R^{e1}. In some embodiments, each R^{b1} is —C(═O)(C₁₋₆ alkyl). In some embodiments, each R^{b1} is —C(═O)O(C₁₋₆ alkyl). In some embodiments, one R^{b1} is —NR^{d1}R^{e1}. In some embodiments, one R^{b1} is —(C₁₋₆ alkylene)-NR^{d1}R^{e1}. In some embodiments, one R^{b1} is —(C₁₋₂ alkylene)-NR^{d1}R^{e1}.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, Q-A is selected from the group consisting of Ai, Aii or Aiii:

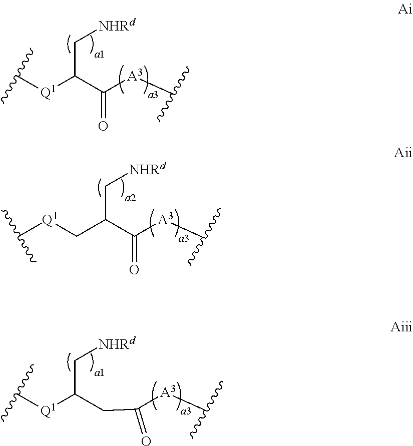

In some embodiments, Q is Q¹. In some embodiments, Q¹ is selected from the group consisting of:

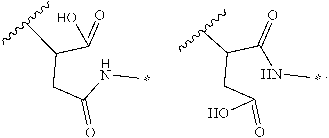

In some embodiments, Q-A has the formula of Aiv:

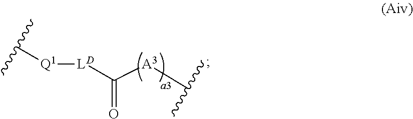

wherein the wavy line adjacent to Q¹ represents covalent attachment to (M)ₓ;

subscript a1 is 1-4; subscript a2 is 0-3; subscript a3 is 0 or 1;

L^{D} is a C₁₋₆ alkylene;

A³ is —NH—(C₁₋₁₀ alkylene)-C(═O)—, or —NH-(2-20 membered heteroalkylene)-C(═O)—, wherein the C₁₋₆ alkylene is optionally substituted with 1-3 independently selected R^{a}, and the 2-20 membered heteroalkylene is optionally substituted with 1-3 independently selected R^{b}; and

wherein A³ is further optionally substituted with a PEG Unit selected from PEG2 to PEG72.

In some embodiments, Q¹ has the structure of:

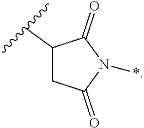

In some embodiments, A³ is further optionally substituted with PEG12 to PEG32 or PEG8 to PEG24.

In some embodiments, subscript a3 is 0. In some embodiments, subscript a3 is 1.

In some embodiments, A³ is —NH—(C₁₋₁₀ alkylene)-C(═O)—.

In some embodiments, A³ is —NH—(CH₂CH₂)—C(═O)—.

In some embodiments, A³ is —NH-(2-20 membered heteroalkylene)-C(═O)—, wherein the 2-20 membered heteroalkylene is optionally substituted with 1-3 independently selected R^{b}.

In some embodiments, A³ is of formula Av

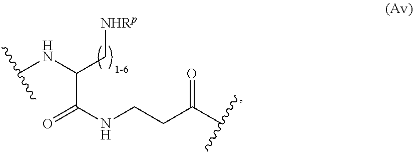

wherein R^{p} is comprised polyethylene glycol chain. In some embodiments, R^{p} is covalently attached to the nitrogen atom via the carbonyl carbon atom of a —(C₁₋₆ alkylene)C(═O)— group, wherein the polyethylene glycol chain and the —(C₁₋₆ alkylene)C(═O)— group form a PEG Unit ranging from PEG2 to PEG72 (e.g., PEG12 or PEG24).

In some embodiments, W is a single amino acid. In some embodiments, W is a single natural amino acid. In some embodiments, W is a peptide including from 2-12 amino acids, wherein each amino acid is independently a natural or unnatural amino acid. In some embodiments, each amino acid is independently a natural amino acid. In some embodiments, W is a dipeptide. In some embodiments, W is a tripeptide. In some embodiments, W is a tetrapeptide. In some embodiments, W is a pentapeptide. In some embodiments, W is a hexapeptide. In some embodiments, W is 7, 8, 9, 10, 11, or 12 amino acids. In some embodiments, each amino acid of W is independently selected from the group consisting of valine, alanine, β-alanine, glycine, lysine, leucine, phenylalanine, proline, aspartic acid, glutamate, arginine, and citrulline. In some embodiments, each amino acid of W is independently selected from the group consisting of valine, alanine, β-alanine, glycine, lysine, leucine, phenylalanine, proline, aspartic acid, serine, glutamic acid, homoserine methyl ether, aspartate methyl ester, N,N-dimethyl lysine, arginine, valine-alanine, valine-citrulline, phenylalanine-lysine, and citrulline. In some embodiments, W is an aspartic acid. In some embodiments, W is a lysine. In some embodiments, W is a glycine. In some embodiments, W is an alanine. In some embodiments, W is aspartate methyl ester. In some embodiments, W is a N,N-dimethyl lysine. In some embodiments, W is a homoserine methyl ether. In some embodiments, W is a serine. In some embodiments, W is a valine-alanine.

In some embodiments, W is from 1-12 amino acids and the bond between W and Y or W and D is enzymatically cleavable by a tumor-associated protease. In some embodiments, W is an amino acid or a dipeptide; and the bond between W and D or between W and Y is enzymatically cleavable by a tumor-associated protease. In some embodiments, the tumor-associated protease is a lysosomal protease such as a cathepsin. In some embodiments, the tumor-associated protease is cathepsin B.

In some embodiments, W is a Glucuronide Unit, having the structure of formula Wi, Wii or Wiii:

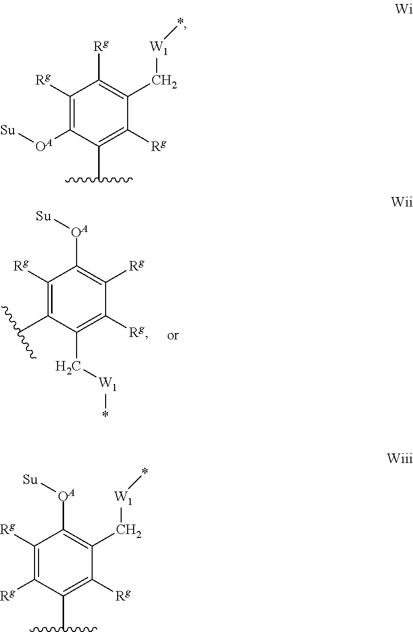

wherein Su is a Sugar moiety;

—O^{A}— represents the oxygen atom of a glycosidic bond;

each R^{g} is independently hydrogen, halogen, —CN, or —NO₂;

W¹ is selected from the group consisting of: a bond, —O—, —C(═O)—, S(O)₀₋₂—, —NH—, —N(C₁₋₆ alkyl)-, —[N(C₁₋₆ alkyl)₂]⁺-, —OC(═O)—, —NHC(═O)—, —C(═O)O—, and —C(═O)NH—;

the wavy line represents the covalent attachment to A, Q, or L¹; and

the * represents the covalent attachment to Y or D.

In some embodiments, —O^{A}— represents the oxygen atom of a glycosidic bond. In some embodiments, the glycosidic bond provides a β-glucuronidase or a α-mannosidase-cleavage site. In some embodiments, the β-glucuronidase or a α-mannosidase-cleavage site is cleavable by human lysosomal β-glucuronidase or by human lysosomal α-mannosidase.

In some embodiments, O^{A}-Su has zero net charge at physiological pH. In some embodiments, O^{A}-Su is uncharged at physiological pH. In some embodiments, O^{A}-Su is mannose. In some embodiments, O^{A}-Su is

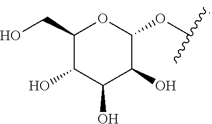 [SMILES: *O[C@H]1O[C@H](CO)[C@@H](O)[C@H](O)[C@@H]1O]

In some embodiments, Su of O^{A}—Su in formula Wi, Wii or Wii comprises a carboxylate moiety. In some embodiments, O^{A}-Su is glucuronic acid moiety. In some embodiments, O^{A}-Su is

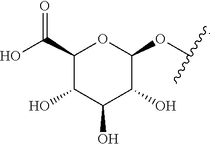 [SMILES: *O[C@@H]1O[C@H](C(=O)O)[C@@H](O)[C@H](O)[C@H]1O]

In some embodiments, each R^{g} is hydrogen. In some embodiments, one R^{g} is hydrogen, and the remaining R^{g} are independently halogen, —CN, or —NO₂. In some embodiments, two R^{g} are hydrogen, and the remaining R^{g} is halogen, —CN, or —NO₂.

In some embodiments, W¹ is a bond. In some embodiments, W¹ is —O—. In some embodiments, W¹ is —C(═O)—. In some embodiments, W¹ is —NH—. In some embodiments, W¹ is —N(C₁₋₆ alkyl)-. In some embodiments, W¹ is —[N(C₁₋₆ alkyl)₂]⁺-.

In some embodiments, W¹ is —OC(═O)—; and O^{A}-Su is charged neutral. In some embodiments, W¹ is a bond; D is conjugated to W through a nitrogen atom which forms an ammonium cation at physiological pH; and Su of O^{A}-Su is a sugar moiety having a carboxylate substituent.

In some embodiments, W is Wi having the structure of:

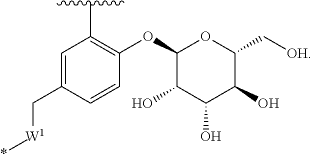

In some embodiments, W is Wii or Wi having the structure of

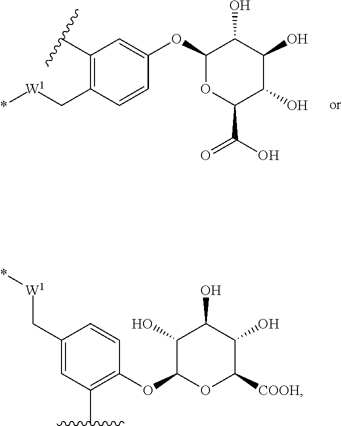

respectively. In some embodiments, W is Wii having the structure of:

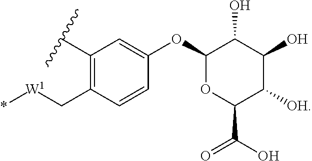

In some embodiments, W is Wi having the structure of:

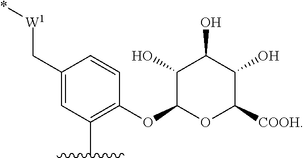

In some embodiments, subscript w is 1 and subscript a is 0.

In some embodiments, W¹ is a bond. In some embodiments, W¹ is —O(C═O)—.

In some embodiments, W is a Peptide Cleavable Unit and subscript y is 0. In some embodiments, W is a Peptide Cleavable Unit and subscript y is 1. In some embodiments, W is a Peptide Cleavable Unit and subscript y is 1. In some embodiments, W is a Peptide Cleavable Unit and subscript y is 0.

A non-self-immolative moiety is one which requires enzymatic cleavage, and in which part or all of the group remains bound to the Drug after cleavage from the ADC. Examples of a non-self-immolative moiety include, but are not limited to: -glycine-; and -glycine-glycine-. In some embodiments, in which Y is -glycine- or -glycine-glycine-, L₂-D undergoes enzymatic cleavage, for example, via a tumor-cell associated-protease, a cancer-cell-associated protease, or a lymphocyte-associated protease to provide a glycine-Drug Unit or glycine-glycine-Drug Unit fragment as the free drug. In some embodiments, an independent hydrolysis or proteolysis reaction takes place within the target cell, further cleaving the glycine-Drug or glycine-glycine-Drug Unit to liberate the parent drug as the free drug.

In some embodiments, in which Y is a p-aminobenzyl alcohol (PAB) optionally substituted with one or more halogen, cyano, or nitro groups, Y undergoes enzymatic cleavage, for example, via a tumor-cell associated-protease, a cancer-cell-associated protease, or a lymphocyte-associated protease, releasing a PAB-Drug Unit fragment further undergoes 1,6-elimination of the PAB to liberate free drug. In some embodiments, enzymatic cleavage of the non-self-immolative moiety, as described herein, directly liberates free drug without any further hydrolysis or proteolysis step(s).

A self-immolative moiety is one which does not require any additional hydrolysis steps to liberate D as free drug. For example, the phenylene moiety of a p-aminobenzyl alcohol (PAB) moiety as previously described, is covalently attached to —W_{w}— via the amino nitrogen atom of the PAB group, and is covalently attached to -D via a carbonate, carbamate or ether group. See, e.g., Told et al., 2002, J. Org. Chem. 67:1866-1872.

Examples of a self-immolative moiety include, but are not limited to, a p-aminobenzyl alcohol (PAB) moiety, the phenylene of which is unsubstituted at the remaining aromatic carbon atoms or is substituted with one or more C₁₋₃ alkoxy, halogen, cyano, or nitro groups. In some embodiments, when subscript w is 1 and W is a Peptide Cleavable Unit, the phenylene of a PAB moiety is optionally substituted with one C₁₋₃ alkoxy group.

Other examples of self-immolative groups include, but are not limited to, aromatic compounds that are electronically similar to the PAB moiety such as 2-aminoimidazol-5-methanol derivatives (see, e.g., Hay et al., 1999, Bioorg. Med. Chem. Lett. 9:2237), ortho or para-aminobenzylacetals, substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), 2-aminophenylpropionic acid amides (see, e.g., Amsberry et al., 1990, J. Org. Chem. 55:5867), elimination of amine-containing drugs that are substituted at the α-position of glycine (see, e.g., Kingsbury et al., 1984, J. Med. Chem. 27:1447), and group such as

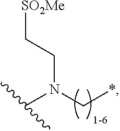

where * represents covalent attachment to D and the nitrogen adjacent to [symbol] forms a carbamate with W.

In some embodiments, Y is a para-aminobenzyloxy-carbonyl (PABC) group optionally substituted with a sugar moiety. In some embodiments, Y is -glycine- or -glycine-glycine-. In some embodiments, Y is a branched bis(hydroxymethyl)styrene (BHMS) unit, which is capable of incorporating (and releasing) multiple Drug Units.

In some embodiments, of L₂-D, subscript w is 1, and -(Q)_{q}-(A)ₐ-(W)_{w}—(Y)_{y} comprises a releasable linker, which provides release of free drug once the ADC has been internalized into the target cell. In some embodiments, subscript w is 1, and -(Q)_{q}-(A)ₐ-(W)_{w}—(Y)_{y} is a releasable linker, which provides release of free drug in the vicinity of targeted cells. Releasable linkers possess a suitable recognition site, such as a peptide cleavage site, sugar cleavage site, or a disulfide cleavage side. In some embodiments, each releasable linker is a di-peptide. In some embodiments, each releasable linker independently comprises succinimido-caproyl (mc), succinimido-caproyl-valine-citrulline (sc-vc), succinimido-caproyl-valine-citrulline-paraaminobenzyloxycarbonyl (sc-vc-PABC), SDPr-vc (where “S” refers to succinimido), -propionyl-valine-citrulline-, Val-Cit-, -Phe-Lys-, or -Val-Ala-.

In some embodiments, each releasable linker is independently selected from Val-Cit-, -Phe-Lys-, and -Val-Ala-. In some embodiments, each releasable linker is independently selected from succinimido-caproyl (mc), succinimido-caproyl-valine-citrulline (sc-vc), succinimido-caproyl-valine-citrulline-paraaminobenzyloxycarbonyl (sc-vc-PABC), SDPr-vc (where “S” refers to succinimido), and -propionyl-valine-citrulline-.

In some embodiments, -(Q)_{q}-(A)ₐ-(W)_{w}—(Y)_{y}— a non-releasable linker, wherein the Drug Unit is released after the ADC has been internalized into the target cell and degraded, liberating free drug.

In some embodiments, -(Q)_{q}-(A)ₐ-(W)_{w}—(Y)_{y} is a releasable linker, wherein subscript y is 1; and Y is

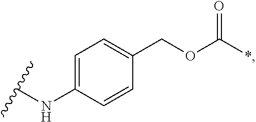

wherein the wavy line represents covalent attachment to W or A; and the * represents covalent attachment to D.

In some embodiments, subscript a is 1; subscript w is 1; and Q-A-W is

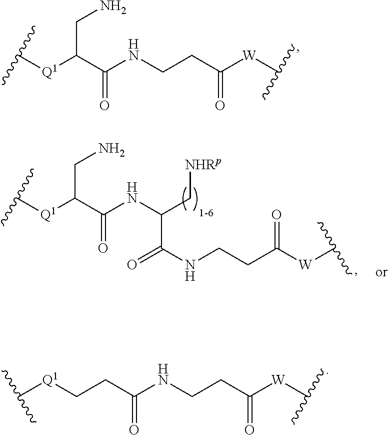

In some embodiments, Q-A-W is

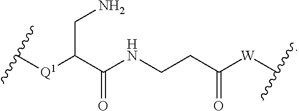

In some embodiments, Q-A-W is

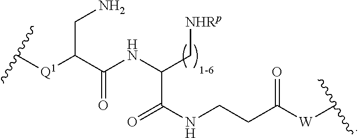

In some embodiments, Q-A-W is

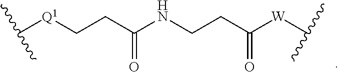

In some embodiments, R^{p} is a PEG Unit ranging from PEG2 to PEG72 (e.g., PEG12 or PEG24). In some embodiments, this PEG Unit comprises a —(C₁₋₆ alkylene)C(═O)—, group wherein the carbonyl carbon atom of the —(C₁₋₆ alkylene)C(═O)—, group is covalently attached to the nitrogen atom substituted by R^{p}.

In some embodiments, W is a Peptide Cleavable Unit or a Glucuronide Unit, A is not comprised of R^{p} substituted with a PEG Unit. In some embodiments, L² is substituted with a PEG Unit ranging from PEG2, PEG4, PEG6, PEG8, PEG10, PEG12, PEG16, PEG20, and PEG24. In some embodiments, W is a Peptide Cleavable Unit or a Glucuronide Unit, A is substituted with a PEG Unit ranging from PEG2 to PEG72, for example, PEG12 to PEG32, or PEG8 to PEG24. In some embodiments, L² is substituted with a PEG Unit selected from PEG2, PEG4, PEG6, PEG8, PEG10, PEG12, PEG16, PEG20, and PEG24.

Upon review of the present disclosure and the examples provided therein, a person of skill in the art will recognize that the operability of the ADCs and intermediates thereof described herein is not dependent on the exact structure of any one linker (L¹ or L²), and the additional structural features that are not explicitly described herein are capable of being incorporated into one or more linkers (L¹ or L²) without departing from the scope of the present disclosure.

Additionally, one of skill in the art will also appreciate that the specific attachment chemistry to an antibody, for example, can alter the synthetic steps leading to a product. In particular, when attachment to the sulfur atom of a thiol group on an antibody is to be carried out by means of a thiol reactive group, that attachment to the antibody will take place prior to reducing the cyclic thiol multiplexing moieties (M) to avoid unwanted or off target reactions between thiols in the linkers (L¹ and L²) and the aforementioned thiol reactive groups.

Drug Units

In some embodiments, D is a Drug Unit that is conjugated to a Drug Linker compound or to an antibody-drug conjugate. In some embodiments, D is free drug (from the corresponding Drug Unit), or a pharmaceutically acceptable salt thereof), and may be useful for pharmaceutical treatment of hyperproliferative diseases and disorders. The substituent designations in this section (R¹, R², R³, and the like) refer only to the Drug Units and corresponding free drugs described in the present application. These designations are not applicable to linkers (as standalone compounds or as components of ADCs) or to linker intermediate compounds, which have distinct substituents designations as described herein.

In some embodiments, D is a cytotoxic, cytostatic, immunosuppressive, immunostimulatory, or immunomodulatory drug. In some embodiments, D is a tubulin disrupting agent, DNA minor groove binder, DNA damaging agent or DNA replication inhibitor.

Useful classes of cytotoxic, cytostatic, immunosuppressive, immunostimulatory, or immunomodulatory agents include, for example, antitubulin agents (which may also be referred to as tubulin disrupting agents), DNA minor groove binders, DNA replication inhibitors, DNA damaging agents, alkylating agents, antibiotics, antifolates, antimetabolites, chemotherapy sensitizers, Toll-like receptor (TLR) agonists, STimulator of Interferon Genes (STING) agonists, Retinoic acid-inducible gene I (RIG-I) agonists, topoisomerase inhibitors (including topoisomerase I and II inhibitors), vinca alkaloids, auristatins, camptothecins, enediynes, lexitropsins, anthracyclins, taxanes, and the like. Particularly examples of useful classes of cytotoxic agents include, for example, DNA minor groove binders (enediynes and lexitropsins), DNA alkylating agents, and tubulin inhibitors. Exemplary agents include, for example, anthracyclines, auristatins (e.g., auristatin T, auristatin E, AFP, monomethyl auristatin F (MMAF), lipophilic monomethyl aurstatin F, monomethyl auristatin E (MMAE)), camptothecins, CC-1065 analogues, calicheamicin, analogues of dolastatin 10, duocarmycins, etoposides, maytansines and maytansinoids, melphalan, methotrexate, mitomycin C, taxanes (e.g., paclitaxel and docetaxel), nicotinamide phosphoribosyltranferase inhibitor (NAMPTi), tubulysin M, benzodiazepines and benzodiazepine containing drugs (e.g., pyrrolo[1,4]-benzodiazepines (PBDs), indolinobenzodiazepines, rhizoxin, paltoxin, and oxazolidinobenzodiazepines) and vinca alkaloids. Select benzodiazepine containing drugs are described in WO 2010/091150, WO 2012/112708, WO 2007/085930, and WO 2011/023883.

Particularly useful classes of cytotoxic agents include, for example, DNA minor groove binders, DNA alkylating agents, tubulin disrupting agents, anthracyclines and topoisomerase II inhibitors. Other particularly useful cytotoxic agents include, for example, auristatins (e.g., auristatin T, auristatin E, AFP, monomethyl auristatin F (MMAF), lipophilic analogs of monomethyl auristatin F, monomethyl auristatin E (MMAE)) and camptothecins (e.g., camptothecin, irinotecan and topotecan).

The cytotoxic agent can be a chemotherapeutic agent such as, for example, doxorubicin, paclitaxel, melphalan, vinca alkaloids, methotrexate, mitomycin C or etoposide. The agent can also be a CC-1065 analogue, calicheamicin, maytansine, an analog of dolastatin 10, rhizoxin, or palytoxin.

The cytotoxic agent can also be an auristatin. The auristatin can be an auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatins include auristatin T, AFP, MMAF, and MMAE. The synthesis and structure of various auristatins are described in, for example, US 2005-0238649 and US2006-0074008.

The cytotoxic agent can be a DNA minor groove binding agent. (See, e.g., U.S. Pat. No. 6,130,237.) For example, the minor groove binding agent can be a CBI compound or an enediyne (e.g., calicheamicin).

The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine), and auristatins (e.g., auristatin E, AFP, MMAF, MMAE, AEB, AEVB). Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be mytansine or a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et al., 1992, Cancer Res.).

In some embodiments, D is a tubulin disrupting agent. In some embodiments, D is an auristatin or a tubulysin. In some embodiments, D is an auristatin. In some embodiments, D is a tubulysin.

In some embodiments, D is a TLR agonist. Exemplary TLR agonists include, but are not limited to, a TLR1 agonist, a TLR2 agonist, a TLR3 agonist, a TLR4 agonist, a TLR5 agonist, a TLR6 agonist, a TLR7 agonist, a TLR8 agonist, a TLR7/8 agonist, a TLR9 agonist, or a TLR10 agonist.

In some embodiments, D is a STING agonist. Exemplary STING agonists include, but are not limited to, cyclic di-nucleotides (CDNs), and non-nucleotide STING agonists.

An auristatin Drug Unit of an antibody-drug conjugate or Drug Linker compound incorporates an auristatin drug through covalent attachment of a Linker Unit of the Conjugate or Drug Linker compound to the secondary amine of an auristatin free drug having structure of D_{E} or D_{F} as follows:

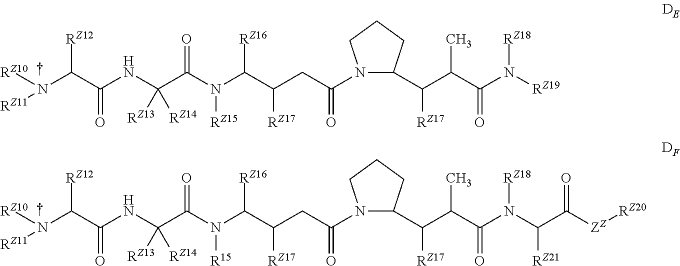

wherein the dagger indicates the site of covalent attachment of the nitrogen atom that provides a carbamate functional group, wherein —OC(═O)— of that functional group is Y^{Z}′ on incorporation of the auristatin drug compound as -D into any one of the drug linker moieties of an antibody-drug conjugate or into any one of the Drug Linker compounds as described herein, so that for either type of compound subscript y is 2; and one R^{Z10} and R^{Z11} is hydrogen and the other is C₁-C₈ alkyl; R^{Z12} is hydrogen, C₁-C₈ alkyl, C₃-C₈ carbocyclyl, C₆-C₂₄ aryl, —X^{Z1}—C₆-C₂₄ aryl, —X^{Z1}—(C₃-C₈ carbocyclyl), C₃-C₈ heterocyclyl or —X^{Z1}—(C₃-C₈ heterocyclyl); R^{Z13} is hydrogen, C₁-C₈ alkyl, C₃-C₈ carbocyclyl, C₆-C₂₄ aryl, —X^{Z1}—C₆-C₂₄ aryl, —X^{Z1}—(C₃-C₈ carbocyclyl), C₃-C₈ heterocyclyl and —X^{Z1}—(C₃-C₈ heterocyclyl); R^{Z14} is hydrogen or methyl, or R^{Z13} and R^{Z14} taken together with the carbon to which they are attached comprise a spiro C₃-C₈ carbocyclo; R^{Z15} is hydrogen or C₁-C₈ alkyl; R^{Z16} is hydrogen, C₁-C₈ alkyl, C₃-C₈ carbocyclyl, C₆-C₂₄ aryl, —C₆-C₂₄—X^{Z1}-aryl, —X^{Z1}—(C₃-C₈ carbocyclyl), C₃-C₈ heterocyclyl and —X^{Z1}—(C₃-C₈ heterocyclyl); R^{Z17} independently are hydrogen, —OH, C₁-C₈ alkyl, C₃-C₈ carbocyclyl and O—(C₁-C₈ alkyl); R^{Z18} is hydrogen or optionally substituted C₁-C₈ alkyl; R^{Z19} is —C(R^{Z19A})₂—C(R^{Z19A})₂—C₆-C₂₄ aryl, —C(R^{Z19A})₂—C(R^{19A})₂—(C₃-C₈ heterocyclyl) or —C(R^{Z19A})₂—C(R^{Z19A})₂—(C₃-C₈ carbocyclyl), wherein C₆-C₂₄ aryl and C₃-C₈ heterocyclyl are optionally substituted; R^{Z19A} independently are hydrogen, optionally substituted C₁-C₈ alkyl, —OH or optionally substituted —O—C₁-C₈ alkyl; R^{Z20} is hydrogen or optionally substituted C₁-C₂₀ alkyl, optionally substituted C₆-C₂₄ aryl or optionally substituted C₃-C₈ heterocyclyl, or —(R^{Z47}O)_{mz}—R⁴⁸, or —(R⁴⁷O)_{mz}—CH(R⁴⁹)₂; R^{Z21} is optionally substituted —C₁-C₈ alkylene-(C₆-C₂₄ aryl) or optionally substituted —C₁-C₈ alkylene-(C₅-C₂₄ heteroaryl), or C₁-C₈ hydroxylalkyl, or optionally substituted C₃-C₈ heterocyclyl; Z^{Z} is O, S, NH, or NR^{Z46}; R^{Z46} is optionally substituted C₁-C₈ alkyl; subscript mz is an integer ranging from 1-1000; R^{Z47} is C₂-C₈ alkyl; R^{Z48} is hydrogen or C₁-C₈ alkyl; R^{Z49} independently are —COOH, —(CH₂)_{nz}—N(R^{Z50})₂, —(CH₂)_{nz}—SO₃H, or —(CH₂)_{nz}—SO₃—C₁-C₈ alkyl; R^{Zs0} independently are C₁-C₈ alkyl, or —(CH₂)_{nz}—COOH; subscript nz is an integer ranging from 0 to 6; and X^{Z1} is C₁-C₁₀ alkylene.

In some embodiments the auristatin drug compound has the structure of Formula D_{E-1}, Formula D_{E-2} or Formula D_{F-1}:

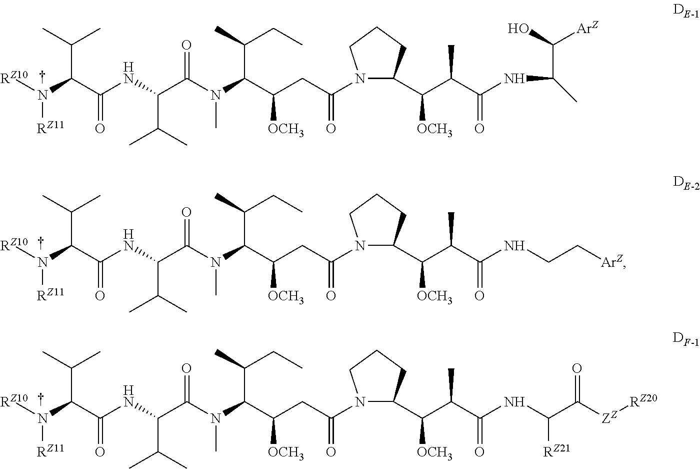

wherein Ar^{Z} in Formula D_{E-1} or Formula D_{E-2} is C₆-C₁₀ aryl or C₅-C₁₀ heteroaryl, and in Formula D_{F-1}, Z^{Z} is —O—, or —NH—; R^{Z20} is hydrogen or optionally substituted C₁-C₆ alkyl, optionally substituted C₆-C₁₀ aryl or optionally substituted C₅-C₁₀ heteroaryl; and R^{Z21} is optionally substituted C₁-C₆ alkyl, optionally substituted —C₁-C₆ alkylene-(C₆-C₁₀ aryl) or optionally substituted —C₁-C₆ alkylene-(C₅-C₁₀ heteroaryl).

In some embodiments of Formula D_{E}, D_{F}, D_{E-1}, D_{E-2} or D_{F-1}, one of R^{Z10} and R^{Z11} is hydrogen and the other is methyl.

In some embodiments of Formula D_{E-1} or D_{E-2}, Ar is phenyl or 2-pyridyl.

In some embodiments of Formula D_{F-1}, R^{Z21} is X^{Z1}—S—R^{Z21a} or X^{Z1}—Ar^{Z}, wherein X^{Z1} is C₁-C₆ alkylene, R^{Z21a} is C₁-C₄ alkyl and Ar^{Z} is phenyl or C₅-C₆ heteroaryl and/or —Z^{Z}— is —O— and R^{Z20} is C₁-C₄ alkyl or Z^{Z} is —NH— and R^{Z20} is phenyl or C₅-C₆ heteroaryl.

In some embodiments the auristatin drug compound has the structure of Formula D_{F/E-3}:

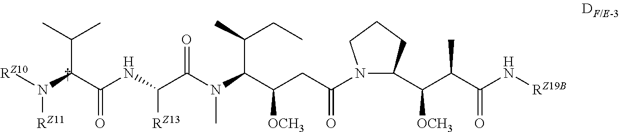

wherein one of R^{Z10} and R^{Z11} is hydrogen and the other is methyl; R^{Z13} is isopropyl or —CH₂—CH(CH₃)₂; and R^{Z19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-C₁-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or R^{Z19B} has the structure of

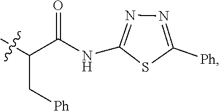

wherein the wavy line indicates covalent attachment to the remainder of the auristatin compound.

In some embodiments the auristatin drug compound incorporated into -D is monomethylauristatin E (MMAE) or monomethylauristatin F (MMAF).

In some embodiments, the free drug that is conjugated within an antibody-drug conjugate or Drug Liker compound is an amine-containing tubulysin compound wherein the nitrogen atom of the amine is the site of covalent attachment to the Linker Unit of the antibody-drug conjugate or Drug Liker compound and the amine-containing tubulysin compound has the structure of Formula D_{G} or D_{H}:

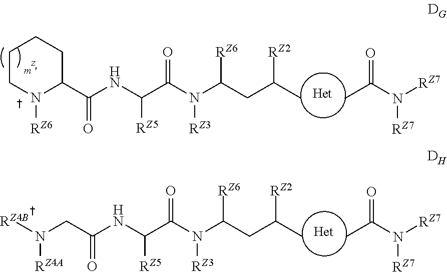

wherein the dagger represents the point of covalent attachment of the Drug AntoheLinker Unit, in which the nitrogen atom so indicated becomes quaternized, in a Drug Linker compound or antibody-drug conjugate and the circle represents an 5-membered or 6-membered nitrogen heteroaryl wherein the indicated required substituents to that heteroaryl are in a 1,3- or meta-relationship to each other with optional substitution at the remaining positions; R^{Z2} is X^{ZA}—R^{Z2A}, wherein X^{ZA} is —O—, —S—, —N(R^{Z2B})—, —CH₂—, —(C═O)N(R^{Z2B})— or —O(C═O)N(R^{Z2B})— wherein R^{Z2B} is hydrogen or optionally substituted alkyl, R^{Z2A} is hydrogen, optionally substituted alkyl, optionally substituted aryl, or —C(═O)R^{ZC}, wherein R^{C} is hydrogen, optionally substituted alkyl, or optionally substituted aryl or R^{Z2} is an O-linked substituent; R^{Z3} is hydrogen or optionally substituted alkyl; R^{Z4}, R^{Z4A}, R^{Z4B}, R^{Z5} and R^{Z6} are optionally substituted alkyl, independently selected, one R^{Z7} is hydrogen or optionally substituted alkyl and the other R^{Z7} is optionally substituted arylalkyl or optionally substituted heteroarylalkyl, and m^{Z} is 0 or 1. In other embodiments the quaternized drug is a tubulysin represented by structure D_{G} wherein one R^{Z7} is hydrogen or optionally substituted alkyl, the other R^{Z7} is an independently selected optionally substituted alkyl, and subscript mz′ is 0 or 1, wherein the other variable groups are as previously defined. In some embodiments, one R^{Z7} is hydrogen or optionally substituted lower alkyl, the other R^{Z7} is an independently selected optionally substituted C₁-C₆ alkyl, and subscript mz′ is 1, wherein the other variable groups are as previously defined.

In some embodiments, R^{Z2} is X^{ZA}—R^{Z2A}, wherein X^{ZA} is —O—, —S—, —N(R^{Z2B})—. —CH₂—, or —O(C═O)N(R^{2B})— wherein R^{Z2B} is hydrogen or optionally substituted alkyl, R^{Z2A} is hydrogen, optionally substituted alkyl, optionally substituted aryl, or —C(═O)R^{ZC}, wherein R^{ZC} is hydrogen, optionally substituted alkyl, or optionally substituted aryl or R^{Z2} is an O-linked substituent.

In some embodiments, R^{Z2} is X^{ZA}—R^{Z2A}, wherein X^{ZA} is —O—, —S—, —N(R^{Z2B})— or —(C═O)N(R^{Z2B})— wherein R^{Z2A} and R^{Z2B} are independently hydrogen or optionally substituted alkyl, or R^{Z2} is an O-linked substituent.

In some embodiments —N(R^{Z7})(R^{Z7}) in D_{G} or D_{H} is replaced by —N(R^{Z7})—CH(R^{Z10})(CH₂R^{Z11}) to define tubulysin compounds of formula D_{H}′ and D_{G}′:

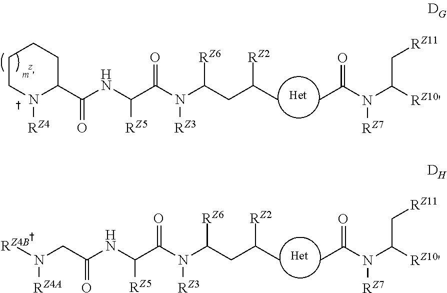

wherein the dagger represents the point of covalent attachment to the Linker Unit, in which the nitrogen atom so indicated becomes quaternized, in a Drug Linker compound or antibody-drug conjugate; R^{Z10} is C₁-C₆ alkyl substituted with —CO₂H, or ester thereof, and R^{Z7} is hydrogen or a C₁-C₆ alkyl independently selected from R^{Z10}, or R^{Z7} and R^{Z10} together with the atoms to which they are attached define a 5 or 6-membered heterocycle; and R^{Z11} is aryl or 5- or 6-membered heteroaryl, optionally substituted with one or more, substituent(s) independently selected from the group consisting of halogen, lower alkyl, —OH and —O—C₁-C₆ alkyl; and the remaining variable groups are as defined for D_{G} and D_{H}. In some embodiments, R^{Z11} is substituted with one or two substituents selected from the group consisting of halogen, lower alkyl, —OH and —O—C₁-C₆ alkyl. In some embodiments, R^{Z11} is substituted with one substitutent selected from the group consisting of halogen, lower alkyl, —OH and —O—C₁-C₆ alkyl. In some embodiments, the halogen is F. In some embodiments, the —O—C₁-C₆ alkyl is —OCH₃. In some embodiments, the lower alkyl is —CH₃.

In still other embodiments one R^{Z7} in —N(R^{Z7})(R^{Z7}) in D_{G} or D_{H} is hydrogen or C₁-C₆ alkyl, and the other R^{Z7} is an independently selected C₁-C₆ alkyl optionally substituted by —CO₂H or an ester thereof, or by an optionally substituted phenyl.

In some embodiments of structure D_{G} and D_{H}, one R^{Z7} is hydrogen and the other R^{Z7} is an optionally substituted arylalkyl having the structure of:

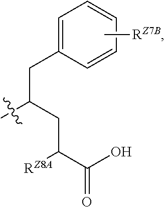

wherein R^{Z7B} is hydrogen or an O-linked substituent, and R^{Z8A} is hydrogen or lower alkyl; and wherein the wavy line indicates the point of attachment to the remainder of D_{G} or D_{H}. In some embodiments, R^{Z7B} is hydrogen or —OH in the para position. In some embodiments, R^{Z8A} is methyl.

In some embodiments of structure D_{G} or D_{H}, one R^{Z7} is hydrogen, and the other R^{Z7} is an optionally substituted arylalkyl having the structure of

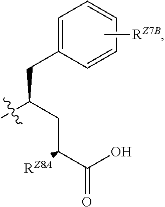

wherein R^{Z7B} is —H or —OH; and wherein the wavy line indicates the point of attachment to the remainder of D_{G} or D_{H}.

In some embodiments of structure D_{G} and D_{H}, one R^{Z7} is hydrogen or lower alkyl, and the other R^{Z7} is optionally substituted arylalkyl having the structure of one of:

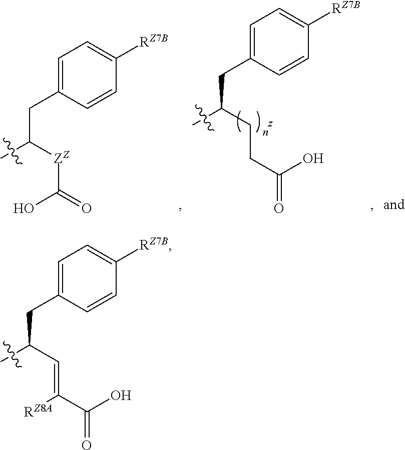

wherein Z^{Z} an optionally substituted alkylene or an optionally substituted alkenylene, R^{Z7B} is hydrogen or an O-linked substituent, R^{Z8A} is hydrogen or lower alkyl, and the subscript nz is 0, 1 or 2; and wherein the wavy line indicates the point of attachment to the remainder of D_{G} or D_{H}. In some embodiments, subscript nz is 0 or 1. In still other embodiments of structure D_{G} and D_{H} —N(R^{Z7})(R^{Z7}) is —NH(C₁-C₆ alkyl) wherein the C₁-C₆ alkyl is optionally substituted by —CO₂H or an ester thereof, or by an optionally substituted phenyl. In some embodiments —N(R^{Z7})(R^{Z7}) is selected from the group consisting of —NH(CH₃), —CH₂CH₂Ph, —CH₂—CO₂H, —CH₂CH₂CO₂H and —CH₂CH₂CH₂CO₂H. In some embodiments, one R^{Z7} is hydrogen or methyl and the other R^{Z7} is an optionally substituted arylalkyl having the structure of:

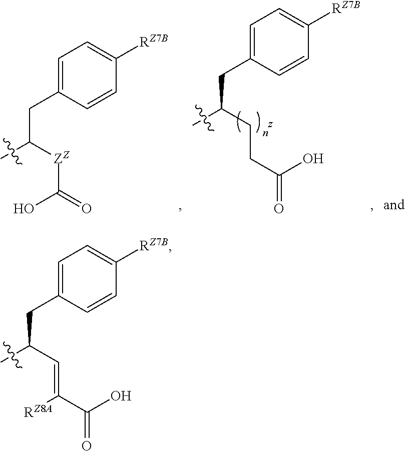

wherein Z^{Z} is an optionally substituted alkylene or an optionally substituted alkenylene, R^{Z7B} is hydrogen or —OH in the para position, R^{Z8A} is hydrogen or methyl, and the subscript nz is 0, 1 or 2 In some embodiments of structure D_{G}′ and D_{H}′, R^{Z7} and R^{Z10} together with the atoms to which they are attached define an optionally substituted 5 or 6-membered heterocycle wherein —N(R^{Z7})—CH(R^{Z10})(CH₂R^{Z11}) has the structure of:

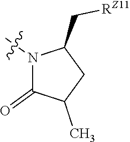

wherein the wavy line indicates the point of attachment to the remainder of D_{G}′ or D_{H}′.

In some embodiments, the tubulysin compound is represented by the following formula wherein the indicated nitrogen (†) is the site of quaternization when such compounds are incorporated into an ADC as a quaternized drug unit (D⁺):

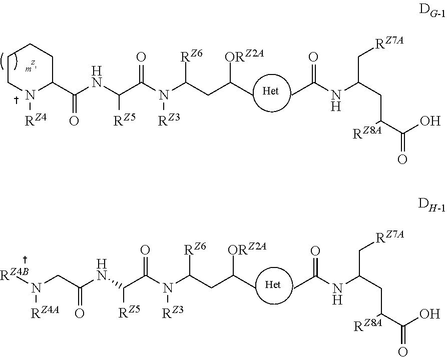

wherein the dagger represents the point of attachment of the Drug Unit to the Linker Unit in a Drug Linker compound or antibody-drug conjugate in which the nitrogen atom so indicated becomes quaternized, and the circle represents an 5-membered or 6-membered nitrogen-heteroaryl wherein the indicated required substituents to that heteroaryl are in a 1,3- or meta-relationship to each other with optional substitution at the remaining positions; R^{Z2A} is hydrogen or optionally substituted alkyl or R^{Z2A} along with the oxygen atom to which it is attached defines an O-linked substituent; R^{Z3} is hydrogen or optionally substituted alkyl; R^{Z4}, R^{Z4A}, R^{Z4B}, R^{Z5} and R^{Z6} are optionally substituted alkyl, independently selected; R^{Z7A} is optionally substituted aryl or optionally substituted heteroaryl, R^{Z8A} is hydrogen or optionally substituted alkyl and subscript mz′ is 0 or 1.

In some embodiments of structure D_{G}, D_{G-1}, D_{H}, or D_{H-1}, R^{Z4} is methyl or R^{Z4A} and R^{Z4B} are methyl. In other embodiments of structure D_{G}′ or D_{H}′ R^{Z4} is methyl or R^{Z4A} and R^{Z4B} are methyl. In other embodiments, R^{Z7A} is optionally substituted phenyl. In some embodiments R^{Z8A} is methyl in the (S)-configuration. In other embodiments, R^{Z2A} along with the oxygen atom to which it is attached defines an O-linked substituent other than —OH. In some embodiments, R^{Z2A} along with the oxygen atom to which it is attached defines an ester, ether, or an O-linked carbamate. In some embodiments the circle represents a 5-membered nitrogen-heteroarylene. Some embodiments, the circle represents a divalent oxazole or thiazole moiety. In some embodiments R^{Z4} is methyl or R^{Z4A} and R^{Z4B} are methyl. In some embodiments R^{Z7} is optionally substituted arylalkyl, wherein aryl is phenyl and R^{Z7A} is optionally substituted phenyl.

In other embodiments of D_{G}, D_{G}′, D_{G-1}, D_{H}, D_{H}′ or D_{H-1} the circle represents a 5-membered nitrogen heteroarylene. In some embodiments, the 5-membered heteroarylene is represented by the structure

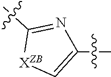

wherein X^{ZB} is O, S, or N—R^{ZB} wherein R^{ZB} is hydrogen or lower alkyl. In some embodiments, the quaternized drug is a tubulysin represented by structure D_{G}, D_{G}′ or D_{G-1}, wherein m is 1. In some embodiments, the tubulysins are represented by structure D_{G}, wherein m is 1 and the circle represents an optionally substituted divalent thiazole moiety.

In some embodiments, the tubulysin compound is represented by the following formula wherein the indicated nitrogen atom (†) is the site of quaternization when such compounds are incorporated into an ADC as a quaternized drug unit (D⁺):

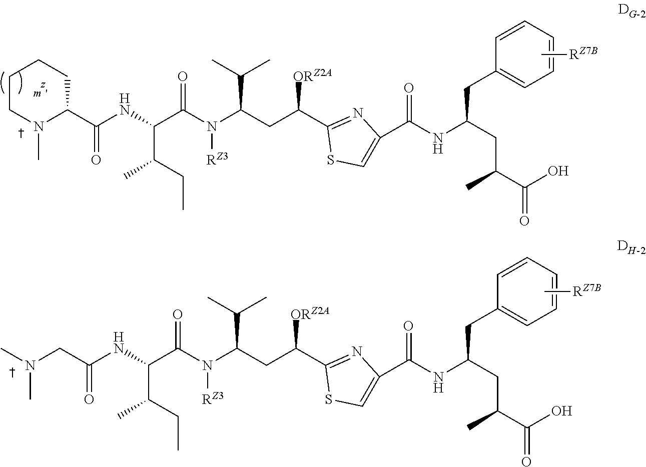

wherein R^{Z2A} along with the oxygen atom to which it is attached defines an O-linked substituent, R^{Z3} is lower alkyl or —CH₂OC(═O)R^{Z3A} wherein R^{Z3A} is optionally substituted lower alkyl, and R^{Z7B} is hydrogen or an O-linked substituent. In some embodiments, R^{Z2A} along with the oxygen atom to which it is attached defines an ester, ether or O-linked carbamate. In some embodiments, R^{Z7B} is an O-linked substituent in the para position. In some embodiments, R^{Z3} is methyl or R^{Z3A} is methyl, ethyl, propyl, iso-propyl, iso-butyl or —CH₂C═(CH₃)₂. In some embodiments R^{Z2A} is methyl, ethyl, propyl (i.e., —OR^{Z2A} is an ether) or is —C(═O)R^{Z2B} (i.e., —OR^{Z2A} is an ester) wherein R^{Z2B} is lower alkyl. In some embodiments, R^{Z2B} is methyl (i.e., —OR^{Z2A} is acetate).

In some embodiments, the tubulysin compound that is incorporated into an antibody-drug conjugate or Drug Linker compound has the structure of one of the following formulae:

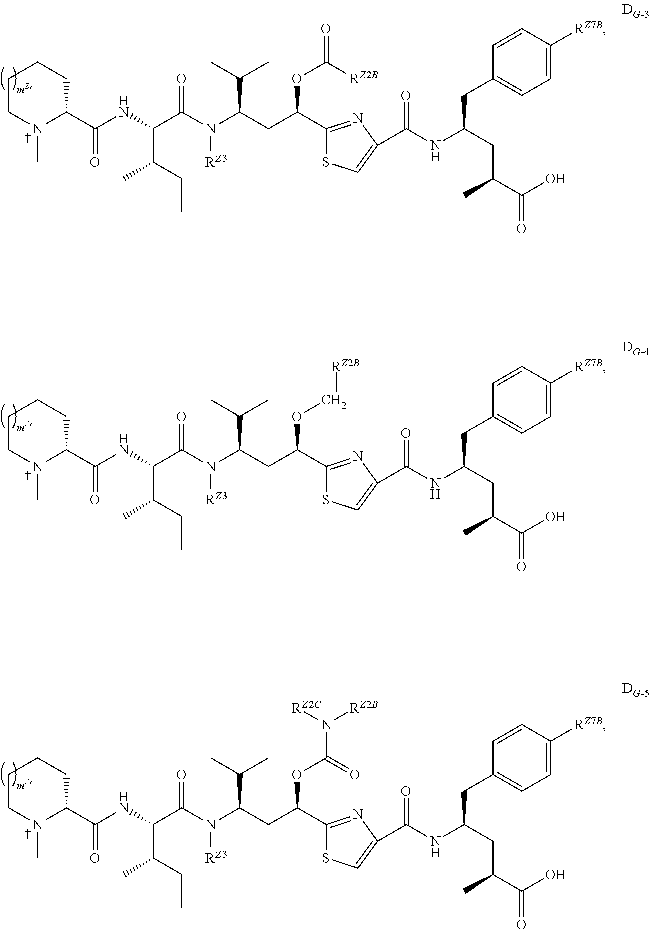

wherein R^{Z7B} is hydrogen or —OH, R^{Z3} is lower alkyl, and R^{Z2B} and R^{Z2}c are independently hydrogen or lower alkyl. In some embodiments, R^{Z3} is methyl or ethyl. In some embodiments of any one of structures D_{G}, D_{G-1}, D_{G-2}, D_{G-3}, D_{G-4}, D_{G-5}, D_{H}, D_{H-1} and D_{H-2}, R^{Z3} is methyl or is —CH₂OC(═O)R^{Z3A}, wherein R^{Z3A} is optionally substituted alkyl. In some embodiments of any one of structures D_{G}′ and D_{H}′, R^{Z3} is methyl or is —CH₂OC(═O)R^{Z3A}, wherein R^{Z3A} is optionally substituted alkyl. In some embodiments of any one of those structures R^{Z3} is —C(R^{Z3A})(R^{Z3A})C(═O)—X^{ZC}, wherein X^{ZC} is —OR^{Z3B} or —N(R^{Z3C})(R^{Z3C}), wherein each R^{Z3A}, R^{Z3B} and R^{Z3C} independently is hydrogen, optionally substituted alkyl or optionally substituted cycloalkyl. In some embodiments, R³ is —C(R^{Z3A})(R^{Z3A})C(═O)—N(R^{Z3C})(R^{Z3C}), with each R^{Z3A} hydrogen, one R^{Z3C} hydrogen and the other R^{Z3C} n-butyl or isopropyl.

In some embodiments of any one of structures D_{G}, D_{G}′, D_{G-1}, D_{G-2}, D_{G-3}, D_{G-4}, D_{G-5}, D_{H}, D_{H}′, D_{H-1} and D_{H-2}, R^{Z3} is ethyl or propyl.

In some embodiments of any one of structures D_{G-1}, D_{G-2}, D_{G-3}, D_{G-4}, D_{G-5}, D_{G-6}, D_{H-1} and D_{H-2}, the thiazole core heterocycle

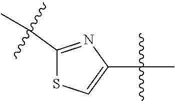 [SMILES: *c1csc(*)n1]

is replaced with

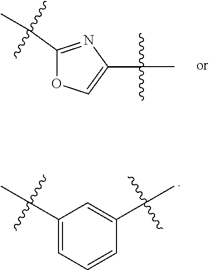 [SMILES: *c1coc(*)n1 ; *c1cccc(*)c1]

In some embodiments of any one of structures D_{G}, D_{G-1}, D_{G-2}, D_{G-3}, D_{G-4}, D_{G-5}, D_{H}, D_{H-1}, D_{H-2}, D_{H-3} and D_{H-4}, R^{Z3} is methyl or is —CH₂OC(═O)R^{Z3A}, wherein R^{Z3A} is optionally substituted alkyl. In some embodiments of any one of those structures R^{Z3} is —C(R^{Z3A})(R^{Z3A})C(═O)—X^{ZC}, wherein X^{ZC} is —OR^{3B} or —N(R^{3C})(R^{3C}), wherein each R^{3A}, R^{3B} and R^{3C} independently is hydrogen, optionally substituted alkyl or optionally substituted cycloalkyl. In some embodiments, R^{Z3} is —C(R^{Z3A})(R^{Z3A})C(═O)—N(R^{Z3C})(R^{Z3C}), with each R^{Z3A} hydrogen, one R^{Z3C} hydrogen and the other R^{Z3C} is optionally substituted alkyl or optionally substituted cycloalkyl. In some embodiments, R^{Z3} is —C(R^{Z3A})(R^{Z3A})C(═O)—N(R^{Z3C})(R^{Z3C}), with each R^{Z3A} hydrogen, one R^{Z3C} hydrogen and the other R^{Z3C} is n-butyl or isopropyl.

In some embodiments of any one of structures D_{G-3}, D_{G-4}, D_{G-5}, D_{H-3} and D_{H-4}, the thiazole core heterocycle

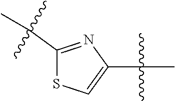 [SMILES: *c1csc(*)n1]

is replaced with

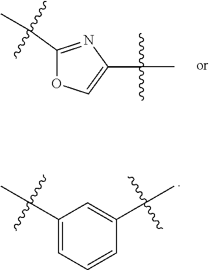 [SMILES: *c1coc(*)n1 ; *c1cccc(*)c1]

In some embodiments, the tubulysin has structure D_{G-3} or D_{G-4} wherein m is 1, R^{Z3} is optionally substituted methyl, ethyl or propyl. In some embodiments, R^{Z3} is unsubstituted methyl, ethyl or propyl.

In some embodiments, the tubulysin compound has structure D_{G-3}, wherein subscript mz′ is 1, R^{Z3} is methyl, ethyl or propyl, —OC(O)R^{Z2B} is —O—C(O)H, O—C(O)—C₁-C₆ alkyl, or —OC₂-C₆ alkenyl, optionally substituted. In some embodiments, —OC(O)R^{Z2B} is —OC(O)CH₃, —OC(O)CH₂CH₃, —OC(O)CH(CH₃)₂, —OC(O)C(CH₃)₃, or —OC(O)CH═CH₂.

In some embodiments, the tubulysin compound has structure D_{G-4}, wherein subscript mz′ is 1, R^{Z3} is methyl, ethyl or propyl and —OCH₂R^{Z2B} is —OCH₃, —OCH₂CH₃, —OCH₂CH₂CH₃ or —OCH₂OCH₃.

In some embodiments, the tubulysin compound has structure D_{G-3}, wherein subscript mz′ is 1, R^{Z3} is methyl, ethyl or propyl, —OC(O)R^{Z2B} is —O—C(O)H, O—C(O)—C₁-C₆ alkyl, or —OC₂-C₆ alkenyl, optionally substituted. In some embodiments, —OC(O)R^{Z2B} is —OC(O)CH₃, —OC(O)CH₂CH₃, —OC(O)CH(CH₃)₂, —OC(O)C(CH₃)₃, or —OC(O)CH═CH₂.

In some embodiments, the tubulysin compound has structure D_{G-4}, wherein subscript mz′ is 1, R^{Z3} is methyl, ethyl or propyl and —OCH₂R^{Z2B} is —OCH₃, —OCH₂CH₃, —OCH₂CH₂CH₃ or —OCH₂OCH₃.

In some embodiments, the tubulysin has the structure of

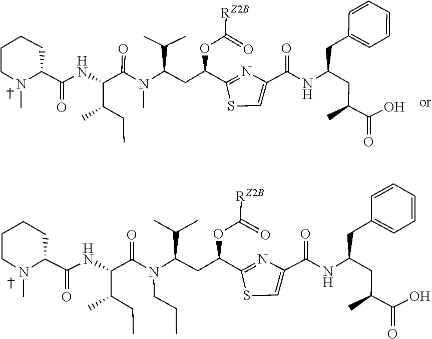

wherein R^{Z2B} is —CH₃, —CH₂CH₃, —CH₂CH₂CH₃, —CH(CH₃)₂, —CH₂CH(CH₃)₂, —CH₂C(CH₃)₃ and the indicated nitrogen atom (†) is the site of quaternization when such compounds are incorporated into an ADC or Drug Linker compound as a quaternized drug unit (D⁺).

In some embodiments, the tubulysin has the structure of

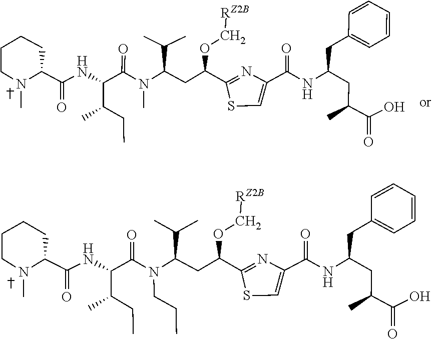

wherein R^{Z2B} is hydrogen, methyl or —OCH₃ (i.e., —OCH₂R^{Z2B} is a methyl ethyl, methoxymethyl ether substituent).

In some embodiments, the tubulysin incorporated as D⁺ in an ADC is a naturally occurring tubulysin including Tubulysin A, Tubulysin B, Tubulysin C, Tubulysin D, Tubulysin E, Tubulysin F, Tubulysin G, Tubulysin H, Tubulysin I, Tubulysin U, Tubulysin V, Tubulysin W, Tubulysin X or Tubulysin Z, whose structures are given by the following structure and variable group definitions wherein the indicated nitrogen atom (†) is the site of quaternization when such compounds are incorporated into an ADC or Drug Linker compound as a quaternized drug unit (D⁺).

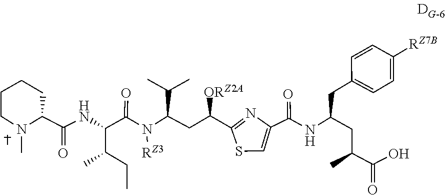

TABLE 1
Some Naturally Occurring Tubulysins

| Tubulysin | R^{Z7B} | R^{Z2A} | R^{Z3} |
|---|---|---|---|
| A | OH | C(═O)CH₃ | CH₂OC═O)i-Bu |
| B | OH | C(═O)CH₃ | CH₂OC═O)n-Pr |
| C | OH | C(═O)CH₃ | CH₂OC═O)Et |
| D | H | C(═O)CH₃ | CH₂OC═O)i-Bu |
| E | H | C(═O)CH₃ | CH₂OC═O)n-Pr |
| F | H | C(═O)CH₃ | CH₂OC═O)Et |
| G | OH | C(═O)CH₃ | CH₂OC═O)CH═CH₂ |
| H | H | C(═O)CH₃ | CH₂OC═O)Me |
| I | OH | C(═O)CH₃ | CH₂OC═O)Me |
| U | H | C(═O)CH₃ | H |
| V | H | OH | H |
| Z | OH | OH | H |

In some embodiments of structure D_{G-6} the tubulysin compound incorporated into an ADC or Drug Linker compound as a quaternized Drug Unit is Tubulysin M, wherein R^{Z3} is —CH₃, R^{Z2} is C(═O)CH₃ and R^{Z7B} is hydrogen.

In some embodiments, D incorporates the structure of a DNA damaging agent. In some embodiments, D incorporates the structure of a DNA replication inhibitor. In some embodiments, D incorporates the structure of acamptothecin. In some embodiments, that camptothecin compound has a formula selected from the group consisting of:

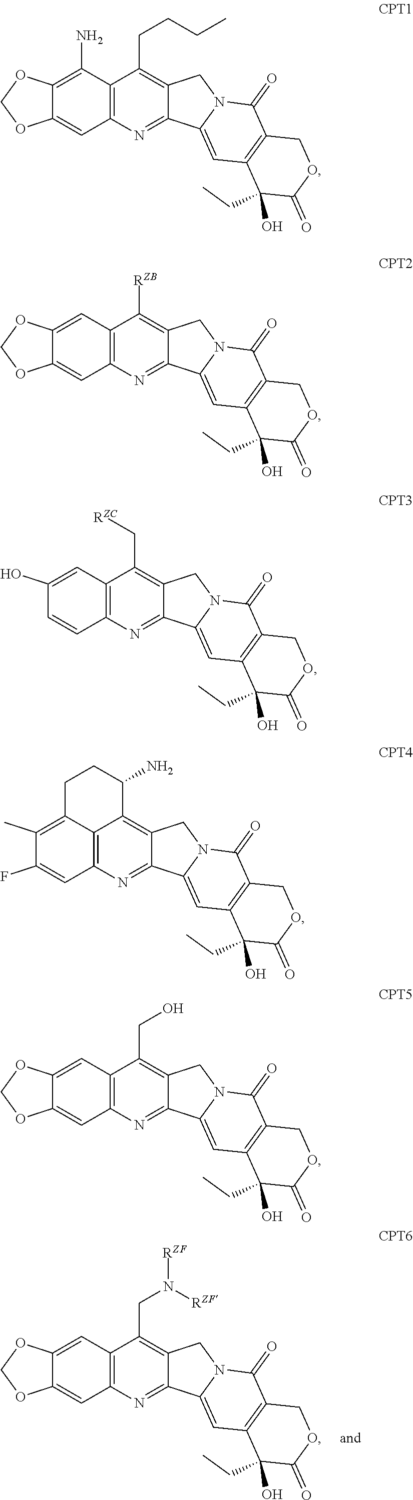

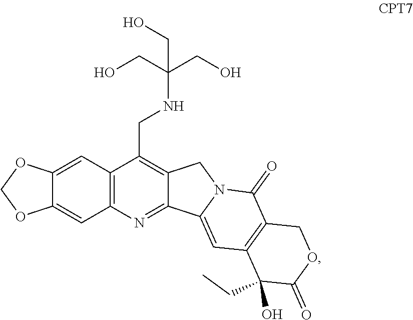

wherein R^{ZB} is selected from the group consisting of H, C₁-C₈ alkyl, C₁-C₈ haloalkyl, C₃-C₈ cycloalkyl, (C₃-C₈ cycloalkyl)-C₁-C₄ alkyl, phenyl, and phenyl-C₁-C₄ alkyl;
R^{ZC} is selected from the group consisting of C₁-C₆ alkyl and C₃-C₆ cycloalkyl; and
each R^{ZF} and R^{ZF′} is independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, or

R^{ZF} and R^{ZF′} are combined with the nitrogen atom to which each is attached to form a 5-, 6- or 7-membered ring having 0 to 3 substituents selected from the group consisting of halogen, C₁-C₄ alkyl, —OH, —OC₁-C₄ alkyl, —NH₂, —NH—C₁-C₄ alkyl, —N(C₁-C₄ alkyl)₂; and

wherein the cycloalkyl, heterocycloalkyl, phenyl and heteroaryl portions of R^{ZB}, R^{ZC}, R^{ZF} and R^{ZF′} are substituted with from 0 to 3 substituents selected from the group consisting of halogen, C₁-C₄ alkyl, —OH, —OC₁-C₄ alkyl, —NH₂, —NHC₁-C₄ alkyl, and —N(C₁-C₄ alkyl)₂.

In some embodiments, the camptothecin compound, whose structure is incorporated as a Drug Unit in an ADC or Drug Linker compound, has the formula CPT1, the structure of which is:

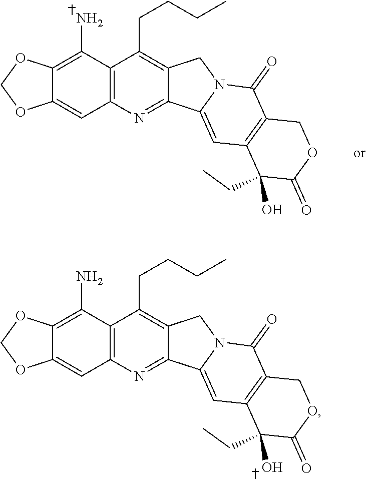

wherein the dagger represents the point of attachment of the Drug Unit to the Linker Unit in a Drug Linker compound or antibody-drug conjugate.

In some embodiments, the camptothecin compound, whose structure is incorporated as a Drug Unit in an ADC or Drug Linker compound, has the formula CPT2, the structure of which is:

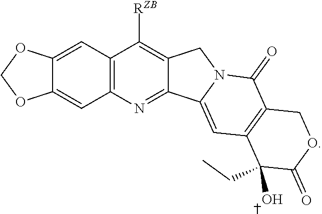

wherein the dagger represents the point of attachment of the Drug Unit to the Linker Unit in a Drug Linker compound or antibody-drug conjugate.

In some embodiments, the camptothecin compound, whose structure is incorporated as a Drug Unit in an ADC or Drug Linker compound, has the formula CPT3, the structure of which is:

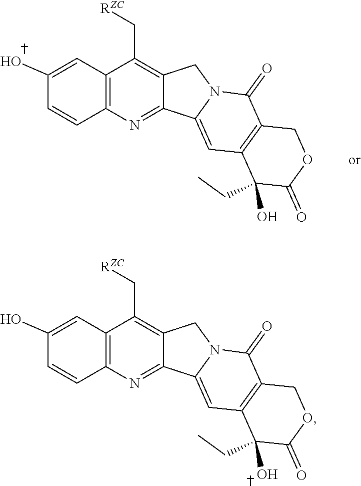

wherein the dagger represents the point of attachment of the Drug Unit to the Linker Unit in a Drug Linker compound or antibody-drug conjugate.

In some embodiments, the camptothecin compound, whose structure is incorporated as a Drug Unit in an ADC or Drug Linker compound, has the formula CPT4, the structure of which is:

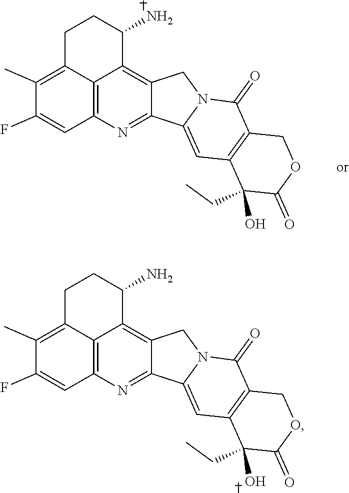 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](N)CC3]

wherein the dagger represents the point of covalent attachment of the Drug Unit to the Linker Unit when the formula CPT4 compound is in the form of a Drug Unit in a Drug Linker compound or antibody-drug conjugate. In some embodiments, D incorporates the structure of exatecan.

In some embodiments, the camptothecin compound, whose structure is incorporated as a Drug Unit in an ADC or Drug Linker compound, has the formula CPT5, the structure of which is:

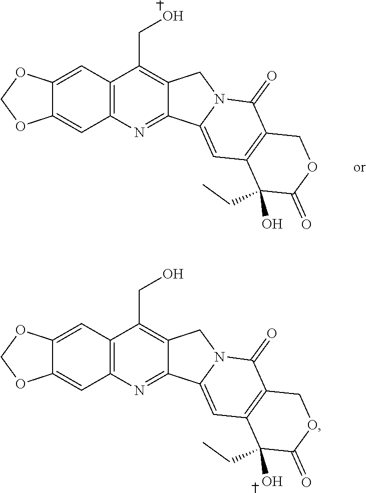

wherein the dagger represents the point of attachment to the Linker Unit when the formula CPT5 compound is in the form of a Drug Unit in a Drug Linker compound or antibody-drug conjugate.

In some embodiments, the camptothecin compound, whose structure is incorporated as a Drug Unit in an ADC or Drug Linker compound, has the formula CPT6, the structure of which is:

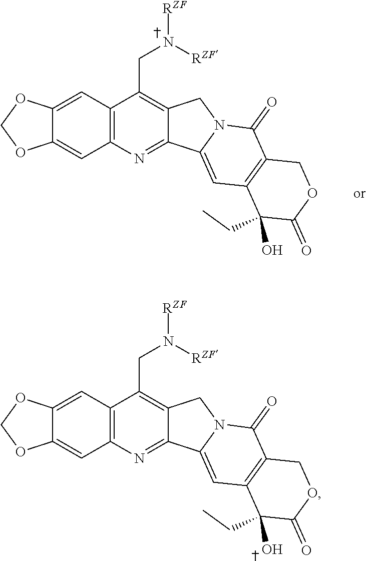

wherein the dagger represents the point of attachment to the Linker Unit when the formula CPT6 compound is in the form of a Drug Unit in a Drug Linker compound or antibody-drug conjugate.

In some embodiments, CPT6 has the structure of:

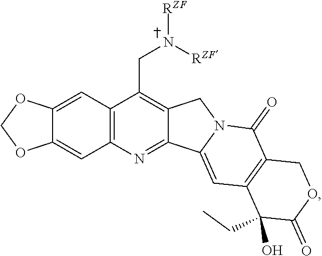

wherein the dagger represents the point of attachment to the Linker Unit when the formula CPT6 compound is in the form of a Drug Unit in a Drug Linker compound or antibody-drug conjugate.

In some embodiments, the camptothecin compound, whose structure is incorporated as a Drug Unit in an ADC or Drug Linker compound, has the formula CPT7 the structure of which is:

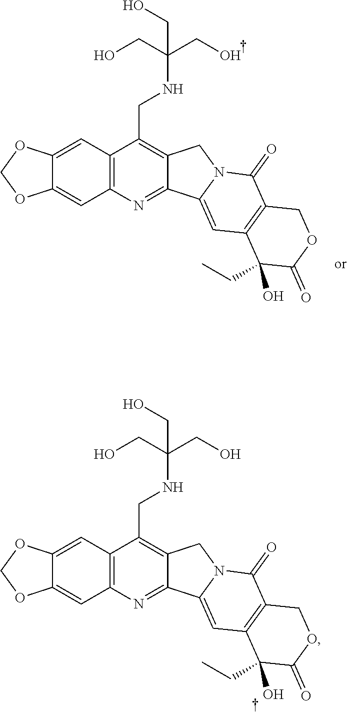

wherein the dagger represents the point of attachment to the Linker Unit in a Drug Linker compound or antibody-drug conjugate when the formula CPT7 compound is in the form of a Drug Unit.

In some embodiments, the camptothecin compound, whose structure is incorporated as a Drug Unit in an ADC or Drug Linker compound, has the formula

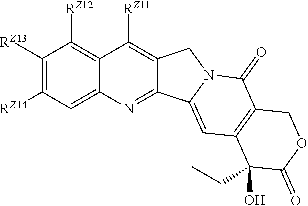

wherein one of R^{Z11} is n-butyl and one of R^{Z12}—R^{Z14} is —NH₂′ and the other are hydrogen, or R^{Z12} is —NH₂ and R^{Z13} and R^{Z14} together are —OCHO—.

In some embodiments, R^{ZB} is selected from the group consisting of C₃-C₈ cycloalkyl, (C₃-C₈ cycloalkyl)-C₁-C₄ alkyl, phenyl, and phenyl-C₁-C₄ alkyl, and wherein the cycloalkyl and phenyl portions of R^{ZB} are substituted with from 0 to 3 substituents selected from halogen, C₁-C₄ alkyl, OH, —O—C₁-C₄ alkyl, NH₂, —NH—C₁-C₄ alkyl and —N(C₁-C₄ alkyl)₂. In some embodiments, R^{ZB} is selected from the group consisting of H, C₁-C₈ alkyl, and C₁-C₈ haloalkyl. In some embodiments, R^{ZB} is H, methyl, ethyl, propyl, isopropyl, butyl, isobutyl, sec-butyl, tert-butyl, pentyl, isopentyl, 1-ethylpropyl, or hexyl. In some embodiments, R^{ZB} is chloromethyl or bromomethyl. In some embodiments, R^{ZB} is phenyl or halo-substituted phenyl. In some embodiments, R^{ZB} is phenyl or fluorophenyl.

In some embodiments, R^{ZC} is C₁-C₆ alkyl. In some embodiments, R^{ZC} is methyl. In some embodiments, R^{ZC} is C₃-C₆ cycloalkyl.

In some embodiments, R^{ZF} and R^{ZF′} are both H. In some embodiments, at least one of R^{ZF} and R^{ZF′} is selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-. In some embodiments, one of R^{ZF} and R^{ZF′} is H and the other is selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-. In some embodiments, one of R^{ZF} and R^{ZF′} is selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-, and the other is selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-. In some embodiments, R^{ZF} and R^{ZF′} are both independently selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydroxyalkyl-C(O)—, C₁-C₈ aminoalkyl-C(O)—, C₃-C₁₀ cycloalkyl, (C₃—C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-.

In some embodiments, the cycloalkyl, heterocycloalkyl, phenyl and heteroaryl moieties of R^{ZF} or R^{ZF′} are substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂.

In some embodiments, R^{ZF} and R^{ZF′} are combined with the nitrogen atom to which each is attached to form a 5-, 6- or 7-membered ring having 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂.

In some embodiments, D incorporates the structure of AMDCPT:

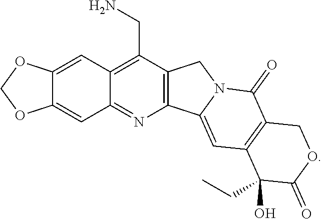 [SMILES: CC[C@@]1(O)C(=O)OCc2c1cc1n(c2=O)Cc2c-1nc1cc3c(cc1c2CN)OCO3]

In some embodiments, D incorporates the structure of exatecan:

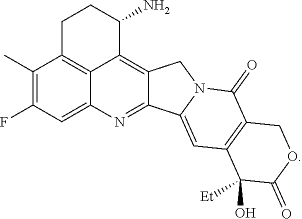 [SMILES: CC[C@@]1(O)C(=O)OCc2c1cc1n(c2=O)Cc2c-1nc1cc(F)c(C)c3c1c2[C@@H](N)CC3]

In some embodiments, D incorporates the structure of irinotecan:

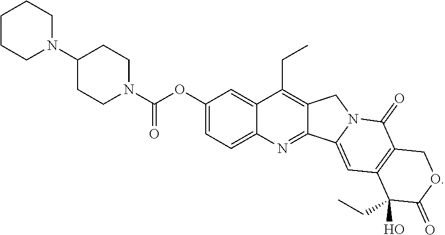 [SMILES: CCc1c2c(nc3ccc(OC(=O)N4CCC(N5CCCCC5)CC4)cc13)-c1cc3c(c(=O)n1C2)COC(=O)[C@]3(O)CC]

In some embodiments, a camptothecin Drug Unit of an antibody-drug conjugate or Drug Linker compound incorporates a camptothecin drug through covalent attachment of a Linker Unit of the Conjugate or Drug Linker compound to an amine or hydroxyl of a camptothecin free drug having structure of D₁ₐ or D_{1b} as follows:

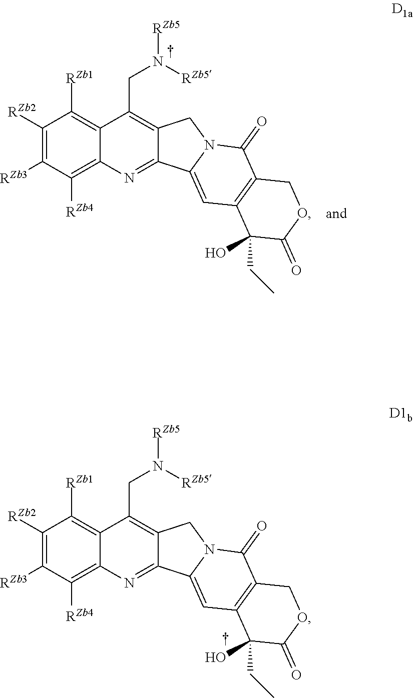

or a salt thereof, wherein the dagger indicates the site of covalent attachment of D to the drug linker moiety,

R^{Zb1} is selected from the group consisting of H, halogen, C₁-C₆ alkyl, C₁-C₆ haloalkyl, C₁-C₆ alkenyl, (C₆-C₁₂ aryl)-C₁-C₆ alkenyl- optionally substituted with —OR^{Za}, —OR^{Za}, —NHR^{Za} and —SR^{Za}, or is combined with R^{b2} or R^{Z5} and the intervening atoms to form a 5- or 6-membered carbocyclo or heterocyclo;

R^{Zb2} is selected from the group consisting of H, halogen, C₁-C₆ alkyl, C₁-C₆ haloalkyl, —OR^{Za}, —NH^{Za} and —SR^{Za}, or is combined with R^{Zb1} or R^{Zb3} and the intervening atoms to form a 5- or 6-membered carbocyclo or heterocyclo;

R^{Zb3} is selected from the group consisting of H, halogen, C₁-C₆ alkyl, C₁-C₆ haloalkyl, —OR^{Za}, —NR^{Za} and —SR^{Za}, or is combined with R^{b2} or R^{Z4} and the intervening atoms to form a 5- or 6-membered carbocyclo or heterocyclo;

R^{Zb4} is selected from the group consisting of H or halogen, or is combined with R^{Zb3} and the intervening atoms to form a 5- or 6-membered carbocyclo or heterocyclo;

each R^{Zb5} and R^{Zb5′} is independently selected from the group consisting of H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydroxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl, and heteroaryl-C₁-C₄ alkyl-, C₁-C₆ alkoxy-C(O)—C₁-C₈ aminoalkyl-, C₁-C₆ alkoxy-C(O)—N—(C₁-C₄ alkyl)amino-C₁-C₈ alkyl-, C₁-C₆ alkoxy-C(O)—(C₃-C₁₀ heterocycloalkyl)-, C₁-C₆ alkoxy-C(O)—(C₃-C₁₀ heterocycloalkyl)-C₁-C₈ alkyl-, C₁-C₄ alkyl-SO₂—C₁-C₈ alkyl-, NH₂—SO₂—C₁-C₈ alkyl-, (C₃-C₁₀ heterocycloalkyl)-C₁-C₄ hydroxyalkyl-, C₁-C₆ alkoxy-C(O)—(C₃-C₁₀ heterocycloalkyl)-C₁-C₈ alkyl-, phenyl-C(O)—, phenyl-SO₂—, and C₁-C₈ hydroxyalkyl-C₃-C₁₀ heterocycloalkyl-, or

R^{Zb5} and R^{Zb5′} are combined with the nitrogen atom to which they are attached to form a 5-, 6- or 7-membered ring having 0 to 3 substituents independently selected from the group consisting of halogen, C₁-C₄ alkyl, —OH, —OC₁-C₄ alkyl, —NH₂, —NH—C₁-C₄ alkyl, —N(C₁-C₄ alkyl)₂, C₁-C₆ alkoxy-C(O)—NH—, C₁-C₆ alkoxy-C(O)—C₁-C₈ aminoalkyl-, and C₁-C₈ aminoalkyl; or

R^{Zb5′} is H and R^{Zb5} is combined with R^{Zb1} and the intervening atoms to form a 5- or 6-membered carbocyclo or heterocyclo;

wherein the cycloalkyl, carbocyclo, heterocycloalkyl, heterocyclo, phenyl and heteroaryl portions of R^{Zb1}, R^{Zb2}, R^{Zb3}, R^{Zb4}, R^{Zb5} and R^{Zb5′} are substituted with from 0 to 3 substituents independently selected from the group consisting of halogen, C₁-C₄ alkyl, —OH, —OC₁-C₄ alkyl, —NH₂, —NHC₁-C₄ alkyl, and —N(C₁-C₄ alkyl)₂; and

each R^{Za} is independently selected from the group consisting of H, C₁-C₆ alkyl, and C₁-C₆ haloalkyl.

In some embodiments of Formula D₁ₐ or Formula D_{1b}, R^{Zb1}, R^{Zb2}, R^{Zb3}, and R^{Zb4} are each hydrogen.

In some embodiments of Formula D₁ₐ or Formula D_{1b}, R^{Zb1}, R^{Zb2}, and R^{Zb4} are hydrogen, and R^{Z3} is halogen. In some embodiments, R^{b3} is fluoro.

In some embodiments of Formula D₁ₐ or Formula D_{1b}, R^{Zb2}, R^{Zb3}, and R^{Zb4} are hydrogen, and R^{Z3} is halogen. In some embodiments, R^{Zb1} is fluoro.

In some embodiments of Formula D₁ₐ or Formula D_{1b}, R^{Zb2} and R^{Zb4} are hydrogen, and R^{Zb1} and R^{Zb3} are both halogen. In some embodiments, R^{Zb1} and R^{Zb3} are both fluoro. In some embodiments of Formula D₁ₐ or Formula D_{1b}, R^{Zb1}, R^{Zb3} and R^{Zb4} are hydrogen, and R^{Zb2} is C₁-C₆ alkyl, C₁-C₆ haloalkyl, halogen, —OR^{Za} or —SR^{Za}. In some embodiments, R^{Zb2} is C₁-C₆ alkyl or halogen. In some embodiments, R^{b2} is C₁-C₆ alkyl. In some embodiments, R^{Zb2} is methyl. In some embodiments, R^{Zb2} is C₁-C₆ alkoxy. In some embodiments, R^{Zb2} is methoxy. In some embodiments, R^{Zb2} is halogen. In some embodiments, R^{Zb2} is fluoro. In some embodiments, R^{Zb2} is chloro. In some embodiments, R^{Zb2} is bromo. In some embodiments, R^{Zb2} is C₁-C₆ haloalkyl. In some embodiments, R^{Zb2} is trifluoromethyl. In some embodiments, R^{Zb2} is C₁-C₆ haloalkylthio. In some embodiments, R^{Zb2} is trifluoromethylthio. In some embodiments, R^{Zb2} is hydroxyl.

In some embodiments of Formula D₁ₐ or Formula D_{1b}, R^{Zb1} and R^{Zb4} are hydrogen, R^{Zb2} is C₁-C₆ alkyl, C₁-C₆ haloalkyl, halogen, —OR^{Za} or —SR^{Za}; and R^{Zb3} is C₁-C₆ alkyl or halogen. In some embodiments, R^{Zb2} is C₁-C₆ alkyl, C₁-C₆ alkoxy, halogen or hydroxy, and R^{Zb3} is C₁-C₆ alkyl or halogen. In some embodiments, R^{Zb2} is C₁-C₆ alkyl. In some embodiments, R^{Zb2} is methyl. In some embodiments, R^{Zb2} is C₁-C₆ alkoxy. In some embodiments, R^{b2} is halogen. In some embodiments, R^{Zb2} is fluoro. In some embodiments, R^{Zb2} is methoxy. In some embodiments, R^{Zb2} is hydroxyl. In some embodiments, R^{Zb3} is C₁-C₆ alkyl. In some embodiments, R^{Zb3} is methyl. In some embodiments, R^{Zb3} is halogen. In some embodiments, R^{Zb3} is fluoro. In some embodiments, R^{Zb2} is C₁-C₆ alkyl and R^{Zb3} is halogen. In some embodiments, R^{Zb2} is methyl and R^{Zb3} is fluoro. In some embodiments, R^{b2} is C₁-C₆ alkoxy and R^{Zb3} is halogen. In some embodiments, R^{Zb2} is methoxy and R^{Zb3} is fluoro. In some embodiments, R^{b2} and R^{Zb3} are halogen. In some embodiments, R^{Zb2} and R^{Zb3} are both fluoro. In some embodiments, R^{Zb2} is halogen and R^{Zb3} is C₁-C₆ alkyl. In some embodiments, R^{Zb2} is fluoro and R^{Zb3} is methyl. In some embodiments, R^{Zb2} is hydroxyl and R^{Zb3} is halogen. In some embodiments, R^{Zb2} is hydroxyl and R^{Zb3} is fluoro.

In some embodiments of Formula D₁ₐ or Formula D_{1b}, R^{Zb2} is C₁-C₆ alkyl, C₁-C₆ haloalkyl, halogen, —OR^{Za} or —SR^{Za}; both R^{Zb1} and R^{Zb3} are independently selected from the group consisting of C₁-C₆ alkyl, halogen, C₁-C₆ alkenyl, (C₆-C₁₂ aryl)-C₁-C₆ alkenyl- optionally substituted with —OR^{Za}, or —OR^{Za}; and R^{Zb4} is hydrogen. In some embodiments, R^{Zb1} is C₁-C₆ alkyl. In some embodiments, R^{Zb1} is methyl. In some embodiments, R^{Zb1} is halogen. In some embodiments, R^{Zb1} is fluoro. In some embodiments, R^{Zb1} is chloro. In some embodiments, R^{Zb1} is bromo. In some embodiments, R^{Zb1} is (C₆-C₁₂ aryl)-C₁-C₆ alkenyl-, optionally substituted with —OR^{Za}. In some embodiments, R^{Zb1} is 4-methoxystyryl. In some embodiments, R^{Zb1} is C₁-C₆ alkenyl. In some embodiments, R^{Zb1} is vinyl. In some embodiments, R^{Zb1} is 1-methylvinyl. In some embodiments, R^{Zb1} is 1-methylvinyl. In some embodiments, R² is C₁-C₆ alkyl. In some embodiments, R^{Zb2} is methyl. In some embodiments, R^{Zb2} is C₁-C₆ alkoxy. In some embodiments, R^{Zb2} is methoxy. In some embodiments, R^{Zb2} is hydroxyl. In some embodiments, R^{Zb3} is C₁-C₆ alkyl. In some embodiments, R^{Zb3} is methyl. In some embodiments, R^{Zb3} is ethyl. In some embodiments, R^{Zb3} is C₁-C₆ alkoxy. In some embodiments, R^{Zb3} is methoxy. In some embodiments, R^{Zb3} is halogen. In some embodiments, R^{Zb3} is fluoro. In some embodiments, R^{Zb3} is chloro. In some embodiments, R^{Zb3} is bromo. In some embodiments, R² is C₁-C₆ alkyl and R^{Zb1} and R^{Zb3} are halogen. In some embodiments, R^{Zb2} is methyl and R^{Zb1} and R^{Zb3} are both fluoro. In some embodiments, R^{Zb2} is methyl, R^{Zb1} is fluoro and R^{Zb3} is bromo. In some embodiments, R^{Zb2} is methyl, R^{Zb1} is bromo and R^{Zb3} is fluoro. In some embodiments, R^{Zb2} is methyl, R^{Zb1} is chloro and R^{Zb3} is fluoro. In some embodiments, R^{Zb2} is methyl, R^{Zb1} is fluoro and R^{Zb3} is chloro. In some embodiments, R^{Zb2} is C₁-C₆ alkoxy and R^{Zb1} and R^{Zb3} is halogen. In some embodiments, R^{Zb2} is methoxy and R^{Zb1} and R^{b3} are both fluoro. In some embodiments, R^{Zb2} is methoxy, R^{Zb1} is bromo and R^{Zb3} is fluoro. In some embodiments, R^{Zb2} is methoxy, R^{Zb1} is fluoro and R^{Zb3} is bromo. In some embodiments, R^{Zb2} is hydroxyl and R^{Zb1} and R^{Zb3} are halogen. In some embodiments, R^{Zb2} is hydroxyl and R^{Zb1} and R^{b3} are both fluoro. In some embodiments, R^{Zb1} is halogen and R^{Zb2} and R^{Zb3} are both C₁-C₆ alkyl. In some embodiments, R^{Zb1} is fluoro and R^{Zb2} and R^{Zb3} are both methyl. In some embodiments, R^{Zb1} is fluoro, R^{Zb2} is methyl and R^{Zb3} is ethyl. In some embodiments, R^{Zb1} and R^{Zb2} are both C₁-C₆ alkyl and R^{Zb3} is halogen. In some embodiments, R^{Zb1} and R^{Zb2} are both methyl and R^{Zb3} is fluoro.

In some embodiments of Formula D₁ₐ or Formula D_{1b}, R^{Zb1} is combined with R^{Zb2} and the intervening atoms to form a 5- or 6-membered carbocyclo or heterocyclo ring. In some embodiments, the drug has the structure of Formula D_{1a/b}-I, Formula D_{1a/b}-II, or Formula D_{1a/b}-III as follows:

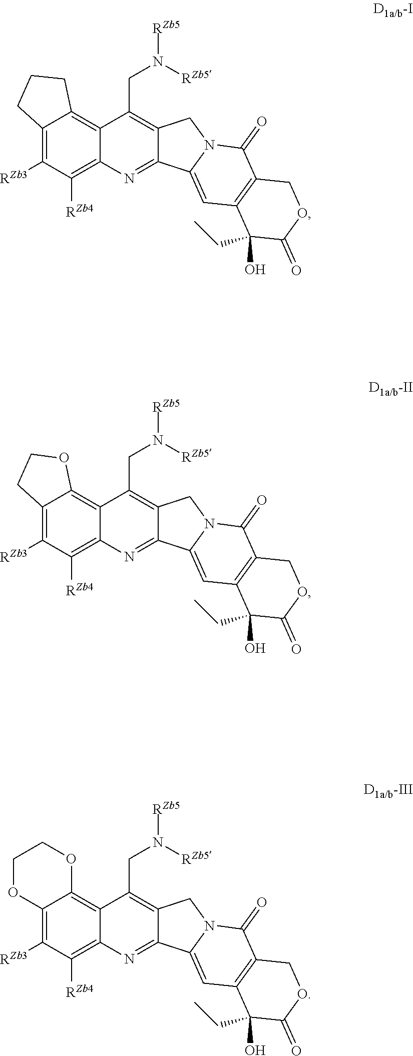

In some embodiments of Formula D₁ₐ or Formula D_{1b}, R^{Zb2} is combined with R^{Zb3} and the intervening atoms to form a 5- or 6-membered carbocyclo or heterocyclo ring; wherein one or more hydrogens are optionally replaced with deuterium. In some embodiments, the drug has the structure of Formula D_{1a/b}-IV, D_{1a/b}-V, D_{1a/b}-VI, D_{1a/b}-VII, D_{1a/b}-VIII or D_{1a/b}-IX as follows:

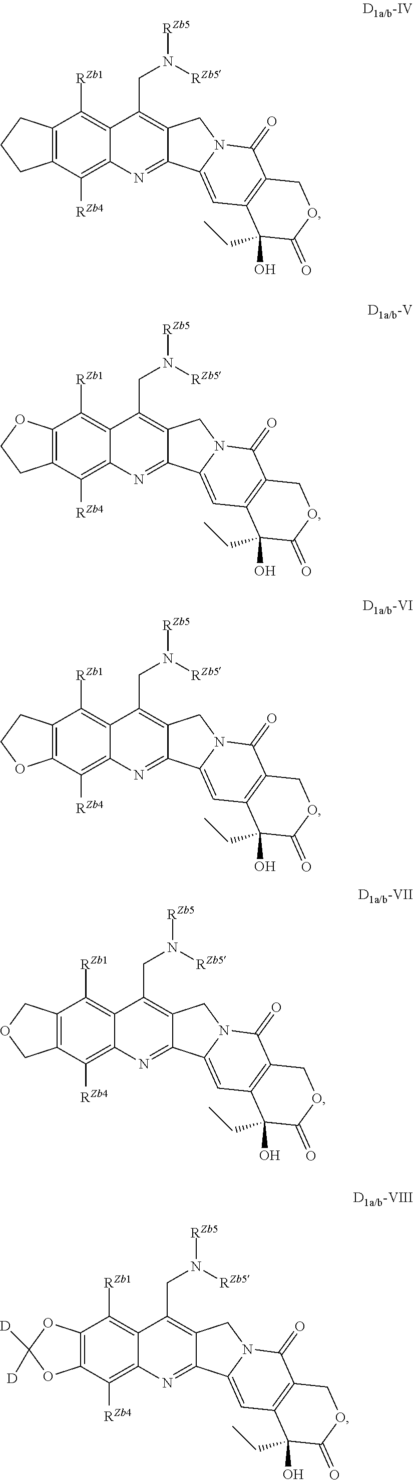

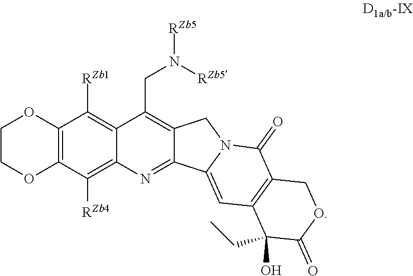

In some embodiments of Formula D₁, R^{Zb5} and R^{Zb5′} are both H. In some embodiments, R^{Zb5} is C₁-C₆ alkyl (e.g., methyl, ethyl) and R^{Zb5′} is H.

In some embodiments of Formula D₁ₐ or Formula D_{1b}, R^{Zb1} is combined with R^{Zb5} and the intervening atoms to form a 5- or 6-membered carbocyclo or heterocyclo ring. In some embodiments, the drug has the structure of Formula D_{1a/b}-X as follows:

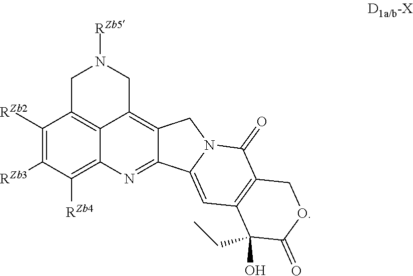

In some embodiments, D incorporates the structure of a DNA minor groove binder. In some embodiments, D incorporates the structure of a pyrrolobenzodiazepine (PBD) compound with the following structure:

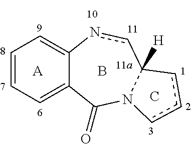

In some embodiments, D is a PBD Drug Unit that incorporates a Drug PBD dimer that is a DNA minor groove binder and has the general structure of Formula X:

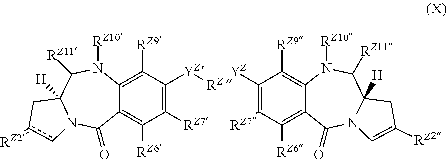

or a salt thereof, wherein: the dotted lines represent a tautomeric double bond; R^{Z2″} is of formula XI:

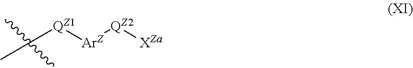

wherein the wavy line indicates the site of covalent attachment to the remainder of the Formula X structure; Ar^{Z} is an optionally substituted C₅₋₇ arylene; X^{Za} is from a reactive or activatable group for conjugation to a Linker Unit, wherein X^{Za} is selected from the group comprising: —O—, —S—, —C(O)O—, —C(O)—, —NHC(O)—, and —N(R^{ZN})—, wherein R^{ZN} is H or C₁-C₄ alkyl, and (C₂H₄O)_{mz}CH₃, where subscript mz is 1, 2 or 3; and either:

Q^{Z1} is a single bond; and Q^{Z2} is a single bond or —Z^{Z}—(CH₂)_{nz}—, wherein Z^{Z} is selected from the group consisting of a single bond, O, S, and NH; and subscript nz is 1, 2 or 3, or (ii) Q^{Z1} is —CH═CH—, and Q^{Z2} is a single bond; and

R^{Z2′} is a optionally substituted C₁-C₄ alkyl or a C₅₋₁₀ aryl group, optionally substituted by one or more substituents selected from the group consisting of halo, nitro, cyano, C₁-C₆ ether, C₁-C₇ alkyl, C₃-C₇ heterocyclyl and bis-oxy-C₁-C₃ alkylene, in particular by one such substituent, wherein the dotted lines indicate a single bond to R^{Z2′}, or R^{Z2′} an optionally substituted C₁-C₄ alkenylene, wherein the dotted lines indicate a double bond to R^{Z2′}; R^{Z6″} and R^{Z9″} are independently selected from the group consisting of H, R^{Z}, OH, OR^{Z}, SH, SR^{Z}, NH, NHR^{Z} NR^{Z}R^{Z′}, nitro, Me₃Sn and halo; R^{Z7″} is selected from the group consisting of H, R^{Z}, OH, OR^{Z}, SH, SR^{Z}, NH₂, NHR^{Z}, NR^{Z}R^{Z′}, nitro, Me₃Sn and halo; and R^{Z} and R^{Z′} are independently selected from the group consisting of optionally substituted C₁-C₁₂ alkyl, optionally substituted C₃-C₂₀ heterocyclyl and optionally substituted C₅-C₂₀ aryl; either:

R^{Z10″} is H, and R^{Z11″} is OH or OR^{ZA}, wherein R^{ZA} is C₁-C₄ alkyl, (b) R^{Z10″} and R^{Z11″} form a nitrogen-carbon double bond between the nitrogen and carbon atoms to which they are bound, or (c) R^{Z10″} is H and R^{Z11″} is SO_{z}M^{Z}, wherein subscript z is 2 or 3 and M^{Z} is a monovalent pharmaceutically acceptable cation, or (d) R^{Z10′}, R^{Z11′} and R^{Z10″} are each H and R^{Z11″} is SO_{z}M^{Z}, or R^{Z10′} and R^{Z11′} are each H and R^{Z10″} and R^{Z11″} form a nitrogen-carbon double bond between the nitrogen and carbon atoms to which they are bound, or R^{Z10″}, R^{Z11″} and R^{Z10′} are each H and R^{Z11′} is SO_{z}M^{Z}, or R^{Z10″} and R^{Z11″} are each H and R^{Z10′} and R^{Z11′} form a nitrogen-carbon double bond between the nitrogen and carbon atoms to which they are bound; wherein subscript z is 2 or 3 and M^{Z} is a monovalent pharmaceutically acceptable cation; and

R^{Z″} is a C₃₋₁₂ alkylene group, the carbon chain of which is optionally interrupted by one or more heteroatoms, in particular by one of O, S or NR^{ZN2} (where R^{ZN2} is H or C₁-C₄ alkyl), and/or by aromatic rings, in particular by one of benzene or pyridine; Y^{Z} and Y^{Z}′ are selected from the group consisting of O, S, and NH; R^{Z6′}, R^{Z7′}, R^{Z9′} are selected from the same groups as R^{Z6″}, R^{Z7″} and R^{Z9″}, respectively, and R^{Z10′} and R^{Z11′} are the same as R^{Z10″} and R^{Z11″}, respectively, wherein if R^{Z11″} and R^{Z11′} are SO_{z}M^{Z}, each M^{Z} is either a monovalent pharmaceutically acceptable cation or together represent a divalent pharmaceutically acceptable cation.

In some embodiments, a PBD Drug Unit that incorporates a PBD dimer that is a DNA minor groove binder has the general structure of Formula XI or XII:

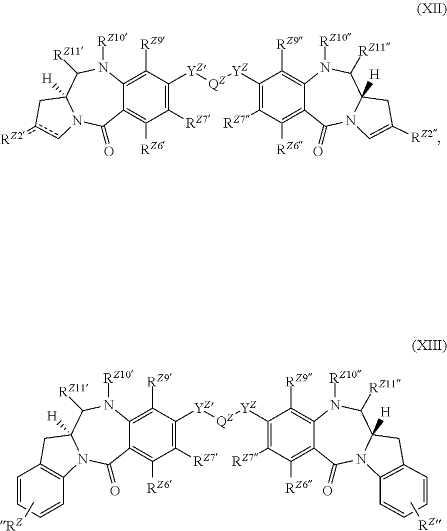

or a salt thereof, wherein: the dotted lines indicate a tautomeric double bond; Q is of formula XIV:

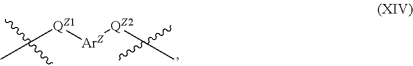

wherein the wavy lines indicate the sites of covalent attachment to Y^{Z}′ and Y^{Z} in either orientation; Ar is a C₅₋₇ arylene group substituted by X^{Za} and is otherwise optionally substituted, wherein X^{Za} is from an activatable group for conjugation to a Linker Unit, wherein X^{Za} is selected from the group comprising: —O—, —S—, —C(O)O—, —C(O)—, —NHC(O)—, and —N(R^{ZN})—, wherein R^{ZN} is H or C₁-C₄ alkyl, and (C₂H₄O)_{mz}CH₃, where subscript m is 1, 2 or 3; and either:

Q^{Z1} is a single bond; and Q^{Z2} is a single bond or —(CH₂)_{nz}—, wherein subscript nz is 1, 2 or 3, or (ii) Q^{Z1} is —CH═CH—, and Q^{Z2} is a single bond or —CH═CH—; and

R^{Z2′} is a optionally substituted C₁-C₄ alkyl or a C₅₋₁₀ aryl group, optionally substituted by one or more substituents selected from the group consisting of halo, nitro, cyano, C₁-C₆ ether, C₁-C₇ alkyl, C₃-C₇ heterocyclyl and bis-oxy-C₁-C₃ alkylene, in particular by one such substituent, wherein the dotted lines indicate a single bond to R^{Z2′}, or R^{Z2′} an optionally substituted C₁-C₄ alkenylene wherein the dotted lines indicate a double bond to R^{Z2′}; and

R^{Z2″} is an optionally substituted C₁-C₄ alkyl or a C₅₋₁₀ aryl group, optionally substituted by one or more substituents selected from the group consisting of halo, nitro, cyano, C₁-C₆ ether, C₁-C₇ alkyl, C₃-C₇ heterocyclyl and bis-oxy-C₁-C₃ alkylene, in particular by one such substituent; R^{Z6″} and R^{Z9″} are independently selected from the group consisting of H, R^{Z}, OH, OR^{Z}, SH, SR^{Z}, NH₂, NHR^{Z}, NR^{Z}R^{Z′}, nitro, Me₃Sn and halo; R^{Z7″} is selected from the group consisting of H, R^{Z}, OH, OR, SH, SR^{Z}, NH₂, NHR^{Z} NR^{Z}R^{Z′}, nitro, Me₃Sn and halo; and R^{Z} and R^{Z′} are independently selected from the group consisting of optionally substituted C₁-C₁₂ alkyl, optionally substituted C₃-C₂₀ heterocyclyl and optionally substituted C₅-C₂₀ aryl; and either:

R^{Z10″} is H, and R^{Z11″} is OH or OR^{ZA}, wherein R^{ZA} is C₁-C₄ alkyl, or (b) R^{Z10″} and R^{Z11″} form a nitrogen-carbon double bond between the nitrogen and carbon atoms to which they are bound, or (c) R^{Z10}, is H and R^{Z11″} is SO_{z}M^{Z}, wherein subscript z is 2 or 3 and M^{Z} is a monovalent pharmaceutically acceptable cation, or (d) R^{Z10′}, R^{Z11′} and R^{Z10″} are each H and R^{Z11″} is SO_{z}M^{Z}, or R^{Z10′} and R^{Z11′} are each H and R^{Z10}, and R^{Z11″} form a nitrogen-carbon double bond between the nitrogen and carbon atoms to which they are bound, or R^{Z10″}, R^{Z11″} and R^{Z10′} are each H and R^{Z11′} is SO_{z}M^{Z}, or R^{Z10″} and R^{Z11″} are each H and R^{Z10′} and R^{Z11′} form a nitrogen-carbon double bond between the nitrogen and carbon atoms to which they are bound; wherein subscript z is 2 or 3 and M^{Z} is a monovalent pharmaceutically acceptable cation; and

Y^{Z} and Y^{Z}′ are selected from the group consisting of O, S, and NH; R^{Z}″=represents one or more optional substituents; and R^{Z6′}, R^{Z7′}, R^{Z9′} are selected from the same groups as R^{Z6″}, R^{Z7″} and R^{Z9″}, respectively, and R^{Z10′} and R^{Z11′} are the same as R^{Z10}, and R^{Z11″}, respectively, wherein if R^{Z11″} and R^{Z11′} are SO_{z}M^{Z}, each M^{Z} is either a monovalent pharmaceutically acceptable cation or together represent a divalent pharmaceutically acceptable cation.

In some embodiments, the PBD dimer has the general structure of Formula X, Formula XII or Formula XIII in which one, R^{Z7″} is selected from the group consisting of H, OH and OR^{Z}, wherein R^{Z} is a previously defined for each of the formula, or is a C₁₋₄ alkyloxy group, in particular R^{Z7″} is —OCH₃. In some embodiments, Y^{Z} and Y^{Z}′ are O, R^{Z9″} is H, or R^{Z6″} is selected from the group consisting of H and halo.

In some embodiments, the PBD dimer has the general structure of Formula X in which Ar^{Z} is phenylene; X^{Za} is selected from the group consisting of —O—, —S— and —NH—; and Q^{Z1} is a single bond, and in some embodiments of Formula XII Ar^{Z} is phenylene, Xz is selected from the group consisting of —O—, —S—, and —NH—, Q^{Z1}-CH₂— and Q^{Z2} is —CH₂—.

In some embodiments, the PBD dimer has the general structure of Formula X in which X^{Za} is NH. In some embodiments, the PBD Drug Units are of Formula X in which Q^{Z1} is a single bond and Q^{Z2} is a single bond.

In some embodiments, the PBD dimer has the general structure of Formula X, Formula XII or Formula XIII in which R^{Z2′} is an optionally substituted C₅₋₇ aryl group so that the dotted lines indicate a single bond to R^{Z2′} and the substituents when present are independently selected from the group consisting of halo, nitro, cyano, C₁₋₇ alkoxy, C₅₋₂₀ aryloxy, C₃₋₂₀ heterocyclyoxy, C₁₋₇ alkyl, C₃₋₇ heterocyclyl and bis-oxy-C₁₋₃ alkylene wherein the C₁₋₇ alkoxy group is optionally substituted by an amino group, and if the C₃₋₇ heterocyclyl group is a C₆ nitrogen containing heterocyclyl group, it is optionally substituted by a C₁₋₄ alkyl group.

In some embodiments, the PBD dimer has the general structure of Formula X, Formula XI or Formula XII in which Ar^{Z} is an optionally substituted phenyl that has one to three such substituents when substituted.

In some embodiments, the PBD dimer has the general structure of Formula X, Formula XI or Formula XII in which R^{Z10}, and R^{Z11″} form a nitrogen-carbon double bond and/or R^{Z6′}, R^{Z7′} R^{Z9′}, and Y^{Z′} are the same as R^{Z6″}, R^{Z7″}, R^{Z9″}, and Y^{Z} respectively.

In some embodiments, the PBD Drug Unit has the structure of:

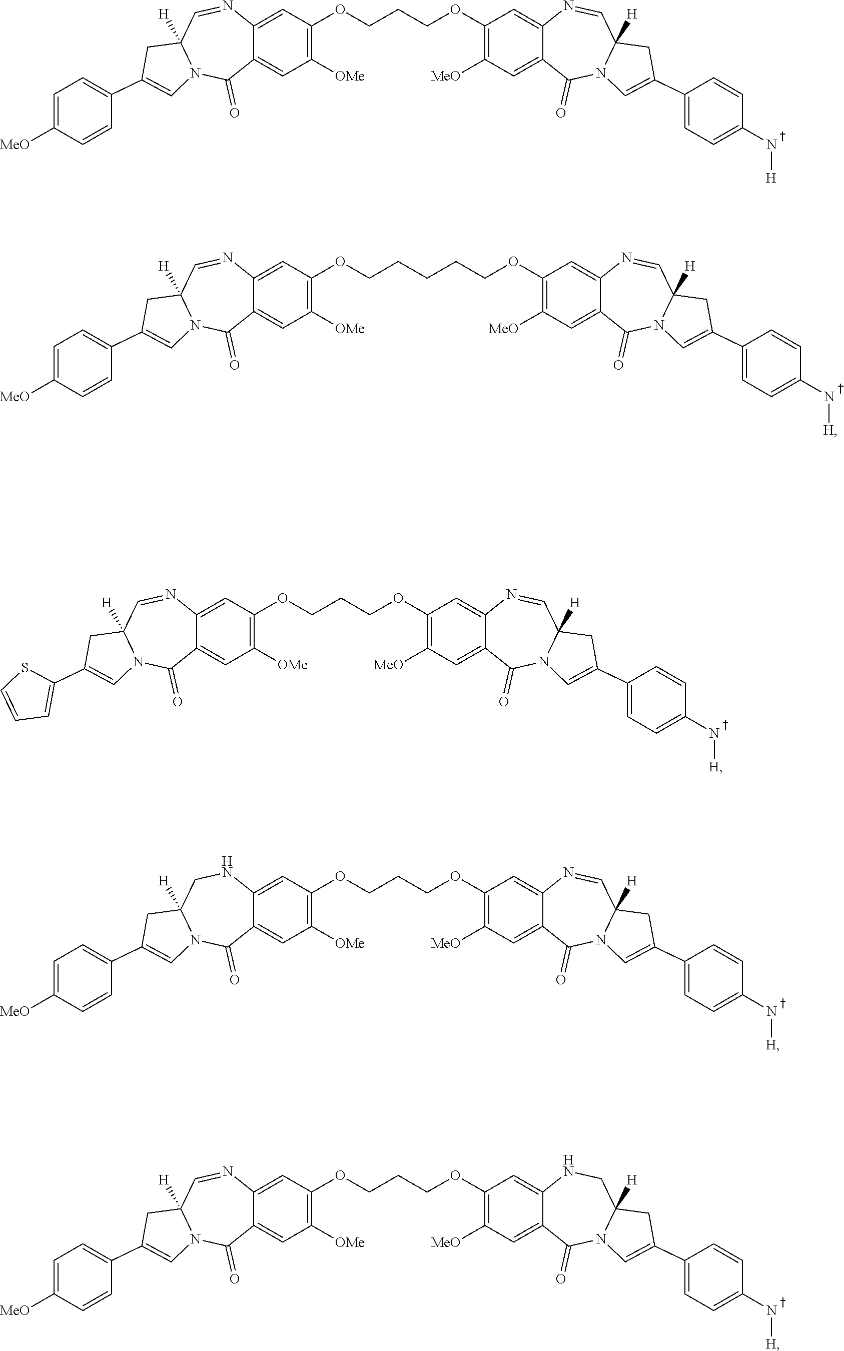

or a salt thereof, wherein the dagger represents the point of attachment of the Drug Unit to the Linker Unit in a Drug Linker compound or antibody-drug conjugate.

In some embodiments, the PBD Drug Unit has the structure of:

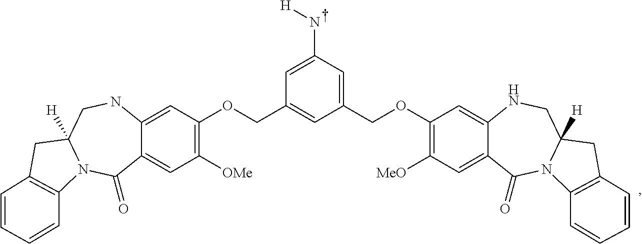

or a salt thereof, wherein the dagger represents the point of attachment of the Drug Unit to the Linker Unit in a Drug Linker compound or antibody-drug conjugate.

In some embodiments, the Drug Unit incorporates the structure of an anthracyclin compound. Without being bound by theory, the cytotoxicity of those compounds to some extent may also be due to topoisomerase inhibition. In some of those embodiments the anthracyclin compound has a structure disclosed in Minotti, G., et al., “Anthracyclins: molecular advances and pharmacologic developments in antitumor activity and cardiotoxicity” Pharmacol Rev. (2004) 56(2): 185-229. In some embodiments, the anthracyclin compound is doxorubicin, idarubicin, daunorubicin, doxorubicin propyloxazoline (DPO), morpholino-doxorubicin, or cyanomorpholino-doxorubicin.

In some embodiments, the Drug Unit (D) is from a cytostatic agent. In some embodiments, D is from a compound having cellular cytostatic activity ranging from 1 to 100 nM. In some embodiments, the Drug Unit (D) is from a cytotoxic agent. In some embodiments, D is from a cytotoxic agent having an IC₅₀ value for cellular cytotoxic activity ranging from 1 to 100 nM. There are several methods for determining whether an ADC exerts a cytostatic or cytotoxic effect on a cell line. In one example for determining whether an ADC exerts a cytostatic or cytotoxic effect on a cell line, a thymidine incorporation assay is used. For example, cells at a density of 5,000 cells/well of a 96-well plated is cultured for a 72-hour period and exposed to 0.5 μCi of ³H-thymidine during the final 8 hours of the 72-hour period, and the incorporation of ³H-thymidine into cells of the culture is measured in the presence and absence of ADC. The ADC has a cytostatic or cytotoxic effect on the cell line if the cells of the culture have reduced ³H-thymidine incorporation compared to cells of the same cell line cultured under the same conditions but not contacted with the ADC.

In another example, for determining whether an ADC exerts a cytostatic or cytotoxic effect on a cell line, cell viability is measured by determining in a cell the uptake of a dye such as neutral red, trypan blue, or ALAMAR™ blue (see, e.g., Page et al., 1993, Intl. J of Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) is useful for measuring cytotoxicity (Skehan et al., 1990, J. Nat'l Cancer Inst. 82:1107-12). Preferred ADCs include those with an IC₅₀ value (defined as the mAB concentration that gives 50% cell kill) of less than 1000 ng/mL, for example, less than 500 ng/mL, less than 100 ng/ml, or less than 50 or even less than 10 ng/mL on the cell line.

In some embodiments, D is from a cytotoxic or cytostatic agent having a cellular potency that would not be expected to provide a sufficiently active ADC in vitro in which the DAR is 8.

In some embodiments, D is from a hydrophilic cytotoxic or cytostatic agent (i.e., D has a c Log P≤1). In some embodiments, D is from a hydrophobic cytotoxic or cytostatic agent (i.e., D has a c Log P>1). In some embodiments, D is from a cytotoxic or cytostatic agent having a c Log P of about −3 to about 3, for example, about −3, about −2.5, about −2, about −1.5, about −1, about −0.5, about 0, about 0.5, about 1, about 1.5, about 2, about 2.5, about 3, or any value in between. In some embodiments, D is from a cytotoxic or cytostatic agent having a c Log P of about −3 to about 1, for example, about −3, about −2.5, about −2, about −1.5, about −1, about −0.5, about 0, about 0.5, about 1, or any value in between. In some embodiments, D is from a cytotoxic or cytostatic agent having a c Log P of about −1 to about 1, for example, about −1, about −0.75, about −0.5, about −0.25, about 0, about 0.25, about 0.5, about 0.75, about 1, or any value in between. In some embodiments, D is from a cytotoxic or cytostatic agent having a c Log P of about 0 to about 1, for example, about 0, about 0.1, about 0.2, about 0.3, about 0.4, about 0.5, about 0.6, about 0.7, about 0.8, about 0.9, about 1, or any value in between. In some embodiments, D is from a cytotoxic or cytostatic agent having a c Log P of about 1 to about 6, for example, about 1, about 1.5, about 2, about 2.5, about 3, about 3.5, about 4, about 4.5, about 5, about 5.5, about 6, or any value in between. In some embodiments, D is from a cytotoxic or cytostatic agent has a c Log P of about 3 to about 6, for example, about 3, about 3.5, about 4, about 4.5, about 5, about 5.5, about 6, or any value in between.

In some embodiments, D is from a cytotoxic or cytostatic agent having a polar surface area of about 80 Å² to about 150 Å², for example, about 80 Å², about 90 Å², about 100 Å², about 110 Å², about 120 Å², about 130 Å², about 140 Å², about 150 Å², or any value in between. In some embodiments, D is from a cytotoxic or cytostatic agent having a polar surface area of about 80 Å² to about 120 Å², for example, about 80 Å², about 90 Å², about 100 Å², about 110 Å², about 120 Å², or any value in between. In some embodiments, D is from a cytotoxic or cytostatic agent having has a polar surface area of about 90 Å² to about 130 Å², for example, about 90 Å², about 100 Å², about 110 Å², about 120 Å², about 130 Å², or any value in between. In some embodiments, D is from a cytotoxic or cytostatic agent having has a polar surface area of about 110 Å² to about 150 Å², for example, about 110 Å², about 120 Å², about 130 Å², about 140 Å², about 150 Å², or any value in between. In some embodiments, D is from a cytotoxic or cytostatic agent having a polar surface area of about 130 Å² to about 150 Å², for example, about 130 Å², about 140 Å², about 150 Å², or any value in between.

In some embodiments, D is from a DNA replication inhibitors such as gemcitabine, or a tubulin disrupting agent such as MMAE, or MMAF. In some embodiments, D is from gemcitabine. In some embodiments, D is from MMAE. In some embodiments, D is form MMAF. In some embodiments, D is from an inhibitor or ATP production such as a NAMPT inhibitor.

In some embodiments, D is from a NAMPT inhibitor having the following formula:

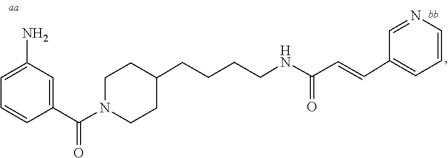

wherein D is covalently attached to L² at the aa or bb nitrogen atom.

Drug-Linker Compounds

In some embodiments, D has an atom that forms a bond with L¹ (when M and L² are both absent), with M (when L² is absent) or with L². In some embodiments, the atom from D forming the bond with L¹, M, or L² is a nitrogen atom. In some embodiments, the atom from D forming the bond with L¹, M, or L² is a nitrogen atom that is quaternized upon forming the bond. In some embodiments, the atom from D forming the bond with L¹, M, or L² is a sulfur atom from a thiol group. In some embodiments, the atom from D forming the bond with L¹, M, or L² is an oxygen atom from a hydroxyl group. In some embodiments, the hydroxyl group is present in the free drug. In some embodiments, the hydroxyl group is produced by reduction of a carbonyl group present in the free drug. In some embodiments, the atom from D forming the bond with L¹, M, or L² is a carbon atom attached to a hydroxyl group that, prior to forming the bond, was a carbonyl group in the free drug. In some embodiments, D forms a bond with L¹, M, or L² via a carboxylic acid group.

In some embodiments, D comprises a functional group that is negatively charged at physiological pH, for example, a carboxylic acid or a phosphate. In some embodiments, D comprises a functional group that is positively charged at physiological pH, for example, an amine. In some embodiments, when D comprises a negatively charged functional group at physiological pH, L¹ (when M and L² are both absent), M (when L² is absent) or L² (when present) comprise a functional group that is positively charged at physiological pH. In some embodiments, when D comprises a positively charged functional group at physiological pH, L¹ (when M and L² are both absent), M (when L² is absent) or L² (when present) comprise a functional group that is negatively charged at physiological pH. In some embodiments, D is uncharged at physiological pH. In some embodiments, D has zero net charge at physiological pH. In some embodiments, when D is uncharged or has zero net charge at physiological pH, L¹ (when M and L² are both absent), M (when L² is absent) or L² (when present) are uncharged or have zero net charge at physiological pH.

In some embodiments, each L²-D is uncharged or has a net zero charge at physiological pH. In some embodiments, each L²-D has no charged species (i.e., is uncharged) at physiological pH. In some embodiments, each L²-D is zwitterionic at physiological pH. In some embodiments, each L²-D comprises a carboxylate and an ammonium-containing moiety. In some embodiments, the ammonium-containing moiety is a quaternary ammonium-containing moiety. In some embodiments, the quaternary ammonium-containing moiety is pyridinium. In some embodiments, L² is anionic; and D is cationic. In some embodiments, L² comprises a carboxylate-containing moiety; and D comprises an ammonium-containing moiety.

In some embodiments, each L¹-(M)ₓ-(D)_{y} (when L² is absent) has no charged species at physiological pH. In some embodiments, each L¹-(M)ₓ-(D)_{y} (when L² is absent) is zwitterionic at physiological pH. In some embodiments, each L¹-(M)ₓ-(D)_{y} (when L² is absent) comprises a carboxylate and an ammonium-containing moiety. In some embodiments, the ammonium-containing moiety is a quaternary ammonium-containing moiety. In some embodiments, the quaternary ammonium moiety is pyridinium. In some embodiments, L¹-(M)ₓ is anionic; and D is cationic. In some embodiments, L¹-(M)ₓ comprises a carboxylate-containing moiety; and D comprises an ammonium-containing moiety.

In some embodiments, each L¹-D (when M and L² are absent) has no charged species at physiological pH. In some embodiments, each L¹-D (when M and L² are absent) is zwitterionic at physiological pH. In some embodiments, each L¹-D (when M and L² are absent) comprises a carboxylate and an ammonium-containing moiety. In some embodiments, the ammonium moiety is a quaternary ammonium moiety. In some embodiments, the quaternary ammonium-containing moiety is pyridinium. In some embodiments, L¹ is anionic; and D is cationic. In some embodiments, L¹ comprises a carboxylate-containing moiety; and D comprises an ammonium-containing moiety.

General procedures for linking a drug to linkers are known in the art. See, for example, U.S. Pat. Nos. 8,163,888, 7,659,241, 7,498,298, U.S. Publication No. US20110256157 and International Application Nos. WO2011023883, and WO2005112919, each of which is incorporated by reference herein, particularly in regards to the aforementioned general procedures.

In some embodiments, D has a charge of +1 at physiological pH; and L² is selected from the group consisting of:

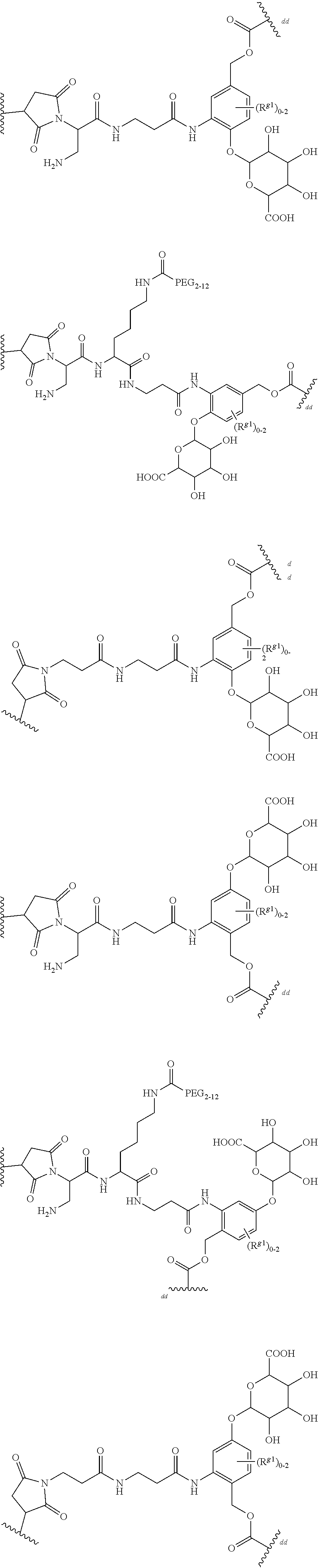

wherein dd is the point of covalent attachment to D; and R^{g1} is halogen, —CN, or —NO₂.

In some embodiments, D is uncharged at physiological pH; and L² is selected from the group consisting of

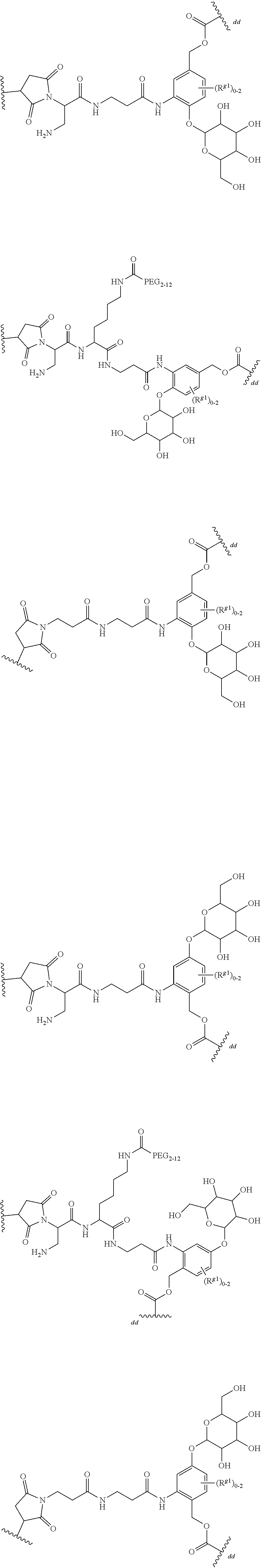

wherein dd is the point of covalent attachment to D; and R^{g1} is halogen, —CN, or —NO₂.

In some embodiments, L² is selected from the group consisting of:

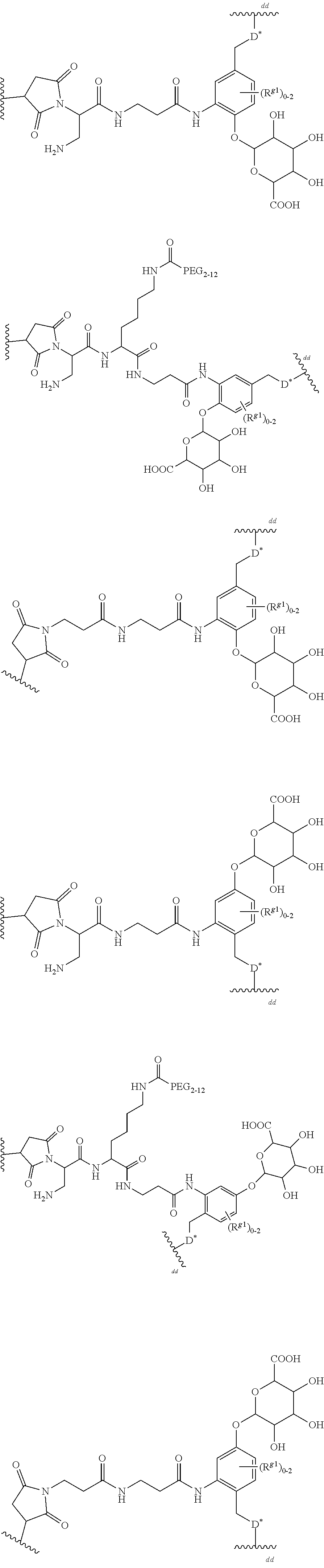

wherein R^{g1} is halogen, —CN, or —NO₂; D* is a cation that is part of the D moiety; dd represents the point of covalent attachment to the rest of D; and D (inclusive of D*) has a charge of +1 at physiological pH.

In some embodiments, D* is pyridinium. For example, D* can be

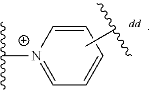

In some other embodiments, D* is

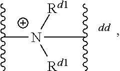

wherein each R^{d1} is independently C₁₋₆ alkyl.

In some embodiments, L² is selected from the group consisting of:

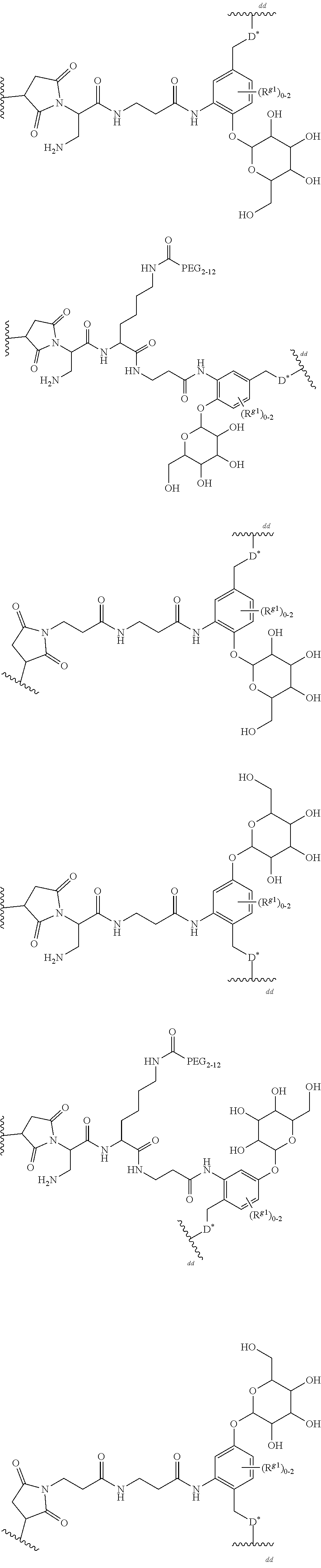

wherein R^{g} is halogen, —CN, or —NO₂; D* is a cation that is part of the D moiety; dd represents point of covalent attachment to the rest of D; and D (inclusive of D*) is zwitterionic at physiological pH.

In some embodiments of the ADCs described herein, the ratio of D to Ab is 8:1 to 64:1. In some embodiments, the ratio of D to Ab is 8:1 to 16:1. In some embodiments, the ratio of D to Ab is 8:1 to 32:1. In some embodiments, the ratio of D to Ab is 16:1 to 64:1. In some embodiments, the ratio of D to Ab is 16:1 to 32:1. In some embodiments, the ratio of D to Ab is 32:1 to 64:1. In some embodiments, the ratio of D to Ab is 8:1. In some embodiments, the ratio of D to Ab is 16:1. In some embodiments, the ratio of D to Ab is 32:1. In some embodiments, the ratio of D to Ab is 64:1.

In some embodiments of the ADCs described herein, the ratio of D to Ab is 8:1; subscript y is 4; and subscript p is 2. In some embodiments, the ratio of D to Ab is 8:1; subscript y is 2; and subscript p is 4. In some embodiments, the ratio of D to Ab is 16:1; subscript y is 8; and subscript p is 2. In some embodiments, the ratio of D to Ab is 16:1; subscript y is 4; and subscript p is 4. In some embodiments, the ratio of D to Ab is 16:1; subscript y is 2; and subscript p is 8.

### Polyethyleneglycol (PEG) Units

Polydisperse PEGs, monodisperse PEGs and discrete PEGs can be used to make the ADCs and intermediates thereof described herein. Polydisperse PEGs are a heterogeneous mixture of sizes and molecular weights whereas monodisperse PEGs are typically purified from heterogeneous mixtures and therefore provide a single chain length and molecular weight. Discrete PEGs are synthesized in step-wise fashion and not via a polymerization process. Discrete PEGs provide a single molecule with defined and specified chain length. The number of —CH₂CH₂O— subunits of a PEG Unit ranges, for example, from 2 to 72, from 8 to 24 or from 12 to 24, referred to as PEG2 to PEG72, PEG8 to PEG24 and PEG12 to PEG24, respectively.

The PEGs provided herein, which are also referred to as PEG Units, comprise one or multiple polyethylene glycol chains. The polyethylene glycol chains are linked together, for example, in a linear, branched, or star shaped configuration. Typically, at least one of the polyethylene glycol chains of a PEG Unit is derivatized at one end for covalent attachment to an appropriate site on a component of the ADC (e.g., L). Exemplary attachments to ADCs are by means of non-conditionally cleavable linkages or via conditionally cleavable linkages. Exemplary attachments are via amide linkage, ether linkages, ester linkages, hydrazone linkages, oxime linkages, disulfide linkages, peptide linkages, or triazole linkages.

Generally, at least one of the polyethylene glycol chains that make up the PEG Unit is functionalized to provide covalent attachment to the ADC. Functionalization of the polyethylene glycol-containing compound that is the precursor to the PEG Unit includes, for example, via an amine, thiol, NHS ester, maleimide, alkyne, azide, carbonyl, or other functional group. In some embodiments, the PEG Unit further comprises non-PEG material (i.e., material not comprised of —CH₂CH₂O—) that provides coupling to the ADC or in constructing the polyethylene glycol-containing compound or PEG facilitates coupling of two or more polyethylene glycol chains.

In some embodiments, attachment to the ADC is by means of a non-conditionally cleavable linkage. In some embodiments, attachment to the ADC is not via an ester linkage, hydrazone linkage, oxime linkage, or disulfide linkage. In some embodiments, attachment to the ADC is not via a hydrazone linkage. If a high DAR ADC having uncharged or net zero charged drug-linker moieties, as described herein, still exhibits one or more unsatisfactory biophysical property(ies), addition of a PEG Unit, may improve these one or more property(ies). For example, a branched PEG Unit as described herein and by WO 2015/057699 (the disclosure of which is incorporated by reference in its entirety).

A conditionally cleavable linkage refers to a linkage that is not substantially sensitive to cleavage while circulating in plasma but is sensitive to cleavage in an intracellular or intratumoral environment. A non-conditionally cleavable linkage is one that is not substantially sensitive to cleavage in any biologically relevant environment in a subject that is administered the ADC. Chemical hydrolysis of a hydrazone, reduction of a disulfide bond, and enzymatic cleavage of a peptide bond or glycosidic bond of a Glucuronide Unit as described herein, and by WO 2007/011968 (the disclosure of which is incorporated by reference in its entirety) are examples of conditionally cleavable linkages.

In some embodiments, the PEG Unit is directly attached to the ADC at L¹, M, and/or L². In some embodiments, the other terminus (or termini) of the PEG Unit is free and untethered (i.e., not covalently attached) and in some embodiments, takes the form of a methoxy, carboxylic acid, alcohol, or other suitable functional group. The methoxy, carboxylic acid, alcohol, or other suitable functional group acts as a cap for the terminal polyethylene glycol subunit of the PEG Unit. By untethered, it is meant that the PEG Unit will not be covalently attached at that untethered site to a Drug Unit, to an antibody, or to a linking component to a Drug Unit and/or an antibody. Such an arrangement permits a PEG Unit of sufficient length to assume a parallel orientation with respect to the drug in conjugated form, i.e., as a Drug Unit (D). Without being bound by theory, that orientation is believed to mask the hydrophobicity of the conjugated drug in those instances in which the free drug has insufficient hydrophilicity, thus facilitating the higher loading provided by multiplexers within drug linker moieties that are uncharged or have net zero charge, as described herein. In some embodiments, each polyethylene glycol chain in a PEG Unit may be independently chosen, e.g., be the same or different chemical moieties (e.g., polyethylene glycol chains of different molecular weight or number of —CH₂CH₂O— subunits). A PEG Unit having multiple polyethylene glycol chains is attached to the ADC at a single attachment site. The skilled artisan will understand that the PEG Unit in addition to comprising repeating polyethylene glycol subunits may also contain non-PEG material (e.g., to facilitate coupling of multiple polyethylene glycol chains to each other or to facilitate coupling to the ADC). Non-PEG material refers to the atoms in the PEG Unit that are not part of the repeating —CH₂CH₂O— subunits. In some embodiments, the PEG Unit comprises two monomeric polyethylene glycol chains attached to each other via non-PEG elements. In other embodiments provided herein, the PEG Unit comprises two linear polyethylene glycol chains attached to a central core that is attached to the ADC (i.e., the PEG Unit itself is branched).

There are a number of PEG attachment methods available to those skilled in the art: for example, Goodson, et al. (1990) Bio Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal.alpha.-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation).

In some embodiments, a PEG Unit may be covalently bound to an amino acid residue via reactive groups of a polyethylene glycol-containing compound and the amino acid residue. Reactive groups of the amino acid residue include those that are reactive to an activated PEG molecule (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for forming a covalent attachment to a PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J Biol. Chem. 269:7224).

In some embodiments, a polyethylene glycol-containing compound forms a covalent attachment to an amino group using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG—SPA); mPEG-N-hydroxy-succinimide (mPEG-NHS); mPEG₂-N-hydroxy-succinimide (mPEG₂—NHS); mPEG-cyanuric chloride; mPEG₂-cyanuric chloride; mPEG₂-Lysinol-NPC, and mPEG₂-Lys-NHS.

In some embodiments, the presence of the PEG Unit in an ADC is capable of having two potential impacts upon the pharmacokinetics of the resulting ADC. One impact is a decrease in clearance (and consequent increase in exposure) that arises from the reduction in non-specific interactions induced by the exposed hydrophobic elements of the Drug Unit (such as a Drug Unit comprising a hydrophobic free drug). The second impact is a decrease in volume and rate of distribution that sometimes arises from the increase in the molecular weight of the ADC. Increasing the number of polyethylene glycol subunits also increases the hydrodynamic radius of a conjugate, typically resulting in decreased diffusivity. In turn, decreased diffusivity typically diminishes the ability of the ADC to penetrate into a tumor (Schmidt and Wittrup, Mol Cancer Ther 2009; 8:2861-2871). Because of these two competing pharmacokinetic effects, it can be desirable to use a PEG Unit that is sufficiently large to decrease the ADC clearance thus increasing plasma exposure, but not so large as to greatly diminish its diffusivity to an extent that it interferes with the ability of the ADC to reach the intended target cell population. See, e.g., Examples 1, 18, and 21 of U.S. Publ. No. 2016/0310612, which is incorporated by reference herein, for methodology for selecting an optimal size of a PEG Unit for a particular hydrophobic drug-linker moiety.

In some embodiments, the PEG Unit comprises one or more linear polyethylene glycol chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG comprises a combined total of at least 8 subunits, at least 10 subunits, or at least 12 subunits. In some such embodiments, the PEG comprises no more than a combined total of about 72 subunits. In some such embodiments, the PEG comprises no more than a combined total of about 36 subunits. In some embodiments, the PEG comprises about 8 to about 24 subunits (referred to as PEG8 to PEG24).

In some embodiments, the PEG Unit comprises a combined total of from 2 to 72, 2 to 60, 2 to 48, 2 to 36 or 2 to 24 subunits, from 3 to 72, 3 to 60, 3 to 48, 3 to 36 or 3 to 24 subunits, from 4 to 72, 8 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits. In some embodiments, the PEG Unit comprises a combined total of from 2 to 24 subunits, 2 to 16 subunits, 2 to 12 subunits, 2 to 8 subunits, or 2 to 6 subunits.

Illustrative linear PEGs that can be used in any of the embodiments provided herein are as follows:

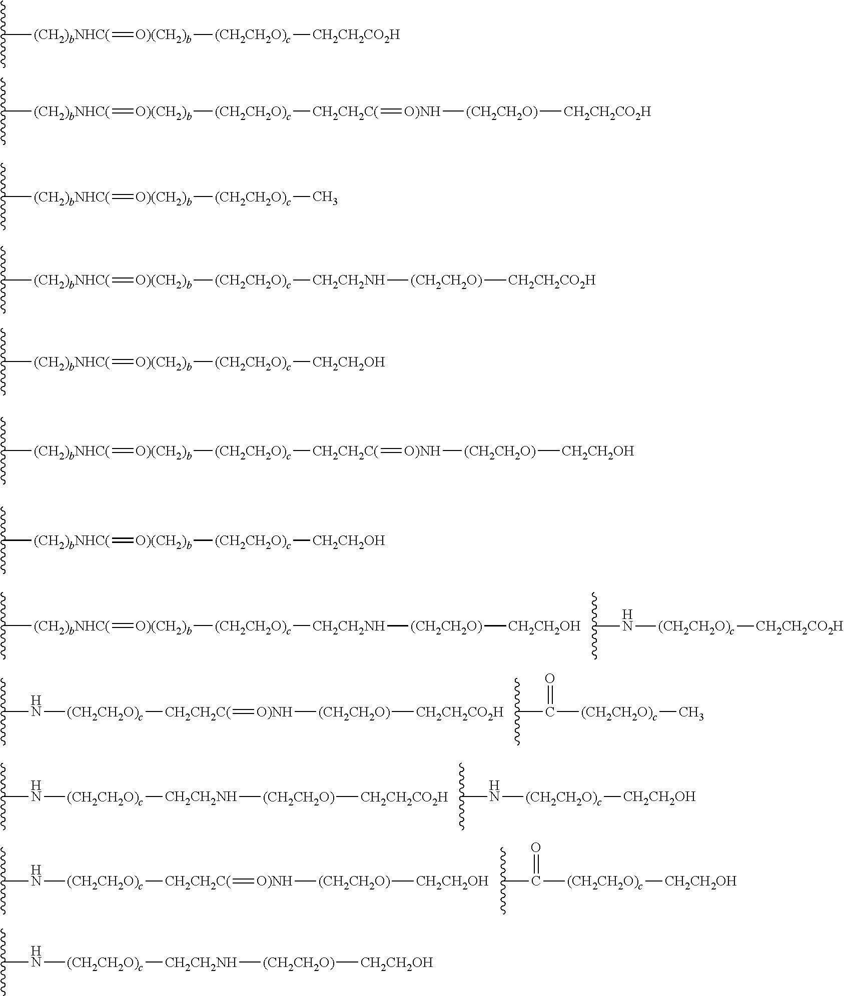

wherein the wavy line indicates the site of attachment to the ADC; each subscript b is independently selected from the group consisting of 2 to 12; and each subscript c is independently selected from the group consisting of 1 to 72, 8 to 72, 10 to 72, 12 to 72, 6 to 24, or 8 to 24. In some embodiments, each subscript b is 2 to 6. In some embodiments, each subscript c is about 2, about 4, about 8, about 12, or about 24.

As described herein, the PEG Unit can be selected such that it improves clearance of the resultant ADC but does not significantly impact the ability of the ADC to penetrate into a tumor. In embodiments in which the Drug Unit and the collective linker/multiplexer conjugate of the ADC has a S log P value comparable to that of a maleimido-derived glucuronide MMAE Drug Unit, the PEG Unit has from about 8 subunits to about 24 subunits. In embodiments, the PEG Unit has about 12 subunits. In embodiments in which the Drug Unit and the collective linker/multiplexer conjugate of the ADC has a S log P value greater than that of a maleimido-derived glucuronide MMAE Drug Unit, a PEG Unit with more subunits is sometimes required.

In some embodiments, the PEG Unit is from about 300 daltons to about 5 kilodaltons; from about 300 daltons to about 4 kilodaltons; from about 300 daltons to about 3 kilodaltons; from about 300 daltons to about 2 kilodaltons; from about 300 daltons to about 1 kilodalton; or any value in between. In some embodiments, the PEG has at least 8, 10 or 12 subunits. In some embodiments, the PEG Unit is PEG2 to PEG72, for example, PEG2, PEG4, PEG8, PEG10, PEG12, PEG16, PEG20, PEG24, PEG28, PEG32, PEG36, PEG48, or PEG72.

In some embodiments, apart from the PEGylation of the ADC, there are no other PEG subunits present in the ADC (i.e., no PEG subunits are present as part of any of the other components of the conjugates and linkers provided herein). In some embodiments, apart from the PEG, there are no more than 8, no more than 7, no more than 6, no more than 5, no more than 4, no more than 3, no more than 2 or no more than 1 other polyethylene glycol (—CH₂CH₂O—) subunits present in the ADC (i.e., no more than 8, 7, 6, 5, 4, 3, 2, or 1 other polyethylene glycol subunits in other components of the ADCs provided herein).

It will be appreciated that when referring to polyethylene glycol subunits of a PEG Unit, and depending on context, the number of subunits can represent an average number, e.g., when referring to a population of ADCs and/or using polydisperse PEGs.

### Antibodies

The term “antibody” as used herein covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds are disrupted, that exhibit the desired biological activity and provided that the antigen binding antibody fragments have the requisite number of attachment sites for the desired number of attached groups, such as a linker (L), as described herein. In some aspects, the linkers are attached to an antibody via a succinimide or hydrolyzed succinimide to the sulfur atoms of cysteine residues of reduced interchain disulfide bonds and/or cysteine residues introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. (see, e.g., Janeway et al., 2001, Immuno. Biology, 5th Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some aspects, the antibody is of human or murine origin, and in some aspects the antibody is a human, humanized or chimeric antibody. Antibodies can be fucosylated to varying extents or afucosylated.

An “intact antibody” is one which comprises an antigen-binding variable region as well as light chain constant domains (CL) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variants thereof.

An “antibody fragment” comprises a portion of an intact antibody, comprising the antigen-binding or variable region thereof. Antibody fragments of the present disclosure include at least one cysteine residue (natural or engineered) and/or at least one lysine residue (natural or engineered) that provides a site for attachment of a linker and/or linker-drug compound. In some embodiments, an antibody fragment includes Fab, Fab′, or F(ab′)₂.

As used herein the term “engineered cysteine residue” or “eCys residue” refers to a cysteine amino acid or a derivative thereof that is incorporated into an antibody. In those aspects one or more eCys residues can be incorporated into an antibody, and typically, the eCys residues are incorporated into either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Further information can be found in U.S. Pat. No. 9,000,130, the contents of which are incorporated herein in its entirety. Derivatives of cysteine (Cys) include but are not limited to beta-2-Cys, beta-3-Cys, homocysteine, and N-methyl cysteine.

In some embodiments, the antibodies of the present disclosure include those having one or more engineered cysteine (eCys) residues. In some embodiments, one of more eCys residues are derivatives of cysteine, for example, beta-2-Cys, beta-3-Cys, homocysteine, or N-methyl-Cys.

In some embodiments, the antibodies of the present disclosure include those having one or more engineered lysine (eLys) residues. In some embodiments, one or more native lysine and/or eLys residues are activated prior to conjugation with a drug-linker intermediate (to form an ADC, as described herein). In some embodiments, the activation comprises contacting the antibody with a compound comprising a succinimydyl ester and a functional group selected from the group consisting of: maleimido, pyridyldisulfidem, and iodoacetamido.

An “antigen” is an entity to which an antibody specifically binds.

The terms “specific binding” and “specifically binds” mean that the antibody or antibody fragment thereof will bind, in a selective manner, with its corresponding target antigen and not with a multitude of other antigens. Typically, the antibody or antibody fragment binds with an affinity of at least about 1×10⁻⁷ M, for example, 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “amino acid” as used herein, refers to natural and non-natural, and proteogenic amino acids. Exemplary amino acids include, but are not limited to alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, ornithine, β-alanine, citrulline, serine methyl ether, aspartate methyl ester, glutamate methyl ester, homoserine methyl ether, and N,N-dimethyl lysine.

In some embodiments, an antibody is a polyclonal antibody. In some embodiments, an antibody is a monoclonal antibody. In some embodiments, an antibody is chimeric. In some embodiments, an antibody is humanized. In some embodiments, an antibody is an antigen binding fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

Useful polyclonal antibodies are heterogeneous populations of antibody molecules derived from the sera of immunized animals. Useful monoclonal antibodies are homogeneous populations of antibodies to a particular antigenic determinant (e.g., a cancer or immune cell antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art. See, e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16.

In some embodiments, an antibody includes a functionally active fragment, derivative or analog of an antibody that binds specifically to target cells (e.g., cancer cell antigens) or other antibodies bound to cancer cells or matrix. In this regard, “functionally active” means that the fragment, derivative or analog is able to bind specifically to target cells. To determine which CDR sequences bind the antigen, synthetic peptides containing the CDR sequences are typically used in binding assays with the antigen by any binding assay method known in the art (e.g., the Biacore assay). See, e.g., Kabat et al., 1991, Sequences of Proteins ofImmunological Interest, 5^{th} Ed., NIH, Bethesda, Md.; and Kabat, et al., 1980, J. Immunology 125(3):961-969.

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and a constant region derived from a human immunoglobulin. See, e.g., U.S. Pat. Nos. 4,816,567; and 4,816,397, which are each incorporated herein by reference in their entireties. Humanized antibodies are antibody molecules from non-human species having one or more CDRs from the non-human species and a framework region from a human immunoglobulin molecule. See, e.g., U.S. Pat. No. 5,585,089, which is incorporated herein by reference in its entirety. Such chimeric and humanized monoclonal antibodies can be produced by recombinant DNA techniques known in the art, for example using methods described in International Publ. No. WO 87/02671; European Publ. No. 0 184 187; European Publ. No. 0171496; European Publ. No. 0173494; International Publ. No. WO 86/01533; U.S. Pat. No. 4,816,567; European Publ. No. 012023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321: 522-525; Verhoeyan et al., 1988, Science 239:1534; and Beidler et al., 1988, J. Immunol. 141:4053-4060; each of which is incorporated herein by reference in its entirety.

In some embodiments, an antibody is a completely human antibody. In some embodiments, an antibody is produced using transgenic mice that are incapable of expressing endogenous immunoglobulin heavy and light chain genes, but which are capable of expressing human heavy and light chain genes.

In some embodiments, the antibodies are those that are intact or fully-reduced antibodies. The term ‘fully-reduced’ is meant to refer to antibodies in which all four inter-chain disulfide linkages have been reduced to provide eight thiols that are capable of attachment to a linker (L¹).

Attachment to the antibody can be via thioether linkages from native and/or engineered cysteine residues, or from an amino acid residue engineered to participate in a cycloaddition reaction (such as a click reaction) with the corresponding linker intermediate, as described herein. In some embodiments, the antibodies are those that are intact or fully-reduced antibodies, or are antibodies bearing engineered cysteine groups that are modified with a functional group that are capable of participating in, for example, click chemistry or other cycloaddition reactions for attachment of other components of the ADC as described herein (e.g., Diels-Alder reactions or other [3+2] or [4+2] cycloadditions). See, e.g., Agard, et al., J. Am. Chem. Soc. Vol. 126, pp. 15046-15047 (2004); Laughlin, et al., Science, Vol. 320, pp. 664-667 (2008); Beatty, et al., ChemBioChem, Vol. 11, pp. 2092-2095 (2010); and Van Geel, et al., Bioconjug. Chem. Vol. 26, pp. 2233-2242 (2015).

Antibodies that bind specifically to a cancer or immune cell antigen are available commercially or produced by any method known to one of skill in the art such as, e.g., chemical synthesis or recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, the antibody can be used for the treatment of a cancer (e.g., an antibody approved by the FDA and/or EMA). Antibodies that bind specifically to a cancer or immune cell antigen are available commercially or produced by any method known to one of skill in the art such as, e.g., recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, an antibody can bind specifically to a receptor or a receptor complex expressed on lymphocytes. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein or other immune cell expressed surface receptor.

In some embodiments, an antibody can bind specifically to a cancer cell antigen. In some embodiments, an antibody can bind specifically to an immune cell antigen. It will be understood that the antibody component in an ADC is an antibody in residue form such that “Ab” in the ADC structures described herein incorporates the structure of the antibody.

Non-limiting examples of antibodies that can be used for treatment of cancer and antibodies that bind specifically to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

In some embodiments, the antibodies for the treatment of an autoimmune disorder are used in accordance with the compositions and methods described herein. Antibodies immunospecific for an antigen of a cell that is responsible for producing autoimmune antibodies are obtainable if not commercially or otherwise available by any method known to one of skill in the art such as, e.g., chemical synthesis or recombinant expression techniques.

In some embodiments, the antibodies are to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein.

Examples of antibodies available for the treatment of cancer to and internalizing antibodies that bind to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

Exemplary antigens are provided below. Exemplary antibodies that bind the indicated antigen are shown in parentheses.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab tiuxetan), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SIT1, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAMI, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, Gp100, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS. In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGMI, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AM4HR2 and androgen receptor.

In some embodiments, the tumor-associated antigen is a transmembrane or membrane-associated protease. For example, the following antigens are transmembrane or membrane-associated proteases: ADAM12, ADAM9, TMPRSS11D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab tiuxetan), CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD112, CD155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD 115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

In some embodiments, the antigen is CD30. In some embodiments, the antibody is an antibody or antigen-binding fragment that binds to CD30, such as described in International Patent Publication No. WO 02/43661. In some embodiments, the anti-CD30 antibody is cAC10, which is described in International Patent Publication No. WO 02/43661. cAC10 is also known as brentuximab. In some embodiments, the anti-CD30 antibody comprises the CDRs of cAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some embodiments, the antigen is CD70. In some embodiments, the antibody is an antibody or antigen-binding fragment that binds to CD70, such as described in International Patent Publication No. WO 2006/113909. In some embodiments, the antibody is a h1F6 anti-CD70 antibody, which is described in International Patent Publication No. WO 2006/113909. hlF6 is also known as vorsetuzumab. In some embodiments, the anti-CD70 antibody comprises a heavy chain variable region comprising the three CDRs of SEQ ID NO:12 and a light chain variable region comprising the three CDRs of SEQ ID NO:13. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD70 antibody comprises a heavy chain variable region comprising an amino acid sequence that is at least 95%, at least 96%, at least 97%, at last 98%, at least 99%, or 100% identical to the amino acid sequence of SEQ ID NO: 12 and a light chain variable region comprising an amino acid sequence that is at least 95% at least 96%, at least 97%, at last 98%, at least 99%, or 100% identical to the amino acid sequence of SEQ ID NO: 13. In some embodiments, the anti-CD30 antibody comprises a heavy chain comprising the amino acid sequence of SEQ ID NO: 14 and a light chain comprising the amino acid sequence of SEQ ID NO: 15.

In some embodiments, the antigen is interleukin-1 receptor accessory protein (IL1RAP). IL1RAP is a co-receptor of the IL1 receptor (IL1R1) and is required for interleukin-1 (IL1) signaling. IL1 has been implicated in the resistance to certain chemotherapy regimens. IL1RAP is overexpressed in various solid tumors, both on cancer cells and in the tumor microenvironment, but has low expression on normal cells. IL1RAP is also overexpressed in hematopoietic stem and progenitor cells, making it a candidate to target for chronic myeloid leukemia (CML). IL1RAP has also been shown to be overexpressed in acute myeloid leukemia (AML). Antibody binding to IL1RAP could block signal transduction from IL-1 and IL-33 into cells and allow NK-cells to recognize tumor cells and subsequent killing by antibody dependent cellular cytotoxicity (ADCC).

In some embodiments, the antigen is ASCT2. ASCT2 is also known as SLC1A5. ASCT2 is a ubiquitously expressed, broad-specificity, sodium-dependent neutral amino acid exchanger.

ASCT2 is involved in glutamine transport. ASCT2 is overexpressed in different cancers and is closely related to poor prognosis. Downregulating ASCT2 has been shown to suppress intracellular glutamine levels and downstream glutamine metabolism, including glutathione production. Due to its high expression in many cancers, ASCT2 is a potential therapeutic target. These effects attenuated growth and proliferation, increased apoptosis and autophagy, and increased oxidative stress and mTORC1 pathway suppression in head and neck squamous cell carcinoma (HNSCC). Additionally, silencing ASCT2 improved the response to cetuximab in HNSCC.

In some embodiments, an antibody-drug conjugate provided herein binds to TROP2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 16, 17, 18, 19, 20, and 21, respectively. In some embodiments, the antibody of the antibody drug conjugate comprises a heavy chain variable region comprising the amino acid sequence of SEQ ID NO: 22 and a light chain variable region comprising the amino acid sequence of SEQ ID NO: 23. In some embodiments, the antibody of the antibody drug conjugate is sacituzumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 24, 25, 26, 27, 28, and 29, respectively. In some embodiments, the antibody of the antibody drug conjugate comprises a heavy chain variable region comprising the amino acid sequence of SEQ ID NO: 30 and a light chain variable region comprising the amino acid sequence of SEQ ID NO: 31. In some embodiments, the antibody of the antibody drug conjugate is datopotamab.

In some embodiments, an antibody-drug conjugate provided herein binds to MICA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 32, 33, 34, 35, 36, and 37, respectively. In some embodiments, the antibody of the antibody drug conjugate comprises a heavy chain variable region comprising the amino acid sequence of SEQ ID NO: 38 and a light chain variable region comprising the amino acid sequence of SEQ ID NO: 39. In some embodiments, the antibody of the antibody drug conjugate is h1D5v11 hIgG1K. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 40, 41, 42, 43, 44, and 45, respectively. In some embodiments, the antibody of the antibody drug conjugate comprises a heavy chain variable region comprising the amino acid sequence of SEQ ID NO: 46 and a light chain variable region comprising the amino acid sequence of SEQ ID NO: 47. In some embodiments, the antibody of the antibody drug conjugate is MICA.36 hIgG1K G236A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 48, 49, 50, 51, 52, and 53, respectively. In some embodiments, the antibody of the antibody drug conjugate comprises a heavy chain variable region comprising the amino acid sequence of SEQ ID NO: 54 and a light chain variable region comprising the amino acid sequence of SEQ ID NO: 55. In some embodiments, the antibody of the antibody drug conjugate is h3F9 H1L3 hIgG1K. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 56, 57, 58, 59, 60, and 61, respectively. In some embodiments, the antibody of the antibody drug conjugate comprises a heavy chain variable region comprising the amino acid sequence of SEQ ID NO: 62 and a light chain variable region comprising the amino acid sequence of SEQ ID NO: 63. In some embodiments, the antibody of the antibody drug conjugate is CM33322 Ab28 hIgG1K.

In some embodiments, an antibody-drug conjugate provided herein binds to CD24. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 64, 65, 66, 67, 68, and 69, respectively. In some embodiments, the antibody of the antibody drug conjugate comprises a heavy chain variable region comprising the amino acid sequence of SEQ ID NO: 70 and a light chain variable region comprising the amino acid sequence of SEQ ID NO: 71. In some embodiments, the antibody of the antibody drug conjugate is SWA11.

In some embodiments, an antibody-drug conjugate provided herein binds to ITGav. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 72, 73, 74, 75, 76, and 77, respectively. In some embodiments, the antibody of the antibody drug conjugate comprises a heavy chain variable region comprising the amino acid sequence of SEQ ID NO: 78 and a light chain variable region comprising the amino acid sequence of SEQ ID NO: 79. In some embodiments, the antibody of the antibody drug conjugate is intetumumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 80, 81, 82, 83, 84, and 85, respectively. In some embodiments, the antibody of the antibody drug conjugate comprises a heavy chain variable region comprising the amino acid sequence of SEQ ID NO: 86 and a light chain variable region comprising the amino acid sequence of SEQ ID NO: 87. In some embodiments, the antibody of the antibody drug conjugate is abituzumab.

In some embodiments, an antibody-drug conjugate provided herein binds to gpA33. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 88, 89, 90, 91, 92, and 93, respectively. In some embodiments, the antibody of the antibody drug conjugate comprises a heavy chain variable region comprising the amino acid sequence of SEQ ID NO: 94 and a light chain variable region comprising the amino acid sequence of SEQ ID NO: 95.

In some embodiments, an antibody-drug conjugate provided herein binds to IL1Rap. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 96, 97, 98, 99, 100, and 101, respectively. In some embodiments, the antibody of the antibody drug conjugate comprises a heavy chain variable region comprising the amino acid sequence of SEQ ID NO: 102 and a light chain variable region comprising the amino acid sequence of SEQ ID NO: 103. In some embodiments, the antibody of the antibody drug conjugate is nidanilimab.

In some embodiments, an antibody-drug conjugate provided herein binds to EpCAM. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 104, 105, 106, 017, 108, and 109, respectively. In some embodiments, the antibody of the antibody drug conjugate comprises a heavy chain variable region comprising the amino acid sequence of SEQ ID NO: 110 and a light chain variable region comprising the amino acid sequence of SEQ ID NO: 111. In some embodiments, the antibody of the antibody drug conjugate is adecatumumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 112, 113, 114, 115, 116, and 117, respectively. In some embodiments, the antibody of the antibody drug conjugate comprises a heavy chain variable region comprising the amino acid sequence of SEQ ID NO: 118 and a light chain variable region comprising the amino acid sequence of SEQ ID NO: 119. In some embodiments, the antibody of the antibody drug conjugate is Ep157305. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 120, 121, 122, 123, 124, and 125, respectively. In some embodiments, the antibody of the antibody drug conjugate comprises a heavy chain variable region comprising the amino acid sequence of SEQ ID NO: 126 and a light chain variable region comprising the amino acid sequence of SEQ ID NO: 127. In some embodiments, the antibody of the antibody drug conjugate is Ep3-171. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 128, 129, 130, 131, 132, and 133, respectively. In some embodiments, the antibody of the antibody drug conjugate comprises a heavy chain variable region comprising the amino acid sequence of SEQ ID NO: 134 and a light chain variable region comprising the amino acid sequence of SEQ ID NO: 135. In some embodiments, the antibody of the antibody drug conjugate is Ep3622w94. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 136, 137, 138, 139, 140, and 141, respectively. In some embodiments, the antibody of the antibody drug conjugate comprises a heavy chain variable region comprising the amino acid sequence of SEQ ID NO: 142 and a light chain variable region comprising the amino acid sequence of SEQ ID NO: 143. In some embodiments, the antibody of the antibody drug conjugate is EpINGI. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 144, 145, 146, 147, 148, and 149, respectively. In some embodiments, the antibody of the antibody drug conjugate comprises a heavy chain variable region comprising the amino acid sequence of SEQ ID NO: 150 and a light chain variable region comprising the amino acid sequence of SEQ ID NO: 151. In some embodiments, the antibody of the antibody drug conjugate is EpAb2-6.

In some embodiments, an antibody-drug conjugate provided herein binds to CD352. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 152, 153, 154, 155, 156, and 157, respectively. In some embodiments, the antibody of the antibody drug conjugate comprises a heavy chain variable region comprising the amino acid sequence of SEQ ID NO: 158 and a light chain variable region comprising the amino acid sequence of SEQ ID NO: 159. In some embodiments, the antibody of the antibody drug conjugate is h20F3.

In some embodiments, an antibody-drug conjugate provided herein binds to CS1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 160, 161, 162, 163, 164, and 165, respectively. In some embodiments, the antibody of the antibody drug conjugate comprises a heavy chain variable region comprising the amino acid sequence of SEQ ID NO: 166 and a light chain variable region comprising the amino acid sequence of SEQ ID NO: 167. In some embodiments, the antibody of the antibody drug conjugate is elotuzumab.

In some embodiments, an antibody-drug conjugate provided herein binds to CD38. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 168, 169, 170, 171, 172, and 173, respectively. In some embodiments, the antibody of the antibody drug conjugate comprises a heavy chain variable region comprising the amino acid sequence of SEQ ID NO: 174 and a light chain variable region comprising the amino acid sequence of SEQ ID NO: 175. In some embodiments, the antibody of the antibody drug conjugate is daratumumab.

In some embodiments, an antibody-drug conjugate provided herein binds to CD25. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 176, 177, 178, 179, 180, and 181, respectively. In some embodiments, the antibody of the antibody drug conjugate comprises a heavy chain variable region comprising the amino acid sequence of SEQ ID NO: 182 and a light chain variable region comprising the amino acid sequence of SEQ ID NO: 183. In some embodiments, the antibody of the antibody drug conjugate is daclizumab.

In some embodiments, an antibody-drug conjugate provided herein binds to ADAM9. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 184, 185, 186, 187, 188, and 189, respectively. In some embodiments, the antibody of the antibody drug conjugate comprises a heavy chain variable region comprising the amino acid sequence of SEQ ID NO: 190 and a light chain variable region comprising the amino acid sequence of SEQ ID NO: 191. In some embodiments, the antibody of the antibody drug conjugate is chMAbA9-A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 192, 193, 194, 195, 196, and 197, respectively. In some embodiments, the antibody of the antibody drug conjugate comprises a heavy chain variable region comprising the amino acid sequence of SEQ ID NO: 198 and a light chain variable region comprising the amino acid sequence of SEQ ID NO: 199. In some embodiments, the antibody of the antibody drug conjugate is hMAbA9-A.

In some embodiments, an antibody-drug conjugate provided herein binds to CD59. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 200, 201, 202, 203, 204, and 205, respectively. In some embodiments, the antibody of the antibody drug conjugate comprises a heavy chain variable region comprising the amino acid sequence of SEQ ID NO: 206 and a light chain variable region comprising the amino acid sequence of SEQ ID NO: 207.

In some embodiments, an antibody-drug conjugate provided herein binds to CD25. In some embodiments, the antibody of the antibody drug conjugate is Clone123.

In some embodiments, an antibody-drug conjugate provided herein binds to CD229. In some embodiments, the antibody of the antibody drug conjugate is h8A10.

In some embodiments, an antibody-drug conjugate provided herein binds to CD19. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 208, 209, 210, 211, 212, and 213, respectively. In some embodiments, the antibody of the antibody drug conjugate comprises a heavy chain variable region comprising the amino acid sequence of SEQ ID NO: 214 and a light chain variable region comprising the amino acid sequence of SEQ ID NO: 215. In some embodiments, the antibody of the antibody drug conjugate is denintuzumab, which is also known as hBU12. See WO2009052431.

In some embodiments, an antibody-drug conjugate provided herein binds to CD70. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 216, 217, 218, 219, 220, and 221, respectively. In some embodiments, the antibody of the antibody drug conjugate comprises a heavy chain variable region comprising the amino acid sequence of SEQ ID NO: 222 and a light chain variable region comprising the amino acid sequence of SEQ ID NO: 223. In some embodiments, the antibody of the antibody drug conjugate is vorsetuzumab.

In some embodiments, an antibody-drug conjugate provided herein binds to B7H4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 224, 225, 226, 227, 228, and 229, respectively. In some embodiments, the antibody of the antibody drug conjugate comprises a heavy chain variable region comprising the amino acid sequence of SEQ ID NO: 230 and a light chain variable region comprising the amino acid sequence of SEQ ID NO: 231. In some embodiments, the antibody of the antibody drug conjugate is mirzotamab.

In some embodiments, an antibody-drug conjugate provided herein binds to CD138. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 232, 233, 234, 235, 236, and 237, respectively. In some embodiments, the antibody of the antibody drug conjugate comprises a heavy chain variable region comprising the amino acid sequence of SEQ ID NO: 238 and a light chain variable region comprising the amino acid sequence of SEQ ID NO: 239. In some embodiments, the antibody of the antibody drug conjugate is indatuxumab.

In some embodiments, an antibody-drug conjugate provided herein binds to CD166. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 240, 241, 242, 243, 244, and 245, respectively. In some embodiments, the antibody of the antibody drug conjugate comprises a heavy chain variable region comprising the amino acid sequence of SEQ ID NO: 246 and a light chain variable region comprising the amino acid sequence of SEQ ID NO: 247. In some embodiments, the antibody of the antibody drug conjugate is praluzatamab.

In some embodiments, an antibody-drug conjugate provided herein binds to CD51. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 248, 249, 250, 251, 252, and 253, respectively. In some embodiments, the antibody of the antibody drug conjugate comprises a heavy chain variable region comprising the amino acid sequence of SEQ ID NO: 254 and a light chain variable region comprising the amino acid sequence of SEQ ID NO: 255. In some embodiments, the antibody of the antibody drug conjugate is intetumumab.

In some embodiments, an antibody-drug conjugate provided herein binds to CD56. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 256, 257, 258, 259, 260, and 261, respectively. In some embodiments, the antibody of the antibody drug conjugate comprises a heavy chain variable region comprising the amino acid sequence of SEQ ID NO: 262 and a light chain variable region comprising the amino acid sequence of SEQ ID NO: 263. In some embodiments, the antibody of the antibody drug conjugate is lorvotuzumab.

In some embodiments, an antibody-drug conjugate provided herein binds to CD74. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 264, 265, 266, 267, 268, and 269, respectively. In some embodiments, the antibody of the antibody drug conjugate comprises a heavy chain variable region comprising the amino acid sequence of SEQ ID NO: 270 and a light chain variable region comprising the amino acid sequence of SEQ ID NO: 271. In some embodiments, the antibody of the antibody drug conjugate is milatuzumab.

In some embodiments, an antibody-drug conjugate provided herein binds to CEACAM5. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 272, 273 274, 275, 276, and 277, respectively. In some embodiments, the antibody of the antibody drug conjugate comprises a heavy chain variable region comprising the amino acid sequence of SEQ ID NO: 278 and a light chain variable region comprising the amino acid sequence of SEQ ID NO: 279. In some embodiments, the antibody of the antibody drug conjugate is labetuzumab.

In some embodiments, an antibody-drug conjugate provided herein binds to CanAg. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 280, 281, 282, 283, 284, and 285, respectively. In some embodiments, the antibody of the antibody drug conjugate comprises a heavy chain variable region comprising the amino acid sequence of SEQ ID NO: 286 and a light chain variable region comprising the amino acid sequence of SEQ ID NO: 287. In some embodiments, the antibody of the antibody drug conjugate is cantuzumab.

In some embodiments, an antibody-drug conjugate provided herein binds to DLL-3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 288, 289, 290, 291, 292, and 293, respectively. In some embodiments, the antibody of the antibody drug conjugate comprises a heavy chain variable region comprising the amino acid sequence of SEQ ID NO: 294 and a light chain variable region comprising the amino acid sequence of SEQ ID NO: 295. In some embodiments, the antibody of the antibody drug conjugate is rovalpituzumab.

In some embodiments, an antibody-drug conjugate provided herein binds to DPEP-3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 296, 297, 298, 299, 300, and 301, respectively. In some embodiments, the antibody of the antibody drug conjugate comprises a heavy chain variable region comprising the amino acid sequence of SEQ ID NO: 302 and a light chain variable region comprising the amino acid sequence of SEQ ID NO: 303. In some embodiments, the antibody of the antibody drug conjugate is tamrintamab.

In some embodiments, an antibody-drug conjugate provided herein binds to EGFR. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 304, 305, 306, 307, 308, and 309, respectively. In some embodiments, the antibody of the antibody drug conjugate comprises a heavy chain variable region comprising the amino acid sequence of SEQ ID NO: 310 and a light chain variable region comprising the amino acid sequence of SEQ ID NO: 311. In some embodiments, the antibody of the antibody drug conjugate is laprituximab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 312, 313, 314, 315, 316, and 317, respectively. In some embodiments, the antibody of the antibody drug conjugate comprises a heavy chain variable region comprising the amino acid sequence of SEQ ID NO: 318 and a light chain variable region comprising the amino acid sequence of SEQ ID NO: 319. In some embodiments, the antibody of the antibody drug conjugate is losatuxizumab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 320, 321, 322, 323, 324, and 325, respectively. In some embodiments, the antibody of the antibody drug conjugate comprises a heavy chain variable region comprising the amino acid sequence of SEQ ID NO: 326 and a light chain variable region comprising the amino acid sequence of SEQ ID NO: 327. In some embodiments, the antibody of the antibody drug conjugate is serclutamab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 328, 329, 330, 331, 332, and 333, respectively. In some embodiments, the antibody of the antibody drug conjugate comprises a heavy chain variable region comprising the amino acid sequence of SEQ ID NO: 334 and a light chain variable region comprising the amino acid sequence of SEQ ID NO: 335. In some embodiments, the antibody of the antibody drug conjugate is cetuximab.

In some embodiments, an antibody-drug conjugate provided herein binds to FRa. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 336, 337, 338, 339, 340, and 341, respectively. In some embodiments, the antibody of the antibody drug conjugate comprises a heavy chain variable region comprising the amino acid sequence of SEQ ID NO: 342 and a light chain variable region comprising the amino acid sequence of SEQ ID NO: 343. In some embodiments, the antibody of the antibody drug conjugate is mirvetuximab. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 344, 345, 346, 347, 348, and 349, respectively. In some embodiments, the antibody of the antibody drug conjugate comprises a heavy chain variable region comprising the amino acid sequence of SEQ ID NO: 350 and a light chain variable region comprising the amino acid sequence of SEQ ID NO: 351. In some embodiments, the antibody of the antibody drug conjugate is farletuzumab.

In some embodiments, an antibody-drug conjugate provided herein binds to MUC-1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 352, 353, 354, 355, 356, and 357, respectively. In some embodiments, the antibody of the antibody drug conjugate comprises a heavy chain variable region comprising the amino acid sequence of SEQ ID NO: 358 and a light chain variable region comprising the amino acid sequence of SEQ ID NO: 359. In some embodiments, the antibody of the antibody drug conjugate is gatipotuzumab.

In some embodiments, an antibody-drug conjugate provided herein binds to mesothelin. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 360, 361, 362, 363, 364, and 365, respectively. In some embodiments, the antibody of the antibody drug conjugate comprises a heavy chain variable region comprising the amino acid sequence of SEQ ID NO: 366 and a light chain variable region comprising the amino acid sequence of SEQ ID NO: 367. In some embodiments, the antibody of the antibody drug conjugate is anetumab.

In some embodiments, an antibody-drug conjugate provided herein binds to ROR-1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 368, 369, 370, 371, 372, and 373, respectively. In some embodiments, the antibody of the antibody drug conjugate comprises a heavy chain variable region comprising the amino acid sequence of SEQ ID NO: 374 and a light chain variable region comprising the amino acid sequence of SEQ ID NO: 375. In some embodiments, the antibody of the antibody drug conjugate is zilovertamab.

In some embodiments, an antibody-drug conjugate provided herein binds to ASCT2.

In some embodiments, an antibody-drug conjugate provided herein binds to B7H4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 376, 377, 378, 379, 380, and 381, respectively. In some embodiments, the antibody of the antibody drug conjugate comprises a heavy chain variable region comprising the amino acid sequence of SEQ ID NO: 382 and a light chain variable region comprising the amino acid sequence of SEQ ID NO: 383. In some embodiments, the antibody of the antibody drug conjugate is 20502. See WO2019040780.

In some embodiments, an antibody-drug conjugate provided herein binds to B7-H3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 384, 385, 386, 387, 388, and 389, respectively. In some embodiments, the antibody of the antibody drug conjugate comprises a heavy chain variable region comprising the amino acid sequence of SEQ ID NO: 390 and a light chain variable region comprising the amino acid sequence of SEQ ID NO: 391. In some embodiments, the antibody of the antibody drug conjugate is chAb-A (BRCA84D). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 392, 393, 394, 395, 396, and 397, respectively. In some embodiments, the antibody of the antibody drug conjugate comprises a heavy chain variable region comprising the amino acid sequence of SEQ ID NO: 398 and a light chain variable region comprising the amino acid sequence of SEQ ID NO: 399. In some embodiments, the antibody of the antibody drug conjugate is hAb-B. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 400, 401, 402, 403, 404, and 405, respectively. In some embodiments, the antibody of the antibody drug conjugate comprises a heavy chain variable region comprising the amino acid sequence of SEQ ID NO: 406 and a light chain variable region comprising the amino acid sequence of SEQ ID NO: 407. In some embodiments, the antibody of the antibody drug conjugate is hAb-C. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 408, 409, 410, 411, 412, and 413, respectively. In some embodiments, the antibody of the antibody drug conjugate comprises a heavy chain variable region comprising the amino acid sequence of SEQ ID NO: 414 and a light chain variable region comprising the amino acid sequence of SEQ ID NO: 415. In some embodiments, the antibody of the antibody drug conjugate is hAb-D. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 416, 417, 418, 419, 420, and 421, respectively. In some embodiments, the antibody of the antibody drug conjugate comprises a heavy chain variable region comprising the amino acid sequence of SEQ ID NO: 422 and a light chain variable region comprising the amino acid sequence of SEQ ID NO: 423. In some embodiments, the antibody of the antibody drug conjugate is chM30. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 424, 425, 426, 427, 428, and 429, respectively. In some embodiments, the antibody of the antibody drug conjugate comprises a heavy chain variable region comprising the amino acid sequence of SEQ ID NO: 430 and a light chain variable region comprising the amino acid sequence of SEQ ID NO: 431. In some embodiments, the antibody of the antibody drug conjugate is hM30-H1-L4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 432, 433, 434, 435, 436, and 437, respectively. In some embodiments, the antibody of the antibody drug conjugate comprises a heavy chain variable region comprising the amino acid sequence of SEQ ID NO: 438 and a light chain variable region comprising the amino acid sequence of SEQ ID NO: 439. In some embodiments, the antibody of the antibody drug conjugate is AbV_huAb18-v4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 440, 441, 442, 443, 444, and 445, respectively. In some embodiments, the antibody of the antibody drug conjugate comprises a heavy chain variable region comprising the amino acid sequence of SEQ ID NO: 446 and a light chain variable region comprising the amino acid sequence of SEQ ID NO: 447. In some embodiments, the antibody of the antibody drug conjugate is AbV_huAb3-v6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 448, 449, 450, 451, 452, and 453, respectively. In some embodiments, the antibody of the antibody drug conjugate comprises a heavy chain variable region comprising the amino acid sequence of SEQ ID NO: 454 and a light chain variable region comprising the amino acid sequence of SEQ ID NO: 455. In some embodiments, the antibody of the antibody drug conjugate is AbV_huAb3-v2.6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 456, 457, 458, 459, 460, and 461, respectively. In some embodiments, the antibody of the antibody drug conjugate comprises a heavy chain variable region comprising the amino acid sequence of SEQ ID NO: 462 and a light chain variable region comprising the amino acid sequence of SEQ ID NO: 463. In some embodiments, the antibody of the antibody drug conjugate is AbV_huAb13-v1-CR. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 464, 465, 466, 467, 468, and 469, respectively. In some embodiments, the antibody of the antibody drug conjugate comprises a heavy chain variable region comprising the amino acid sequence of SEQ ID NO: 470 and a light chain variable region comprising the amino acid sequence of SEQ ID NO: 471. In some embodiments, the antibody of the antibody drug conjugate is 8H9-6m. In some embodiments, the antibody of the antibody drug conjugate comprises a heavy chain variable region comprising the amino acid sequence of SEQ ID NO: 472 and a light chain variable region comprising the amino acid sequence of SEQ ID NO: 473. In some embodiments, the antibody of the antibody drug conjugate is m8517. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 474, 475, 476, 477, 478, and 479, respectively. In some embodiments, the antibody of the antibody drug conjugate comprises a heavy chain variable region comprising the amino acid sequence of SEQ ID NO: 480 and a light chain variable region comprising the amino acid sequence of SEQ ID NO: 481. In some embodiments, the antibody of the antibody drug conjugate is TPP-5706. In some embodiments, the antibody of the antibody drug conjugate comprises a heavy chain variable region comprising the amino acid sequence of SEQ ID NO: 482 and a light chain variable region comprising the amino acid sequence of SEQ ID NO: 483. In some embodiments, the antibody of the antibody drug conjugate is TPP-6642. In some embodiments, the antibody of the antibody drug conjugate comprises a heavy chain variable region comprising the amino acid sequence of SEQ ID NO: 484 and a light chain variable region comprising the amino acid sequence of SEQ ID NO: 485. In some embodiments, the antibody of the antibody drug conjugate is TPP-6850.

In some embodiments, an antibody-drug conjugate provided herein binds to CDCP1. In some embodiments, the antibody of the antibody drug conjugate is 10D7.

In some embodiments, an antibody-drug conjugate provided herein binds to HER3. In some embodiments, the antibody of the antibody drug conjugate comprises a heavy chain comprising the amino acid sequence of SEQ ID NO: 486 and a light chain comprising the amino acid sequence of SEQ ID NO: 487. In some embodiments, the antibody of the antibody drug conjugate is patritumab. In some embodiments, the antibody of the antibody drug conjugate comprises a heavy chain comprising the amino acid sequence of SEQ ID NO: 488 and a light chain comprising the amino acid sequence of SEQ ID NO: 489. In some embodiments, the antibody of the antibody drug conjugate is seribantumab. In some embodiments, the antibody of the antibody drug conjugate comprises a heavy chain comprising the amino acid sequence of SEQ ID NO: 490 and a light chain comprising the amino acid sequence of SEQ ID NO: 491. In some embodiments, the antibody of the antibody drug conjugate is elgemtumab. In some embodiments, the antibody of the antibody drug conjugate comprises a heavy chain the amino acid sequence of SEQ ID NO: 492 and a light chain comprising the amino acid sequence of SEQ ID NO: 493. In some embodiments, the antibody of the antibody drug conjugate is lumretuzumab.

In some embodiments, an antibody-drug conjugate provided herein binds to RON. In some embodiments, the antibody of the antibody drug conjugate is Zt/g4.

In some embodiments, an antibody-drug conjugate provided herein binds to claudin-2.

In some embodiments, an antibody-drug conjugate provided herein binds to HLA-G.

In some embodiments, an antibody-drug conjugate provided herein binds to PTK7. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 494, 495, 496, 497, 498, and 499, respectively. In some embodiments, the antibody of the antibody drug conjugate comprises a heavy chain variable region comprising the amino acid sequence of SEQ ID NO: 500 and a light chain variable region comprising the amino acid sequence of SEQ ID NO: 501. In some embodiments, the antibody of the antibody drug conjugate is PTK7 mab 1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 502, 503, 504, 505, 506, and 507, respectively. In some embodiments, the antibody of the antibody drug conjugate comprises a heavy chain variable region comprising the amino acid sequence of SEQ ID NO: 508 and a light chain variable region comprising the amino acid sequence of SEQ ID NO: 509. In some embodiments, the antibody of the antibody drug conjugate is PTK7 mab 2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 510, 511, 512, 513, 514, and 515, respectively. In some embodiments, the antibody of the antibody drug conjugate comprises a heavy chain variable region comprising the amino acid sequence of SEQ ID NO: 516 and a light chain variable region comprising the amino acid sequence of SEQ ID NO: 517. In some embodiments, the antibody of the antibody drug conjugate is PTK7 mab 3.

In some embodiments, an antibody-drug conjugate provided herein binds to LIV1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 518, 519, 520, 521, 522, and 523, respectively. In some embodiments, the antibody of the antibody drug conjugate comprises a heavy chain variable region comprising the amino acid sequence of SEQ ID NO: 524 and a light chain variable region comprising the amino acid sequence of SEQ ID NO: 525. In some embodiments, the antibody of the antibody drug conjugate is ladiratuzumab, which is also known as hLIV22 and hglg. See WO2012078668.

In some embodiments, an antibody-drug conjugate provided herein binds to avb6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 526, 527, 528, 529, 530, and 531, respectively. In some embodiments, the antibody of the antibody drug conjugate comprises a heavy chain variable region comprising the amino acid sequence of SEQ ID NO: 532 and a light chain variable region comprising the amino acid sequence of SEQ ID NO: 533. In some embodiments, the antibody of the antibody drug conjugate is h2A2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 534, 535, 536, 537, 538, and 539, respectively. In some embodiments, the antibody of the antibody drug conjugate comprises a heavy chain variable region comprising the amino acid sequence of SEQ ID NO: 540 and a light chain variable region comprising the amino acid sequence of SEQ ID NO: 541. In some embodiments, the antibody of the antibody drug conjugate is h15H3.

In some embodiments, an antibody-drug conjugate provided herein binds to CD48. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 542, 543, 544, 545, 546, and 547, respectively. In some embodiments, the antibody of the antibody drug conjugate comprises a heavy chain variable region comprising the amino acid sequence of SEQ ID NO: 548 and a light chain variable region comprising the amino acid sequence of SEQ ID NO: 549. In some embodiments, the antibody of the antibody drug conjugate is hMEM102. See WO2016149535.

In some embodiments, an antibody-drug conjugate provided herein binds to PD-L1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 550, 551, 552, 553, 554, and 555, respectively. In some embodiments, the antibody of the antibody drug conjugate comprises a heavy chain variable region comprising the amino acid sequence of SEQ ID NO: 556 and a light chain variable region comprising the amino acid sequence of SEQ ID NO: 557. In some embodiments, the antibody of the antibody drug conjugate is SG-559-01 LALA mAb.

In some embodiments, an antibody-drug conjugate provided herein binds to IGF-1R. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 558, 559, 560, 561, 562, and 563, respectively. In some embodiments, the antibody of the antibody drug conjugate comprises a heavy chain variable region comprising the amino acid sequence of SEQ ID NO: 564 and a light chain variable region comprising the amino acid sequence of SEQ ID NO: 565. In some embodiments, the antibody of the antibody drug conjugate is cixutumumab.

In some embodiments, an antibody-drug conjugate provided herein binds to claudin-18.2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 566, 567, 568, 569, 570, and 571, respectively. In some embodiments, the antibody of the antibody drug conjugate comprises a heavy chain variable region comprising the amino acid sequence of SEQ ID NO: 572 and a light chain variable region comprising the amino acid sequence of SEQ ID NO: 573. In some embodiments, the antibody of the antibody drug conjugate is zolbetuximab (175D10). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 574, 575, 576, 577, 578, and 579, respectively. In some embodiments, the antibody of the antibody drug conjugate comprises a heavy chain variable region comprising the amino acid sequence of SEQ ID NO: 580 and a light chain variable region comprising the amino acid sequence of SEQ ID NO: 581. In some embodiments, the antibody of the antibody drug conjugate is 163E12.

In some embodiments, an antibody-drug conjugate provided herein binds to Nectin-4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 582, 583, 584, 585, 586, and 587, respectively. In some embodiments, the antibody of the antibody drug conjugate comprises a heavy chain variable region comprising the amino acid sequence of SEQ ID NO: 588 and a light chain variable region comprising the amino acid sequence of SEQ ID NO: 589. In some embodiments, the antibody of the antibody drug conjugate is enfortumab. See WO 2012047724.

In some embodiments, an antibody-drug conjugate provided herein binds to SLTRK6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 590, 591, 592, 593, 594, and 595, respectively. In some embodiments, the antibody of the antibody drug conjugate comprises a heavy chain variable region comprising the amino acid sequence of SEQ ID NO: 596 and a light chain variable region comprising the amino acid sequence of SEQ ID NO: 597. In some embodiments, the antibody of the antibody drug conjugate is sirtratumab.

In some embodiments, an antibody-drug conjugate provided herein binds to CD228. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 598, 599, 600, 601, 602, and 603, respectively. In some embodiments, the antibody of the antibody drug conjugate comprises a heavy chain variable region comprising the amino acid sequence of SEQ ID NO: 604 and a light chain variable region comprising the amino acid sequence of SEQ ID NO: 605. In some embodiments, the antibody of the antibody drug conjugate is hL49. See WO 2020/163225.

In some embodiments, an antibody-drug conjugate provided herein binds to CD142 (tissue factor; TF). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 606, 607, 608, 609, 610, and 611, respectively. In some embodiments, the antibody of the antibody drug conjugate comprises a heavy chain variable region comprising the amino acid sequence of SEQ ID NO: 612 and a light chain variable region comprising the amino acid sequence of SEQ ID NO: 613. In some embodiments, the antibody of the antibody drug conjugate is tisotumab. See WO 2010/066803.

In some embodiments, an antibody-drug conjugate provided herein binds to STn. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 614, 615, 616, 617, 618, and 619, respectively. In some embodiments, the antibody of the antibody drug conjugate comprises a heavy chain variable region comprising the amino acid sequence of SEQ ID NO: 620 and a light chain variable region comprising the amino acid sequence of SEQ ID NO: 621. In some embodiments, the antibody of the antibody drug conjugate is h2G12.

In some embodiments, an antibody-drug conjugate provided herein binds to CD20. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 622, 623, 624, 625, 626, and 627, respectively. In some embodiments, the antibody of the antibody drug conjugate comprises a heavy chain variable region comprising the amino acid sequence of SEQ ID NO: 628 and a light chain variable region comprising the amino acid sequence of SEQ ID NO: 629. In some embodiments, the antibody of the antibody drug conjugate is rituximab.

In some embodiments, an antibody-drug conjugate provided herein binds to HER2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 630, 631, 632, 633, 634, and 635, respectively. In some embodiments, the antibody of the antibody drug conjugate comprises a heavy chain variable region comprising the amino acid sequence of SEQ ID NO: 636 and a light chain variable region comprising the amino acid sequence of SEQ ID NO: 637. In some embodiments, the antibody of the antibody drug conjugate is trastuzumab.

In some embodiments, an antibody-drug conjugate provided herein binds to FLT3.

In some embodiments, an antibody-drug conjugate provided herein binds to CD46.

In some embodiments, an antibody-drug conjugate provided herein binds to GloboH.

In some embodiments, an antibody-drug conjugate provided herein binds to AG7.

In some embodiments, an antibody-drug conjugate provided herein binds to mesothelin.

In some embodiments, an antibody-drug conjugate provided herein binds to FCRH5.

In some embodiments, an antibody-drug conjugate provided herein binds to ETBR.

In some embodiments, an antibody-drug conjugate provided herein binds to Tim-1.

In some embodiments, an antibody-drug conjugate provided herein binds to SLC44A4.

In some embodiments, an antibody-drug conjugate provided herein binds to ENPP3.

In some embodiments, an antibody-drug conjugate provided herein binds to CD37.

In some embodiments, an antibody-drug conjugate provided herein binds to CA9.

In some embodiments, an antibody-drug conjugate provided herein binds to Notch3.

In some embodiments, an antibody-drug conjugate provided herein binds to EphA2.

In some embodiments, an antibody-drug conjugate provided herein binds to TRFC.

In some embodiments, an antibody-drug conjugate provided herein binds to PSMA.

In some embodiments, an antibody-drug conjugate provided herein binds to LRRC15.

In some embodiments, an antibody-drug conjugate provided herein binds to 5T4.

In some embodiments, an antibody-drug conjugate provided herein binds to CD79b. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 638, 639, 640, 641, 642, and 643, respectively. In some embodiments, the antibody of the antibody drug conjugate comprises a heavy chain variable region comprising the amino acid sequence of SEQ ID NO: 644 and a light chain variable region comprising the amino acid sequence of SEQ ID NO: 645. In some embodiments, the antibody of the antibody drug conjugate is polatuzumab.

In some embodiments, an antibody-drug conjugate provided herein binds to NaPi2B. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 646, 647, 648, 649, 650, and 651, respectively. In some embodiments, the antibody of the antibody drug conjugate comprises a heavy chain variable region comprising the amino acid sequence of SEQ ID NO: 652 and a light chain variable region comprising the amino acid sequence of SEQ ID NO: 653. In some embodiments, the antibody of the antibody drug conjugate is lifastuzumab.

In some embodiments, an antibody-drug conjugate provided herein binds to Muc16. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 654, 655, 656, 657, 658, and 659, respectively. In some embodiments, the antibody of the antibody drug conjugate comprises a heavy chain variable region comprising the amino acid sequence of SEQ ID NO: 660 and a light chain variable region comprising the amino acid sequence of SEQ ID NO: 661. In some embodiments, the antibody of the antibody drug conjugate is sofituzumab.

In some embodiments, an antibody-drug conjugate provided herein binds to STEAP1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 662, 663, 664, 665, 666, and 667, respectively. In some embodiments, the antibody of the antibody drug conjugate comprises a heavy chain variable region comprising the amino acid sequence of SEQ ID NO: 668 and a light chain variable region comprising the amino acid sequence of SEQ ID NO: 669. In some embodiments, the antibody of the antibody drug conjugate is vandortuzumab.

In some embodiments, an antibody-drug conjugate provided herein binds to BCMA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 670, 671, 672, 673, 674, and 675, respectively. In some embodiments, the antibody of the antibody drug conjugate comprises a heavy chain variable region comprising the amino acid sequence of SEQ ID NO: 676 and a light chain variable region comprising the amino acid sequence of SEQ ID NO: 677. In some embodiments, the antibody of the antibody drug conjugate is belantamab.

In some embodiments, an antibody-drug conjugate provided herein binds to c-Met. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 678, 679, 680, 681, 682, and 683, respectively. In some embodiments, the antibody of the antibody drug conjugate comprises a heavy chain variable region comprising the amino acid sequence of SEQ ID NO: 684 and a light chain variable region comprising the amino acid sequence of SEQ ID NO: 685. In some embodiments, the antibody of the antibody drug conjugate is telisotuzumab.

In some embodiments, an antibody-drug conjugate provided herein binds to EGFR. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 686, 687, 688, 689, 690, and 691, respectively. In some embodiments, the antibody of the antibody drug conjugate comprises a heavy chain variable region comprising the amino acid sequence of SEQ ID NO: 692 and a light chain variable region comprising the amino acid sequence of SEQ ID NO: 693. In some embodiments, the antibody of the antibody drug conjugate is depatuxizumab.

In some embodiments, an antibody-drug conjugate provided herein binds to SLAMF7. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 694, 695, 696, 697, 698, and 699, respectively. In some embodiments, the antibody of the antibody drug conjugate comprises a heavy chain variable region comprising the amino acid sequence of SEQ ID NO: 700 and a light chain variable region comprising the amino acid sequence of SEQ ID NO: 701. In some embodiments, the antibody of the antibody drug conjugate is azintuxizumab.

In some embodiments, an antibody-drug conjugate provided herein binds to SLITRK6. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 702, 703, 704, 705, 706, and 707, respectively. In some embodiments, the antibody of the antibody drug conjugate comprises a heavy chain variable region comprising the amino acid sequence of SEQ ID NO: 708 and a light chain variable region comprising the amino acid sequence of SEQ ID NO: 709. In some embodiments, the antibody of the antibody drug conjugate is sirtratumab.

In some embodiments, an antibody-drug conjugate provided herein binds to C4.4a. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 710, 711, 712, 713, 714, and 715, respectively. In some embodiments, the antibody of the antibody drug conjugate comprises a heavy chain variable region comprising the amino acid sequence of SEQ ID NO: 716 and a light chain variable region comprising the amino acid sequence of SEQ ID NO: 717. In some embodiments, the antibody of the antibody drug conjugate is lupartumab.

In some embodiments, an antibody-drug conjugate provided herein binds to GCC. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 718, 719, 720, 721, 722, and 723, respectively. In some embodiments, the antibody of the antibody drug conjugate comprises a heavy chain variable region comprising the amino acid sequence of SEQ ID NO: 724 and a light chain variable region comprising the amino acid sequence of SEQ ID NO: 725. In some embodiments, the antibody of the antibody drug conjugate is indusatumab.

In some embodiments, an antibody-drug conjugate provided herein binds to Axl. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 726, 727, 728, 729, 730, and 731, respectively. In some embodiments, the antibody of the antibody drug conjugate comprises a heavy chain variable region comprising the amino acid sequence of SEQ ID NO: 732 and a light chain variable region comprising the amino acid sequence of SEQ ID NO: 733. In some embodiments, the antibody of the antibody drug conjugate is enapotamab.

In some embodiments, an antibody-drug conjugate provided herein binds to gpNMB. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 734, 735, 736, 737, 738, and 739, respectively. In some embodiments, the antibody of the antibody drug conjugate comprises a heavy chain variable region comprising the amino acid sequence of SEQ ID NO: 740 and a light chain variable region comprising the amino acid sequence of SEQ ID NO: 741. In some embodiments, the antibody of the antibody drug conjugate is glembatumumab.

In some embodiments, an antibody-drug conjugate provided herein binds to Prolactin receptor. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 742, 743, 744, 745, 746, and 747, respectively. In some embodiments, the antibody of the antibody drug conjugate comprises a heavy chain variable region comprising the amino acid sequence of SEQ ID NO: 748 and a light chain variable region comprising the amino acid sequence of SEQ ID NO: 749. In some embodiments, the antibody of the antibody drug conjugate is rolinsatamab.

In some embodiments, an antibody-drug conjugate provided herein binds to FGFR2. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 750, 751, 752, 753, 754, and 755, respectively. In some embodiments, the antibody of the antibody drug conjugate comprises a heavy chain variable region comprising the amino acid sequence of SEQ ID NO: 756 and a light chain variable region comprising the amino acid sequence of SEQ ID NO: 757. In some embodiments, the antibody of the antibody drug conjugate is aprutumab.

In some embodiments, an antibody-drug conjugate provided herein binds to CDCP1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 758, 759, 760, 761, 762, and 763, respectively. In some embodiments, the antibody of the antibody drug conjugate comprises a heavy chain variable region comprising the amino acid sequence of SEQ ID NO: 764 and a light chain variable region comprising the amino acid sequence of SEQ ID NO: 765. In some embodiments, the antibody of the antibody drug conjugate is Humanized CUB4 #135 HC4-H. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 766, 767, 768, 769, 770, and 771, respectively. In some embodiments, the antibody of the antibody drug conjugate comprises a heavy chain variable region comprising the amino acid sequence of SEQ ID NO: 772 and a light chain variable region comprising the amino acid sequence of SEQ ID NO: 773. In some embodiments, the antibody of the antibody drug conjugate is CUB4. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 774, 775, 776, 777, 778, 779, respectively. In some embodiments, the antibody of the antibody drug conjugate comprises a heavy chain variable region comprising the amino acid sequence of SEQ ID NO: 780 and a light chain variable region comprising the amino acid sequence of SEQ ID NO: 781. In some embodiments, the antibody of the antibody drug conjugate is CP13E10-WT. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 782, 783, 784, 785, 786, and 787, respectively. In some embodiments, the antibody of the antibody drug conjugate comprises a heavy chain variable region comprising the amino acid sequence of SEQ ID NO: 788 and a light chain variable region comprising the amino acid sequence of SEQ ID NO: 789. In some embodiments, the antibody of the antibody drug conjugate is CP13E10-54HCv13-89LCv1.

In some embodiments, an antibody-drug conjugate provided herein binds to ASCT2. In some embodiments, the antibody of the antibody drug conjugate comprises a heavy chain variable region comprising the amino acid sequence of SEQ ID NO: 790 and a light chain variable region comprising the amino acid sequence of SEQ ID NO: 791. In some embodiments, the antibody of the antibody drug conjugate is KM8094a. In some embodiments, the antibody of the antibody drug conjugate comprises a heavy chain variable region comprising the amino acid sequence of SEQ ID NO: 792 and a light chain variable region comprising the amino acid sequence of SEQ ID NO: 793. In some embodiments, the antibody of the antibody drug conjugate is KM8094b. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 794, 795, 796, 797, 798, and 799, respectively. In some embodiments, the antibody of the antibody drug conjugate comprises a heavy chain variable region comprising the amino acid sequence of SEQ ID NO: 800 and a light chain variable region comprising the amino acid sequence of SEQ ID NO: 801. In some embodiments, the antibody of the antibody drug conjugate is KM4018.

In some embodiments, an antibody-drug conjugate provided herein binds to CD123. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 802, 803, 804, 805, 806, and 807, respectively. In some embodiments, the antibody of the antibody drug conjugate comprises a heavy chain variable region comprising the amino acid sequence of SEQ ID NO: 808 and a light chain variable region comprising the amino acid sequence of SEQ ID NO: 809. In some embodiments, the antibody of the antibody drug conjugate is h7G3. See WO 2016201065.

In some embodiments, an antibody-drug conjugate provided herein binds to GPC3. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 810, 811, 812, 813, 814, and 815, respectively. In some embodiments, the antibody of the antibody drug conjugate comprises a heavy chain variable region comprising the amino acid sequence of SEQ ID NO: 816 and a light chain variable region comprising the amino acid sequence of SEQ ID NO: 817. In some embodiments, the antibody of the antibody drug conjugate is hGPC3-1. See WO 2019161174.

In some embodiments, an antibody-drug conjugate provided herein binds to B6A. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 818, 819, 820, 821, 822, and 823, respectively. In some embodiments, the antibody of the antibody drug conjugate comprises a heavy chain variable region comprising the amino acid sequence of SEQ ID NO: 824 and a light chain variable region comprising the amino acid sequence of SEQ ID NO: 825. In some embodiments, the antibody of the antibody drug conjugate is h2A2. See PCT/US20/63390. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 826, 827, 828, 829, 830, and 831, respectively. In some embodiments, the antibody of the antibody drug conjugate comprises a heavy chain variable region comprising the amino acid sequence of SEQ ID NO: 832 and a light chain variable region comprising the amino acid sequence of SEQ ID NO: 833. In some embodiments, the antibody of the antibody drug conjugate is h15H3. See WO 2013/123152.

In some embodiments, an antibody-drug conjugate provided herein binds to PD-L1. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 834, 835, 836, 837, 838, and 839, respectively. In some embodiments, the antibody of the antibody drug conjugate comprises a heavy chain variable region comprising the amino acid sequence of SEQ ID NO: 840 and a light chain variable region comprising the amino acid sequence of SEQ ID NO: 841. In some embodiments, the antibody of the antibody drug conjugate is SG-559-01. See PCT/US2020/054037.

In some embodiments, an antibody-drug conjugate provided herein binds to TIGIT. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 842, 843, 844, 845, 846, and 847, respectively. In some embodiments, the antibody of the antibody drug conjugate comprises a heavy chain variable region comprising the amino acid sequence of SEQ ID NO: 848 and a light chain variable region comprising the amino acid sequence of SEQ ID NO: 849. In some embodiments, the antibody of the antibody drug conjugate is Clone 13 (also known as ADI-23674 or mAb13). See WO 2020041541.

In some embodiments, an antibody-drug conjugate provided herein binds to STN. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 850, 851, 852, 853, 854, and 855, respectively. In some embodiments, the antibody of the antibody drug conjugate comprises a heavy chain variable region comprising the amino acid sequence of SEQ ID NO: 856 and a light chain variable region comprising the amino acid sequence of SEQ ID NO: 857. In some embodiments, the antibody of the antibody drug conjugate is 2G12-2B2. See WO 2017083582.

In some embodiments, an antibody-drug conjugate provided herein binds to CD33. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 858, 859, 860, 861, 862, and 863, respectively. In some embodiments, the antibody of the antibody drug conjugate comprises a heavy chain variable region comprising the amino acid sequence of SEQ ID NO: 864 and a light chain variable region comprising the amino acid sequence of SEQ ID NO: 865. In some embodiments, the antibody of the antibody drug conjugate is h2H12. See WO2013173496.

In some embodiments, an antibody-drug conjugate provided herein binds to NTBA (also known as CD352). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 866, 867, 868, 869, 870, and 871, respectively. In some embodiments, the antibody of the antibody drug conjugate comprises a heavy chain variable region comprising the amino acid sequence of SEQ ID NO: 872 and a light chain variable region comprising the amino acid sequence of SEQ ID NO: 873. In some embodiments, the antibody of the antibody drug conjugate is h20F3 HDLD. See WO 2017004330.

In some embodiments, an antibody-drug conjugate provided herein binds to BCMA. In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 874, 875, 876, 877, 878, and 879, respectively. In some embodiments, the antibody of the antibody drug conjugate comprises a heavy chain variable region comprising the amino acid sequence of SEQ TD NO: 880 and a light chain variable region comprising the amino acid sequence of SEQ ID NO: 881. In some embodiments, the antibody of the antibody drug conjugate is SEA-BCMA (also known as hSG16.17). See WO 2017/143069.

In some embodiments, an antibody-drug conjugate provided herein binds to Tissue Factor (also known as TF). In some embodiments, the antibody of the antibody drug conjugate comprises CDR-H1, CDR-H2, CDR-H3, CDR-L1, CDR-L2, and CDR-L3 comprising the amino acid sequences of SEQ ID NOs: 882, 883, 884, 885, 886, and 887, respectively. In some embodiments, the antibody of the antibody drug conjugate comprises a heavy chain variable region comprising the amino acid sequence of SEQ TD NO: 888 and a light chain variable region comprising the amino acid sequence of SEQ TD NO: 889. In some embodiments, the antibody of the antibody drug conjugate is tisotumab. See WO 2010/066803 and U.S. Pat. No. 9,150,658.

Table of Sequences

| SEQ | | |
|---|---|---|
| ID NO | Description | Sequence |
| 1 | cAC10 CDR-H1 | DYYIT |
| 2 | cAC10 CDR-H2 | WIYPGSGNTKYNEKFKG |
| 3 | cAC10 CDR-H3 | YGNYWFAY |
| 4 | cAC10 CDR-Ll | KASQSVDFDGDSYMN |
| 5 | cAC10 CDR-L2 | AASNLES |
| 6 | cAC10 CDR-L3 | QQSNEDPWT |
| 7 | cAC10 VH | QIQLQQSGPEVVKPGASVKISCKASGYTFTDYYITWVKQKP |
| | | GQGLEWIGWIYPGSGNTKY |
| | | NEKFKGKATLTVDTSSSTAFMQLSSLTSEDTAVYFCANYG |
| | | NYWFAYWGQGTQVTVSA |
| 8 | cAC10 VL | DIVLTQSPASLAVSLGQRATISCKASQSVDFDGDSYMNWY |
| | | QQKPGQPPKVLIYAASNLES |
| | | GIPARFSGSGSGTDFTLNIHPVEEEDAATYYCQQSNEDPWT |
| | | FGGGTKLEIK |
| 9 | cAC10 HC | QIQLQQSGPEVVKPGASVKISCKASGYTFTDYYITWVKQKP |
| | | GQGLEWIGWIYPGSGNTKY |
| | | NEKFKGKATLTVDTSSSTAFMQLSSLTSEDTAVYFCANYG |
| | | NYWFAYWGQGTQVTVSAAST |
| | | KGPSVFPLAPSSKSTSGGTAALGCLVKDYFPEPVTVSWNSG |
| | | ALTSGVHTFPAVLQSS |
| | | GLYSLSSVVTVPSSSLGTQTYICNVNHKPSNTKVDKKVEPK |
| | | SCDKTHTCPPCPAPELLGG |
| | | PSVFLFPPKPKDTLMISRTPEVTCVVVDVSHEDPEVKFNWY |
| | | VDGVEVHNAKTKPREEQYN |
| | | STYRVVSVLTVLHQDWLNGKEYKCKVSNKALPAPIEKTIS |
| | | KAKGQPREPQVYTLPPSRDE |
| | | LTKNQVSLTCLVKGFYPSDIAVEWESNGQPENNYKTTPPVL |
| | | DSDGSFFLYSKLTVDKSRW |
| | | QQGNVFSCSVMHEALHNHYTQKSLSLSPGK |
| 10 | cAC10 HC v2 | QIQLQQSGPEVVKPGASVKISCKASGYTFTDYYITWVKQKP |
| | | GQGLEWIGWIYPGSGNTKY |
| | | NEKFKGKATLTVDTSSSTAFMQLSSLTSEDTAVYFCANYG |
| | | NYWFAYWGQGTQVTVSAAST |
| | | KGPSVFPLAPSSKSTSGGTAALGCLVKDYFPEPVTVSWNSG |
| | | ALTSGVHTFPAVLQSS |
| | | GLYSLSSVVTVPSSSLGTQTYICNVNHKPSNTKVDKKVEPK |
| | | SCDKTHTCPPCPAPELLGG |
| | | PSVFLFPPKPKDTLMISRTPEVTCVVVDVSHEDPEVKFNWY |
| | | VDGVEVHNAKTKPREEQYN |
| | | STYRVVSVLTVLHQDWLNGKEYKCKVSNKALPAPIEKTIS |
| | | KAKGQPREPQVYTLPPSRDE |
| | | LTKNQVSLTCLVKGFYPSDIAVEWESNGQPENNYKTTPPVL |
| | | DSDGSFFLYSKLTVDKSRW |
| | | QQGNVFSCSVMHEALHNHYTQKSLSLSPG |
| 11 | cAC10 LC | DIVLTQSPASLAVSLGQRATISCKASQSVDFDGDSYMNWY |
| | | QQKPGQPPKVLIYAASNLES |
| | | GIPARFSGSGSGTDFTLNIHPVEEEDAATYYCQQSNEDPWT |
| | | FGGGTKLEIKR |
| | | TVAAPSVFIFPPSDEQLKSGTASVVCLLNNFYPREAKVQWK |
| | | VDNALQSGNSQESVTEQDS |
| | | KDSTYSLSSTLTLSKADYEKHKVYACEVTHQGLSSPVTKSF |
| | | NRGEC |
| 12 | h1F6 VH | QVQLVQSGAEVKKPGASVKVSCKASGYTFTNYGMNWVR |
| | | QAPGQGLKWMGWINTYTGEPTY |
| | | ADAFKGRVTMTRDTSISTAYMELSRLRSDDTAVYYCARDY |
| | | GDYGMDYWGQGTTVTVSS |
| 13 | h1F6 VL | DIVMTQSPDSLAVSLGERATINCRASKSVSTSGYSFMHWY |
| | | QQKPGQPPKLLIYLASNLES |
| | | GVPDRFSGSGSGTDFTLTISSLQAEDVAVYYCQHSREVPWT |
| | | FGQGTKVEIK |
| 14 | h1F6 HC | QVQLVQSGAEVKKPGASVKVSCKASGYTFTNYGMNWVR |
| | | QAPGQGLKWMGWINTYTGEPTY |
| | | ADAFKGRVTMTRDTSISTAYMELSRLRSDDTAVYYCARDY |
| | | GDYGMDYWGQGTTVTVSSAS |
| | | TKGPSVFPLAPSSKSTSGGTAALGCLVKDYFPEPVTVSWNS |
| | | GALTSGVHTFPAVLQSSGL |
| | | YSLSSWTVPSSSLGTQTYICNVNHKPSNTKVDKKVEPKSC |
| | | DKTHTCPPCPAPELLGGPS |
| | | VFLFPPKPKDTLMISRTPEVTCVVVDVSHEDPEVKFNWYV |
| | | DGVEVHNAKTKPREEQYNST |
| | | YRVVSVLTVLHQDWLNGKEYKCKVSNKALPAPIEKTISKA |
| | | KGQPREPQVYTLPPSRDELT |
| | | KNQVSLTCLVKGFYPSDIAVEWESNGQPENNYKTTPPVLD |
| | | SDGSFFLYSKLTVDKSRWQQ |
| | | GNVFSCSVMHEALHNHYTQKSLSLSPGK |
| 15 | h1F6 LC | DIVMTQSPDSLAVSLGERATINCRASKSVSTSGYSFMHWY |
| | | QQKPGQPPKLLIYLASNLES |
| | | GVPDRFSGSGSGTDFTLTISSLQAEDVAVYYCQHSREVPWT |
| | | FGQGTKVEIKRTVAAPSVF |
| | | IFPPSDEQLKSGTASVVCLLNNFYPREAKVQWKVDNALQS |
| | | GNSQESVTEQDSKDSTYSLS |
| | | STLTLSKADYEKHKVYACEVTHQGLSSPVTKSFNRGEC |
| 16 | TROP2 CDR-H1 | NYGMN |
| 17 | TROP2 CDR-H2 | WINTYTGEPTYTDDFKG |
| 18 | TROP2 CDR-H3 | GGFGSSYWYFDV |
| 19 | TROP2 CDR-L1 | KASQDVSIAVA |
| 20 | TROP2 CDR-L2 | SASYRYT |
| 21 | TROP2 CDR-L3 | QQHYITPLT |
| 22 | TROP2 VH | QVQLQQSGSELKKPGASVKVSCKASGYTFTNYGMNWVKQ |
| | | APGQGLKWMGWINTYTGEPT |
| | | YTDDFKGRFAFSLDTSVSTAYLQISSLKADDTAVYFCARGG |
| | | FGSSYWYFDVWGQGSLVTVSS |
| 23 | TROP2 VL | DIQLTQSPSSLSASVGDRVSITCKASQDVSIAVAWYQQKPG |
| | | KAPKLLIYSASYRYTGVP |
| | | DRFSGSGSGTDFTLTISSLQPEDFAVYYCQQHYITPLTFGAG |
| | | TKVEIK |
| 24 | TROP2 CDR-H1 | TAGMQ |
| 25 | TROP2 CDR-H2 | WINTHSGVPKYAEDFKG |
| 26 | TROP2 CDR-H3 | SGFGSSYWYFDV |
| 27 | TROP2 CDR-L1 | KASQDVSTAVA |
| 28 | TROP2 CDR-L2 | SASYRYT |
| 29 | TROP2 CDR-L3 | QQHYITPLT |
| 30 | TROP2 VH | QVQLVQSGAEVKKPGASVKVSCKASGYTFTTAGMQWVR |
| | | QAPGQGLEWMGWINTHSGVPKYAEDFKGRVTISADTSTST |
| | | AYLQLSSLKSEDTAVYYCARSGFGSSYWYFDVWGQGTLV |
| | | TVSS |
| 31 | TROP2 VL | DIQMTQSPSSLSASVGDRVTITCKASQDVSTAVAWYQQKP |
| | | GKAPKLLIYSASYRYTGVPSRFSGSGSGTDFTLTISSLQPEDF |
| | | AVYYCQQHYITPLTFGQGTKLEIK |
| 32 | MICA CDR-H1 | SQNIY |
| 33 | MICA CDR-H2 | YIEPYNVVPMYNPKFKG |
| 34 | MICA CDR-H3 | SGSSNFDY |
| 35 | MICA CDR-L1 | SASSSISSHYLH |
| 36 | MICA CDR-L2 | RTSNLAS |
| 37 | MICA CDR-L3 | QQGSSLPLT |
| 38 | MICA VH | EIQLVQSGAEVKKPGASVKVSCKASGYAFTSQNIYWVRQA |
| | | PGQGLEWIGYIEPYNVVPMYNPKFKGRATLTVDKSTSTAY |
| | | LELSSLRSEDTAVYYCARSGSSNFDYWGQGTLVTVSS |
| 39 | MICA VL | DIQLTQSPSSLSASVGDRVTITCSASSSISSHYLHWYQQKPG |
| | | KSPKLLIYRTSNLASGVPSRFSGSGSGTDYTLTISSLQPEDFA |
| | | TYYCQQGSSLPLTFGQGTKVEIK |
| 40 | MICA CDR-H1 | NYAMH |
| 41 | MICA CDR-H2 | LIWYDGSNKFYGDSVKG |
| 42 | MICA CDR-H3 | EGSGHY |
| 43 | MICA CDR-L1 | RASQGISSALA |
| 44 | MICA CDR-L2 | DASSLES |
| 45 | MICA CDR-L3 | QQFNSYPIT |
| 46 | MICA VH | QVQLVESGGGVVQPGRSLRLSCAASGFTFSNYAMHWVRQ |
| | | APGEGLEWVALIWYDGSNKFYGDSVKGRFTISRDNSKNTL |
| | | YLQMNSLSAEDTAVYYCAREGSGHYWGQGTLVTVSS |
| 47 | MICA VL | AIQLTQSPSSLSASVGDRVTITCRASQGISSALAWYQQKPG |
| | | KVPKSLIYDASSLESGVPSRFSGSGSGTDFTLTISSLQPEDFA |
| | | TYYCQQFNSYPITFGQGTRLEIK |
| 48 | MICA CDR-H1 | NYAMS |
| 49 | MICA CDR-H2 | YISPGGDYIYYADSVKG |
| 50 | MICA CDR-H3 | DRRHYGSYAMDY |
| 51 | MICA CDR-L1 | RSSKSLLHSNLNTYLY |
| 52 | MICA CDR-L2 | RMSNLAS |
| 53 | MICA CDR-L3 | MQHLEYPFT |
| 54 | MICA VH | QVQLVESGGGLVKPGGSLRLSCAASGFTFSNYAMSWIRQA |
| | | PGKGLEWVSYISPGGDYIYYADSVKGRFTISRDNAKNSLYL |
| | | QMNSLRAEDTAVYYCTTDRRHYGSYAMDYWGQGTLVTV |
| | | SS |
| 55 | MICA VL | DIVMTQSPLSLPVTPGEPASISCRSSKSLLHSNLNTYLYWFL |
| | | QKPGQSPQILIYRMSNLASGVPDRFSGSGSGTAFTLKISRVE |
| | | AEDVGVYYCMQHLEYPFTFGPGTKLEIK |
| 56 | MICA CDR-H1 | TYAFH |
| 57 | MICA CDR-H2 | GIVPIFGTLKYAQKFQD |
| 58 | MICA CDR-H3 | AIQLEGRPFDH |
| 59 | MICA CDR-L1 | RASQGITSYLA |
| 60 | MICA CDR-L2 | AASALQS |
| 61 | MICA CDR-L3 | QQVNRGAAIT |
| 62 | MICA VH | QVQLVQSGAEVKKPGSSVRVSCRASGGSSTTYAFHWVRQ |
| | | APGQGLEWMGGIVPIFGTLKYAQKFQDRVTLTADKSTGTA |
| | | YMELNSLRLDDTAVYYCARAIQLEGRPFDHWGQGTQVTV |
| | | SA |
| 63 | MICA VL | DIQLTQSPSFLSASVGDRVTITCRASQGITSYLAWYQQKPG |
| | | KAPKLLIYAASALQSGVPSRFSGRGSGTEFTLTISSLQPEDF |
| | | ATYYCQQVNRGAAITFGHGTRLDIK |
| 64 | CD24 CDR-H1 | TYAFH |
| 65 | CD24 CDR-H2 | GIVPIFGTLKYAQKFQD |
| 66 | CD24 CDR-H3 | AIQLEGRPFDH |
| 67 | CD24 CDR-L1 | RASQGITSYLA |
| 68 | CD24 CDR-L2 | AASALQS |
| 69 | CD24 CDR-L3 | QQVNRGAAIT |
| 70 | CD24 VH | QVQLVQSGAEVKKPGSSVRVSCRASGGSSTTYAFHWVRQ |
| | | APGQGLEWMGGIVPIFGTLKYAQKFQDRVTLTADKSTGTA |
| | | YMELNSLRLDDTAVYYCARAIQLEGRPFDHWGQGTQVTV |
| | | SA |
| 71 | CD24 VL | DIQLTQSPSFLSASVGDRVTITCRASQGITSYLAWYQQKPG |
| | | KAPKLLIYAASALQSGVPS |
| | | RFSGRGSGTEFTLTISSLQPEDFATYYCQQVNRGAAITFGHG |
| | | TRLDIK |
| 72 | ITGav CDR-H1 | RYTMH |
| 73 | ITGav CDR-H2 | VISFDGSNKYYVDSVKG |
| 74 | ITGav CDR-H3 | EARGSYAFDI |
| 75 | ITGav CDR-L1 | RASQSVSSYLA |
| 76 | ITGav CDR-L2 | DASNRAT |
| 77 | ITGav CDR-L3 | QQRSNWPPFT |
| 78 | ITGav VH | QVQLVESGGGVVQPGRSRRLSCAASGFTFSRYTMHWVRQ |
| | | APGKGLEWVAVISFDGSNKYYVDSVKGRFTISRDNSENTL |
| | | YLQVNILRAEDTAVYYCAREARGSYAFDIWGQGTMVTVSS |
| 79 | ITGav VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPPFTFGPGTKVDIK |
| 80 | ITGav CDR-H1 | SFWMH |
| 81 | ITGav CDR-H2 | YINPRSGYTEYNEIFRD |
| 82 | ITGav CDR-H3 | FLGRGAMDY |
| 83 | ITGav CDR-L1 | RASQDISNYLA |
| 84 | ITGav CDR-L2 | YTSKIHS |
| 85 | ITGav CDR-L3 | QQGNTFPYT |
| 86 | ITGav VH | QVQLQQSGGELAKPGASVKVSCKASGYTFSSFWMHWVRQ |
| | | APGQGLEWIGYINPRSGYTEYNEIFRDKATMTTDTSTSTAY |
| | | MELSSLRSEDTAVYYCASFLGRGAMDYWGQGTTVTVSS |
| 87 | ITGav VL | DIQMTQSPSSLSASVGDRVTITCRASQDISNYLAWYQQKPG |
| | | KAPKLLIYYTSKIHSGVPSRFSGSGSGTDYTFTISSLQPEDIA |
| | | TYYCQQGNTFPYTFGQGTKVEIK |
| 88 | gpA33 CDR-H1 | TSSYYWG |
| 89 | gpA33 CDR-H2 | TIYYNGSTYYSPSLKS |
| 90 | gpA33 CDR-H3 | QGYDIKINIDV |
| 91 | gpA33 CDR-L1 | RASQSVSSYLA |
| 92 | gpA33 CDR-L2 | VASNRAT |
| 93 | gpA33 CDR-L3 | QQRSNWPLT |
| 94 | gpA33 VH | QLQLQESGPGLVKPSETLSLTCTVSGGSISTSSYYWGWIRQP |
| | | PGKGLEWIGTIYYNGSTYYSPSLKSRVSISVDTSKNQFSLKL |
| | | SSVTAADTSVYYCARQGYDIKINIDVWGQGTTVTVSS |
| 95 | gpA33 VL | EIVLTQSPATLSLSPGERATLSCRASQSVSSYLAWYQQKPG |
| | | QAPRLLIYVASNRATGIPARFSGSGSGTDFTLTISSLEPEDFA |
| | | VYYCQQRSNWPLTFGGGTKVEIK |
| 96 | IL1Rap CDR-H1 | SSWMN |
| 97 | IL1Rap CDR-H2 | RIYPGDGNTHYAQKFQG |
| 98 | IL1Rap CDR-H3 | GYLDPMDY |
| 99 | IL1Rap CDR-L1 | QASQGINNYLN |
| 100 | IL1Rap CDR-L2 | YTSGLHA |
| 101 | IL1Rap CDR-L3 | QQYSILPWT |
| 102 | IL1Rap VH | QVQLVQSGAEVKKPGSSVKVSCKASGYAFTSSWMNWVRQ |
| | | APGQGLEWMGRIYPGDGNTHYAQKFQGRVTLTADKSTST |
| | | AYMELSSLRSEDTAVYYCGEGYLDPMDYWGQGTLVTVSS |
| 103 | IL1Rap VL | DIQMTQSPSSLSASVGDRVTITCQASQGINNYLNWYQQKPG |
| | | KAPKLLIHYTSGLHAGVPSRFSGSGSGTDYTLTISSLEPEDV |
| | | ATYYCQQYSILPWTFGGGTKVEIK |
| 104 | EpCAM CDR-H1 | SYGMH |
| 105 | EpCAM CDR-H2 | VISYDGSNKYYADSVKG |
| 106 | EpCAM CDR-H3 | DMG |
| 107 | EpCAM CDR-L1 | RTSQSISSYLN |
| 108 | EpCAM CDR-L2 | WASTRES |
| 109 | EpCAM CDR-L3 | QQSYDIPYT |
| 110 | EpCAM VH | EVQLLESGGGVVQPGRSLRLSCAASGFTFSSYGMHWVRQA |
| | | PGKGLEWVAVISYDGSNKYYADSVKGRFTISRDNSKNTLY |
| | | LQMNSLRAEDTAVYYCAKDMGWGSGWRPYYYYGMDVW |
| | | GQGTTVTVSS |
| 111 | EpCAM VL | ELQMTQSPSSLSASVGDRVTITCRTSQSISSYLNWYQQKPG |
| | | QPPKLLIYWASTRESGVPDRFSGSGSGTDFTLTISSLQPEDS |
| | | ATYYCQQSYDIPYTFGQGTKLEIK |
| 112 | EpCAM CDR-H1 | NYWMS |
| 113 | EpCAM CDR-H2 | NIKQDGSEKFYADSVKG |
| 114 | EpCAM CDR-H3 | VGPSWEQDY |
| 115 | EpCAM CDR-L1 | TGSSSNIGSYYGVH |
| 116 | EpCAM CDR-L2 | SDTNRPS |
| 117 | EpCAM CDR-L3 | QSYDKGFGHRV |
| 118 | EpCAM VH | EVQLVESGGGLVQPGGSLRLSCAASGFTFSNYWMSWVRQ |
| | | APGKGLEWVANIKQDGSEKFYADSVKGRFTISRDNAKNSL |
| | | YLQMNSLRAEDTAVYYCARVGPSWEQDYWGQGTLVTVS |
| | | A |
| 119 | EpCAM VL | QSVLTQPPSVSGAPGQRVTISCTGSSSNIGSYYGVHWYQQL |
| | | PGTAPKLLIYSDTNRPSGVPDRFSGSKSGTSASLAITGLQAE |
| | | DEADYYCQSYD |
| 120 | EpCAM CDR-H1 | SYAIS |
| 121 | EpCAM CDR-H2 | GIIPIFGTANYAQKFQG |
| 122 | EpCAM CDR-H3 | GLLWNY |
| 123 | EpCAM CDR-L1 | RASQSVSSNLA |
| 124 | EpCAM CDR-L2 | GASTTAS |
| 125 | EpCAM CDR-L3 | QQYNNWPPAYT |
| 126 | EpCAM VH | QVQLVQSGAEVKKPGSSVKVSCKASGGTFSSYAISWVRQA |
| | | PGQGLEWMGGIIPIFGTANYAQKFQGRVTITADESTSTAYM |
| | | ELSSLRSEDTAVYYCARGLLWNYWGQGTLVTVSS |
| 127 | EpCAM VL | EIVMTQSPATLSVSPGERATLSCRASQSVSSNLAWYQQKPG |
| | | QAPRLIIYGASTTASGIPARFSASGSGTDFTLTISSLQSEDFA |
| | | VYYCQQYNNWPPAYTFGQGTKLEIK |
| 128 | EpCAM CDR-H1 | NYGMN |
| 129 | EpCAM CDR-H2 | WINTYTGEPTYGEDFKG |
| 130 | EpCAM CDR-H3 | FGNYVDY |
| 131 | EpCAM CDR-L1 | RSSKNLLHSNGITYLY |
| 132 | EpCAM CDR-L2 | QMSNLAS |
| 133 | EpCAM CDR-L3 | AQNLEIPRT |
| 134 | EpCAM VH | QVQLVQSGPEVKKPGASVKVSCKASGYTFTNYGMNWVRQ |
| | | APGQGLEWMGWINTYTGEPTYGEDFKGRFAFSLDTSASTA |
| | | YMELSSLRSEDTAVYFCARFGNYVDYWGQGSLVTVSS |
| 135 | EpCAM VL | DIVMTQSPLSLPVTPGEPASISCRSSKNLLHSNGITYLYWYL |
| | | QKPGQSPQLLIYQMSNLASGVPDRFSSSGSGTDFTLKISRVE |
| | | AEDVGVYYCAQNLEIPRTFGQGTKVEIK |
| 136 | EpCAM CDR-H1 | KYGMN |
| 137 | EpCAM CDR-H2 | WINTYTEEPTYGDDFKG |
| 138 | EpCAM CDR-H3 | FGSAVDY |
| 139 | EpCAM CDR-L1 | RSSKSLLHSNGITYLY |
| 140 | EpCAM CDR-L2 | QMSNRAS |
| 141 | EpCAM CDR-L3 | AQNLELPRT |
| 142 | EpCAM VH | QIQLVQSGPEVKKPGESVKISCKASGYTFTKYGMNWVKQA |
| | | PGQGLKWMGWINTYTEEPTYGDDFKGRFTFTLDTSTSTAY |
| | | LEISSLRSEDTATYFCARFGSAVDYWGQGTLVTVSS |
| 143 | EpCAM VL | DIVMTQSALSNPVTLGESGSISCRSSKSLLHSNGITYLYWYL |
| | | QKPGQSPQLLIYQMSNRASGVPDRFSSSGSGTDFTLKISRVE |
| | | AEDVGVYYCAQNLELPRTFGQGTKLEMKR |
| 144 | EpCAM CDR-H1 | DYSMH |
| 145 | EpCAM CDR-H2 | WINTETGEPTYADDFKG |
| 146 | EpCAM CDR-H3 | TAVY |
| 147 | EpCAM CDR-L1 | RASQEISVSLS |
| 148 | EpCAM CDR-L2 | ATSTLDS |
| 149 | EpCAM CDR-L3 | LQYASYPWT |
| 150 | EpCAM VH | QVKLQESGPELKKPGETVKISCKASGYTFTDYSMHWVKQA |
| | | PGKGLKWMGWINTETGEPTYADDFKGRFAFSLETSASTAY |
| | | LQINNLKNEDTATYFCARTAVYWGQGTTVTVSS |
| 151 | EpCAM VL | DIQMTQSPSSLSASLGERVSLTCRASQEISVSLSWLQQEPDG |
| | | TIKRLIYATSTLDSGVPKRFSGSRSGSDYSLTISSLESEDFVD |
| | | YYCLQYASYPWTFGGGTKLEIKR |
| 152 | CD352 CDR-H1 | NYGMN |
| 153 | CD352 CDR-H2 | WINTYSGEPRYADDFKG |
| 154 | CD352 CDR-H3 | DYGRWYFDV |
| 155 | CD352 CDR-L1 | RASSSVSHMH |
| 156 | CD352 CDR-L2 | ATSNLAS |
| 157 | CD352 CDR-L3 | QQWSSTPRT |
| 158 | CD352 VH | QIQLVQSGSELKKPGASVKVSCKASGYTFTNYGMNWVRQ |
| | | APGQDLKWMGWINTYSGEPRYADDFKGRFVFSLDKSVNT |
| | | AYLQISSLKAEDTAVYYCARDYGRWYFDVWGQGTTVTVS |
| | | s |
| 159 | CD352 VL | QIVLSQSPATLSLSPGERATMSCRASSSVSHMHWYQQKPG |
| | | QAPRPWIYATSNLASGVPARFSGSGSGTDYTLTISSLEPEDF |
| | | AVYYCQQWSSTPRTFGGGTKVEIKR |
| 160 | CS1 CDR-H1 | RYWMS |
| 161 | CS1 CDR-H2 | EINPDSSTINYAPSLKD |
| 162 | CS1 CDR-H3 | PDGNYWYFDV |
| 163 | CS1 CDR-L1 | KASQDVGIAVA |
| 164 | CSI CDR-L2 | WASTRHT |
| 165 | CS1 CDR-L3 | QQYSSYPYT |
| 166 | CSI VH | EVQLVESGGGLVQPGGSLRLSCAASGFDFSRYWMSWVRQ |
| | | APGKGLEWIGEINPDSSTINYAPSLKDKFIISRDNAKNSLYL |
| | | QMNSLRAEDTAVYYCARPDGNYWYFDVWGQGTLVTVSS |
| 167 | CSI VL | DIQMTQSPSSLSASVGDRVTITCKASQDVGIAVAWYQQKP |
| | | GKVPKLLIYWASTRHTGVPDRFSGSGSGTDFTLTISSLQPED |
| | | VATYYCQQYSSYPYTFGQGTKVEIKR |
| 168 | CD38 CDR-H1 | SFAMS |
| 169 | CD38 CDR-H2 | AISGSGGGTYYADSVKG |
| 170 | CD38 CDR-H3 | DKILWFGEPVFDY |
| 171 | CD38 CDR-L1 | RASQSVSSYLA |
| 172 | CD38 CDR-L2 | DASNRAT |
| 173 | CD38 CDR-L3 | QQRSNWPPT |
| 174 | CD38 VH | EVQLLESGGGLVQPGGSLRLSCAVSGFTFNSFAMSWVRQA |
| | | PGKGLEWVSAISGSGGGTYYADSVKGRFTISRDNSKNTLYL |
| | | QMNSLRAEDTAVYFCAKDKILWFGEPVFDYWGQGTLVTV |
| | | SS |
| 175 | CD38 VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPPTFGQGTKVEIKR |
| 176 | CD25 CDR-H1 | SYRMH |
| 177 | CD25 CDR-H2 | YINPSTGYTEYNQKFKD |
| 178 | CD25 CDR-H3 | GGGVFDY |
| 179 | CD25 CDR-L1 | SASSSISYMH |
| 180 | CD25 CDR-L2 | TTSNLAS |
| 181 | CD25 CDR-L3 | HQRSTYPLT |
| 182 | CD25 VH | QVQLVQSGAEVKKPGSSVKVSCKASGYTFTSYRMHWVRQ |
| | | APGQGLEWIGYINPSTGYTEYNQKFKDKATITADESTNTAY |
| | | MELSSLRSEDTAVYYCARGGGVFDYWGQGTLVTVSS |
| 183 | CD25 VL | DIQMTQSPSTLSASVGDRVTITCSASSSISYMHWYQQKPGK |
| | | APKLLIYTTSNLASGVPARFSGSGSGTEFTLTISSLQPDDFAT |
| | | YYCHQRSTYPLTFGQGTKVEVK |
| 184 | ADAM9 CDR-H1 | SYWM |
| 185 | ADAM9 CDR-H2 | EIIPINGHTNYNEKFKS |
| 186 | ADAM9 CDR-H3 | GGYYYYGSRDYFDY |
| 187 | ADAM9 CDR-L1 | KASQSVDYDGDSYMN |
| 188 | ADAM9 CDR-L2 | AASDLES |
| 189 | ADAM9 CDR-L3 | QQSHEDPFT |
| 190 | ADAM9 VH | QVQLQQPGAELVKPGASVKLSCKASGYTFTSYWMHWVK |
| | | QRPGQGLEWIGEIIPINGHTNYNEKFKSKATLTLDKSSSTAY |
| | | MQLSSLASEDSAVYYCARGGYYYYGSRDYFDYWGQGTTL |
| | | TVSS |
| 191 | ADAM9VL | DIVLTQSPASLAVSLGQRATISCKASQSVDYDGDSYMNWY |
| | | QQIPGQPPKLLIYAASDLESGIPARFSGSGSGTDFTLNIHPVE |
| | | EEDAATYYCQQSHEDPFTFGGGTKLEIK |
| 192 | ADAM9 CDR-H1 | SYWM |
| 193 | ADAM9 CDR-H2 | EIIPIFGHTNYNEKFKS |
| 194 | ADAM9 CDR-H3 | GGYYYYPRQGFLDY |
| 195 | ADAM9 CDR-L1 | KASQSVDYDSGDSYMN |
| 196 | ADAM9 CDR-L2 | AASDLES |
| 197 | ADAM9 CDR-L3 | QQSHEDPFT |
| 198 | ADAM9 VH | EVQLVESGGGLVKPGGSLRLSCAASGFTFSSYWMHWVRQ |
| | | APGKGLEWVGEIIPIFGHTNYNEKFKSRFTISLDNSKNTLYL |
| | | QMGSLRAEDTAVYYCARGGYYYYPRQGFLDYWGQGTTV |
| | | TVSS |
| 199 | ADAM9VL | DIVMTQSPDSLAVSLGERATISCKASQSVDYSGDSYMNWY |
| | | QQKPGQPPKLLIYAASDLESGIPARFSGSGSGTDFTLTISSLE |
| | | PEDFATYYCQQSHEDPFTFGQGTKLEIK |
| 200 | CD59 CDR-H1 | YGMN |
| 201 | CD59 CDR-H2 | YISSSSSTIYADSVKG |
| 202 | CD59 CDR-H3 | GPGMDV |
| 203 | CD59 CDR-L1 | KSSQSVLYSSNNKNYLA |
| 204 | CD59 CDR-L2 | WASTRES |
| 205 | CD59 CDR-L3 | QQYYSTPQLT |
| 206 | CD59 VH | QVQLQQSGGGVVQPGRSLGLSCAASFTFSSYGMNWVRQA |
| | | PGKGLEWVSYISSSSSTIYADSVKGRFTISRDNSKNTLYLQM |
| | | NSLRAEDTAVYYCARGPGMDVWGQGTTVTVS |
| 207 | CD59 VL | DIVLTQSPDSLAVSLGERATINCKSSQSVLYSSNNKNYLAW |
| | | YQQKPGQPPKLLIYWASTRESGVPDRFSGSGSGTDFTPAISS |
| | | LQAEDVAVYYCQQYYSTPQLTFGGGTKVDIK |
| 208 | CD 19 CDR-H1 | TSGMGVG |
| 209 | CD 19 CDR-H2 | HIWWDDDKRYNPALKS |
| 210 | CD 19 CDR-H3 | MELWSYYFDY |
| 211 | CD 19 CDR-L1 | SASSSVSYMH |
| 212 | CD 19 CDR-L2 | DTSKLAS |
| 213 | CD 19 CDR-L3 | FQGSVYPFT |
| 214 | CD19 VH | QVQLQESGPGLVKPSQTLSLTCTVSGGSISTSGMGVGWIRQ |
| | | HPGKGLEWIGHIWWDDDKRYNPALKSRVTISVDTSKNQFS |
| | | LKLSSVTAADTAVYYCARMELWSYYFDYWGQGTLVTVSS |
| 215 | CD19 VL | EIVLTQSPATLSLSPGERATLSCSASSSVSYMHWYQQKPGQ |
| | | APRLLIYDTSKLASGIPARFSGSGSGTDFTLTISSLEPEDVAV |
| | | YYCFQGSVYPFTFGQGTKLEIKR |
| 216 | CD70 CDR-H1 | NYGMN |
| 217 | CD70 CDR-H2 | WINTYTGEPTYADAFKG |
| 218 | CD70 CDR-H3 | DYGDYGMDY |
| 219 | CD70 CDR-L1 | RASKSVSTSGYSFMH |
| 220 | CD70 CDR-L2 | LASNLES |
| 221 | CD70 CDR-L3 | QHSREVPWT |
| 222 | CD70 VH | QVQLVQSGAEVKKPGASVKVSCKASGYTFTNYGMNWVR |
| | | QAPGQGLKWMGWINTYTGEPTYADAFKGRVTMTRDTSIS |
| | | TAYMELSRLRSDDTAVYYCARDYGDYGMDYWGQGTTVT |
| | | VSS |
| 223 | CD70 VL | DIVMTQSPDSLAVSLGERATINCRASKSVSTSGYSFMHWY |
| | | QQKPGQPPKLLIYLASNLESGVPDRFSGSGSGTDFTLTISSL |
| | | QAEDVAVYYCQHSREVPWTFGQGTKVEIK |
| 224 | B7H4 CDR-H1 | SGYSWH |
| 225 | B7H4 CDR-H2 | YIHSSGSTNYNPSLKS |
| 226 | B7H4 CDR-H3 | YDDYFEY |
| 227 | B7H4 CDR-L1 | KASQNVGFNVA |
| 228 | B7H4 CDR-L2 | SASYRYS |
| 229 | B7H4 CDR-L3 | QQYNWYPFT |
| 230 | B7H4 VH | EVQLQESGPGLVKPSETLSLTCAVTGYSITSGYSWHWIRQF |
| | | PGNGLEWMGYIHSSGSTNYNPSLKSRISISRDTSKNQFFLKL |
| | | SSVTAADTAVYYCAGYDDYFEYWGQGTTVTVSS |
| 231 | B7H4 VL | DIQMTQSPSSLSASVGDRVTITCKASQNVGFNVAWYQQKP |
| | | GKSPKALIYSASYRYSGVPSRFSGSGSGTDFTLTISSLQPEDF |
| | | AEYFCQQYNWYPFTFGQGTKLEIK |
| 232 | CD 138 CDR-H1 | NYWIE |
| 233 | CD 138 CDR-H2 | EILPGTGRTIYNEKFKG |
| 234 | CD 138 CDR-H3 | RDYYGNFYYAMDY |
| 235 | CD 138 CDR-L1 | SASQGINNYLN |
| 236 | CD 138 CDR-L2 | YTSTLQS |
| 237 | CD 138 CDR-L3 | QQYSKLPRT |
| 238 | CD138 VH | QVQLQQSGSELMMPGASVKISCKATGYTFSNYWIEWVKQ |
| | | RPGHGLEWIGEILPGTGRTIY |
| | | NEKFKGKATFTADISSNTVQMQLSSLTSEDSAVYYCARRD |
| | | YYGNFYYAMDYWGQGTSVTVSS |
| 239 | CD138 VL | DIQMTQSTSSLSASLGDRVTISCSASQGINNYLNWYQQKPD |
| | | GTVELLIYYTSTLQSGVP |
| | | SRFSGSGSGTDYSLTISNLEPEDIGTYYCQQYSKLPRTFGGG |
| | | TKLEIK |
| 240 | CD 166 CDR-H1 | TYGMGVG |
| 241 | CD 166 CDR-H2 | NIWWSEDKHYSPSLKS |
| 242 | CD 166 CDR-H3 | IDYGNDYAFTY |
| 243 | CD 166 CDR-L1 | RSSKSLLHSNGITYLY |
| 244 | CD 166 CDR-L2 | QMSNLAS |
| 245 | CD 166 CDR-L3 | AQNLELPYT |
| 246 | CD166 VH | QITLKESGPTLVKPTQTLTLTCTFSGFSLSTYGMGVGWIRQP |
| | | PGKALEWLANIWWSEDKHYSPSLKSRLTITKDTSKNQVVL |
| | | TITNVDPVDTATYYCVQIDYGNDYAFTYWGQGTLVTVSS |
| 247 | CD166 VL | DIVMTQSPLSLPVTPGEPASISCRSSKSLLHSNGITYLYWYL |
| | | QKPGQSPQLLIYQMSNLASGVPDRFSGSGSGTDFTLKISRVE |
| | | AEDVGVYYCAQNLELPYTFGQGTKLEIK |
| 248 | CD51 CDR-H1 | RYTMH |
| 249 | CD51 CDR-H2 | VISFDGSNKYYVDSVKG |
| 250 | CD51 CDR-H3 | EARGSYAFDI |
| 251 | CD51 CDR-L1 | RASQSVSSYLA |
| 252 | CD51 CDR-L2 | DASNRAT |
| 253 | CD51 CDR-L3 | QQRSNWPPFT |
| 254 | CD51 VH | QVQLVESGGGVVQPGRSRRLSCAASGFTFSRYTMHWVRQ |
| | | APGKGLEWVAVISFDGSNKYYVDSVKGRFTISRDNSENTL |
| | | YLQVNILRAEDTAVYYCAREARGSYAFDIWGQGTMVTVSS |
| 255 | CD51 VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPPFTFGPGTKVDIK |
| 256 | CD56 CDR-H1 | SFGMH |
| 257 | CD56 CDR-H2 | YISSGSFTIYYADSVKG |
| 258 | CD56 CDR-H3 | MRKGYAMDY |
| 259 | CD56 CDR-L1 | RSSQIIIHSDGNTYLE |
| 260 | CD56 CDR-L2 | KVSNRFS |
| 261 | CD56 CDR-L3 | FQGSHVPHT |
| 262 | CD56 VH | QVQLVESGGGVVQPGRSLRLSCAASGFTFSSFGMHWVRQA |
| | | PGKGLEWVAYISSGSFTIYYADSVKGRFTISRDNSKNTLYL |
| | | QMNSLRAEDTAVYYCARMRKGYAMDYWGQGTLVTVSS |
| 263 | CD56 VL | DVVMTQSPLSLPVTLGQPASISCRSSQIIIHSDGNTYLEWFQ |
| | | QRPGQSPRRLIYKVSNRFSGVPDRFSGSGSGTDFTLKISRVE |
| | | AEDVGVYYCFQGSHVPHTFGQGTKVEIK |
| 264 | CD74 CDR-H1 | NYGVN |
| 265 | CD74 CDR-H2 | WINPNTGEPTFDDDFKG |
| 266 | CD74 CDR-H3 | SRGKNEAWFAY |
| 267 | CD74 CDR-L1 | RSSQSLVHRNGNTYLH |
| 268 | CD74 CDR-L2 | TVSNRFS |
| 269 | CD74 CDR-L3 | SQSSHVPPT |
| 270 | CD74 VH | QVQLQQSGSELKKPGASVKVSCKASGYTFTNYGVNWIKQ |
| | | APGQGLQWMGWINPNTGEPTFDDDFKGRFAFSLDTSVSTA |
| | | YLQISSLKADDTAVYFCSRSRGKNEAWFAYWGQGTLVTVS |
| | | S |
| 271 | CD74 VL | DIQLTQSPLSLPVTLGQPASISCRSSQSLVHRNGNTYLHWFQ |
| | | QRPGQSPRLLIYTVSNRFSGVPDRFSGSGSGTDFTLKISRVE |
| | | AEDVGVYFCSQSSHVPPTFGAGTRLEIK |
| 272 | CEACAM5 CDR- | TYWMS |
| | H1 | |
| 273 | CEACAM5 CDR- | EIHPDSSTINYAPSLKD |
| | H2 | |
| 274 | CEACAM5 CDR- | LYFGFPWFAY |
| | H3 | |
| 275 | CEACAM5 CDR- | KASQDVGTSVA |
| | L1 | |
| 276 | CEACAM5 CDR- | WTSTRHT |
| | L2 | |
| 277 | CEACAM5 CDR- | QQYSLYRS |
| | L3 | |
| 278 | CEACAM5 VH | EVQLVESGGGVVQPGRSLRLSCSASGFDFTTYWMSWVRQ |
| | | APGKGLEWIGEIHPDSSTINYAPSLKDRFTISRDNAKNTLFL |
| | | QMDSLRPEDTGVYFCASLYFGFPWFAYWGQGTPVTVSS |
| 279 | CEACAM5 VL | DIQLTQSPSSLSASVGDRVTITCKASQDVGTSVAWYQQKPG |
| | | KAPKLLIYWTSTRHTGVPSRFSGSGSGTDFTFTISSLQPEDIA |
| | | TYYCQQYSLYRSFGQGTKVEIK |
| 280 | CanAg CDR-H1 | YYGMN |
| 281 | CanAg CDR-H2 | WIDTTTGEPTYAQKFQG |
| 282 | CanAg CDR-H3 | RGPYNWYFDV |
| 283 | CanAg CDR-L1 | RSSKSLLHSNGNTYLY |
| 284 | CanAg CDR-L2 | RMSNLVS |
| 285 | CanAg CDR-L3 | LQHLEYPFT |
| 286 | CanAg VH | QVQLVQSGAEVKKPGETVKISCKASDYTFTYYGMNWVKQ |
| | | APGQGLKWMGWIDTTTGEPTYAQKFQGRIAFSLETSASTA |
| | | YLQIKSLKSEDTATYFCARRGPYNWYFDVWGQGTTVTVSS |
| 287 | CanAg VL | DIVMTQSPLSVPVTPGEPVSISCRSSKSLLHSNGNTYLYWFL |
| | | QRPGQSPQLLIYRMSNLVSGVPDRFSGSGSGTAFTLRISRVE |
| | | AEDVGVYYCLQHLEYPFTFGPGTKLELK |
| 288 | DLL-3 CDR-H1 | NYGMN |
| 289 | DLL-3 CDR-H2 | WINTYTGEPTYADDFKG |
| 290 | DLL-3 CDR-H3 | IGDSSPSDY |
| 291 | DLL-3 CDR-L1 | KASQSVSNDVV |
| 292 | DLL-3 CDR-L2 | YASNRYT |
| 293 | DLL-3 CDR-L3 | QQDYTSPWT |
| 294 | DLL-3 VH | QVQLVQSGAEVKKPGASVKVSCKASGYTFTNYGMNWVR |
| | | QAPGQGLEWMGWINTYTGEPTY |
| | | ADDFKGRVTMTTDTSTSTAYMELRSLRSDDTAVYYCARIG |
| | | DSSPSDYWGQGTLVTVSS |
| 295 | DLL-3 VL | EIVMTQSPATLSVSPGERATLSCKASQSVSNDVVWYQQKP |
| | | GQAPRLLIYYASNRYTGIPA |
| | | RFSGSGSGTEFTLTISSLQSEDFAVYYCQQDYTSPWTFGQG |
| | | TKLEIK |
| 296 | DPEP-3 CDR-H1 | SYWIE |
| 297 | DPEP-3 CDR-H2 | EILPGSGNTYYNERFKD |
| 298 | DPEP-3 CDR-H3 | RAAAYYSNPEWFAY |
| 299 | DPEP-3 CDR-L1 | TASSSVNSFYLH |
| 300 | DPEP-3 CDR-L2 | STSNLAS |
| 301 | DPEP-3 CDR-L3 | HQYHRSPYT |
| 302 | DPEP-3 VH | QVQLVQSGAEVKKPGSSVKVSCKASGGTFSSYWIEWVRQ |
| | | APGQGLEWMGEILPGSGNTYYNERFKDRVTITADESTSTA |
| | | YMELSSLRSEDTAVYYCARRAAAYYSNPEWFAYWGQGTL |
| | | VTVSS |
| 303 | DPEP-3 VL | EIVLTQSPATLSLSPGERATLSCTASSSVNSFYLHWYQQKPG |
| | | LAPRLLIYSTSNLASGIPDRFSGSGSGTDFTLTISRLEPEDFA |
| | | VYYCHQYHRSPYTFGQGTKLEIK |
| 304 | EGFR CDR-H1 | SYWMQ |
| 305 | EGFR CDR-H2 | TIYPGDGDTTYTQKFQG |
| 306 | EGFR CDR-H3 | YDAPGYAMDY |
| 307 | EGFR CDR-L1 | RASQDINNYLA |
| 308 | EGFR CDR-L2 | YTSTLHP |
| 309 | EGFR CDR-L3 | LQYDNLLYT |
| 310 | EGFR VH | QVQLVQSGAEVAKPGASVKLSCKASGYTFTSYWMQWVK |
| | | QRPGQGLECIGTIYPGDGDTTYTQKFQGKATLTADKSSSTA |
| | | YMQLSSLRSEDSAVYYCARYDAPGYAMDYWGQGTLVTV |
| | | SS |
| 311 | EGFR VL | DIQMTQSPSSLSASVGDRVTITCRASQDINNYLAWYQHKPG |
| | | KGPKLLIHYTSTLHPGIPSRFSGSGSGRDYSFSISSLEPEDIAT |
| | | YYCLQYDNLLYTFGQGTKLEIK |
| 312 | EGFR CDR-H1 | RDFAWN |
| 313 | EGFR CDR-H2 | YISYNGNTRYQPSLKS |
| 314 | EGFR CDR-H3 | ASRGFPY |
| 315 | EGFR CDR-L1 | HSSQDINSNIG |
| 316 | EGFR CDR-L2 | HGTNLDD |
| 317 | EGFR CDR-L3 | VQYAQFPWT |
| 318 | EGFR VH | EVQLQESGPGLVKPSQTLSLTCTVSGYSISRDFAWNWIRQP |
| | | PGKGLEWMGYISYNGNTRYQPSLKSRITISRDTSKNQFFLK |
| | | LNSVTAADTATYYCVTASRGFPYWGQGTLVTVSS |
| 319 | EGFR VL | DIQMTQSPSSMSVSVGDRVTITCHSSQDINSNIGWLQQKPG |
| | | KSFKGLIYHGTNLDDGVPSRFSGSGSGTDYTLTISSLQPEDF |
| | | ATYYCVQYAQFPWTFGGGTKLEIK |
| 320 | EGFR CDR-H1 | RDFAWN |
| 321 | EGFR CDR-H2 | YISYNGNTRYQPSLKS |
| 322 | EGFR CDR-H3 | ASRGFPY |
| 323 | EGFR CDR-L1 | HSSQDINSNIG |
| 324 | EGFR CDR-L2 | HGTNLDD |
| 325 | EGFR CDR-L3 | VQYAQFPWT |
| 326 | EGFR VH | EVQLQESGPGLVKPSQTLSLTCTVSGYSISRDFAWNWIRQP |
| | | PGKGLEWMGYISYNGNTRYQPSLKSRITISRDTSKNQFFLK |
| | | LNSVTAADTATYYCVTASRGFPYWGQGTLVTVSS |
| 327 | EGFR VL | DIQMTQSPSSMSVSVGDRVTITCHSSQDINSNIGWLQQKPG |
| | | KSFKGLIYHGTNLDDGVPSRFSGSGSGTDYTLTISSLQPEDF |
| | | ATYYCVQYAQFPWTFGGGTKLEIK |
| 328 | EGFR CDR-H1 | NYGVH |
| 329 | EGFR CDR-H2 | VIWSGGNTDYNTPFTS |
| 330 | EGFR CDR-H3 | ALTYYDYEFAY |
| 331 | EGFR CDR-L1 | RASQSIGTNIH |
| 332 | EGFR CDR-L2 | YASESIS |
| 333 | EGFR CDR-L3 | QQNNNWPTT |
| 334 | EGFR VH | QVQLKQSGPGLVQPSQSLSITCTVSGFSLTNYGVHWVRQSP |
| | | GKGLEWLGVIWSGGNTDYNTPFTSRLSINKDNSKSQVFFK |
| | | MNSLQSNDTAIYYCARALTYYDYEFAYWGQGTLVTVSA |
| 335 | EGFR VL | DILLTQSPVILSVSPGERVSFSCRASQSIGTNIHWYQQRTNG |
| | | SPRLLIKYASESISGIPSRFSGSGSGTDFTLSINSVESEDIADY |
| | | YCQQNNNWPTTFGAGTKLELK |
| 336 | FRa CDR-H1 | GYFMN |
| 337 | FRa CDR-H2 | RIHPYDGDTFYNQKFQG |
| 338 | FRa CDR-H3 | YDGSRAMDY |
| 339 | FRaCDR-Ll | KASQSVSFAGTSLMH |
| 340 | FRa CDR-L2 | RASNLEA |
| 341 | FRa CDR-L3 | QQSREYPYT |
| 342 | FRa VH | QVQLVQSGAEVVKPGASVKISCKASGYTFTGYFMNWVKQ |
| | | SPGQSLEWIGRIHPYDGDTFY |
| | | NQKFQGKATLTVDKSSNTAHMELLSLTSEDFAVYYCTRYD |
| | | GSRAMDYWGQGTTVTVSS |
| 343 | FRaVL | DIVLTQSPLSLAVSLGQPAIISCKASQSVSFAGTSLMHWYH |
| | | QKPGQQPRLLIYRASNLEAGVPDRFSGSGSKTDFTLTISPVE |
| | | AEDAATYYCQQSREYPYTFGGGTKLEIK |
| 344 | FRa CDR-H1 | GYGLS |
| 345 | FRa CDR-H2 | MISSGGSYTYYADSVKG |
| 346 | FRa CDR-H3 | HGDDPAWFAY |
| 347 | FRaCDR-Ll | SVSSSISSNNLH |
| 348 | FRa CDR-L2 | GTSNLAS |
| 349 | FRa CDR-L3 | QQWSSYPYMYT |
| 350 | FRa VH | EVQLVESGGGVVQPGRSLRLSCSASGFTFSGYGLSWVRQA |
| | | PGKGLEWVAMISSGGSYTYY |
| | | ADSVKGRFAISRDNAKNTLFLQMDSLRPEDTGVYFCARHG |
| | | DDPAWFAYWGQGTPVTVSS |
| 351 | FRaVL | DIQLTQSPSSLSASVGDRVTITCSVSSSISSNNLHWYQQKPG |
| | | KAPKPWIYGTSNLASGVPSRFSGSGSGTDYTFTISSLQPEDI |
| | | ATYYCQQWSSYPYMYTFGQGTKVEIK |
| 352 | MUC-1 CDR-H1 | NYWMN |
| 353 | MUC-1 CDR-H2 | EIRLKSNNYTTHYAESVKG |
| 354 | MUC-1 CDR-H3 | HYYFDY |
| 355 | MUC-1 CDR-L1 | RSSKSLLHSNGITYFF |
| 356 | MUC-1 CDR-L2 | QMSNLAS |
| 357 | MUC-1 CDR-L3 | AQNLELPPT |
| 358 | MUC-1 VH | EVQLVESGGGLVQPGGSMRLSCVASGFPFSNYWMNWVRQ |
| | | APGKGLEWVGEIRLKSNNYTTHYAESVKGRFTISRDDSKNS |
| | | LYLQMNSLKTEDTAVYYCTRHYYFDYWGQGTLVTVSS |
| 359 | MUC-1 VL | DIVMTQSPLSNPVTPGEPASISCRSSKSLLHSNGITYFFWYL |
| | | QKPGQSPQLLIYQMSNLASGVPDRFSGSGSGTDFTLRISRVE |
| | | AEDVGVYYCAQNLELPPTFGQGTKVEIK |
| 360 | Mesothelin CDR-H1 | SYWIG |
| 361 | Mesothelin CDR-H2 | IIDPGDSRTRYSPSFQG |
| 362 | Mesothelin CDR-H3 | GQLYGGTYMDG |
| 363 | Mesothelin CDR-L1 | TGTSSDIGGYNSVS |
| 364 | Mesothelin CDR-L2 | GVNNRPS |
| 365 | Mesothelin CDR-L3 | SSYDIESATPV |
| 366 | Mesothelin VH | QVELVQSGAEVKKPGESLKISCKGSGYSFTSYWIGWVRQA |
| | | PGKGLEWMGIIDPGDSRTRYSPSFQGQVTISADKSISTAYLQ |
| | | WSSLKASDTAMYYCARGQLYGGTYMDGWGQGTLVTVSS |
| 367 | Mesothelin VL | DIALTQPASVSGSPGQSITISCTGTSSDIGGYNSVSWYQQHP |
| | | GKAPKLMIYGVNNRPSGV |
| | | SNRFSGSKSGNTASLTISGLQAEDEADYYCSSYDIESATPVF |
| | | GGGTKLTVL |
| 368 | ROR-1 CDR-H1 | AYNIH |
| 369 | ROR-1 CDR-H2 | SFDPYDGGSSYNQKFKD |
| 370 | ROR-1 CDR-H3 | GWYYFDY |
| 371 | ROR-1 CDR-L1 | RASKSISKYLA |
| 372 | ROR-1 CDR-L2 | SGSTLQS |
| 373 | ROR-1 CDR-L3 | QQHDESPYT |
| 374 | ROR-1 VH | QVQLQESGPGLVKPSQTLSLTCTVSGYAFTAYNIHWVRQA |
| | | PGQGLEWMGSFDPYDGGSSYNQKFKDRLTISKDTSKNQVV |
| | | LTMTNMDPVDTATYYCARGWYYFDYWGHGTLVTVSS |
| 375 | ROR-1 VL | DIVMTQTPLSLPVTPGEPASISCRASKSISKYLAWYQQKPGQ |
| | | APRLLIYSGSTLQSGIPPRFSGSGYGTDFTLTINNIESEDAAY |
| | | YFCQQHDESPYTFGEGTKVEIK |
| 376 | B7H4 CDR-H1 | GSIKSGSYYWG |
| 377 | B7H4 CDR-H2 | NIYYSGSTYYNPSLRS |
| 378 | B7H4 CDR-H3 | AREGSYPNQFDP |
| 379 | B7H4 CDR-L1 | RASQSVSSNLA |
| 380 | B7H4 CDR-L2 | GASTRAT |
| 381 | B7H4 CDR-L3 | QQYHSFPFT |
| 382 | B7H4 VH | QLQLQESGPGLVKPSETLSLTCTVSGGSIKSGSYYWGWIRQ |
| | | PPGKGLEWIGNIYYSGSTY |
| | | YNPSLRSRVTISVDTSKNQFSLKLSSVTAADTAVYYCAREG |
| | | SYPNQFDPWGQGTLVTVSS |
| 383 | B7H4 VL | EIVMTQSPATLSVSPGERATLSCRASQSVSSNLAWYQQKPG |
| | | QAPRLLIYGASTRATGIPA |
| | | RFSGSGSGTEFTLTISSLQSEDFAVYYCQQYHSFPFTFGGGT |
| | | KVEIK |
| 384 | B7-H3 CDR-H1 | SFGMH |
| 385 | B7-H3 CDR-H2 | YISSDSSAIYY |
| 386 | B7-H3 CDR-H3 | GRENIYYGSRLD |
| 387 | B7-H3 CDR-L1 | KASQNVD |
| 388 | B7-H3 CDR-L2 | SASYRYSGVPD |
| 389 | B7-H3 CDR-L3 | QQYNNYPFTFGS |
| 390 | B7-H3 VH | DVQLVESGGGLVQPGGSRKLSCAASGFTFSSFGMHWVRQ |
| | | APEKGLEWVAYISSDSSAIYY |
| | | ADTVKGRFTISRDNPKNTLFLQMTSLRSEDTAMYYCGRGR |
| | | ENIYYGSRLDYWGQGTTLTVSS |
| 391 | B7-H3 VL | DIAMTQSQKFMSTSVGDRVSVTCKASQNVDTNVAWYQQK |
| | | PGQSPKALIYSASYRYSGVPD |
| | | RFTGSGSGTDFTLTINNVQSEDLAEYFCQQYNNYPFTFGSG |
| | | TKLEIK |
| 392 | B7-H3 CDR-H1 | SYWMQWVRQA |
| 393 | B7-H3 CDR-H2 | TIYPGDGDTRY |
| 394 | B7-H3 CDR-H3 | RGIPRLWYFDVM |
| 395 | B7-H3 CDR-L1 | ITCRASQDIS |
| 396 | B7-H3 CDR-L2 | YTSRLHSGVPS |
| 397 | B7-H3 CDR-L3 | QQGNTLPPFTGG |
| 398 | B7-H3 VH | DVQLVESGGGLVQPGGSRKLSCAASGFTFSSFGMHWVRQ |
| | | APEKGLEWVAYISSDSSAIYY |
| | | ADTVKGRFTISRDNPKNTLFLQMTSLRSEDTAMYYCGRGR |
| | | ENIYYGSRLDYWGQGTTLTVSS |
| 399 | B7-H3 VL | DIAMTQSQKFMSTSVGDRVSVTCKASQNVDTNVAWYQQK |
| | | PGQSPKALIYSASYRYSGVPD |
| | | RFTGSGSGTDFTLTINNVQSEDLAEYFCQQYNNYPFTFGSG |
| | | TKLEIK |
| 400 | B7-H3 CDR-H1 | SYGMSWVRQA |
| 401 | B7-H3 CDR-H2 | INSGGSNTYY |
| 402 | B7-H3 CDR-H3 | HDGGAMDYW |
| 403 | B7-H3 CDR-L1 | ITCRASESIYSYLA |
| 404 | B7-H3 CDR-L2 | NTKTLPE |
| 405 | B7-H3 CDR-L3 | HHYGTPPWTFG |
| 406 | B7-H3 VH | EVQLVESGGGLVKPGGSLRLSCAASGFTFSSYGMSWVRQA |
| | | PGKGLEWVATINSGGSNTYY |
| | | PDSLKGRFTISRDNAKNSLYLQMNSLRAEDTAVYYCARHD |
| | | GGAMDYWGQGTTVTVSS |
| 407 | B7-H3 VL | DIQMTQSPSSLSASVGDRVTITCRASESIYSYLAWYQQKPG |
| | | KAPKLLVYNTKTLPEGVPSRFSGSGSGTDFTLTISSLQPEDF |
| | | ATYYCQHHYGTPPWTFGQGTRLEIK |
| 408 | B7-H3 CDR-H1 | SFGMHWVRQA |
| 409 | B7-H3 CDR-H2 | ISSGSGTIYYADTVKGRFTI |
| 410 | B7-H3 CDR-H3 | HGYRYEGFDYWG |
| 411 | B7-H3 CDR-L1 | ITCKASQNVDTNVA |
| 412 | B7-H3 CDR-L2 | SASYRYSGVPS |
| 413 | B7-H3 CDR-L3 | QQYNNYPFTFGQ |
| 414 | B7-H3 VH | EVQLVESGGGLVQPGGSLRLSCAASGFTFSSFGMHWVRQA |
| | | PGKGLEWVAYISSGSGTIY |
| | | YADTVKGRFTISRDNAKNSLYLQMNSLRAEDTAVYYCAR |
| | | HGYRYEGFDYWGQGTTVTVSS |
| 415 | B7-H3 VL | DIQMTQSPSFLSASVGDRVTITCKASQNVDTNVAWYQQKP |
| | | GKAPKALIYSASYRYSGVPSRFSGSGSGTDFTLTISSLQPED |
| | | FAEYFCQQYNNYPFTFGQGTKLEIK |
| 416 | B7-H3 CDR-H1 | NYVMH |
| 417 | B7-H3 CDR-H2 | YINPYNDDVKYNEKFKG |
| 418 | B7-H3 CDR-H3 | WGYYGSPLYYFDY |
| 419 | B7-H3 CDR-L1 | RASSRLIYMH |
| 420 | B7-H3 CDR-L2 | ATSNLAS |
| 421 | B7-H3 CDR-L3 | QQWNSNPPT |
| 422 | B7-H3 VH | EVQLQQSGPELVKPGASVKMSCKASGYTFTNYVMHWVKQ |
| | | KPGQGLEWIGYINPYNDDVKYNEKFKGKATQTSDKSSSTA |
| | | YMELSSLTSEDSAVYYCARWGYYGSPLYYFDYWGQGTTL |
| | | TVSS |
| 423 | B7-H3 VL | QIVLSQSPTILSASPGEKVTMTCRASSRLIYMHWYQQKPGS |
| | | SPKPWIYATSNLASGVPAR |
| | | FSGSGSGTSYSLTISRVEAEDAATYYCQQWNSNPPTFGTGT |
| | | KLELK |
| 424 | B7-H3 CDR-H1 | NYVMH |
| 425 | B7-H3 CDR-H2 | YINPYNDDVKYNEKFKG |
| 426 | B7-H3 CDR-H3 | WGYYGSPLYYFDY |
| 427 | B7-H3 CDR-L1 | RASSRLIYMH |
| 428 | B7-H3 CDR-L2 | ATSNLAS |
| 429 | B7-H3 CDR-L3 | QQWNSNPPT |
| 430 | B7-H3 VH | QVQLVQSGAEVKKPGSSVKVSCKASGYTFTNYVMHWVRQ |
| | | APGQGLEWMGYINPYNDDVKYNE |
| | | KFKGRVTITADESTSTAYMELSSLRSEDTAVYYCARWGYY |
| | | GSPLYYFDYWGQGTLVTVSS |
| 431 | B7-H3 VL | EIVLTQSPATLSLSPGERATLSCRASSRLIYMHWYQQKPGQ |
| | | APRPLIYATSNLASGIPARFSGSGSGTDFTLTISSLEPEDFAV |
| | | YYCQQWNSNPPTFGQGTKVEIK |
| 432 | B7-H3 CDR-H1 | GYSFTSYTIH |
| 433 | B7-H3 CDR-H2 | YINPNSRNTDYAQKFQG |
| 434 | B7-H3 CDR-H3 | YSGSTPYWYFDV |
| 435 | B7-H3 CDR-L1 | RASSSVSYMN |
| 436 | B7-H3 CDR-L2 | ATSNLAS |
| 437 | B7-H3 CDR-L3 | QQWSSNPLT |
| 438 | B7-H3 VH | EVQLVQSGAEVKKPGSSVKVSCKASGYSFTSYTIHWVRQA |
| | | PGQGLEWMGYINPNSRNTDYAQKFQGRVTLTADKSTSTA |
| | | YMELSSLRSEDTAVYYCARYSGSTPYWYFDVWGQGTTVT |
| | | VSS |
| 439 | B7-H3 VL | DIQMTQSPSSLSASVGDRVTITCKASQNVGFNVAWYQQKP |
| | | GKSPKALIYSASYRYSGVPSRFSGSGSGTDFTLTISSLQPEDF |
| | | AEYFCQQYNWYPFTFGQGTKLEIK |
| 440 | B7-H3 CDR-H1 | GYTFSSYWMH |
| 441 | B7-H3 CDR-H2 | LIHPDSGSTNYNEMFKN |
| 442 | B7-H3 CDR-H3 | GGRLYFD |
| 443 | B7-H3 CDR-L1 | RSSQSLVHSNGDTYLR |
| 444 | B7-H3 CDR-L2 | KVSNRFS |
| 445 | B7-H3 CDR-L3 | SQSTHVPYT |
| 446 | B7-H3 VH | EVQLVQSGAEVKKPGSSVKVSCKASGYTFSSYWMHWVRQ |
| | | APGQGLEWIGLIHPDSGSTNYNEMFKNRATLTVDRSTSTAY |
| | | VELSSLRSEDTAVYFCAGGGRLYFDYWGQGTTVTVSS |
| 447 | B7-H3 VL | DVVMTQSPLSLPVTPGEPASISCRSSQSLVHSNGDTYLRWY |
| | | LQKPGQSPQLLIYKVSNRFSGVPDRFSGSGSGTDFTLKISRV |
| | | EAEDVGVYYCSQSTHVPYTFGGGTKVEIK |
| 448 | B7-H3 CDR-H1 | GYTFSSYWMH |
| 449 | B7-H3 CDR-H2 | LIHPESGSTNYNEMFKN |
| 450 | B7-H3 CDR-H3 | GGRLYFDY |
| 451 | B7-H3 CDR-L1 | RSSQSLVHSNQDTYLR |
| 452 | B7-H3 CDR-L2 | KVSNRFS |
| 453 | B7-H3 CDR-L3 | SQSTHVPYT |
| 454 | B7-H3 VH | EVQLVQSGAEVKKPGSSVKVSCKASGYTFSSYWMHWVRQ |
| | | APGQGLEWIGLIHPESGSTNY |
| | | NEMFKNRATLTVDRSTSTAYMELSSLRSEDTAVYYCAGGG |
| | | RLYFDYWGQGTTVTVSS |
| 455 | B7-H3 VL | DIVMTQSPLSLPVTPGEPASISCRSSQSLVHSNQDTYLRWYL |
| | | QKPGQSPQLLIYKVSNRF |
| | | SGVPDRFSGSGSGTDFTLKKISRVEAEDVGVYYCSQSTHVP |
| | | YTFGGGTKVEIK |
| 456 | B7-H3 CDR-H1 | TGYSITSGYSWH |
| 457 | B7-H3 CDR-H2 | YIHSSGSTNYNPSLKS |
| 458 | B7-H3 CDR-H3 | YDDYFEY |
| 459 | B7-H3 CDR-L1 | KASQNVGFNVAW |
| 460 | B7-H3 CDR-L2 | SASYRYS |
| 461 | B7-H3 CDR-L3 | QQYNWYPFT |
| 462 | B7-H3 VH | EVQLQESGPGLVKPSETLSLTCAVTGYSITSGYSWHWIRQF |
| | | PGNGLEWMGYIHSSGSTNY |
| | | NPSLKSRISISRDTSKNQFFLKLSSVTAADTAVYYCAGYDD |
| | | YFEYWGQGTTVTVSS |
| 463 | B7-H3 VL | DIQMTQSPSSLSASVGDRVTITCKASQNVGGFNVAWYQQK |
| | | PGKSPKALIYSASYRYSGV |
| | | PSRFSGSGSGTDFTLTISSLQPEDFAEYFCQQYNWYPFTFGQ |
| | | GTKLEIK |
| 464 | B7-H3 CDR-H1 | NYDIN |
| 465 | B7-H3 CDR-H2 | WIGWIFPGDDSTQYNEKFKG |
| 466 | B7-H3 CDR-H3 | QTTGTWFAY |
| 467 | B7-H3 CDR-L1 | RASQSISDYLY |
| 468 | B7-H3 CDR-L2 | YASQSIS |
| 469 | B7-H3 CDR-L3 | CQNGHSFPL |
| 470 | B7-H3 VH | QVQLVQSGAEVVKPGASVKLSCKTSGYTFTNYDINWVRQ |
| | | RPGQGLEWIGWIFPGDDSTQY |
| | | NEKFKGKATLTTDTSTSTAYMELSSLRSEDTAVYFCARQTT |
| | | GTWFAYWGQGTLVTVSS |
| 471 | B7-H3 VL | EIVMTQSPATLSVSPGERVTLSCRASQSISDYLYWYQQKSH |
| | | ESPRLLIKYASQSISGIPA |
| | | RFSGSGSGSEFTLTINSVEPEDVGVYYCQNGHSFPLTFGQGT |
| | | KLELK |
| 472 | B7-H3 VH | QVQLQQSGAEVKKPGSSVKVSCKASGGTFSSYAISWVRQA |
| | | PGQGLEWMGGIIPILGIAN |
| | | YAQKFQGRVTITADESTSTAYMELSSLRSEDTAVYYCARG |
| | | GSGSYHMDVWGKGTTVTVSS |
| 473 | B7-H3 VL | EIVLTQSPATLSLSPGERATLSCRASQSVSSYLAWYQQKPG |
| | | QAPRLLIYDASNRATGIP |
| | | ARFSGSGSGTDFTLTISSLEPEDFAVYYCQQRSNWPPRITFG |
| | | QGTRLEIK |
| 474 | B7-H3 CDR-H1 | IYNVH |
| 475 | B7-H3 CDR-H2 | TIFPGNGDTSYNQKFKD |
| 476 | B7-H3 CDR-H3 | WDDGNVGFAH |
| 477 | B7-H3 CDR-L1 | RASENINNYLT |
| 478 | B7-H3 CDR-L2 | HAKTLAE |
| 479 | B7-H3 CDR-L3 | QHHYGTPPT |
| 480 | B7-H3 VH | QVQLQQPGAELVKPGASVKMSCKASGYTFTIYNVHWIKQT |
| | | PGQGLEWMGTIFPGNGDTSY |
| | | NQKFKDKATLTTDKSSKTAYMQLNSLTSEDSAVYYCARW |
| | | DDGNVGFAHWGQGTLVTVSA |
| 481 | B7-H3 VL | DIQMTQSPASLSASVGETVTITCRASENINNYLTWFQQKQG |
| | | KSPQLLVYHAKTLAEGVPS |
| | | RFSGSGSGTQFSLKINSLQPEDFGSYYCQHHYGTPPTFGGG |
| | | TKLEIK |
| 482 | B7-H3 VH | EVQLVQSGAEVKKPGASVKVSCKASGYTFTIYNVHWVRQ |
| | | APGQGLEWMGTIFPGNGDTS |
| | | YNQKFKDKVTMTTDTSTSTAYMELSSLRSEDTAVYYCAR |
| | | WDDGNVGFAHWGQGTLVTVSS |
| 483 | B7-H3 VL | DIQMTQSPSSLSASVGDRVTITCRASENINNYLTWFQQKQG |
| | | KSPQLLIYHAKTLAEGVP |
| | | SRFSGSGSGTDFTLTISSLQPEDFATYYCQHHYGTPPTFGGG |
| | | TKVEIK |
| 484 | B7-H3 VH | EVQLVQSGAEVKKPGASVKVSCKASGYTFTIYNVHWIRQA |
| | | PGQGLEWMGTIFPGNGDTSY |
| | | NQKFKDRATLTTDKSTKTAYMELRSLRSDDTAVYYCARW |
| | | DDGNVGFAHWGQGTLVTVSS |
| 485 | B7-H3 VL | DIQMTQSPSSLSASVGDRVTITCRASENINNYLTWFQQKPG |
| | | I<API<LLVYHAI<TLAEGVPS |
| | | RFSGSGSGTQFTLTISSLQPEDFATYYCQHHYGTPPTFGQGT |
| | | KLEIK |
| 486 | HER3 H | QVQLQQWGAGLLKPSETLSLTCAVYGGSFSGYYWSWIRQP |
| | | PGKGLEWIGEINHSGSTNYN |
| | | PSLKSRVTISVETSKNQFSLKLSSVTAADTAVYYCARDKWT |
| | | WYFDLWGRGTLVTVSSAST |
| | | KGPSVFPLAPSSKSTSGGTAALGCLVKDYFPEPVTVSWNSG |
| | | ALTSGVHTFPAVLQSSGL |
| | | YSLSSVVTVPSSSLGTQTYICNVNHKPSNTKVDKRVEPKSC |
| | | DKTHTCPPCPAPELLGGPSVFLFPPKPKDTLMISRTPEVTCV |
| | | VVDVSHEDPEVI<FNWYVDGVEVHNAI<TI<PREEQYNSTYR |
| | | VVSVLTVLHQDWLNGKEYKCKVSNKALPAPIEKTISKAKG |
| | | QPREPQVYTLPPSREEMTKNQVSLTCLVKGFYPSDIAVEWE |
| | | SNGQPENNYKTTPPVLDSDGSFFLYSKLTVDKSRWQQGNV |
| | | FSCSVMHEALHNHYTQKSLSLSPGK |
| 487 | HER3 L | DIEMTQSPDSLAVSLGERATINCRSSQSVLYSSSNRNYLAW |
| | | YQQNPGQPPKLLIYWASTRESGVPDRFSGSGSGTDFTLTISS |
| | | LQAEDVAVYYCQQYYSTPRTFGQGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| 488 | HER3 H | EVQLLESGGGLVQPGGSLRLSCAASGFTFSHYVMAWVRQ |
| | | APGKGLEWVSSISSSGGWTLY |
| | | ADSVKGRFTISRDNSKNTLYLQMNSLRAEDTAVYYCTRGL |
| | | KMATIFDYWGQGTLVTVSSA |
| | | STKGPSVFPLAPCSRSTSESTAALGCLVKDYFPEPVTVSWN |
| | | SGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDGV |
| | | EVHNAKTKPREEQFNSTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSDG |
| | | SFFLYSKLTVDKSRWQQGNV |
| | | FSCSVMHEALHNHYTQKSLSLSPGK |
| 489 | HER3 L | QSALTQPASVSGSPGQSITISCTGTSSDVGSYNVVSWYQQH |
| | | PGKAPKLIIYEVSQRPSGVSNRFSGSKSGNTASLTISGLQTE |
| | | DEADYYCCSYAGSSIFVIFGGGTKVTVLGQPKAAPSVTLFP |
| | | PSSEELQANKATLVCLVSDFYPGAVTVAWKADGSPVKVG |
| | | VETTKPSKQSNNKYAASSYLSLTPEQWKSHRSYSCRVTHE |
| | | GSTVEKTVAPAECS |
| 490 | HER3 H | EVQLLESGGGLVQPGGSLRLSCAASGFTFSSYAMSWVRQA |
| | | PGKGLEWVSAINSQGKSTYYADSVKGRFTISRDNSKNTLYL |
| | | QMNSLRAEDTAVYYCARWGDEGFDIWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSREEMTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSP |
| | | GK |
| 491 | HER3 L | DIQMTQSPSSLSASVGDRVTITCRASQGISNWLAWYQQKPG |
| | | KAPKLLIYGASSLQSGVPSRFSGSGSGTDFTLTISSLQPEDFA |
| | | TYYCQQYSSFPTTFGQGTKVEIKRTVAAPSVFIFPPSDEQLK |
| | | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| 492 | HER3 H | QVQLVQSGAEVKKPGASVKVSCKASGYTFRSSYISWVRQA |
| | | PGQGLEWMGWIYAGTGSPSYNQKLQGRVTMTTDTSTSTA |
| | | YMELRSLRSDDTAVYYCARHRDYYSNSLTYWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPAPIEKTISKAKGQPREPQVYTLPPSRDELT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPG |
| 493 | HER3 L | DIVMTQSPDSLAVSLGERATINCKSSQSVLNSGNQKNYLT |
| | | WYQQKPGQPPKLLIYWASTRESGVPDRFSGSGSGTDFTLTI |
| | | SSLQAEDVAVYYCQSDYSYPYTFGQGTKLEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| 494 | PTK7 CDR-H1 | TSNMGVG |
| 495 | PTK7 CDR-H2 | HIWWDDDKYYSPSLKS |
| 496 | PTK7 CDR-H3 | SNYGYAWFAY |
| 497 | PTK7 CDR-L1 | KASQDIYPYLN |
| 498 | PTK7 CDR-L2 | RTNRLLD |
| 499 | PTK7 CDR-L3 | LQYDEFPLT |
| 500 | PTK7 VH | QITLKESGPTLVKPTQTLTLTCTFSGFSLSTSNMGVGWIRQP |
| | | PGKALEWLAHIWWDDDKYYSPSLKSRLTITKDTSKNQVVL |
| | | TMTNMDPVDTATYYCVRSNYGYAWFAYWGQGTLVTVSS |
| 501 | PTK7 VL | DIQMTQSPSSLSASVGDRVTITCKASQDIYPYLNWFQQKPG |
| | | KAPKTLIYRTNRLLDGVPS |
| | | RFSGSGSGTDFTFTISSLQPEDIATYYCLQYDEFPLTFGAGT |
| | | KLEIK |
| 502 | PTK7 CDR-H1 | DYAVH |
| 503 | PTK7 CDR-H2 | VISTYNDYTYNNQDFKG |
| 504 | PTK7 CDR-H3 | GNSYFYALDY |
| 505 | PTK7 CDR-L1 | RASESVDSYGKSFMH |
| 506 | PTK7 CDR-L2 | RASNLES |
| 507 | PTK7 CDR-L3 | QQSNEDPWT |
| 508 | PTK7 VH | QVQLVQSGPEVKKPGASVKVSCKASGYTFTDYAVHWVRQ |
| | | APGKRLEWIGVISTYNDYTY |
| | | NNQDFKGRVTMTRDTSASTAYMELSRLRSEDTAVYYCAR |
| | | GNSYFYALDYWGQGTSVTVSS |
| 509 | PTK7 VL | EIVLTQSPATLSLSPGERATLSCRASESVDSYGKSFMHWYQ |
| | | QKPGQAPRLLIYRASNLES |
| | | GIPARFSGSGSGTDFTLTISSLEPEDFAVYYCQQSNEDPWTF |
| | | GGGTKLEIK |
| 510 | PTK7 CDR-H1 | RYWMS |
| 511 | PTK7 CDR-H2 | DLNPDSSAINYVDSVKG |
| 512 | PTK7 CDR-H3 | ITTLVPYTMDF |
| 513 | PTK7 CDR-L1 | ITNTDIDDDMN |
| 514 | PTK7 CDR-L2 | EGNGLRP |
| 515 | PTK7 CDR-L3 | LQSDNLPLT |
| 516 | PTK7 VH | EVQLVESGGGLVQPGGSLRLSCAASGFDFSRYWMSWVRQ |
| | | APGKGLEWIGDLNPDSSAINY |
| | | VDSVKGRFTISRDNAKNSLYLQMNSLRAEDTAVYYCTLITT |
| | | LVPYTMDFWGQGTSVTVSS |
| 517 | PTK7 VL | ETTLTQSPAFMSATPGDKVNISCITNTDIDDDMNWYQQKP |
| | | GEAAILLISEGNGLRPGIPPRFSGSGYGTDFTLTINNIESEDA |
| | | AYYFCLQSDNLPLTFGSGTKLEIK |
| 518 | LIV1 CDR-H1 | DYYMH |
| 519 | LIV1 CDR-H2 | WIDPENGDTEYGPKFQG |
| 520 | LIV1 CDR-H3 | HNAHYGTWFAY |
| 521 | LIV1 CDR-L1 | RSSQSLLHSSGNTYLE |
| 522 | LIV1 CDR-L2 | KISTRFS |
| 523 | LIV1 CDR-L3 | FQGSHVPYT |
| 524 | LIV1 VH | QVQLVQSGAEVKKPGASVKVSCKASGLTIEDYYMHWVRQ |
| | | APGQGLEWMGWIDPENGDTEY |
| | | GPKFQGRVTMTRDTSINTAYMELSRLRSDDTAVYYCAVHN |
| | | AHYGTWFAYWGQGTLVTVSS |
| 525 | LIV1 VL | DVVMTQSPLSLPVTLGQPASISCRSSQSLLHSSGNTYLEWY |
| | | QQRPGQSPRPLIYKISTRFSGVPDRFSGSGSGTDFTLKISRVE |
| | | AEDVGVYYCFQGSHVPYTFGGGTKVEIK |
| 526 | avb6 CDR-H1 | DYNVN |
| 527 | avb6 CDR-H2 | VINPKYGTTRYNQKFKG |
| 528 | avb6 CDR-H3 | GLNAWDY |
| 529 | avb6 CDR-L1 | GASENIYGALN |
| 530 | avb6 CDR-L2 | GATNLED |
| 531 | avb6 CDR-L3 | QNVLTTPYT |
| 532 | avb6 VH | QFQLVQSGAEVKKPGASVKVSCKASGYSFTDYNVNWVRQ |
| | | APGQGLEWIGVINPKYGTTRY |
| | | NQKFKGRATLTVDKSTSTAYMELSSLRSEDTAVYYCTRGL |
| | | NAWDYWGQGTLVTVSS |
| 533 | avb6 VL | DIQMTQSPSSLSASVGDRVTITCGASENIYGALNWYQQKPG |
| | | KAPKLLIYGATNLEDGVPS |
| | | RFSGSGSGRDYTFTISSLQPEDIATYYCQNVLTTPYTFGQGT |
| | | KLEIK |
| 534 | avb6 CDR-H1 | GYFMN |
| 535 | avb6 CDR-H2 | LINPYNGDSFYNQKFKG |
| 536 | avb6 CDR-H3 | GLRRDFDY |
| 537 | avb6 CDR-L1 | KSSQSLLDSDGKTYLN |
| 538 | avb6 CDR-L2 | LVSELDS |
| 539 | avb6 CDR-L3 | WQGTHFPRT |
| 540 | avb6 VH | QVQLVQSGAEVKKPGASVKVSCKASGYSFSGYFMNWVRQ |
| | | APGQGLEWMGLINPYNGDSFY |
| | | NQKFKGRVTMTRQTSTSTVYMELSSLRSEDTAVYYCVRGL |
| | | RRDFDYWGQGTLVTVSS |
| 541 | avb6 VL | DVVMTQSPLSLPVTLGQPASISCKSSQSLLDSDGKTYLNWL |
| | | FQRPGQSPRRLIYLVSELD |
| | | SGVPDRFSGSGSGTDFTLKISRVEAEDVGVYYCWQGTHFP |
| | | RTFGGGTKLEIK |
| 542 | CD48 CDR-H1 | DFGMN |
| 543 | CD48 CDR-H2 | WINTFTGEPSYGNVFKG |
| 544 | CD48 CDR-H3 | RHGNGNVFDS |
| 545 | CD48 CDR-L1 | RASQSIGSNIH |
| 546 | CD48 CDR-L2 | YTSESIS |
| 547 | CD48 CDR-L3 | QQSNSWPLT |
| 548 | CD48 VH | QVQLVQSGSELKKPGASVKVSCKASGYTFTDFGMNWVRQ |
| | | APGQGLEWMGWINTFTGEPSYGNVFKGRFVFSLDTSVSTA |
| | | YLQISSLKAEDTAVYYCARRHGNGNVFDSWGQGTLVTVSS |
| 549 | CD48 VL | EIVLTQSPDFQSVTPKEKVTITCRASQSIGSNIHWYQQKPDQ |
| | | SPKLLIKYTSESISGVPSRFSGSGSGTDFTLTINSLEAEDAAT |
| | | YYCQQSNSWPLTFGGGTKVEIKR |
| 550 | PD-L1 CDR-H1 | TAAIS |
| 551 | PD-L1 CDR-H2 | GIIPIFGKAHYAQKFQG |
| 552 | PD-L1 CDR-H3 | KFHFVSGSPFGMDV |
| 553 | PD-L1 CDR-L1 | RASQSVSSYLA |
| 554 | PD-L1 CDR-L2 | DASNRAT |
| 555 | PD-L1 CDR-L3 | QQRSNWPT |
| 556 | PD-L1 VH | QVQLVQSGAEVKKPGSSVKVSCKTSGDTFSTAAISWVRQA |
| | | PGQGLEWMGGIIPIFGKAHYAQKFQGRVTITADESTSTAYM |
| | | ELSSLRSEDTAVYFCARKFHFVSGSPFGMDVWGQGTTVTV |
| | | SS |
| 557 | PD-L1 VL | EIVLTQSPATLSLSPGERATLSCRASQSVSSYLAWYQQKPG |
| | | QAPRLLIYDASNRATGIPA |
| | | RFSGSGSGTDFTLTISSLEPEDFAVYYCQQRSNWPTFGQGT |
| | | KVEIK |
| 558 | IGF-1R CDR-H1 | SYAIS |
| 559 | IGF-1R CDR-H2 | GIIPIFGTANYAQKFQG |
| 560 | IGF-1R CDR-H3 | APLRFLEWSTQDHYYYYYMDV |
| 561 | IGF-1R CDR-L1 | QGDSLRSYYAT |
| 562 | IGF-1R CDR-L2 | GENKRPS |
| 563 | IGF-1R CDR-L3 | KSRDGSGQHLV |
| 564 | IGF-1R VH | EVQLVQSGAEVKKPGSSVKVSCKASGGTFSSYAISWVRQA |
| | | PGQGLEWMGGIIPIFGTANY |
| | | AQKFQGRVTITADKSTSTAYMELSSLRSEDTAVYYCARAP |
| | | LRFLEWSTQDHYYYYYMDVWGKGTTVTVSS |
| 565 | IGF-1R VL | SSELTQDPAVSVALGQTVRITCQGDSLRSYYATWYQQKPG |
| | | QAPILVIYGENKRPSGIPDR |
| | | FSGSSSGNTASLTITGAQAEDEADYYCKSRDGSGQHLVFGG |
| | | GTKLTVL |
| 566 | Claudin-18.2 CDR- | SYWIN |
| | H1 | |
| 567 | Claudin-18.2 CDR- | NIYPSDSYTNYNQKFKD |
| | H2 | |
| 568 | Claudin-18.2 CDR- | SWRGNSFDY |
| | H3 | |
| 569 | Claudin-18.2 CDR- | KSSQSLLNSGNQKNYLT |
| | L1 | |
| 570 | Claudin-18.2 CDR- | WASTRES |
| | L2 | |
| 571 | Claudin-18.2 CDR- | QNDYSYPFT |
| | L3 | |
| 572 | Claudin-18.2 VH | QVQLQQPGAELVRPGASVKLSCKASGYTFTSYWINWVKQ |
| | | RPGQGLEWIGNIYPSDSYTN |
| | | YNQKFKDKATLTVDKSSSTAYMQLSSPTSEDSAVYYCTRS |
| | | WRGNSFDYWGQGTTLTVSS |
| 573 | Claudin-18.2 VL | DIVMTQSPSSLTVTAGEKVTMSCKSSQSLLNSGNQKNYLT |
| | | WYQQKPGQPPKLLIYWASTR |
| | | ESGVPDRFTGSGSGTDFTLTISSVQAEDLAVYYCQNDYSYP |
| | | FTFGSGTKLEIK |
| 574 | Claudin-18.2 CDR- | NYGMN |
| | H1 | |
| 575 | Claudin-18.2 CDR- | WINTNTGEPTYAEEFKG |
| | H2 | |
| 576 | Claudin-18.2 CDR- | LGFGNAMDY |
| | H3 | |
| 577 | Claudin-18.2 CDR- | KSSQSLLNSGNQKNYLT |
| | L1 | |
| 578 | Claudin-18.2 CDR- | WASTRES |
| | L2 | |
| 579 | Claudin-18.2 CDR- | QNDYSYPLT |
| | L3 | |
| 580 | Claudin-18.2 VH | QIQLVQSGPELKKPGETVKISCKASGYTFTNYGMNWVKQA |
| | | PGKGLKWMGWINTNTGEPTY |
| | | AEEFKGRFAFSLETSASTAYLQINNLKNEDTATYFCARLGF |
| | | GNAMDYWGQGTSVTVSS |
| 581 | Claudin-18.2 VL | DIVMTQSPSSLTVTAGEKVTMSCKSSQSLLNSGNQKNYLT |
| | | WYQQKPGQPPKLLIYWASTR |
| | | ESGVPDRFTGSGSGTDFTLTISSVQAEDLAVYYCQNDYSYP |
| | | LTFGAGTKLELK |
| 582 | Nectin-4 CDR-H1 | SYNMN |
| 583 | Nectin-4 CDR-H2 | YISSSSSTIYYADSVKG |
| 584 | Nectin-4 CDR-H3 | AYYYGMDV |
| 585 | Nectin-4 CDR-L1 | RASQGISGWLA |
| 586 | Nectin-4 CDR-L2 | AASTLQS |
| 587 | Nectin-4 CDR-L3 | QQANSFPPT |
| 588 | Nectin-4 VH | EVQLVESGGGLVQPGGSLRLSCAASGFTFSSYNMNWVRQA |
| | | PGKGLEWVSYISSSSSTIYY |
| | | ADSVKGRFTISRDNAKNSLSLQMNSLRDEDTAVYYCARAY |
| | | YYGMDVWGQGTTVTVSS |
| 589 | Nectin-4 VL | DIQMTQSPSSVSASVGDRVTITCRASQGISGWLAWYQQKP |
| | | GKAPKFLIYAASTLQSGVPS |
| | | RFSGSGSGTDFTLTISSLQPEDFATYYCQQANSFPPTFGGGT |
| | | KVEIK |
| 590 | SLTRK6 CDR-H1 | SYGMH |
| 591 | SLTRK6 CDR-H2 | VIWYDGSNQYYADSVKG |
| 592 | SLTRK6 CDR-H3 | GLTSGRYGMDV |
| 593 | SLTRK6 CDR-L1 | RSSQSLLLSHGFNYLD |
| 594 | SLTRK6 CDR-L2 | LGSSRAS |
| 595 | SLTRK6 CDR-L3 | MQPLQIPWT |
| 596 | SLTRK6 VH | QVQLVESGGGVVQPGRSLRLSCAASGFTFSSYGMHWVRQ |
| | | APGKGLEWVAVIWYDGSNQYY |
| | | ADSVKGRFTISRDNSKNTLFLQMHSLRAEDTAVYYCARGL |
| | | TSGRYGMDVWGQGTTVTVSS |
| 597 | SLTRK6 VL | DIVMTQSPLSLPVTPGEPASISCRSSQSLLLSHGFNYLDWYL |
| | | QKPGQSPQLLIYLGSSRASGVPDRFSGSGSGTDFTLKISRVE |
| | | AEDVGLYYCMQPLQIPWTFGQGTKVEIK |
| 598 | CD228 CDR-H1 | SGYWN |
| 599 | CD228 CDR-H2 | YISDSGITYYNPSLKS |
| 600 | CD228 CDR-H3 | RTLATYYAMDY |
| 601 | CD228 CDR-L1 | RASQSLVHSDGNTYLH |
| 602 | CD228 CDR-L2 | RVSNRFS |
| 603 | CD228 CDR-L3 | SQSTHVPPT |
| 604 | CD228 VH | QVQLQESGPGLVKPSETLSLTCTVSGDSITSGYWNWIRQPP |
| | | GKGLEYIGYISDSGITYYN |
| | | PSLKSRVTISRDTSKNQYSLKLSSVTAADTAVYYCARRTLA |
| | | TYYAMDYWGQGTLVTVSS |
| 605 | CD228 VL | DFVMTQSPLSLPVTLGQPASISCRASQSLVHSDGNTYLHWY |
| | | QQRPGQSPRLLIYRVSNRFSGVPDRFSGSGSGTDFTLKISRV |
| | | EAEDVGVYYCSQSTHVPPTFGQGTKLEIKR |
| 606 | CD142 (TF) CDR- | NYAMS |
| | H1 | |
| 607 | CD142 (TF) CDR- | SISGSGDYTYYTDSVKG |
| | H2 | |
| 608 | CD142 (TF) CDR- | SPWGYYLDS |
| | H3 | |
| 609 | CD142 (TF) CDR- | RASQGISSRLA |
| | L1 | |
| 610 | CD142 (TF) CDR- | AASSLQS |
| | L2 | |
| 611 | CD142 (TF) CDR- | QQYNSYPYT |
| | L3 | |
| 612 | CD142 (TF) VH | EVQLLESGGGLVQPGGSLRLSCAASGFTFSNYAMSWVRQA |
| | | PGKGLEWVSSISGSGDYTY |
| | | YTDSVKGRFTISRDNSKNTLYLQMNSLRAEDTAVYYCARS |
| | | PWGYYLDSWGQGTLVTVSS |
| 613 | CD142 (TF) VL | DIQMTQSPPSLSASAGDRVTITCRASQGISSRLAWYQQKPE |
| | | KAPKSLIYAASSLQSGVPS |
| | | RFSGSGSGTDFTLTISSLQPEDFATYYCQQYNSYPYTFGQGT |
| | | KLEIK |
| 614 | STn CDR-H1 | DHAIH |
| 615 | STn CDR-H2 | YFSPGNDDIKYNEKFRG |
| 616 | STn CDR-H3 | SLSTPY |
| 617 | STn CDR-L1 | KSSQSLLNRGNHKNYLT |
| 618 | STn CDR-L2 | WASTRES |
| 619 | STn CDR-L3 | QNDYTYPYT |
| 620 | STn VH | EVQLVQSGAEVKKPGASVKVSCKASGYTFTDHAIHWVRQ |
| | | APGQGLEWMGYFSPGNDDIKY |
| | | NEKFRGRVTMTADKSSSTAYMELRSLRSDDTAVYFCKRSL |
| | | STPYWGQGTLVTVSS |
| 621 | STn VL | DIVMTQSPDSLAVSLGERATINCKSSQSLLNRGNHKNYLT |
| | | WYQQKPGQPPKLLIYWAST |
| | | RESGVPDRFSGSGSGTDFTLTISSLQAEDVAVYYCQNDYTY |
| | | PYTFGQGTKVEIK |
| 622 | CD20 CDR-H1 | SYNMH |
| 623 | CD20 CDR-H2 | AIYPGNGDTSYNQKFKG |
| 624 | CD20 CDR-H3 | STYYGGDWYFNV |
| 625 | CD20 CDR-L1 | RASSSVSYIH |
| 626 | CD20 CDR-L2 | ATSNLAS |
| 627 | CD20 CDR-L3 | QQWTSNPPT |
| 628 | CD20 VH | QVQLQQPGAELVKPGASVKMSCKASGYTFTSYNMHWVK |
| | | QTPGRGLEWIGAIYPGNGDTSY |
| | | NQKFKGKATLTADKSSSTAYMQLSSLTSEDSAVYYCARST |
| | | YYGGDWYFNVWGAGTTVTVSA |
| 629 | CD20 VL | QIVLSQSPAILSASPGEKVTMTCRASSSVSYIHWFQQKPGSS |
| | | PKPWIYATSNLASGVPVR |
| | | FSGSGSGTSYSLTISRVEAEDAATYYCQQWTSNPPTFGGGT |
| | | KLEIK |
| 630 | HER2 CDR-H1 | DTYIH |
| 631 | HER2 CDR-H2 | RIYPTNGYTRYADSVKG |
| 632 | HER2 CDR-H3 | WGGDGFYAMDY |
| 633 | HER2 CDR-L1 | RASQDVNTAVA |
| 634 | HER2 CDR-L2 | SASFLYS |
| 635 | HER2 CDR-L3 | QQHYTTPPT |
| 636 | HER2 VH | EVQLVESGGGLVQPGGSLRLSCAASGFNIKDTYIHWVRQA |
| | | PGKGLEWVARIYPTNGYTRY |
| | | ADSVKGRFTISADTSKNTAYLQMNSLRAEDTAVYYCSRW |
| | | GGDGFYAMDYWGQGTLVTVSS |
| 637 | HER2 VL | DIQMTQSPSSLSASVGDRVTITCRASQDVNTAVAWYQQKP |
| | | GKAPKLLIYSASFLYSGVPS |
| | | RFSGSRSGTDFTLTISSLQPEDFATYYCQQHYTTPPTFGQGT |
| | | KVEIK |
| 638 | CD79b CDR-H1 | SYWIE |
| 639 | CD79b CDR-H2 | EILPGGGDTNYNEIFKG |
| 640 | CD79b CDR-H3 | RVPIRLDY |
| 641 | CD79b CDR-L1 | KASQSVDYEGDSFLN |
| 642 | CD79b CDR-L2 | AASNLES |
| 643 | CD79b CDR-L3 | QQSNEDPLT |
| 644 | CD79b VH | EVQLVESGGGLVQPGGSLRLSCAASGYTFSSYWIEWVRQA |
| | | PGKGLEWIGEILPGGGDTNYNEIFKGRATFSADTSKNTAYL |
| | | QMNSLRAEDTAVYYCTRRVPIRLDYWGQGTLVTVSS |
| 645 | CD79b VL | DIQLTQSPSSLSASVGDRVTITCKASQSVDYEGDSFLNWYQ |
| | | QKPGKAPKLLIYAASNLES |
| | | GVPSRFSGSGSGTDFTLTISSLQPEDFATYYCQQSNEDPLTF |
| | | GQGTKVEIK |
| 646 | NaPi2B CDR-H1 | DFAMS |
| 647 | NaPi2B CDR-H2 | TIGRVAFHTYYPDSMKG |
| 648 | NaPi2B CDR-H3 | HRGFDVGHFDF |
| 649 | NaPi2B CDR-L1 | RSSETLVHSSGNTYLE |
| 650 | NaPi2B CDR-L2 | RVSNRFS |
| 651 | NaPi2B CDR-L3 | FQGSFNPLT |
| 652 | NaPi2B VH | EVQLVESGGGLVQPGGSLRLSCAASGFSFSDFAMSWVRQA |
| | | PGKGLEWVATIGRVAFHTYY |
| | | PDSMKGRFTISRDNSKNTLYLQMNSLRAEDTAVYYCARHR |
| | | GFDVGHFDFWGQGTLVTVSS |
| 653 | NaPi2B VL | DIQMTQSPSSLSASVGDRVTITCRSSETLVHSSGNTYLEWY |
| | | QQKPGKAPKLLIYRVSNRF |
| | | SGVPSRFSGSGSGTDFTLTISSLQPEDFATYYCFQGSFNPLTF |
| | | GQGTKVEIK |
| 654 | Muc16 CDR-H1 | NDYAWN |
| 655 | Muc16 CDR-H2 | YISYSGYTTYNPSLKS |
| 656 | Muc16 CDR-H3 | WTSGLDY |
| 657 | Muc16 CDR-L1 | KASDLIHNWLA |
| 658 | Muc16 CDR-L2 | GATSLET |
| 659 | Muc16 CDR-L3 | QQYWTTPFT |
| 660 | Muc16 VH | EVQLVESGGGLVQPGGSLRLSCAASGYSITNDYAWNWVR |
| | | QAPGKGLEWVGYISYSGYTTY |
| | | NPSLKSRFTISRDTSKNTLYLQMNSLRAEDTAVYYCARWT |
| | | SGLDYWGQGTLVTVSS |
| 661 | Muc16 VL | DIQMTQSPSSLSASVGDRVTITCKASDLIHNWLAWYQQKP |
| | | GKAPKLLIYGATSLETGVPSRFSGSGSGTDFTLTISSLQPEDF |
| | | ATYYCQQYWTTPFTFGQGTKVEIK |
| 662 | STEAPI CDR-H1 | SDYAWN |
| 663 | STEAPI CDR-H2 | YISNSGSTSYNPSLKS |
| 664 | STEAPI CDR-H3 | ERNYDYDDYYYAMDY |
| 665 | STEAPI CDR-L1 | KSSQSLLYRSNQKNYLA |
| 666 | STEAPI CDR-L2 | WASTRES |
| 667 | STEAPI CDR-L3 | QQYYNYPRT |
| 668 | STEAPI VH | EVQLVESGGGLVQPGGSLRLSCAVSGYSITSDYAWNWVRQ |
| | | APGKGLEWVGYISNSGSTSYNPSLKSRFTISRDTSKNTLYLQ |
| | | MNSLRAEDTAVYYCARERNYDYDDYYYAMDYWGQGTL |
| | | VTVSS |
| 669 | STEAP1 VL | DIQMTQSPSSLSASVGDRVTITCKSSQSLLYRSNQKNYLAW |
| | | YQQKPGKAPKLLIYWASTRESGVPSRFSGSGSGTDFTLTISS |
| | | LQPEDFATYYCQQYYNYPRTFGQGTKVEIK |
| 670 | BCMA CDR-H1 | NYWMH |
| 671 | BCMA CDR-H2 | ATYRGHSDTYYNQKFKG |
| 672 | BCMA CDR-H3 | GAIYDGYDVLDN |
| 673 | BCMA CDR-L1 | SASQDISNYLN |
| 674 | BCMA CDR-L2 | YTSNLHS |
| 675 | BCMA CDR-L3 | QQYRKLPWT |
| 676 | BCMA VH | QVQLVQSGAEVKKPGSSVKVSCKASGGTFSNYWMHWVR |
| | | QAPGQGLEWMGATYRGHSDTYYNQKFKGRVTITADKSTS |
| | | TAYMELSSLRSEDTAVYYCARGAIYDGYDVLDNWGQGTL |
| | | VTVSS |
| 677 | BCMA VL | DIQMTQSPSSLSASVGDRVTITCSASQDISNYLNWYQQKPG |
| | | KAPKLLIYYTSNLHSGVPSRFSGSGSGTDFTLTISSLQPEDFA |
| | | TYYCQQYRKLPWTFGQGTKLEIK |
| 678 | c-Met CDR-H1 | AYTMH |
| 679 | c-Met CDR-H2 | WIKPNNGLANYAQKFQG |
| 680 | c-Met CDR-H3 | SEITTEFDY |
| 681 | c-Met CDR-L1 | KSSESVDSYANSFLH |
| 682 | c-Met CDR-L2 | RASTRES |
| 683 | c-Met CDR-L3 | QQSKEDPLT |
| 684 | c-Met VH | QVQLVQSGAEVKKPGASVKVSCKASGYIFTAYTMHWVRQ |
| | | APGQGLEWMGWIKPNNGLAN |
| | | YAQKFQGRVTMTRDTSISTAYMELSRLRSDDTAVYYCARS |
| | | EITTEFDYWGQGTLVTVSS |
| 685 | c-Met VL | DIVMTQSPDSLAVSLGERATINCKSSESVDSYANSFLHWYQ |
| | | QKPGQPPKLLIYRASTRE |
| | | SGVPDRFSGSGSGTDFTLTISSLQAEDVAVYYCQQSKEDPL |
| | | TFGGGTKVEIK |
| 686 | EGFR CDR-H1 | SDFAWN |
| 687 | EGFR CDR-H2 | YISYSGNTRYQPSLKS |
| 688 | EGFR CDR-H3 | AGRGFPY |
| 689 | EGFR CDR-L1 | HSSQDINSNIG |
| 690 | EGFR CDR-L2 | HGTNLDD |
| 691 | EGFR CDR-L3 | VQYAQFPWT |
| 692 | EGFR VH | QVQLQESGPGLVKPSQTLSLTCTVSGYSISSDFAWNWIRQP |
| | | PGKGLEWMGYISYSGNTRY |
| | | QPSLKSRITISRDTSKNQFFLKLNSVTAADTATYYCVTAGR |
| | | GFPYWGQGTLVTVSS |
| 693 | EGFR VL | DIQMTQSPSSMSVSVGDRVTITCHSSQDINSNIGWLQQKPG |
| | | KSFKGLIYHGTNLDDGVPS |
| | | RFSGSGSGTDYTLTISSLQPEDFATYYCVQYAQFPWTFGGG |
| | | TKLEIK |
| 694 | SLAMF7 CDR-H1 | DYYMA |
| 695 | SLAMF7 CDR-H2 | SINYDGSSTYYVDSVKG |
| 696 | SLAMF7 CDR-H3 | DRGYYFDY |
| 697 | SLAMF7 CDR-L1 | RSSQSLVHSNGNTYLH |
| 698 | SLAMF7 CDR-L2 | KVSNRFS |
| 699 | SLAMF7 CDR-L3 | SQSTHVPPFT |
| 700 | SLAMF7 VH | EVQLVESGGGLVQPGGSLRLSCAASGFTFSDYYMAWVRQ |
| | | APGKGLEWVASINYDGSSTY |
| | | YVDSVKGRFTISRDNAKNSLYLQMNSLRAEDTAVYYCAR |
| | | DRGYYFDYWGQGTTVTVSS |
| 701 | SLAMF7 VL | DVVMTQTPLSLSVTPGQPASISCRSSQSLVHSNGNTYLHWY |
| | | LQKPGQSPQLLIYKVSNRF |
| | | SGVPDRFSGSGSGTDFTLKISRVEAEDVGVYFCSQSTHVPPF |
| | | TFGGGTKVEIK |
| 702 | SLITRK6 CDR-H1 | SYGMH |
| 703 | SLITRK6 CDR-H2 | VIWYDGSNQYYADSVKG |
| 704 | SLITRK6 CDR-H3 | GLTSGRYGMDV |
| 705 | SLITRK6 CDR-L1 | RSSQSLLLSHGFNYLD |
| 706 | SLITRK6 CDR-L2 | LGSSRAS |
| 707 | SLITRK6 CDR-L3 | MQPLQIPWT |
| 708 | SLITRK6 VH | QVQLVESGGGVVQPGRSLRLSCAASGFTFSSYGMHWVRQ |
| | | APGKGLEWVAVIWYDGSNQYY |
| | | ADSVKGRFTISRDNSKNTLFLQMHSLRAEDTAVYYCARGL |
| | | TSGRYGMDVWGQGTTVTVSS |
| 709 | SLITRK6 VL | DIVMTQSPLSLPVTPGEPASISCRSSQSLLLSHGFNYLDWYL |
| | | QKPGQSPQLLIYLGSSRA |
| | | SGVPDRFSGSGSGTDFTLKISRVEAEDVGLYYCMQPLQIPW |
| | | TFGQGTKVEIK |
| 710 | C4.4a CDR-H1 | NAWMS |
| 711 | C4.4a CDR-H2 | YISSSGSTIYYADSVKG |
| 712 | C4.4a CDR-H3 | EGLWAFDY |
| 713 | C4.4a CDR-L1 | TGSSSNIGAGYVVH |
| 714 | C4.4a CDR-L2 | DNNKRPS |
| 715 | C4.4a CDR-L3 | AAWDDRLNGPV |
| 716 | C4.4a VH | EVQLLESGGGLVQPGGSLRLSCAASGFTFSNAWMSWVRQ |
| | | APGKGLEWVSYISSSGSTIYY |
| | | ADSVKGRFTISRDNSKNTLYLQMNSLRAEDTAVYYCAREG |
| | | LWAFDYWGQGTLVTVSS |
| 717 | C4.4a VL | ESVLTQPPSVSGAPGQRVTISCTGSSSNIGAGYVVHWYQQL |
| | | PGTAPKLLIYDNNKRPSGV |
| | | PDRFSGSKSGTSASLAISGLRSEDEADYYCAAWDDRLNGP |
| | | VFGGGTKLTVL |
| 718 | GCC CDR-H1 | GYYWS |
| 719 | GCC CDR-H2 | EINHRGNTNDNPSLKS |
| 720 | GCC CDR-H3 | ERGYTYGNFDH |
| 721 | GCC CDR-L1 | RASQSVSRNLA |
| 722 | GCC CDR-L2 | GASTRAT |
| 723 | GCC CDR-L3 | QQYKTWPRT |
| 724 | GCC VH | QVQLQQWGAGLLKPSETLSLTCAVFGGSFSGYYWSWIRQP |
| | | PGKGLEWIGEINHRGNTNDN |
| | | PSLKSRVTISVDTSKNQFALKLSSVTAADTAVYYCARERGY |
| | | TYGNFDHWGQGTLVTVSS |
| 725 | GCC VL | EIVMTQSPATLSVSPGERATLSCRASQSVSRNLAWYQQKPG |
| | | QAPRLLIYGASTRATGIP |
| | | ARFSGSGSGTEFTLTIGSLQSEDFAVYYCQQYKTWPRTFGQ |
| | | GTNVEIK |
| 726 | Axl CDR-H1 | SYAMN |
| 727 | Axl CDR-H2 | TTSGSGASTYYADSVKG |
| 728 | Axl CDR-H3 | IWIAFDI |
| 729 | Axl CDR-L1 | RASQSVSSSYLA |
| 730 | Axl CDR-L2 | GASSRAT |
| 731 | Axl CDR-L3 | QQYGSSPYT |
| 732 | Axl VH | EVQLLESGGGLVQPGGSLRLSCAASGFTFSSYAMNWVRQA |
| | | PGKGLEWVSTTSGSGASTYY |
| | | ADSVKGRFTISRDNSKNTLYLQMNSLRAEDTAVYYCAKIW |
| | | IAFDIWGQGTMVTVSS |
| 733 | Axl VL | EIVLTQSPGTLSLSPGERATLSCRASQSVSSSYLAWYQQKP |
| | | GQAPRLLIYGASSRATGIP |
| | | DRFSGSGSGTDFTLTISRLEPEDFAVYYCQQYGSSPYTFGQ |
| | | GTKLEIK |
| 734 | gpNMB CDR-H1 | SFNYYWS |
| 735 | gpNMB CDR-H2 | YIYYSGSTYSNPSLKS |
| 736 | gpNMB CDR-H3 | GYNWNYFDY |
| 737 | gpNMB CDR-L1 | RASQSVDNNLV |
| 738 | gpNMB CDR-L2 | GASTRAT |
| 739 | gpNMB CDR-L3 | QQYNNWPPWT |
| 740 | gpNMB VH | QVQLQESGPGLVKPSQTLSLTCTVSGGSISSFNYYWSWIRH |
| | | HPGKGLEWIGYIYYSGSTY |
| | | SNPSLKSRVTISVDTSKNQFSLTLSSVTAADTAVYYCARGY |
| | | NWNYFDYWGQGTLVTVSS |
| 741 | gpNMB VL | EIVMTQSPATLSVSPGERATLSCRASQSVDNNLVWYQQKP |
| | | GQAPRLLIYGASTRATGIPA |
| | | RFSGSGSGTEFTLTISSLQSEDFAVYYCQQYNNWPPWTFGQ |
| | | GTKVEIK |
| 742 | Prolactin receptor | TYWMH |
| | CDR-H1 | |
| 743 | Prolactin receptor | EIDPSDSYSNYNQKFKD |
| | CDR-H2 | |
| 744 | Prolactin receptor | NGGLGPAWFSY |
| | CDR-H3 | |
| 745 | Prolactin receptor | KASQYVGTAVA |
| | CDR-L1 | |
| 746 | Prolactin receptor | SASNRYT |
| | CDR-L2 | |
| 747 | Prolactin receptor | QQYSSYPWT |
| | CDR-L3 | |
| 748 | Prolactin receptor | EVQLVQSGAEVKKPGSSVKVSCKASGYTFTTYWMHWVRQ |
| | VH | APGQGLEWIGEIDPSDSYSNY |
| | | NQKFKDRATLTVDKSTSTAYMELSSLRSEDTAVYYCARNG |
| | | GLGPAWFSYWGQGTLVTVSS |
| 749 | Prolactin receptor | DIQMTQSPSSVSASVGDRVTITCKASQYVGTAVAWYQQKP |
| | VL | GKSPKLLIYSASNRYTGVPS |
| | | RFSDSGSGTDFTLTISSLQPEDFATYFCQQYSSYPWTFGGGT |
| | | KVEIK |
| 750 | FGFR2 CDR-H1 | SYAMS |
| 751 | FGFR2 CDR-H2 | AISGSGTSTYYADSVKG |
| 752 | FGFR2 CDR-H3 | VRYNWNHGDWFDP |
| 753 | FGFR2 CDR-L1 | SGSSSNIGNNYVS |
| 754 | FGFR2 CDR-L2 | ENYNRPA |
| 755 | FGFR2 CDR-L3 | SSWDDSLNYWV |
| 756 | FGFR2 VH | EVQLLESGGGLVQPGGSLRLSCAASGFTFSSYAMSWVRQA |
| | | PGKGLEWVSAISGSGTSTYYADSVKGRFTISRDNSKNTLYL |
| | | QMNSLRAEDTAVYYCARVRYNWNHGDWFDPWGQGTLV |
| | | TVSS |
| 757 | FGFR2 VL | QSVLTQPPSASGTPGQRVTISCSGSSSNIGNNYVSWYQQLP |
| | | GTAPKLLIYENYNRPAGVP |
| | | DRFSGSKSGTSASLAISGLRSEDEADYYCSSWDDSLNYWVF |
| | | GGGTKLTVL |
| 758 | CDCP1 CDR-H1 | SYGMS |
| 759 | CDCP1 CDR-H2 | TISSGGSYKYYVDSVKG |
| 760 | CDCP1 CDR-H3 | HPDYDGVWFAY |
| 761 | CDCP1 CDR-L1 | SVSSSVFYVH |
| 762 | CDCP1 CDR-L2 | DTSKLAS |
| 763 | CDCP1 CDR-L3 | QQWNSNPPT |
| 764 | CDCP1 VH | EVQLVESGGGLVQPGGSLRLSCAASGFTFNSYGMSWVRQA |
| | | PGKGLEWVATISSGGSYKYY |
| | | VDSVKGRFTISRDNAKNSLYLQMNSLRAEDTAVYYCARHP |
| | | DYDGVWFAYWGQGTLVTVSS |
| 765 | CDCP1 VL | DIQMTQSPSSLSASVGDRVTITCSVSSSVFYVHWYQQKPGK |
| | | APKLLIYDTSKLASSGVPS |
| | | RFSGSGSGTDFTFTISSLQPEDIATYYCQQWNSNPPTFGGGT |
| | | KVEIK |
| 766 | CDCP1 CDR-H1 | SYGMS |
| 767 | CDCP1 CDR-H2 | TISSGGSYTYYPDSVKG |
| 768 | CDCP1 CDR-H3 | HPDYDGVWFAY |
| 769 | CDCP1 CDR-L1 | SVSSSVFYVH |
| 770 | CDCP1 CDR-L2 | DTSKLAS |
| 771 | CDCP1 CDR-L3 | QQWNSNPPT |
| 772 | CDCP1 VH | EVQLVESGGDLVKPGGSLKLSCAASGFTFNSYGMSWVRQT |
| | | PDKRLEWVATISSGGSYTYY |
| | | PDSVKGRFTISRDNAKNTLYLQMSSLKSEDTAMYYCARHP |
| | | DYDGVWFAYWGQGTLVTVSA |
| 773 | CDCP1 VL | QIVLTQSPAIMASPGEKVTMTCSVSSSVFYVHWYQQKSGTS |
| | | PKRWIYDTSKLASGVPARF |
| | | SGSGSGTSYSLTISSMEAEDAATYYCQQWNSNPPTFGGGTK |
| | | LEIK |
| 774 | CDCP1 CDR-H1 | SYYMH |
| 775 | CDCP1 CDR-H2 | IINPSGGSTSYAQKFQG |
| 776 | CDCP1 CDR-H3 | DGVLRYFDWLLDYYYY |
| 777 | CDCP1 CDR-L1 | RASQSVGSYLA |
| 778 | CDCP1 CDR-L2 | DASNRAT |
| 779 | CDCP1 CDR-L3 | QQRANVFT |
| 780 | CDCP1 VH | EVQLVQSGAEVKKPGASVKVSCKASGYTFTSYYMHWVRQ |
| | | APGQGLEWMGIINPSGGSTSY |
| | | AQKFQGRVTMTRDTSTSTVYMELSSLRSEDTAVYYCARDG |
| | | VLRYFDWLLDYYYYMDVWGKG |
| | | TTVTVSS |
| 781 | CDCP1 VL | EIVLTQSPATLSLSPGERATLSCRASQSVGSYLAWYQQRPG |
| | | QAPRLLIYDASNRATGIPA |
| | | RFSGSGSGTDFTLTISSLEPEDFAVYYCQQRANVFTFGQGT |
| | | KVEIK |
| 782 | CDCP1 CDR-H1 | SYYMH |
| 783 | CDCP1 CDR-H2 | IINPSGGSTSYAQKFQG |
| 784 | CDCP1 CDR-H3 | DAELRHFDHLLDYHYYMDV |
| 785 | CDCP1 CDR-L1 | RASQSVGSYLA |
| 786 | CDCP1 CDR-L2 | DASNRAT |
| 787 | CDCP1 CDR-L3 | QQRAQEFT |
| 788 | CDCP1 VH | EVQLVQSGAEVKKPGASVKVSCKASGYTFTSYYMHWVRQ |
| | | APGQGLEWMGIINPSGGSTSYAQKFQGRVTMTRDTSTSTV |
| | | YMELSSLRSEDTAVYYCARDAELRHFDHLLDYHYYMDVW |
| | | GQGTTVTVSS |
| 789 | CDCP1 VL | EIVMTQSPATLSLSPGERATLSCRASQSVGSYLAWYQQKPG |
| | | QAPRLLIYDASNRATGIPA |
| | | RFSGSGSGTDFTLTISSLQPEDFAVYYCQQRAQEFTFGQGT |
| | | KVEIK |
| 790 | ASCT2 VH | QVQLVQSGSELKKPGAPVKVSCKASGYTFSTFGMSWVRQ |
| | | APGQGLKWMGWIHTYAGVPIYGDDFKGRFVFSLDTSVSTA |
| | | YLQISSLKAEDTAVYFCARRSDNYRYFFDYWGQGTTVTVS |
| | | S |
| 791 | ASCT2 VL | DIQMTQSPSSLSASLGDRVTITCRASQDIRNYLNWYQQKPG |
| | | KAPKLLIYYTSRLHSGVPSRFSGSGSGTDYTLTISSLQPEDF |
| | | ATYFCQQGHTLPPTFGQGTKLEIK |
| 792 | ASCT2 VH | QIQLVQSGPELKKPGAPVKISCKASGYTFTTFGMSWVKQAP |
| | | GQGLKWMGWIHTYAGVPIYGDDFKGRFVFSLDTSVSTAYL |
| | | QISSVKAEDTATYFCARRSDNYRYFFDYWGQGTTLTVSS |
| 793 | ASCT2 VL | DIQMTQSPSSLSASLGDRVTITCRASQDIRNYLNWYQQKPG |
| | | KAPKLLIYYTSRLHSGVPS |
| | | RFSGSGSGTDYTLTISSLQPEDFATYFCQQGHTLPPTFGQGT |
| | | KLEIK |
| 794 | ASCT2 CDR-H1 | NYYMA |
| 795 | ASCT2 CDR-H2 | SITKGGGNTYYRDSVKG |
| 796 | ASCT2 CDR-H3 | QVTIAAVSTSYFDS |
| 797 | ASCT2 CDR-L1 | KTNQKVDYYGNSYVY |
| 798 | ASCT2 CDR-L2 | LASNLAS |
| 799 | ASCT2 CDR-L3 | QQSRNLPYT |
| 800 | ASCT2 VH | EVQLVESGGGLVQSGRSIRLSCAASGFSFSNYYMAWVRQA |
| | | PSKGLEWVASITKGGGNTYYRDSVKGRFTFSRDNAKSTLY |
| | | LQMDSLRSEDTATYYCARQVTIAAVSTSYFDSWGQGVMV |
| | | TVSS |
| 801 | ASCT2 VL | DIVLTQSPALAVSLGQRATISCKTNQKVDYYGNSYVYWYQ |
| | | QKPGQQPKLLIYLASNLASGIPARFSGRGSGTDFTLTIDPVE |
| | | ADDTATYYCQQSRNLPYTFGAGTKLELK |
| 802 | CD 123 CDR-H1 | DYYMK |
| 803 | CD 123 CDR-H2 | diipsngatfynqkfkg |
| 804 | CD 123 CDR-H3 | shllraswfay |
| 805 | CD 123 CDR-L1 | kssqsllnsgnqknylt |
| 806 | CD 123 CDR-L2 | wastres |
| 807 | CD 123 CDR-L3 | qndysypyt |
| 808 | CD123 VH | qvqlvqsgaevkkpgasvkmsckasgytftdyy |
| | | mkwvkqapgqglewigdiipsngatfynqkfkgk |
| | | atltvdrsistaymhlnrlrsddtavyyctrshll |
| | | raswfaywgqgtlvtvss |
| 809 | CD 123 VL | dfvmtqspdslavslgeratinckssqsllnsgnqknyl |
| | | twylqkpgqppklliywastresgvpdrfsgsgsgtdftl |
| | | tisslqaedvavyycqndysypytfgqgtkleik |
| 810 | GPC3 CDR-H1 | DYEMH |
| 811 | GPC3 CDR-H2 | WIGGIDPETGGTAYNQKFKG |
| 812 | GPC3 CDR-H3 | YYSFAY |
| 813 | GPC3 CDR-L1 | RSSQSIVHSNGNTYLQ |
| 814 | GPC3 CDR-L2 | KVSNRFS |
| 815 | GPC3 CDR-L3 | FQVSHVPYT |
| 816 | GPC3 VH | EVQLVQSGAEVKKPGATVKISCKVSGYTFTDYEMHWVQQ |
| | | APGKGLEWMGGIDPETGGTAYNQKFKGRVTLTADKSTDT |
| | | AYMELSSLRSEDTAVYYCGRYYSFAYWGQGTLVTVSS |
| 817 | GPC3 VL | DVVMTQSPLSLPVTLGQPASISCRSSQSIVHSNANTYLQWF |
| | | QQRPGQSPRLLIYKVSNRFSGVPDRFSGSGSGTDFTLKISRV |
| | | EAEDVGVYYCFQVSHVPYTFGQGTKLEIK |
| 818 | B6A CDR-H1 | DYNVN |
| 819 | B6A CDR-H2 | VINPKYGTTRYNQKFKG |
| 820 | B6A CDR-H3 | GLNAWDY |
| 821 | B6A CDR-L1 | GASENIYGALN |
| 822 | B6A CDR-L2 | GATNLED |
| 823 | B6A CDR-L3 | QNVLTTPYT |
| 824 | B6A VH | QFQLVQSGAEVKKPGASVKVSCKASGYSFTDYNVNWVRQ |
| | | APGQGLEWIGVINPKYGTTRYNQKFKGRATLTVDKSTSTA |
| | | YMELSSLRSEDTAVYYCTRGLNAWDYWGQGTLVTVSS |
| 825 | B6A VL | DIQMTQSPSSLSASVGDRVTITCGASENIYGALNWYQQKPG |
| | | KAPKLLIYGATNLEDGVPSRFSGSGSGRDYTFTISSLQPEDI |
| | | ATYYCQNVLTTPYTFGQGTKLEIK |
| 826 | B6A CDR-H1 | GYFMN |
| 827 | B6A CDR-H2 | linpyngdsfynqkfkg |
| 828 | B6A CDR-H3 | glrrdfdy |
| 829 | B6A CDR-L1 | kssqslldsdgktyln |
| 830 | B6A CDR-L2 | ivselds |
| 831 | B6A CDR-L3 | wqgthfprt |
| 832 | B6A VH | QVQLVQSGAEVKKPGASVKVSCKASGYSFSGYFMNWVRQ |
| | | APGQGLEWMGLINPYNGDSFYNQKFKGRVTMTRQTSTST |
| | | VYMELSSLRSEDTAVYYCVRGLRRDFDYWGQGTLVTVSS |
| 833 | B6A VL | DVVMTQSPLSLPVTLGQPASISCKSSQSLLDSDGKTYLNWL |
| | | FQRPGQSPRRLIYLVSELDSGVPDRFSGSGSGTDFTLKISRV |
| | | EAEDVGVYYCWQGTHFPRTFGGGTKLEIK |
| 834 | PD-L1 CDR-H1 | TAAIS |
| 835 | PD-L1 CDR-H2 | GIIPIFGKAHYAQKFQG |
| 836 | PD-L1 CDR-H3 | KFHFVSGSPFGMDV |
| 837 | PD-L1 CDR-L1 | RASQSVSSYLA |
| 838 | PD-L1 CDR-L2 | DASNRAT |
| 839 | PD-L1 CDR-L3 | QQRSNWPT |
| 840 | PD-L1 VH | QVQLVQSGAEVKKPGSSVKVSCKTSGDTFSTAAISWVRQA |
| | | PGQGLEWMGGIIPIFGKAHYAQKFQGRVTITADESTSTAYM |
| | | ELSSLRSEDTAVYFCARKFHFVSGSPFGMDVWGQGTTVTV |
| | | SS |
| 841 | PD-L1 VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPTFGQGTKVEIK |
| 842 | TIGIT CDR-H1 | GTFSSYAIS |
| 843 | TIGIT CDR-H2 | SIIPIFGTANYAQKFQG |
| 844 | TIGIT CDR-H3 | ARGPSEVGAILGYVWFDP |
| 845 | TIGIT CDR-L1 | RSSQSLLHSNGYNYLD |
| 846 | TIGIT CDR-L2 | LGSNRAS |
| 847 | TIGIT CDR-L3 | MQARRIPIT |
| 848 | TIGIT VH | QVQLVQSGAEVKKPGSSVKVSCKASGGTFSSYAISWVRQA |
| | | PGQGLEWMGSIIPIFGTANYAQKFQGRVTITADESTSTAYM |
| | | ELSSLRSEDTAVYYCARGPSEVGAILGYVWFDPWGQGTLV |
| | | TVSS |
| 849 | TIGIT VL | DIVMTQSPLSLPVTPGEPASISCRSSQSLLHSNGYNYLDWYL |
| | | QKPGQSPQLLIYLGSNRASGVPDRFSGSGSGTDFTLKISRVE |
| | | AEDVGVYYCMQARRIPITFGGGTKVEIK |
| 850 | STN CDR-H1 | GYTFTDHAIHWV |
| 851 | STN CDR-H2 | FSPGNDDIKY |
| 852 | STN CDR-H3 | KRSLSTPY |
| 853 | STN CDR-L1 | QSLLNRGNHKNY |
| 854 | STN CDR-L2 | WASTRES |
| 855 | STN CDR-L3 | QNDYTYPYT |
| 856 | STN VH | EVQLVQSGAEVKKPGASVKVSCKASGYTFTDHAIHWVRQ |
| | | APGQGLEWMGYFSPGNDDIKYNEKFRGRVTMTADKSSST |
| | | AYMELRSLRSDDTAVYFCKRSLSTPYWGQGTLVTVSS |
| 857 | STNVL | DIVMTQSPDSLAVSLGERATINCKSSQSLLNRGNHKNYLT |
| | | WYQQKPGQPPKLLIYWASTRESGVPDRFSGSGSGTDFTLTI |
| | | SSLQAEDVAVYYCQNDYTYPYTFGQGTKVEIK |
| 858 | CD33 CDR-H1 | NYDIN |
| 859 | CD33 CDR-H2 | WIYPGDGSTKYNEKFKA |
| 860 | CD33 CDR-H3 | GYEDAMDY |
| 861 | CD33 CDR-L1 | KASQDINSYLS |
| 862 | CD33 CDR-L2 | RANRLVD |
| 863 | CD33 CDR-L3 | LQYDEFPLT |
| 864 | CD33 VH | QVQLVQSGAE VKKPGASVKV SCKASGYTFT |
| | | NYDINWVRQA PGQGLEWIGW IYPGDGSTKY |
| | | NEKFKAKATL TADTSTSTAY MELRSLRSDD |
| | | TAVYYCASGY EDAMDYWGQGTTVTVSS |
| 865 | CD33 VL | DIQMTQSPS SLSASVGDRVT |
| | | INCKASQDINSYLSWFQQKPGKAPKTL IYRANRLVDGVPS |
| | | RFSGSGSGQDYTLT |
| | | ISSLQPEDFATYYCLQYDEFPLTFGGGTKVE |
| 866 | NTBA CDR-H1 | NYGMN |
| 867 | NTBA CDR-H2 | WINTYSGEPRYADDFKG |
| 868 | NTBA CDR-H3 | DYGRWYFDV |
| 869 | NTBA CDR-L1 | RASSSVSHMH |
| 870 | NTBA CDR-L2 | ATSNLAS |
| 871 | NTBA CDR-L3 | QQWSSTPRT |
| 872 | NTBA VH | QIQLVQSGSELKKPGASVKVSCKASGYTFTNYGMNWVRQ |
| | | APGQDLKWMGWINTYSGEPRYADDFKGRFVFSLDKSVNT |
| | | AYLQISSLKAEDTAVYYCARDYGRWYFDVWGQGTTVTVS |
| | | S |
| 873 | NTBA VL | QIVLSQSPATLSLSPGERATMSCRASSSVSHMHWYQQKPG |
| | | QAPRPWIYATSNLASGVPARFSGSGSGTDYTLTISSLEPEDF |
| | | AVYYCQQWSSTPRTFGGGTKVEIK |
| 874 | BCMA CDR-H1 | DYYIH |
| 875 | BCMA CDR-H2 | YINPNSGYTNYAQKFQG |
| 876 | BCMA CDR-H3 | YMWERVTGFFDF |
| 877 | BCMACDR-L1 | LASEDISDDLA |
| 878 | BCMA CDR-L2 | TTSSLQS |
| 879 | BCMA CDR-L3 | QQTYKFPPT |
| 880 | BCMA VH | QVQLVQSGAEVKKPGASVKLSCKASGYTFTDYYIHWVRQ |
| | | APGQGLEWIGYINPNSGYTNYAQKFQGRATMTADKSINTA |
| | | YVELSRLRSDDTAVYFCTRYMWERVTGFFDFWGQGTMVT |
| | | VSS |
| 881 | BCMA VL | DIQMTQSPSSVSASVGDRVTITCLASEDISDDLAWYQQKPG |
| | | KAPKVLVYTTSSLQSGVPSRFSGSGSGTDFTLTISSLQPEDF |
| | | ATYFCQQTYKFPPTFGGGTKVEIK |
| 882 | TF CDR-H1 | GFTFSNYA |
| 883 | TF CDR-H2 | ISGSGDYT |
| 884 | TF CDR-H3 | ARSPWGYYLDS |
| 885 | TF CDR-L1 | QGISSR |
| 886 | TF CDR-L2 | AAS |
| 887 | TF CDR-L3 | QQYNSYPYT |
| 888 | TF VH | EVQLLESGGGLVQPGGSLRLSCAASGFTFSNYAMSWVRQA |
| | | PGKGLEWVSSISGSGDYTYYTDSVKGRFTISRDNSKNTLYL |
| | | QMNSLRAEDTAVYYCARSPWGYYLDSWGQGTLVTVSS |
| 889 | TF VL | DIQMTQSPPSLSASAGDRVTITCRASQGISSRLAWYQQKPE |
| | | KAPKSLIYAASSLQSGVPSRFSGSGSGTDFTLTISSLQPEDFA |
| | | TYYCQQYNSYPYTFGQGTKLEIK |

### Methods of Use

In some embodiments, the ADCs described herein (e.g., Formula (I), or a pharmaceutically acceptable salt thereof) are used to deliver a drug to a target cell. Without being bound by theory, in some embodiments, an ADC associates with an antigen on the surface of a target cell, and the ADC is then taken up inside a target-cell through receptor-mediated endocytosis. Once inside the cell, the Drug Unit is released as free drug and will induce its biological effect (such as a cytotoxic or cytostatic effect, as defined herein). In some embodiments, the Drug Unit is cleaved from the ADC outside the target cell, and the free drug subsequently penetrates the cell.

Some embodiments provide a method of treating cancer in a subject in need thereof, comprising administering to the subject a therapeutically effective amount of Formula (I), or a pharmaceutically acceptable salt thereof.

Some embodiments provide a method of treating cancer in a subject in need thereof, comprising administering to the subject a therapeutically effective amount of Formula (I), or a pharmaceutically acceptable salt thereof, before, during, or after administration of another anticancer agent to the subject (e.g., an immunotherapy such as nivolumab or pembrolizumab).

Some embodiments provide a method for reversing or preventing acquired resistance to an anticancer agent, comprising administering a therapeutically effective amount of Formula (I), or a pharmaceutically acceptable salt thereof, to a subject at risk for developing or having acquired resistance to an anticancer agent. In some embodiments, the subject is administered a dose of the anticancer agent (e.g., at substantially the same time as a dose of Formula (I), or a pharmaceutically acceptable salt thereof is administered to the subject).

Some embodiments provide a method of delaying and/or preventing development of cancer resistant to an anticancer agent in a subject, comprising administering to the subject a therapeutically effective amount of Formula (I), or a pharmaceutically acceptable salt thereof, before, during, or after administration of a therapeutically effective amount of the anticancer agent.

In some embodiments, the ADCs described herein are useful for inhibiting the multiplication of a tumor cell or cancer cell, causing apoptosis in a tumor or cancer cell, and/or for treating cancer in a subject in need thereof. The ADCs can be used accordingly in a variety of settings for the treatment of cancers. The ADCs can be used to deliver a drug (e.g., cytotoxic or cytostatic drug) to a tumor cell or cancer cell. Without being bound by theory, in some embodiments, the antibody of an ADC binds to or associates with a cancer-cell or a tumor-cell-associated antigen, and the ADC can be taken up (internalized) inside a tumor cell or cancer cell through receptor-mediated endocytosis or other internalization mechanism. The antigen can be attached to a tumor cell or cancer cell or can be an extracellular matrix protein associated with the tumor cell or cancer cell. Once inside the cell, via a cleavable mechanism, the drug is released within the cell. In some embodiments, the Drug Unit is cleaved from the ADC outside the tumor cell or cancer cell, and the free drug subsequently penetrates the cell.

In some embodiments, the antibody binds to the tumor cell or cancer cell. In some embodiments, the antibody binds to a tumor cell or cancer cell antigen which is on the surface of the tumor cell or cancer cell. In some embodiments, the antibody binds to a tumor cell or cancer cell antigen which is an extracellular matrix protein associated with the tumor cell or cancer cell.

The specificity of the antibody of the ADC described herein for a particular tumor cell or cancer cell can be important for determining those tumors or cancers that are most effectively treated. For example, ADCs that target a cancer cell antigen present on hematopoietic cancer cells in some embodiments treat hematologic malignancies. In some embodiments, ADCs that target a cancer cell antigen present on abnormal cells of solid tumors treat such solid tumors. In some embodiments, an ADC are directed against abnormal cells of hematopoietic cancers such as, for example, lymphomas (Hodgkin Lymphoma and Non-Hodgkin Lymphomas) and leukemias and solid tumors.

Cancers, including, but not limited to, a tumor, metastasis, or other disease or disorder characterized by abnormal cells that are characterized by uncontrolled cell growth in some embodiments are treated or inhibited by administration of an ADC.

In some embodiments, the subject has previously undergone treatment for the cancer. In some embodiments, the prior treatment is surgery, radiation therapy, administration of one or more anticancer agents, or a combination of any of the foregoing.

In some embodiments, the cancer is selected from the group of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some embodiments, the subject is concurrently administered one or more additional anticancer agents with Formula (I), or a pharmaceutically acceptable salt thereof. In some embodiments, the subject is concurrently receiving radiation therapy with Formula (I), or a pharmaceutically acceptable salt thereof. In some embodiments, the subject is administered one or more additional anticancer agents after administration of Formula (I), or a pharmaceutically acceptable salt thereof. In some embodiments, the subject receives radiation therapy after administration of Formula (I), or a pharmaceutically acceptable salt thereof.

In some embodiments, the subject has discontinued the prior therapy, for example, due to unacceptable or unbearable side effects, or wherein the prior therapy was too toxic.

Some embodiments provide a method of treating an autoimmune disorder in a subject in need thereof, comprising administering to the subject a therapeutically effective amount of Formula (I), or a pharmaceutically acceptable salt thereof.

Some embodiments provide a method of treating an autoimmune disorder in a subject in need thereof, comprising administering to the subject a therapeutically effective amount of Formula (I), or a pharmaceutically acceptable salt thereof, to the subject before, during, or after administration of an additional therapeutic agent (e.g., methotrexate, adalimumab, or rituxumab).

Some embodiments provide a method of ameliorating one or more symptoms of an autoimmune disorder in a subject in need thereof, comprising administering to the subject a therapeutically effective amount of Formula (I), or a pharmaceutically acceptable salt thereof.

Some embodiments provide a method of ameliorating one or more symptoms of an autoimmune disorder in a subject in need thereof, comprising administering to the subject a therapeutically effective amount of Formula (I), a pharmaceutically acceptable salt thereof, before, during, or after administration of an additional therapeutic agent to the subject (e.g., methotrexate, adalimumab, or rituxumab).

Some embodiments provide a method of reducing the occurrence of flare-ups of an autoimmune disorder in a subject in need thereof, comprising administering to the subject a therapeutically effective amount of Formula (I), or a pharmaceutically acceptable salt thereof.

Some embodiments provide a method of reducing the occurrence of flare-ups an autoimmune disorder in a subject in need thereof, comprising administering to the subject a therapeutically effective amount of Formula (I), or a pharmaceutically acceptable salt thereof, to the subject before, during, or after administration of an additional therapeutic agent (e.g., methotrexate, adalimumab, or rituxumab).

A “flare-up” refers to a sudden onset of symptoms, or sudden increase in severity of symptoms, of a disorder. For example, a flare-up in mild joint pain typically addressed with NSAIDs could result in debilitating joint pain preventing normal locomotion even with NSAIDS.

In some embodiments, the antibody of the ADC binds to an autoimmune antigen. In some embodiments, the antigen is on the surface of a cell involved in an autoimmune disorder. In some embodiments, the antibody binds to an autoimmune antigen which is on the surface of a cell. In some embodiments, the antibody binds to activated lymphocytes that are associated with the autoimmune disorder state. In some embodiments, the ADC kills or inhibits the multiplication of cells that produce an autoimmune antibody associated with a particular autoimmune disorder.

In some embodiments, the subject is concurrently administered one or more additional therapeutic agents with Formula (I), or a pharmaceutically acceptable salt thereof. In some embodiments, one or more additional therapeutic agents are compounds known to treat and/or ameliorate the symptoms of an autoimmune disorder (e.g., compounds that are approved by the FDA or EMA for the treatment of an autoimmune disorder).

In some embodiments, the autoimmune disorders include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

In some embodiments, the one or more symptoms of an autoimmune disorder include, but are not limited to joint pain, joint swelling, skin rash, itching, fever, fatigue, anemia, diarrhea, dry eyes, dry mouth, hair loss, and muscle aches.

### Compositions and Methods of Administration

The present disclosure provides pharmaceutical compositions comprising the ADCs described herein and a pharmaceutically acceptable carrier. The preferred route of administration is parenteral. Parenteral administration includes subcutaneous injections, intravenous, intramuscular, intrasternal injection or infusion techniques. In some embodiments, the compositions are administered parenterally. In one of those embodiments, the conjugates are administered intravenously. Administration is typically through any convenient route, for example by infusion or bolus injection.

Pharmaceutical compositions of an ADC are formulated so as to allow it to be bioavailable upon administration of the composition to a subject. In some embodiments, the compositions will be in the form of one or more injectable dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

In some embodiments, the ADC composition is a solid, for example, as a lyophilized powder, suitable for reconstitution into a liquid formulation prior to administration. In some embodiments, the ADC composition is a liquid composition, such as a solution or a suspension. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments, the liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. Physiological saline is an exemplary adjuvant. An injectable composition is preferably a liquid composition that is sterile.

The amount of the ADC that is effective in the treatment of a particular disorder or condition will depend on the nature of the disorder or condition, which is usually determined by standard clinical techniques. In addition, in vitro and/or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each subject's circumstances.

In some embodiments, the compositions comprise an effective amount of an ADC such that a suitable dosage will be obtained. Typically, this amount is at least about 0.01% of the ADC by weight of the composition.

In some embodiments, the compositions dosage of an ADC administered to a subject is from about 0.01 mg/kg to about 100 mg/kg, from about 1 to about 100 mg of a per kg or from about 0.1 to about 25 mg/kg of the subject's body weight. In some embodiments, the dosage administered to a subject is about 0.01 mg/kg to about 15 mg/kg of the subject's body weight. In some embodiments, the dosage administered to a subject is about 0.1 mg/kg to about 15 mg/kg of the subject's body weight. In some embodiments, the dosage administered to a subject is about 0.1 mg/kg to about 20 mg/kg of the subject's body weight. In some embodiments, the dosage administered is about 0.1 mg/kg to about 5 mg/kg or about 0.1 mg/kg to about 10 mg/kg of the subject's body weight. In some embodiments, the dosage administered is about 1 mg/kg to about 15 mg/kg of the subject's body weight. In some embodiments, the dosage administered is about 1 mg/kg to about 10 mg/kg of the subject's body weight. In some embodiments, the dosage administered is about 0.1 to about 4 mg/kg, about 0.1 to about 3.2 mg/kg, or about 0.1 to about 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the ADCs are formulated in accordance with routine procedures as a pharmaceutical composition adapted for intravenous administration to animals, particularly human beings. Typically, the carriers or vehicles for intravenous administration are sterile isotonic aqueous buffer solutions. In some embodiments, the composition further comprises a local anesthetic, such as lignocaine, to ease pain at the site of the injection. In some embodiments, the ADC and the remainder of the formulation are supplied either separately or mixed together in unit dosage form, for example, as a dry lyophilized powder or water free concentrate in a hermetically sealed container such as an ampoule or sachette indicating the quantity of active agent. Where an ADC is to be administered by infusion, it is sometimes dispensed, for example, with an infusion bottle containing sterile pharmaceutical grade water or saline. Where the conjugate is administered by injection, an ampoule of sterile water for injection or saline is typically provided so that the ingredients are mixed prior to administration.

The pharmaceutical compositions are generally formulated as sterile, substantially isotonic and in full compliance with all Good Manufacturing Practice (GMP) regulations of the U.S. Food and Drug Administration.

### EXAMPLES

### General Information

All commercially available anhydrous solvents were used without further purification. Silica gel chromatography was performed on a Biotage Isolera One flash purification system (Charlotte, N.C.). UPLC-MS was performed on a Waters Xevo G2 ToF mass spectrometer interfaced to a Waters Acquity H-Class Ultra Performance LC equipped with an Acquity UPLC BEH C18 2.1×50 mm, 1.7 μm reverse phase column. The acidic mobile phase (0.1% formic acid) consisted of a gradient of 3% acetonitrile/97% water to 100% acetonitrile (flow rate=0.7 mL/min). Preparative HPLC was carried out on a Waters 2545 solvent delivery system configured with a Waters 2998 PDA detector. Products were purified over a C12 Phenomenex Synergi reverse phase column (10.0-50 mm diameter×250 mm length, 4 μm, 80 Å) eluting with 0.1% trifluoroacetic acid in water (solvent A) and 0.1% trifluoroacetic acid in acetonitrile (solvent B). The purification methods generally consisted of linear gradients of solvent A to solvent B, ramping from 5% aqueous solvent B to 95% solvent B; flow rate was varied depending on column diameter. NMR spectral data were collected on a Varian Mercury 400 MHz spectrometer. Coupling constants (J) are reported in hertz.

Product purification: Products were purified by flash column chromatography utilizing a Biotage Isolera One flash purification system (Charlotte, N.C.). Ultra Performance Liquid Chromatography-Mass Spectrometry (UPLC-MS) was performed on a Waters single quad detector mass spectrometer interfaced to a Waters Acquity UPLC system. Preparative-High Performance Liquid Chromatography (HPLC) was carried out on a Waters 2454 Binary Gradient Module solvent delivery system configured with a Waters 2998 PDA detector. Products were purified with the appropriate diameter of column of a Phenomenex Max-RP 4 μm Synergi 80 Å 250 mm reverse phase column eluting with 0.05% trifluoroacetic acid in water and 0.05% trifluoroacetic acid in acetonitrile unless otherwise specified. All commercially available anhydrous solvents were used without further purification. Starting materials, reagents and solvents were purchased from commercial suppliers (Sigma Aldrich and/or Fischer Scientific).

### Analytical LCMS Methods

Method A: Chromatography was performed on a Waters Acquity H Class UPLC equipped with a C18 column (Phenomenex Luna, 2.1×50 mm, 1.6 μm). Solvent A comprised 0.05% formic acid in water. Solvent B comprised 0.05% formic acid in acetonitrile. The flow rate was 0.7 ml/min, and elution was carried out with the following gradient: 0 to 1.21 min, 3% to 60% solvent B; 1.21 to 1.43 min, 60% to 95% solvent B; 1.43 to 1.79 min, 95% to 3% solvent B. Mass detection was performed on a Waters Xevo G2 TOF by electrospray ionization in positive ion mode.

Method B: Chromatography was performed on a Waters Acquity H Class UPLC equipped with a C8 column (Phenomenex Kinetex, 2.1×50 mm, 1.7 μm). Solvent A comprised 0.05% formic acid in water. Solvent B comprised 0.05% formic acid in acetonitrile. The flow rate was 0.7 ml/min, and elution was carried out with the following gradient: 0 to 1.21 min, 3% to 60% solvent B; 1.21 to 1.43 min, 60% to 95% solvent B; 1.43 to 1.79 min, 95% to 3% solvent B. Mass detection was performed on a Waters Xevo G2 TOF by electrospray ionization in positive ion mode.

Method C: Chromatography was performed on a Waters Acquity H Class UPLC equipped with a C18 column (Phenomenex Luna, 2.1×50 mm, 1.6 μm). Solvent A comprised 0.05% formic acid in water. Solvent B comprised 0.05% formic acid in acetonitrile. The flow rate was 0.6 ml/min, and elution was carried out with the following gradient: 0 to 1.10 min, 3% to 60% solvent B; 1.10 to 1.50 min, 60% to 97% solvent B; 1.50 min to 2.50 min, 97% solvent B; 2.50 min to 2.60 min; 97% to 3% solvent B. Mass detection was performed on a Waters Xevo G2 TOF by electrospray ionization in positive ion mode.

Method D: Chromatography was performed on a Waters Acquity H Class UPLC equipped with a C18 column (Phenomenex Luna, 2.1×50 mm, 1.6 μm). Solvent A comprised 0.05% formic acid in water. Solvent B comprised 0.05% formic acid in acetonitrile. The flow rate was 0.7 ml/min, and elution was carried out with the following gradient: 0 to 1.21 min, 3% to 60% solvent B; 1.21 to 1.43 min, 60% to 97% solvent B; 1.43 min to 4.00 min, 97% to 3% solvent B. Mass detection was performed on a Waters Xevo G2 TOF by electrospray ionization in positive ion mode.

Method E: Chromatography was performed on a Waters Acquity UPLC equipped with a C18 column (Phenomenex Luna, 2.1×50 mm, 1.6 μm). Solvent A comprised 0.1% formic acid in water. Solvent B comprised 0.1% formic acid in acetonitrile. The flow rate was 0.5 ml/min, and elution was carried out with the following gradient: 0 to 1.70 min, 3% to 60% solvent B; 1.70 to 1.2.00 min, 60% to 95% solvent B; 2.00 min to 2.50 min, 97% to 3% solvent B. Mass detection was performed on a Waters Acquity SQ by electrospray ionization in positive ion mode.

### CORTECS C18 General Method:

Column—Waters CORTECS C18 1.6 μm, 2.1×50 mm, reversed-phase column
Solvent A—0.1% aqueous formic acid
Solvent B—acetonitrile with 0.1% formic acid

| Time (min) | Flow (mL/min) | A % | B % | Gradient |
|---|---|---|---|---|
| Initial | 0.6 | 97 | 3 | |
| 1.70 | 0.6 | 40 | 60 | Linear |
| 2.00 | 0.6 | 5 | 95 | Linear |
| 2.50 | 0.6 | 5 | 95 | Linear |
| 2.80 | 0.6 | 97 | 3 | Linear |
| 3.00 | 0.6 | 97 | 3 | Linear |

### CORTECS C18 Hydrophobic Method:

Column—Waters CORTECS C18 1.6 μm, 2.1×50 mm, reversed-phase column
Solvent A—0.1% aqueous formic acid
Solvent B—acetonitrile with 0.1% formic acid

| Time (min) | Flow (mL/min) | A % | B % | Gradient |
|---|---|---|---|---|
| Initial | 0.6 | 97 | 3 | |
| 1.50 | 0.6 | 5 | 95 | Linear |
| 2.40 | 0.6 | 5 | 95 | Linear |
| 2.50 | 0.6 | 97 | 3 | Linear |
| 2.80 | 0.6 | 97 | 3 | Linear |

### CORTECS C18 Hydrophilic Method:

Column—Waters CORTECS C18 1.6 μm, 2.1×50 mm, reversed-phase column
Solvent A—0.1% aqueous formic acid
Solvent B—acetonitrile with 0.1% formic acid

| Time (min) | Flow (mL/min) | A % | B % | Gradient |
|---|---|---|---|---|
| Initial | 0.6 | 97 | 3 | |
| 1.70 | 0.6 | 67 | 33 | Linear |
| 2.00 | 0.6 | 5 | 95 | Linear |
| 2.50 | 0.6 | 97 | 3 | Linear |
| 2.80 | 0.6 | 97 | 3 | Linear |

### Example 2: Synthesis of MC 1 (Glucuronide-Gemcitabine Conjugate)

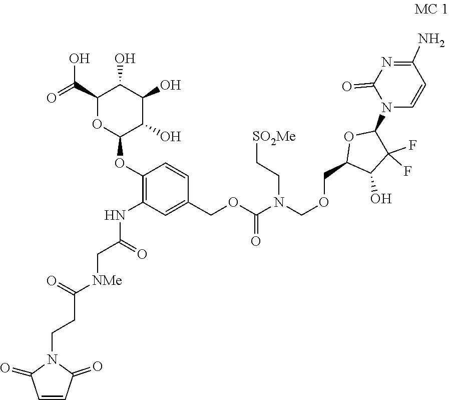 [SMILES: CN(CC(=O)Nc1cc(COC(=O)N(CCS(C)(=O)=O)COC[C@H]2O[C@@H](n3ccc(N)nc3=O)C(F)(F)[C@@H]2O)ccc1O[C@H]1O[C@@H](C(=O)O)[C@H](O)[C@@H](O)[C@@H]1O)C(=O)CCN1C(=O)C=CC1=O]

### Step 1:

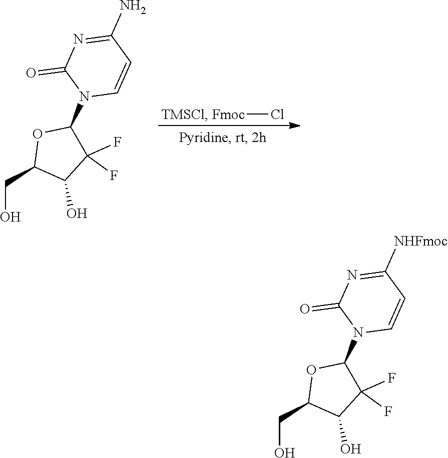

To 10 mL anhydrous pyridine was dissolved 782.6 mg Gemcitabine (2.973 mmol). To this solution, 1.89 mL trimethylsilyl chloride (TMSCl) (14.9 mmol) was added over 5 minutes while continually and vigorously stirred for 15 minutes. To the reaction, 961.5 mg fluorenylmethyloxycarbonyl chloride (Fmoc-Cl) (3.717 mmol) was added where the reaction turned from yellow to colorless over 30 minutes, and a white precipitate persisted over the course of the reaction. To hydrolyze the trimethylsilyl (TMS) groups and excess chloroformate, 2.0 mL H₂O was added, and the reaction was stirred for 2 hours. The reaction mixture was diluted with 100 mL EtOAc, and washed 3 times with 100 mL 1M hydrochloric acid (HCl), dried magnesium sulfate (MgSO4). At this time, the reaction is filtered and concentrated in vacuo. Crude product is purified by flash chromatography 100G KP-Sil 50-100% EtOAc in Hex. R_{f} (product)=0.15 in 1:2 Hex:EtOAc.

Fractions containing the desired product were concentrated in vacuo to produce the product as a white solid (1.169 g, 2.407 mmol, 80.9%). Rt=1.71 min, CORTECS C18 General Method UPLC (as described above in connection with Example 1). MS (m/z) [M+H]⁺ calc. for C₂₄H₂₂F₂N₃O₆ 486.45, found 486.12.

### Step 2:

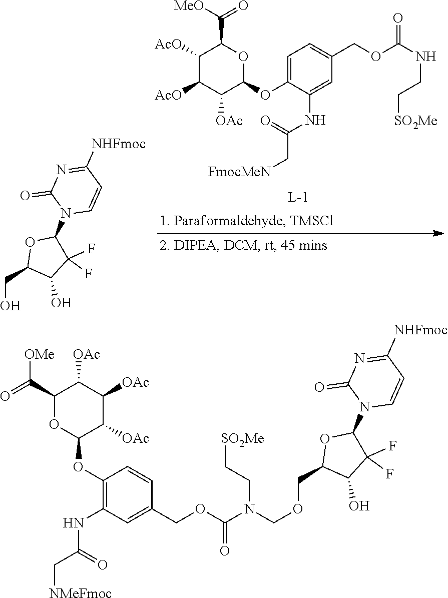

A solution was created of 185 mg Linker (L-1) (0.206 mmol) dissolved in 2 mL dichloromethane (DCM). To this solution, 185 mg paraformaldehyde (6.18 mmol) was added followed by 1.0 mL TMSCl. The reaction was stirred for 10 minutes at which point complete conversion was observed by diluting 2 μL aliquot into 98 μL of MeOH and observing the MeOH adduct by UPLC-MS. The reaction was filtered with a syringe filter, rinsed with 1 mL DCM, and 2 mL toluene was added to azeotrope final mixture upon concentration. The eluent was concentrated in vacuo to afford an activated linker as a colorless solid.

The Fmoc-Gemcitabine (Step 1), was azeotroped with toluene and dried under high vacuum prior to use. After which 100 mg Fmoc-Gemcitabine (0.206 mmol) was suspended in 2 mL anhydrous DCM and 71.8 DIPEA μL (0.412 mmol) was added. The activated linker was dissolved in 2 mL anhydrous DCM and added dropwise to the stirring reaction at a rate of 10 mL/hour. The reaction was stirred for 45 minutes at which point complete conversion was observed. The reaction was quenched with 0.1 mL MeOH, filtered, and the eluent was concentrated in vacuo to afford a colorless solid which was used in the next step without purification (182 mg, 0.130 mmol, crude, 63%). Rt=1.56 min CORTECS C18 Hydrophobic Method UPLC. MS (m/z) [M+H]⁺ calc. for C67H69F2N6O23S 1395.41, found 1395.40.

### Step 3:

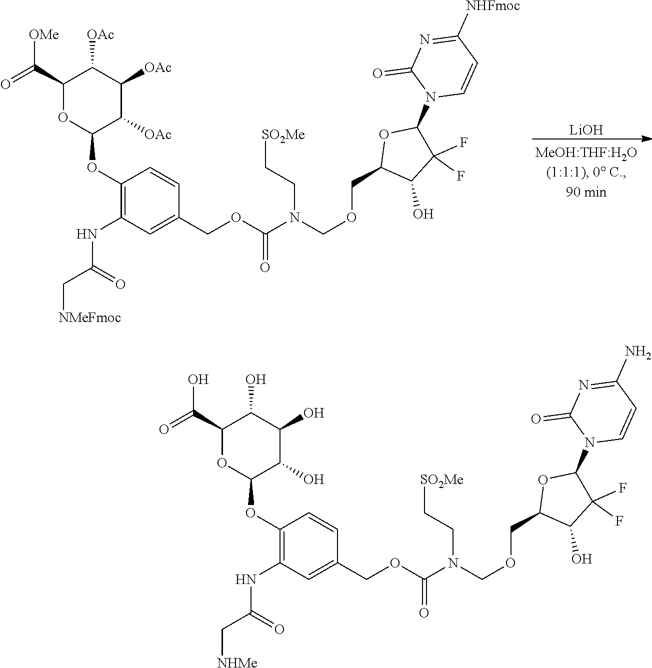

A solution of 2 mL THF:MeOH 1:1 into which was dissolved 182 mg of step 2 product (0.130 mmol). The reaction was cooled with an ice/water bath. After which 31.2 mg LiOH (1.30 mmol) was added and the reaction was stirred for 30 minutes. Conversion to the acetate de-protected product was observed by UPLC-MS (as described in Example 1) and 1 mL H₂O was added to the reaction mixture and the reaction was stirred for 60 minutes. Complete conversion observed by UPLC-MS (as described in Example 1). The reaction was quenched with 30 μL AcOH, concentrated in vacuo and purified by preparative HPLC using a 21.2×250 mm Max-RP column eluted with a gradient of 5-35-95% MeCN in H₂O 0.05% TFA. Fractions containing the desired compound were concentrated in vacuo to afford the desired compound as a colorless solid (65.1 mg, 0.0803 mmol, 62%). Rt=0.82 min CORTECS C18 Hydrophilic Method UPLC. MS (m/z) [M+H]⁺ calc. for C₃₀H₄₁F₂N₆O₁₆S 811.23, found 811.04.

### Step 4: Gemcitabine and Linker and N-Succinimidyl 3-Maleimidopropionate

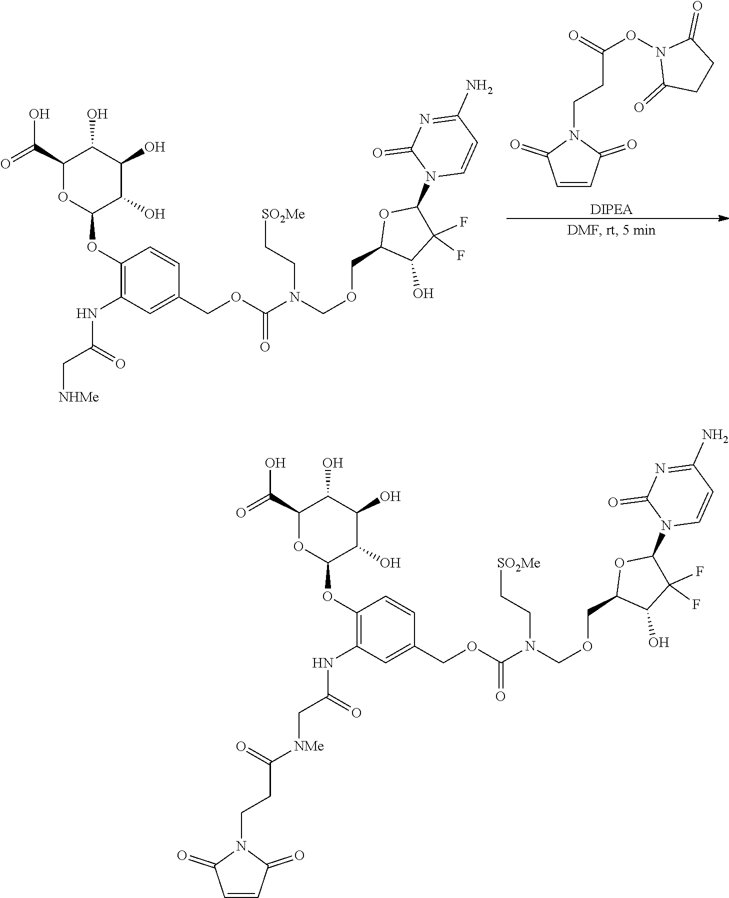

A solution of 0.5 mL anhydrous DMF into which 65.1 mg of the product of step 3 (0.0803 mmol) was dissolved. To the reaction was added 26.5 μL DIPEA (0.160 mmol) was added followed by 23.5 mg N-Succinimidyl 3-Maleimidopropionate (0.0883 mmol, purchased from TCI America product number S0427). The reaction was stirred for 15 minutes. Complete conversion was observed after UPLC-MS. The reaction was quenched with 0.020 mL AcOH and purified by preparative HPLC eluting with 5-35-95% MeCN in H₂O 0.05% TFA on a 21.2×250 mm Max-RP. Fractions containing the desired product were lyophilized to afford desired compound as a colorless powder (41.2 mg, 0.0428 mmol, 53.3%). Rt=1.29 min CORTECS C18 Hydrophilic Method UPLC. MS (m/z) [M+H]⁺ calc. for C₃₇H₄₆F₂N₇O₁₉S 962.25, found 962.06.

### Example 3: Synthesis of Protected Duplexing Agent (S)—N,N′-(((2-(3-(2,5-dioxo-2,5-dihydro-1H-pyrrol-1-yl)propanamido)butane-1,4-diyl)bis(sulfanediyl))bis(methylene)) diacetamide (MC2 diacetamide)

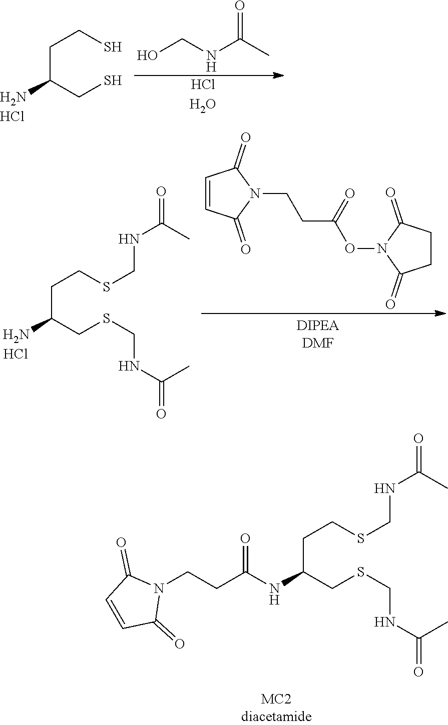

A vial was charged with 200 mg (S)-2-aminobutane-1,4-dithiol hydrochloride (1.15 mmol) and 308 mg N-(hydroxymethyl)acetamide (3.45 mmol) and suspended in 0.6 mL water. The suspension was cooled in an ice water bath and 0.2 mL hydrochloric acid (11.7 M, 2.34 mmol) was added dropwise. The reaction was slowly warmed to room temperature. After stirring overnight, the reaction was concentrated at 45° C. to afford the intermediate (S)—N,N′-(((2-aminobutane-1,4-diyl)bis(sulfanediyl))bis(methylene))diacetamide hydrochloride as a clear semi-solid that was used without further purification. Analytical UPLC-MS: tr=0.57 min, m/z (ES+) calculated 280.1 (M+H)⁺, found 280.0.

Combined in a vial: 232 mg of the intermediate (S)—N,N′-(((2-aminobutane-1,4-diyl)bis(sulfanediyl))bis(methylene))diacetamide hydrochloride (0.73 mmol), and 391 mg 2,5-dioxopyrrolidin-1-yl 3-(2,5-dioxo-2,5-dihydro-1H-pyrrol-1-yl)propanoate (1.47 mmol) dissolved in 2.5 mL DMF, and 0.51 mL DIPEA (2.94 mmol) was added dropwise. After stirring for 2 hours at room temperature, the reaction was quenched with 0.25 mL acetic acid, diluted with methanol, purified by preparative HPLC (as described above in connection with Example 1), and lyophilized to dryness to provide (S)—N,N′-(((2-(3-(2,5-dioxo-2,5-dihydro-1H-pyrrol-1-yl)propanamido)butane-1,4-diyl)bis(sulfanediyl))bis(methylene))diacetamide (42 mg, 13.3%. Analytical UPLC: tr=0.89 min, m/z (ES+) calculated 431.1 (M+H)⁺, found 431.1; calculated 453.1 (M+Na)⁺, found 453.0.

### Example 4: Synthesis of MC9

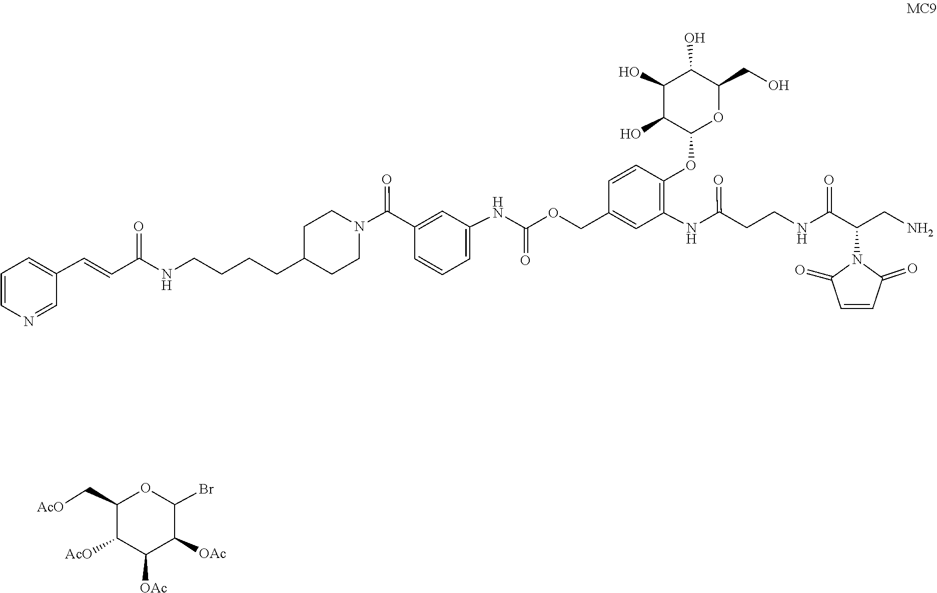 [SMILES: NC[C@@H](C(=O)NCCC(=O)Nc1cc(COC(=O)Nc2cccc(C(=O)N3CCC(CCCCNC(=O)/C=C/c4cccnc4)CC3)c2)ccc1O[C@H]1O[C@H](CO)[C@@H](O)[C@H](O)[C@@H]1O)N1C(=O)C=CC1=O ; CC(=O)OC[C@H]1OC(Br)[C@@H](OC(C)=O)[C@@H](OC(C)=O)[C@@H]1OC(C)=O]

Step 1: (2R,3R,4S,5S)-2-(acetoxymethyl)-6-bromotetrahydro-2H-pyran-3,4,5-triyl triacetate (Compound 5): (2R,3S,4S,5R,6R)-6-(acetoxymethyl)tetrahydro-2H-pyran-2,3,4,5-tetrayl tetraacetate (2.55 g, 6.53 mmol) was dissolved in 11.5 mL CH₂Cl₂ and cooled to 0° C. in ice bath. A solution of 33% HBr in 4.3 mL acetic acid was added dropwise, stirred at 0° C. for 30 min, and allowed to slowly warm to room temperature overnight. Reaction was determined complete by TLC (conditions: 30% EtOAc/hexanes, stained with KMnO₄). The crude reaction mixture was diluted with CH₂Cl₂ and washed once each with water, sat. NaHCO₃ solution, water, and brine, then dried over Na₂SO₄, filtered, and concentrated in vacuo to provide compound 5 (2.68 g, 6.52 mmol, 100%). ¹H NMR (CDCl₃, 400 MHz): δ 2.01 (s, 3H), 2.08 (s, 3H), 2.10 (s, 3H), 2.18 (s, 3H), 4.13 (dd, J=12.5 Hz, 2.2 Hz, 1H), 4.18-4.26 (m, 1H), 4.33 (dd, J=12.5 Hz, 4.8 Hz, 1H), 5.33-5.41 (m, 1H), 5.44 (dd, J=3.5 Hz, 1.6 Hz, 1H), 5.70 (dd, J=10.3 Hz, 3.3 Hz, 1H), 6.33 (dd, J=1.7 Hz, 0.8 Hz, 1H).

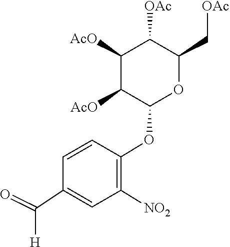 [SMILES: CC(=O)OC[C@H]1O[C@H](Oc2ccc(C=O)cc2[N+](=O)[O-])[C@@H](OC(C)=O)[C@@H](OC(C)=O)[C@@H]1OC(C)=O]

Step 2: (2R,3R,4S,5S,6R)-2-(acetoxymethyl)-6-(4-formyl-2-nitrophenoxy)tetrahydro-2H-pyran-3,4,5-triyl triacetate (Compound 6): Compound 5 (3.227 g, 7.85 mmol) was dissolved in 10 mL acetonitrile and silver oxide (7.82 g, 33.74 mmol) added. Dissolved 4-formyl-2-nitrophenol (1.312 g, 7.85 mmol) in 55 mL acetonitrile was added portion-wise to the reaction mixture. Reaction was determined complete after 2 hours by TLC (conditions: 5% MeOH/DCM, stained with KMnO₄), the solution filtered through celite with ethyl acetate, and the filtrate concentrated in vacuo to provide compound 6 (3.643 g, 7.32 mmol, 93%). LCMS Method A: tr=1.31 min; m/z=520.2 [M+Na]⁺.

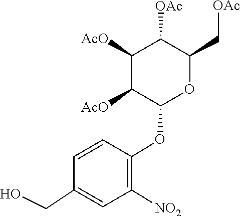 [SMILES: CC(=O)OC[C@H]1O[C@H](Oc2ccc(CO)cc2[N+](=O)[O-])[C@@H](OC(C)=O)[C@@H](OC(C)=O)[C@@H]1OC(C)=O]

Step 3: (2R,3R,4S,5S,6R)-2-(acetoxymethyl)-6-(4-(hydroxymethyl)-2-nitrophenoxy) tetrahydro-2H-pyran-3,4,5-triyl triacetate (Compound 7): compound 6 (3.245 g, 6.52 mmol) suspended in 60 mL 1:1:1 THF:MeOH:AcOH and cooled to 0° C. in ice bath. Sodium borohydride (740 mg, 19.56 mmol) added in portions over 2 hours. Upon completion, the reaction mixture was diluted with methanol, filtered through celite, and concentrated in vacuo. The crude residue was partitioned between DCM and sat. NaHCO₃ solution, the aqueous layer extracted twice with DCM, and the combined organic layers washed once with brine, dried over Na₂SO₄, filtered, and concentrated in vacuo to provide compound 7 (3.09 g, 6.19 mmol, 95%). LCMS Method A: tr=1.14 min; m/z=522.2 [M+Na]⁺.

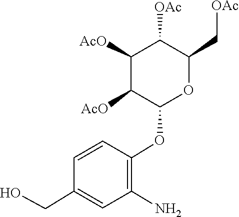 [SMILES: CC(=O)OC[C@H]1O[C@H](Oc2ccc(CO)cc2N)[C@@H](OC(C)=O)[C@@H](OC(C)=O)[C@@H]1OC(C)=O]

Step 4: (2R,3R,4S,5S,6R)-2-(acetoxymethyl)-6-(2-amino-4-(hydroxymethyl)phenoxy) tetrahydro-2H-pyran-3,4,5-triyl triacetate (compound 8): compound 7 (1.376 g, 2.76 mmol) was taken up in 40 mL methanol and cooled to 0° C. in ice bath. Zinc dust (1.80 g, 27.55 mmol) and ammonium chloride (1.474 g, 27.55 mmol) were added sequentially. The reaction was stirred on ice for 15 min. Then the ice bath was removed, and stirring was continued at room temperature for 2 hours. The reaction was filtered through celite with methanol, and the filtrate was concentrated in vacuo. Crude residue was re-suspended in ethyl acetate and washed twice with saturated NaHCO₃ solution and once with brine. Combined aqueous layers were extracted three times with ethyl acetate, the combined organic layers dried over sodium sulfate and concentrated in vacuo. The crude product was purified by silica gel chromatography using a gradient from 10 to 100% ethyl acetate in dichloromethane to provide 410 mg compound 8 (0.87 mmol, 32%). LCMS Method B: tr=0.85 min; m/z=470.2 [M+H]⁺.

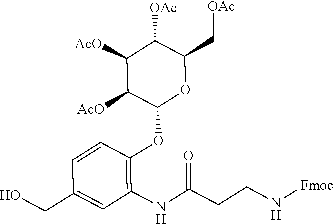

Step 5: (2R,3S,4S,5R,6R)-2-(2-(3-((((9H-fluoren-9-yl)methoxy)carbonyl)amino) propanamido)-4-(hydroxymethyl)phenoxy)-6-(acetoxymethyl)tetrahydro-2H-pyran-3,4,5-triyl triacetate (Compound 9): To a solution of 151 mg compound 8 (0.32 mmol) in 5 mL dichloromethane was added 110 mg 3-((((9H-fluoren-9-yl)methoxy)carbonyl)amino) propanoic acid (0.35 mmol) with addition of 0.2 mL DMF to aid solubility, and 87.5 mg EEDQ (0.35 mmol), and the reaction stirred at room temperature overnight. The reaction mixture was concentrated in vacuo, and the crude product purified by silica gel chromatography using a gradient from 0 to 3% methanol in dichloromethane to provide compound 9 (214 mg, 0.28 mmol, 87%). LCMS Method A: tr=1.43 min; m/z=763.3 [M+H]⁺.

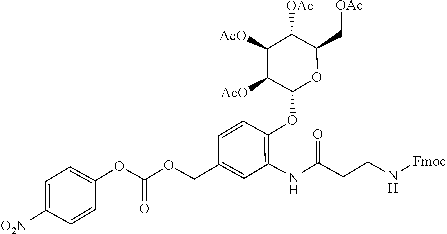

Step 6: (2R,3S,4S,5R,6R)-2-(2-(3-((((9H-fluoren-9-yl)methoxy)carbonyl)amino) propanamido)-4-(((4-nitrobenzoyl)oxy)methyl)phenoxy)-6-(acetoxymethyl)tetrahydro-2H-pyran-3,4,5-triyl triacetate (compound 10): To a solution of compound 9 (258 mg, 0.34 mmol) in 3 mL DMF was added 88.6 μL DIEA (0.51 mmol) and bis(4-nitrophenyl) carbonate (206 mg, 0.68 mmol), and the reaction mixture stirred at room temperature overnight. The reaction mixture was partitioned between water and ethyl acetate, and the organic layer washed three times with brine, dried over MgSO4, filtered and concentrated in vacuo. The crude product was purified by silica gel chromatography using a gradient from 10 to 70% ethyl acetate in hexanes to give 208 mg compound 10 (0.22 mmol, 65%). LCMS Method A: tr=1.61 min; m/z=928.4 [M+H]⁺.

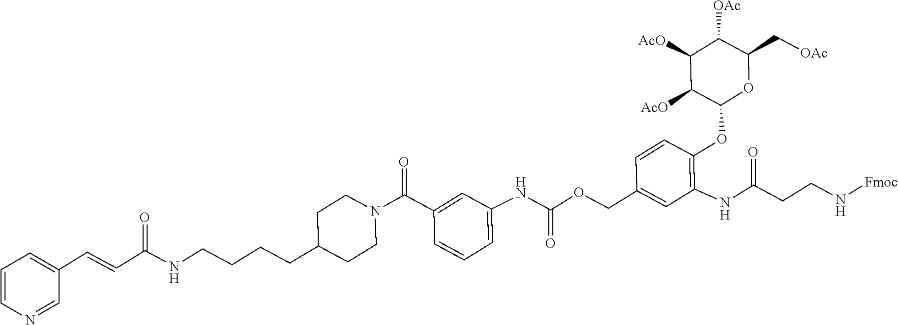

Step 7: (2R,3S,4S,5R,6R)-2-(2-(3-((((9H-fluoren-9-yl)methoxy)carbonyl)amino) propanamido)-4-((((3-(4-(4-((E)-3-(pyridin-3-yl)acrylamido)butyl)piperidine-1-carbonyl)phenyl)carbamoyl)oxy)methyl)phenoxy)-6-(acetoxymethyl)tetrahydro-2H-pyran-3,4,5-triyl triacetate (Compound 11): (E)-N-(4-(1-(3-aminobenzoyl)piperidin-4-yl)butyl)-3-(pyridin-3-yl)acrylamide (581 mg, 0.916 mmol) and 934 mg compound 10 (1.01 mmol) were dissolved in 106 mL DMF and 2.1 mL pyridine. 12.5 mg HOAt (0.092 mmol) was added as a solution in DMF, and the reaction stirred at room temperature overnight. The reaction was poured into EtOAc, and the organic layer washed 2× water, dried over MgSO4 and concentrated in vacuo. The crude product was purified by silica gel chromatography using a gradient from 0 to 10% methanol in dichloromethane to provide 850 mg compound 11 (0.711 mmol, 78%). LCMS Method C: tr=1.84 min; m/z=1195.8 [M+H]⁺.

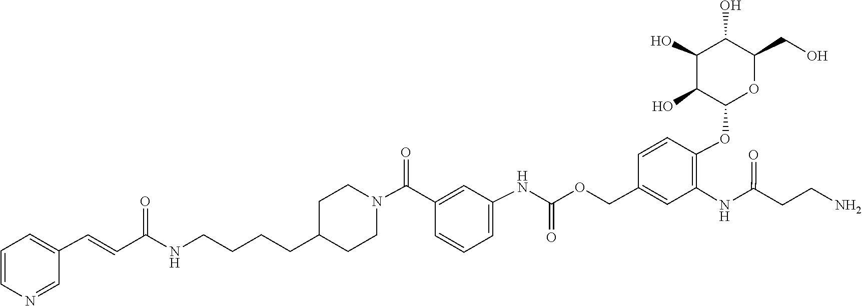 [SMILES: NCCC(=O)Nc1cc(COC(=O)Nc2cccc(C(=O)N3CCC(CCCCNC(=O)/C=C/c4cccnc4)CC3)c2)ccc1O[C@H]1O[C@H](CO)[C@@H](O)[C@H](O)[C@@H]1O]

Step 8: 3-(3-aminopropanamido)-4-(((2R,3S,4S,5S,6R)-3,4,5-trihydroxy-6-(hydroxymethyl) tetrahydro-2H-pyran-2-yl)oxy)benzyl (3-(4-(4-((E)-3-(pyridin-3-yl)acrylamido)butyl) piperidine-1-carbonyl) phenyl)carbamate (Compound 12): 383 mg compound 11 (0.293 mmol) was dissolved in 6 mL THE and 6 mL MeOH and cooled on ice. A solution of 5.9 mL LiOH (0.5M, 2.93 mmol) was slowly added. After 30 minutes, the reaction was removed from ice and allowed to warm to room temperature. After 4 hours, the reaction was quenched with 167.5 μL acetic acid (2.93 mmol) and concentrated in vacuo. Crude residue taken up in DMSO, filtered, and purified by preparative HPLC to give 230 mg compound 12 (0.223 mmol, 76%) as the TFA salt. LCMS Method D: tr=0.79 min; m/z=805.4 [M+H]⁺.

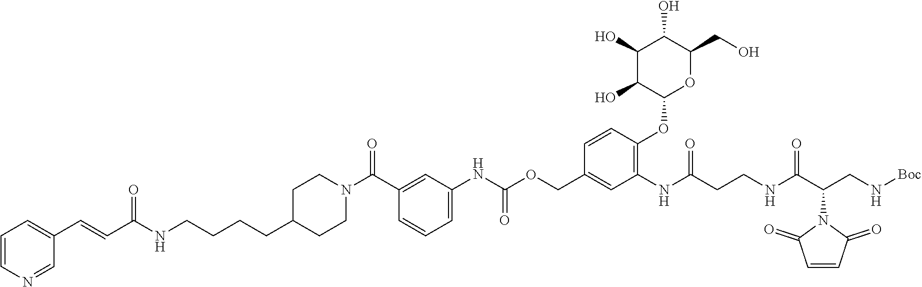 [SMILES: CC(C)(C)OC(=O)NC[C@@H](C(=O)NCCC(=O)Nc1cc(COC(=O)Nc2cccc(C(=O)N3CCC(CCCCNC(=O)/C=C/c4cccnc4)CC3)c2)ccc1O[C@H]1O[C@H](CO)[C@@H](O)[C@H](O)[C@@H]1O)N1C(=O)C=CC1=O]

Step 9: 3-(3-((S)-3-((tert-butoxycarbonyl)amino)-2-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)benzyl (3-(4-(4-((E)-3-(pyridin-3-yl)acrylamido)butyl)piperidine-1-carbonyl)phenyl)carbamate (Compound 13): compound 12 (334 mg, 0.324 mmol) was dissolved in 3.5 mL DMF and 0.17 mL DIPEA (0.971 mmol) followed by addition of 148 mg 2,5-dioxopyrrolidin-1-yl (2S)-3-[(tert-butoxycarbonyl)amino]-2-(2,5-dioxopyrrol-1-yl)propanoate (0.388 mmol). After 3 hours, the reaction was diluted with DMSO and purified by preparative HPLC to give compound 13 (299 mg, 0.253 mmol, 78%) as the TFA salt. LCMS Method C: tr=1.32 min; m/z=1071.7 [M+H]⁺.

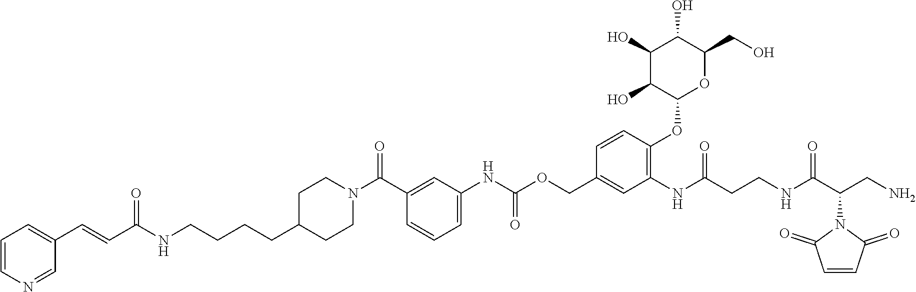 [SMILES: NC[C@@H](C(=O)NCCC(=O)Nc1cc(COC(=O)Nc2cccc(C(=O)N3CCC(CCCCNC(=O)/C=C/c4cccnc4)CC3)c2)ccc1O[C@H]1O[C@H](CO)[C@@H](O)[C@H](O)[C@@H]1O)N1C(=O)C=CC1=O]

Step 10: 3-(3-((S)-3-amino-2-(2,5-dioxo-2,5-dihydro-1H-pyrrol-1-yl)propanamido) propanamido)-4-(((2R,3S,4S,5S,6R)-3,4,5-trihydroxy-6-(hydroxymethyl)tetrahydro-2H-pyran-2-yl)oxy)benzyl (3-(4-(4-((E)-3-(pyridin-3-yl)acrylamido)butyl)piperidine-1-carbonyl) phenyl)carbamate (Compound 14—MC9): compound 13 (299 mg, 0.253 mmol) was treated with 20% TFA in 15 mL DCM for 2 hours. The solvent was removed in vacuo, and the residue dissolved in 50/50 CH₃CN/H₂O and purified by preparative HPLC to provide compound 14 (201 mg, 0.168 mmol, 66%) as the TFA salt. LCMS Method C: tr=1.10 min; m/z=971.6 [M+H]⁺.

### Example 5: Synthesis of MC10

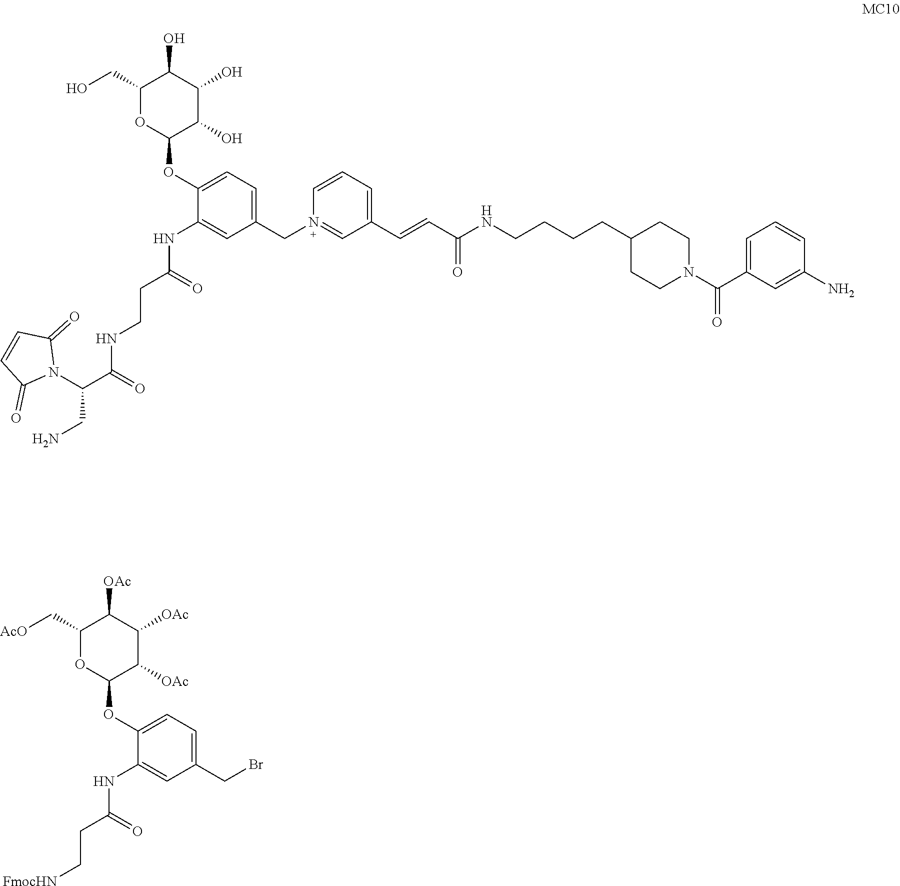

Step 1: (2R,3S,4S,5R,6R)-2-(2-(3-((((9H-fluoren-9-yl)methoxy)carbonyl)amino) propanamido)-4-(bromomethyl)phenoxy)-6-(acetoxymethyl)tetrahydro-2H-pyran-3,4,5-triyl triacetate (Compound 10): the benzyl alcohol analog of compound 10 (200 mg, 0.262 mmol) and 103 mg PPh₃ (0.393 mmol) were dissolved in 8 mL DCM at 0° C. N-bromosuccinimide (70 mg, 0.393 mmol) was added in two portions at the same temperature. Ice bath was then removed and allowed the reaction to slowly warm up to room temperature. After 4 hours the solvent was removed and the crude reaction mixture was purified by flash column chromatography to provide compound 10 (154 mg, 0.187 mmol, 71.0%). LCMS Method E: tᵣ=2.31 min; m/z=825.04 [M+1]⁺.

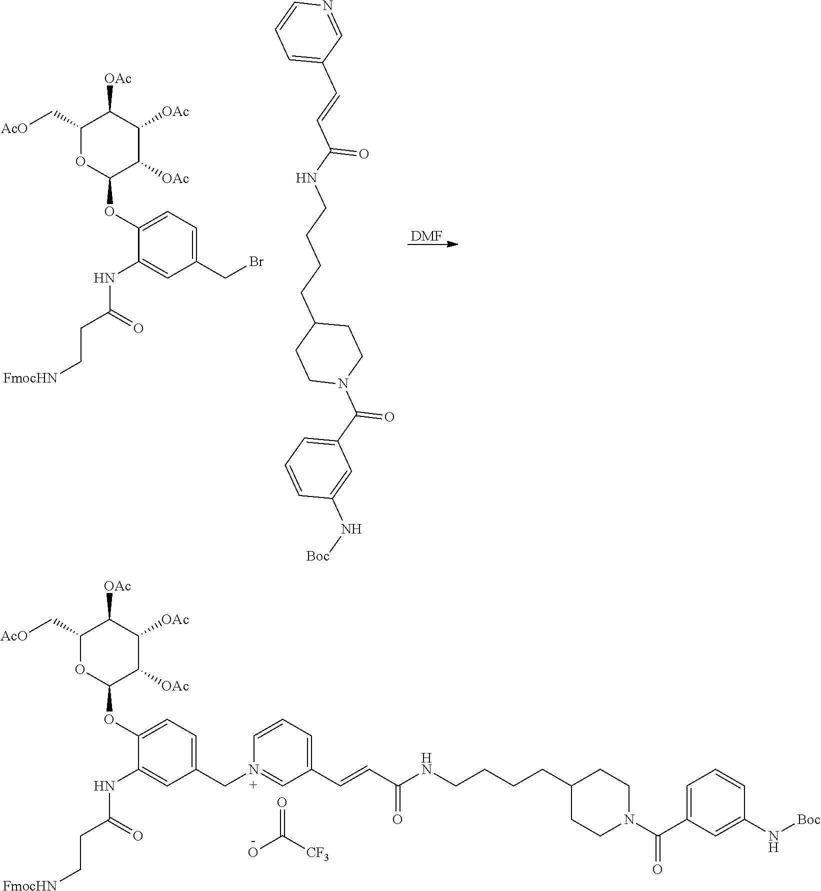

Step 2: 1-(3-(3-((((9H-fluoren-9-yl)methoxy)carbonyl)amino)propanamido)-4-(((2R,3S,4S,5R,6R)-3,4,5-triacetoxy-6-(acetoxymethyl)tetrahydro-2H-pyran-2-yl)oxy)benzyl)-3-((E)-3-((4-(1-(3-((tert-butoxycarbonyl)amino)benzoyl)piperidin-4-yl)butyl)amino)-3-oxoprop-1-en-1-yl)pyridin-1-ium (Compound 1): compound 10 (109.3 mg, 0.132 mmol) and tert-butyl (E)-(3-(4-(4-(3-(pyridin-3-yl)acrylamido)butyl)piperidine-1-carbonyl)phenyl)carbamate (51.6 mg, 0.102 mmol) was dissolved in anhydrous 800 μL DMF and heated up to 55° C. for 2 hours. The reaction was cooled to room temperature, diluted with DMSO and water, purified by preparative HPLC to provide 108.2 mg compound 11 (0.079 mmol, 77.8%). LCMS Method E: tr=2.00 min; m/z=1251.40 [M]⁺.

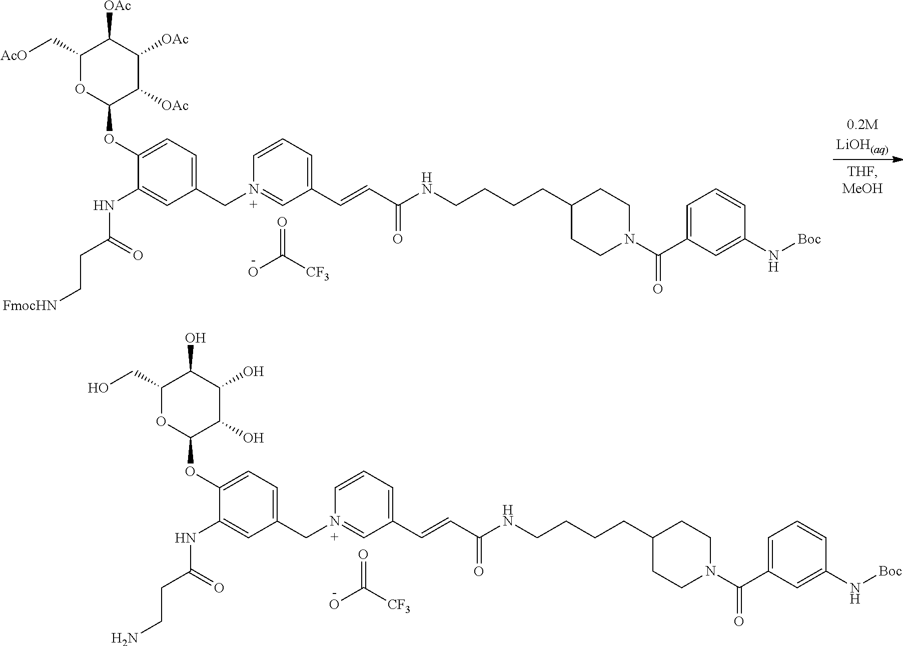

Step 3: 1-(3-(3-aminopropanamido)-4-(((2R,3S,4S,5S,6R)-3,4,5-trihydroxy-6-(hydroxymethyl)tetrahydro-2H-pyran-2-yl)oxy)benzyl)-3-((E)-3-((4-(1-(3-((tert-butoxycarbonyl)amino)benzoyl)piperidin-4-yl)butyl)amino)-3-oxoprop-1-en-1-yl)pyridin-1-ium 2,2,2-trifluoroacetate (Compound 12): compound 11 (508 mg, 0.037 mmol) was dissolved in 1.8 mL of a 1:1 mixture of MeOH and THF. The solution was cooled on ice prior to the addition of LiOH solution (1.86 mL, 0.2 M, 0.372 mmol). The reaction was stirred on ice for 30 mins, and then warmed to room temperature. After 3 hours, the reaction was acidified with 20 μL acetic acid, then diluted with DMSO/water and purified by preparative HPLC to provide 20.6 mg of compound 12 (0.019 mmol, 50.8%). LCMS Method E: tr=0.84 min; m/z=861.39 [M]⁺.

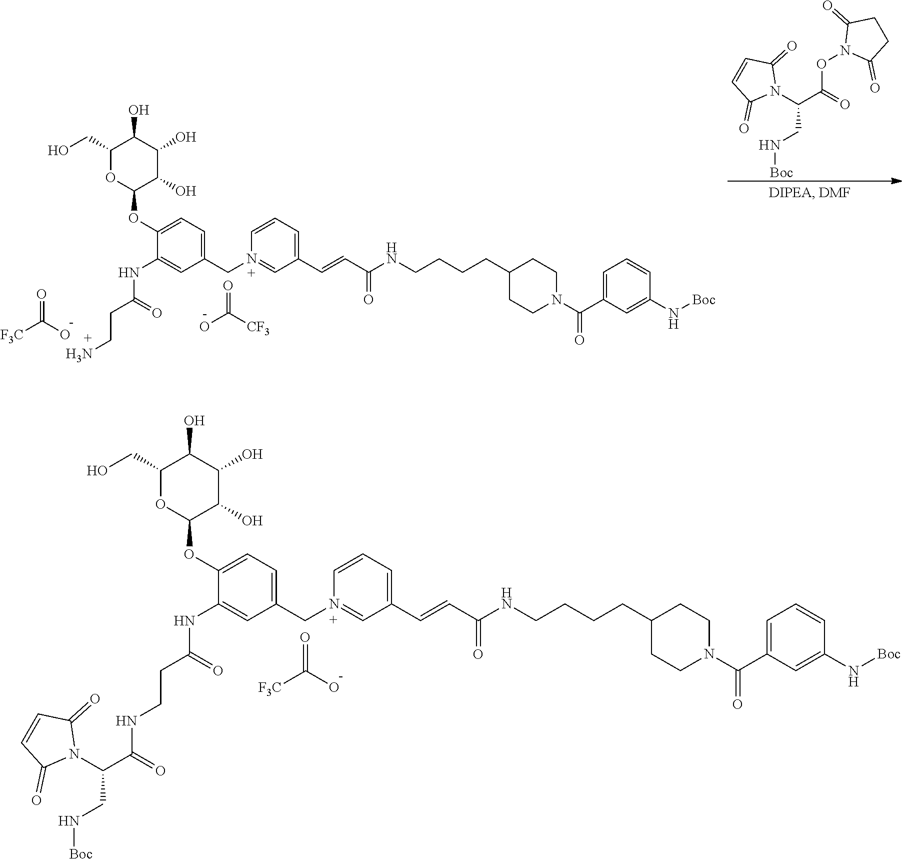

Step 4: 1-(3-(3-((S)-3-((tert-butoxycarbonyl)amino)-2-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)benzyl)-3-((E)-3-((4-(1-(3-((tert-butoxycarbonyl)amino)benzoyl)piperidin-4-yl)butyl)amino)-3-oxoprop-1-en-1-yl)pyridin-1-ium 2,2,2-trifluoroacetate (Compound 13): compound 12 (10.2 mg, 0.011 mmol) was dissolved in anhydrous 300 μL DMF followed by the addition of 9.3 μL DIPEA. 6.12 mg 2,5-Dioxopyrrolidin-1-yl (S)-3-((tert-butoxycarbonyl)amino)-2-(2,5-dioxo-2,5-dihydro-1H-pyrrol-1-yl)propanoate (0.016 mmol) in anhydrous 100 μL DMF was then added. The reaction mixture was stirred at room temperature for 30 min. After 30 min, reaction was acidified with HOAc (10 μL), diluted with DMSO/water and purified by prep-HPLC to provide compound 13 (10.3 mg, 0.008 mmol, 77.5%). LCMS Method E: tᵣ=1.58 min; m/z=1127.79 [M]⁺.

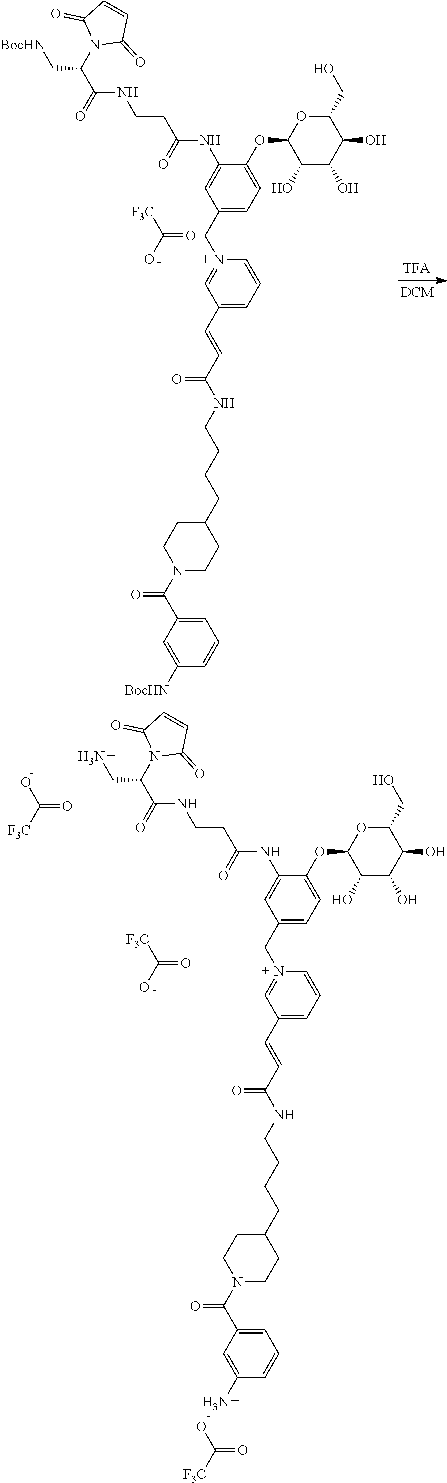

Step 5: 1-(3-(3-((S)-3-ammonio-2-(2,5-dioxo-2,5-dihydro-1H-pyrrol-1-yl)propanamido) propanamido)-4-(((2R,3S,4S,5S,6R)-3,4,5-trihydroxy-6-(hydroxymethyl)tetrahydro-2H-pyran-2-yl)oxy)benzyl)-3-((E)-3-((4-(1-(3-ammoniobenzoyl)piperidin-4-yl)butyl)amino)-3-oxoprop-1-en-1-yl)pyridin-1-ium 2,2,2-trifluoroacetate (Compound 14 MC10): 10.3 mg compound 13 (0.008 mmol) was suspended in 240 μL DCM and 60 μL TFA was added. The reaction mixture turned homogenous after adding TFA. The reaction was stirred at room temperature for 4 hours. After 4 hours, solvent was removed under vacuum and the crude product was diluted with DMSO/water and purified by prep-HPLC to provide compound 14 (MC10) (5.4 mg, 0.004 mmol, 51.3%). LCMS Method E: tr=1.45 min; m/z=927.46 [M]⁺.

### Example 6: Hydrophobic Interaction Chromatography (HIC) of hAC10ec Conjugates with MC1 or MC3

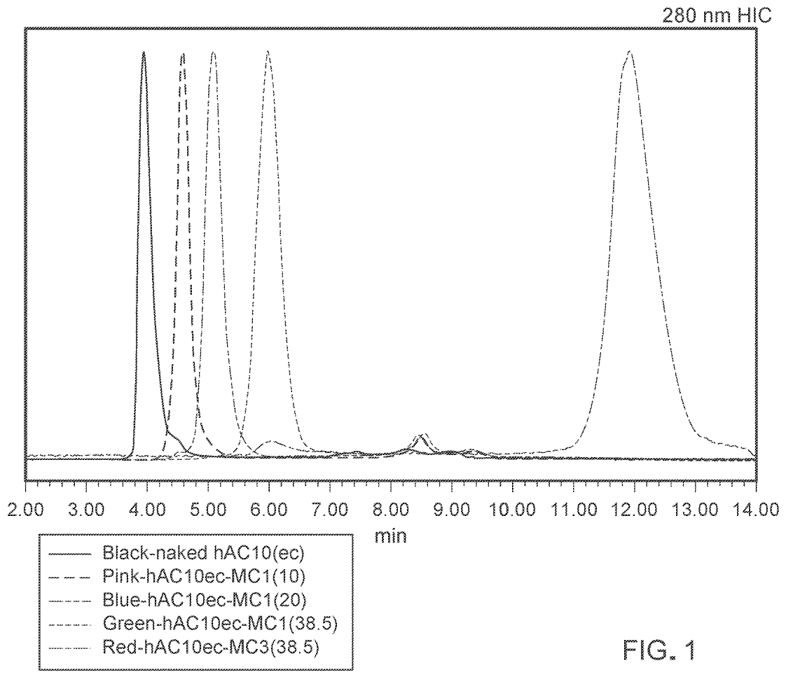

Hydrophobic interaction was measured with HIC (280 nm). Results of the HIC are shown in FIG. 1. The retention time of unconjugated hAC10ec (first peak) was about 4 minutes. The retention time of hAC10ec-MC1(10) (second peak) was about 4.5 minutes. The retention time of hAC10ec-MC1(20) (third peak) was about 5.3 minutes. The retention time of hAC10ec-MC1(38.5) (fourth peak) was about 6.0 minutes. The retention time of hAC10ec-MC3(38.4) (fifth peak) was about 11.8 minutes.

### Example 7: Conjugation with MC2 and N-Ethyl Maleimide (NEM)

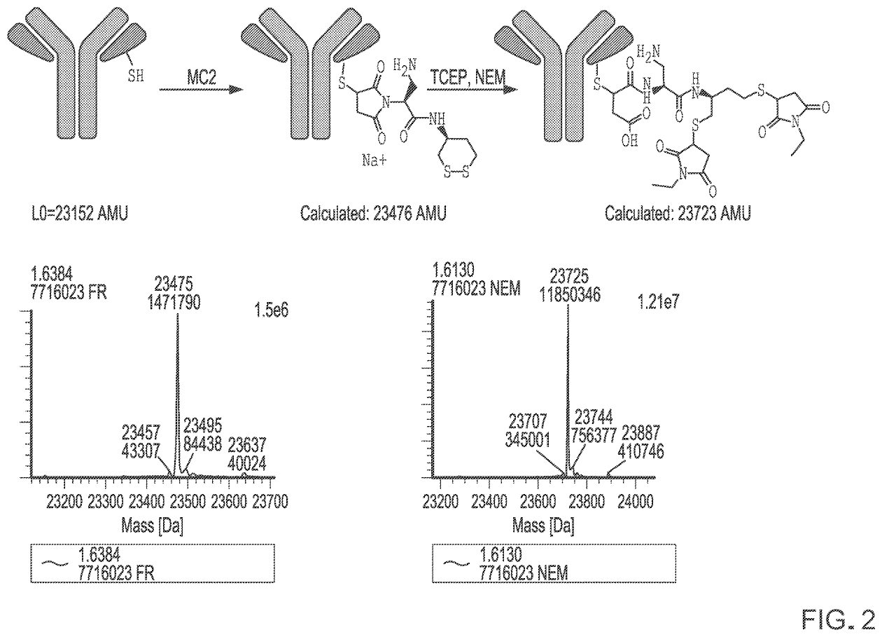

An exemplary embodiment of antibody conjugation with duplexer MC2 and N-Ethyl maleimide and corresponding spectroscopy data is shown in FIG. 2.

Referring to FIG. 2, an Antibody (cAC10) having a L0=23152 was conjugated with duplexer MC2 to form an antibody-duplexer conjugate (see below) (expected mass: 23476; observed mass: 23475).

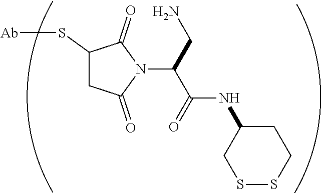

The antibody-duplexer conjugate was then reduced with TCEP, followed by conjugation with N-ethylmaleimide (NEM) to form an antibody-duplexer-NEM conjugate (see below) (expected mass 23723; observed mass 23725).

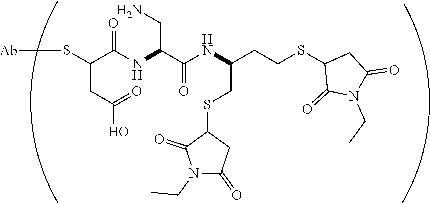

### Example 8: Experimental Procedure for Conjugation of IgG1-MC6(8) to Produce 16-Load ADCs of MC7/-MC8/-MC9/-MC10 (PEG on Duplexer)

Step 1: 15 mg fully reduced antibody IgG1 in 1.16 mL PBS was conjugated with MC6 (13.3 mM solution in DMSO; 1.45 equiv of scaffold per reactive thiol) in PBS at room temperature for 2 hours. Reaction completion was confirmed by PLRP-MS analysis. The reaction mixture was purified by size-exclusion chromatography eluting with PBS. The resulting solution was concentrated to provide the antibody-scaffold conjugate at 11.8 mg/ml. The solution was adjusted to pH 8 using 1M potassium phosphate buffer at pH 8. The scaffold disulfides were reduced using TCEP (2 equiv per disulfide), incubating at 37° C. for 75 min. Complete reduction was verified by reaction of an analytical aliquot with excess N-acetyl maleimide followed by PLRP-MS analysis. The completed reaction was purified by size exclusion chromatography eluting with PBS+2 mM EDTA. The eluent was concentrated to 15.6 mg/mL and stored at −20° C. until further use.

Step 2: 3 mg fully reduced antibody-scaffold conjugate was conjugated with indicated drug linkers (10 mM solutions in DMSO; 1.25-1.45 equiv of drug linker per reactive thiol) in PBS at room temperature for 2 hours. Reaction completion was confirmed by PLRP-MS analysis. The reactions were purified by size-exclusion chromatography eluting with PBS. The eluents were diluted to 4 ml prior to concentration to ˜1 ml. This dilution/concentration procedure was repeated once more prior to final concentration to ˜300 μl. Concentration of the resulting ADCs was determined using the DC Protein Assay (Bio-Rad). The identity of the final conjugates was confirmed by PLRP-MS, and the presence of high-molecular weight species determined by analytical SEC.

### Example 9: Experimental Analytical Data for Antibody-Drug Conjugates

Lₑₓₚ and Hₑₓₚ are predicted masses of antibody light and heavy chains, respectively, excluding hydrolysis of the thiosuccinimide moiety after conjugation. Lobs and Hobs are observed masses of the predominant species as determined by PLRP-MS analysis; the number of additional waters (from thiosuccinimide hydrolysis prior to analysis) are indicated. % HMW indicates the percentage of high molecular weight species as determined by analytical size-exclusion chromatography.

| | Lₑₓₚ | L_{obs} | Hₑₓₚ | H_{obs} | % HMW |
|---|---|---|---|---|---|
| IgG1 | | 23151 | | 50470 | Not |
| | | | | | measured |
| IgG1-MC6(8) | 24679 | 24698 | 55053 | 55110 | Not |
| | | (Lₑₓₚ + 1 H₂0) | | (Hₑₓₚ + 3 H₂0) | measured |
| IgG1-MC6(8)- | 26650 | 26670 | 60965 | 61043 | 3.4% |
| MC7(16) | | (Lₑₓₚ + 1 H₂0) | | (Hₑₓₚ + 4 H₂0) | |
| IgG1-MC6(8)- | 26564 | 26600 | 60707 | 60798 | 2.4% |
| MC8(16) | | (Lₑₓₚ + 1 H₂0) | | (Hₑₓₚ + 5 H₂0) | |
| IgG1-MC6(8)- | 26622 | 26660 | 60881 | 60995 | 7.6% |
| MC9(16) | | (Lₑₓₚ + 2 H₂0) | | (Hₑₓₚ + 6 H₂0) | |
| IgG1-MC6(8)- | 26536 | 26572 | 60623 | 60750 | 1.8% |
| MC10(16) | | (Lₑₓₚ + 2 H₂0) | | (Hₑₓₚ + 7 H₂0) | |
| IgG1-MC2(8) | 23452 | 23471 | 51373 | 51428 | Not |
| | | (Lₑₓₚ + 1 H₂0) | | (Hₑₓₚ + 3 H₂0) | measured |
| IgG1-MC2(8)- | 25337 | 25373 | 57027 | 57115 | 1.2% |
| MC8(16) | | (Lₑₓₚ + 2 H₂0) | | (Hₑₓₚ + 5 H₂0) | |
| cAC10 | | 23724 | | 50320 | |
| cAC10- | 27223 | 27279 | 60817 | 60985 | 2.2% |
| MC6(8)- | | (Lₑₓₚ + 3 H₂0) | | (Hₑₓₚ + 9 H₂0) | |
| MC7(16) | | | | | |
| cAC10- | 27137 | 27190 | 60559 | 60715 | <5% |
| MC6(8)- | | (Lₑₓₚ + 3 H₂0) | | (Hₑₓₚ + 9 H₂0) | |
| MC8(16) | | | | | |
| cAC10- | 27195 | 27251 | 60733 | 60901 | 9.6% |
| MC6(8)- | | (Lₑₓₚ + 3 H₂0) | | (Hₑₓₚ + 9 H₂0) | |
| MC9(16) | | | | | |
| cAC10- | 27109 | 27163 | 60475 | 60640 | <5% |
| MC6(8)- | | (Lₑₓₚ + 3 H₂0) | | (Hₑₓₚ + 9 H₂0) | |
| MC10(16) | | | | | |
| Ab1ec | | 24210 | | 50763 | |
| Ab1ec-MC6- | 27681 | 27732 | 64647 | 64648 | 4.6% |
| MC9 (20) | | (Lₑₓₚ + 3 H₂0) | | (Hₑₓₚ + 0 H₂0) | |

### Example 10: Analytical Characterization of Auristatin Conjugates with cAC10 and Conjugate Intermediates Thereof

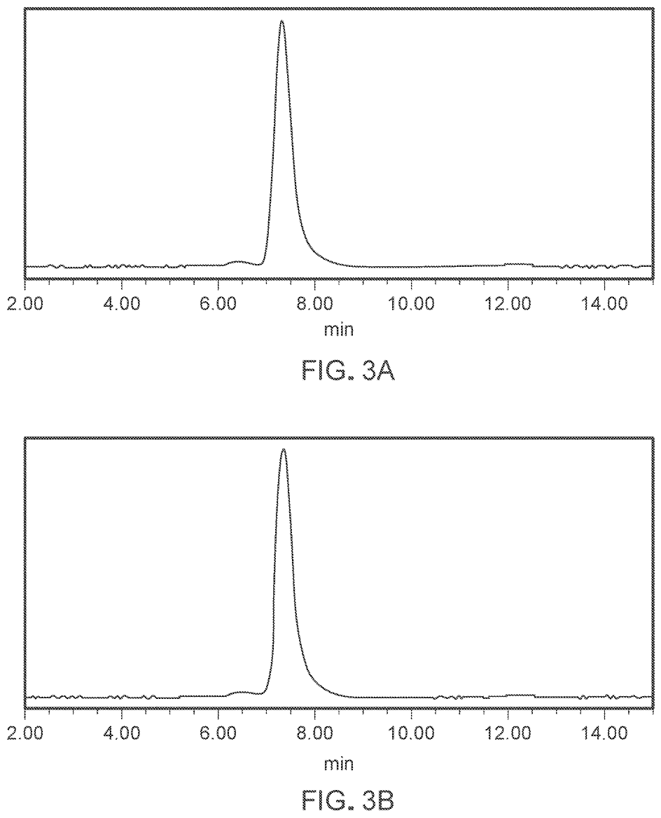

Size exclusion chromatogram of 16-load auristatin ADCs with formula cAC10-MC2(8)-MC4(16) is shown in FIG. 3 (A) (retention time: about 6.6 minutes). Size exclusion chromatography data for 16-load auristatin ADCs with formula cAC10-MC2(8)-MC5(16) is shown in FIG. 3(B) (retention time: about 6.6 minutes).

Chromatography and Mass Spectroscopy Data on Duplexer Conjugates with MC4 (Ab-MC2(8)-MC4(16)).

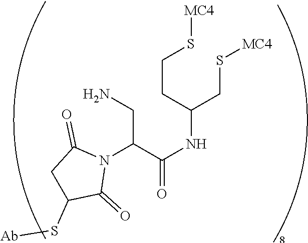

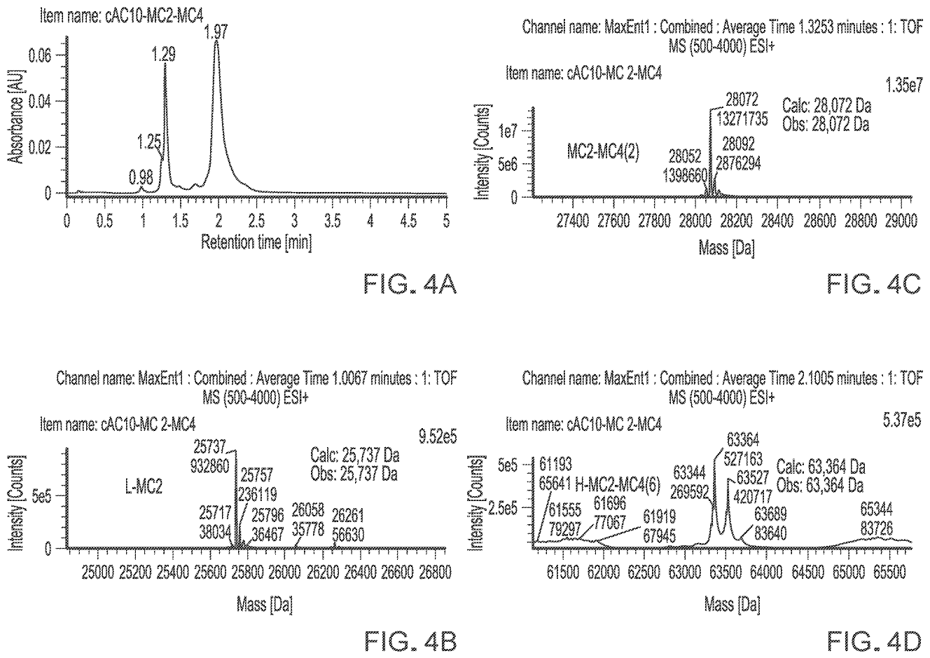

FIG. 4(A) shows the PLRP chromatogram of cAC10 conjugates with MC2 and MC4 (retention time of light chain: about 1.29 minutes; retention time of heavy chain: about 1.97 mins). The mass spectrometry data indicate conjugation of 2 equivalent of MC4 to each light chain and 6 equivalent of MC4 to each heavy chain. As such, the antibody in total was found to be conjugated with 16 equivalents of MC4.

FIG. 4(B) shows the mass spectrum of antibody (cAC10) light chain conjugated to one unit of MC2 (expected: 25,737; observed 25,737).

FIG. 4(C) shows the mass spectrum of antibody (cAC10) light chain conjugated to MC2(1)-MC4(2) (expected: 28,072; observed 28,072).

FIG. 4(D) shows the mass spectrum of antibody (cAC10) heavy chain conjugated to MC2(3)-MC4(6) (expected: 63,364; observed: 63,364). Observation of multiple peaks is attributable to G0, G1 and G2 oligosaccharide forms of the heavy chain.

Chromatography and Mass Spectroscopy Data on Duplexer Conjugates with MC5 (Ab-MC2(8)-MC5(16)).

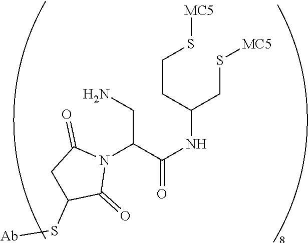

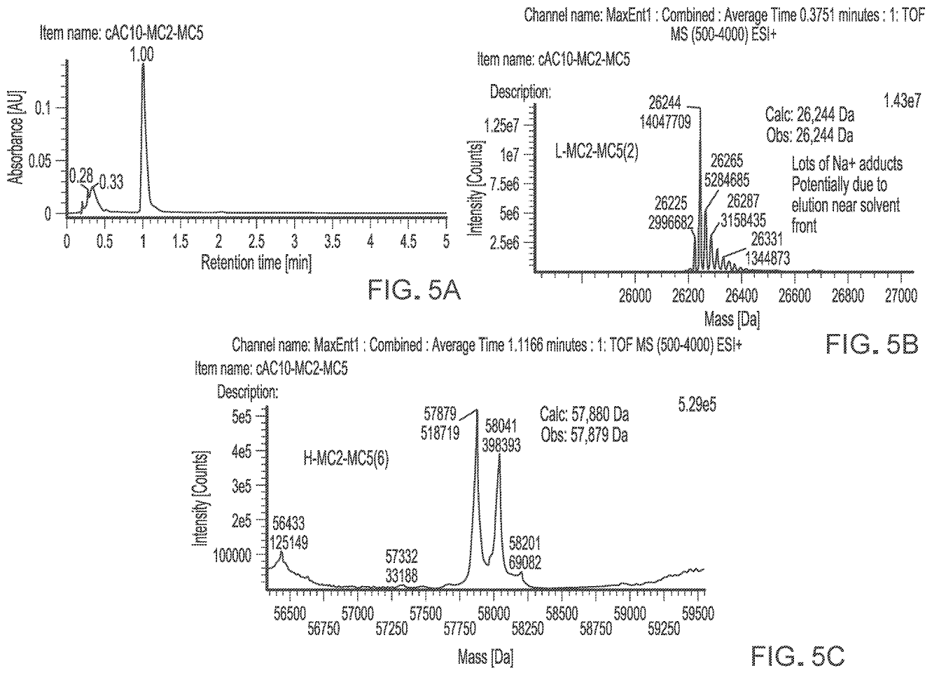

FIG. 5(A) shows the PLRP chromatogram of cAC10 conjugates with MC2 and MC5 (retention time of light chain: about 0.33 minutes; retention time of heavy chain: about 1.0 minutes. The mass spectrometry data indicate conjugation of 2 equivalent of MC4 to each light chain and 6 equivalent of MC5 to each heavy chain. As such, the antibody in total was found to be conjugated with 16 equivalents of MC5.

FIG. 5(B) shows the mass spectrum of antibody (cAC10) light chain conjugated MC2(1)-MC5(2) (expected: 26,244; observed: 26,244).

FIG. 5(C) shows the mass spectrum data of antibody (cAC10) heavy chain conjugated to MC2(3)-MC5(6) (expected: 57,880; observed: 57,879). Observation of multiple peaks is attributable to G0, G1 and G2 oligosaccharide forms of the heavy chain.

### Example 11: Preparation of Dendrimeric ADCs Comprising One or More Multiplexers

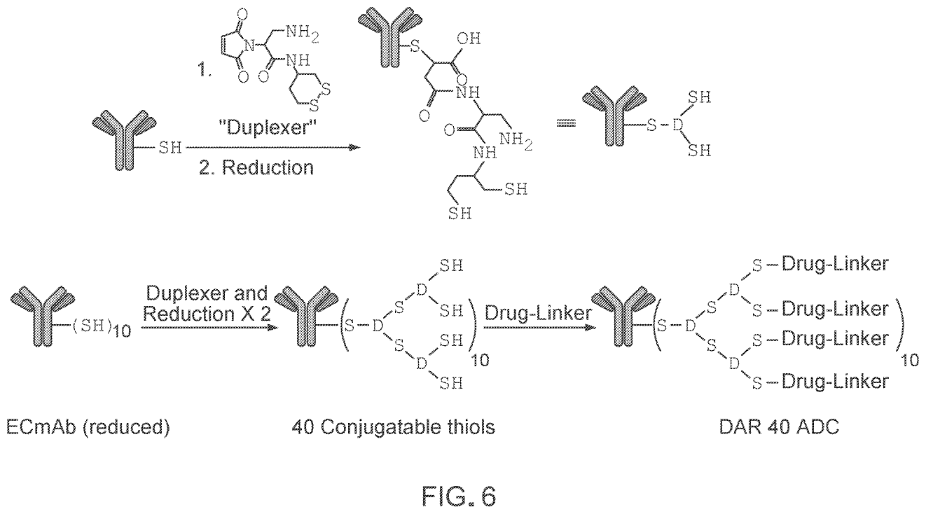

FIG. 6 schematically depicts a method for the preparation of dendrimeric ADCs comprising one or more multiplexer moieties. An individual Ab can be reduced and conjugated with a duplexer MC2. In a reduced cysteine engineered monoclonal antibody (ECmAb) having 10 cysteine moieties, the thiol group of each cysteine can be conjugated to an MC2 unit. Each MC2 unit can then be conjugated further to two MC2 units. Conjugation of L²-D moieties to the terminal MC2 units therefore allow the formation of ADCs with DAR=40. These ADCs have the general formula of Ab-MC2(10)-MC2(20)-(L²-D)₄₀.

### Example 12: Characterization of Hydrophilic Dendrimeric ADCs

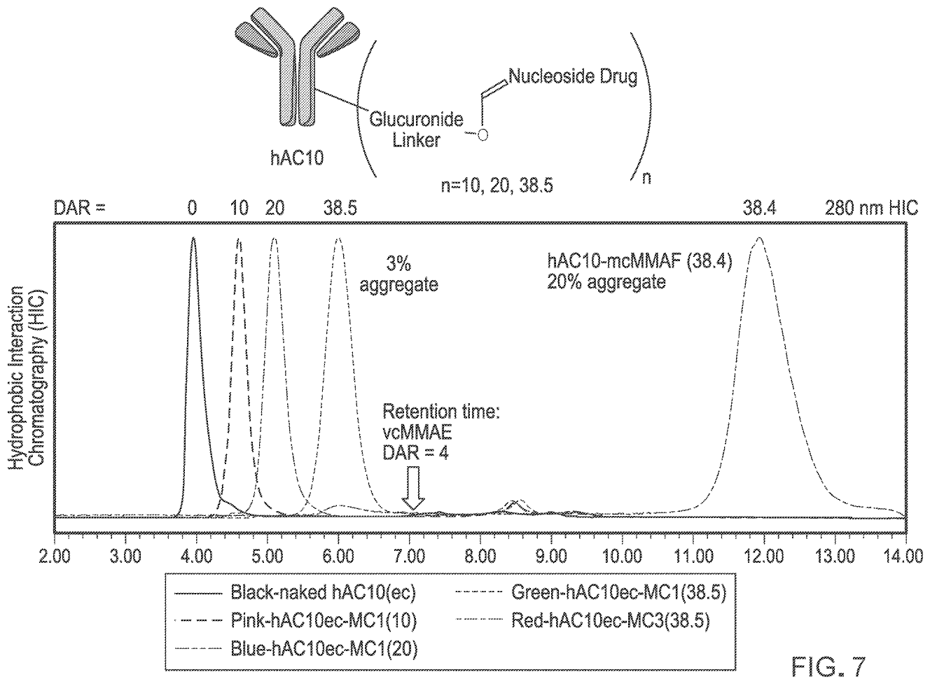

FIG. 7 is the Hydrophobic Interaction Chromatography (HIC) chromatogram of hAC10 conjugates with a drug moiety (MC1 or MC3) having different DARs (DAR=0, 10, 20, and 38.5). Hydrophobic interaction was measured with 280 nm HIC. The retention time of naked hAC10ec (first peak) was about 4 minutes. The retention time of hAC10ec-MC1(10) (second peak) was about 4.5 minutes. The retention time of hAC10ec-MC1(20) (third peak) was about 5.3 minutes. The retention time of hAC10ec-MC1(38.5) (fourth peak) was about 6.0 minutes. The retention time of hAC10ec-MC3(38.4) (fifth peak) was about 11.8 minutes. The retention time for commercial drug linker vcMMAE DAR(4) is about 7 minutes.

### Example 13: Cytotoxicity of Duplexer-Based Gemcitabine ADCs on L540cy Cells

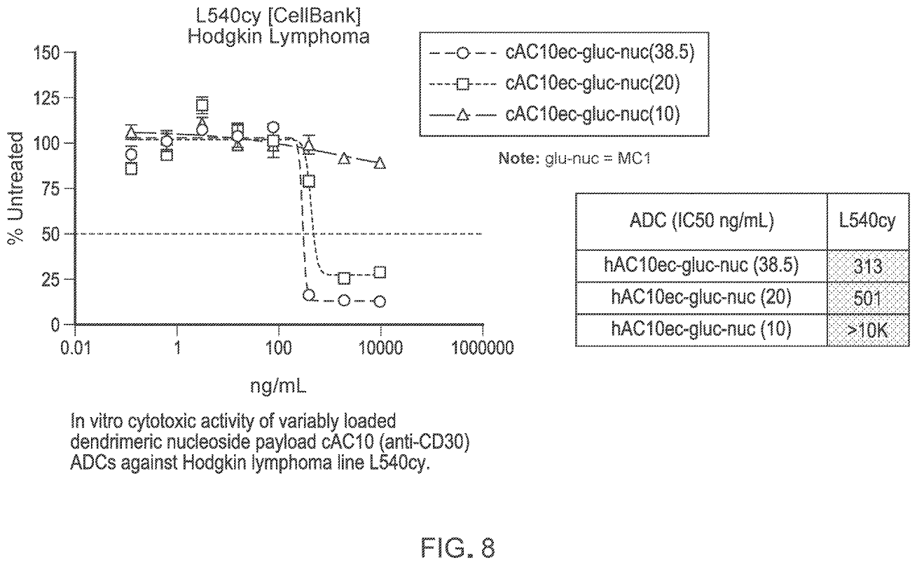

FIG. 8 shows the in vitro cytotoxicity of cAc10ec-MC1 ADCs having different DAR values to Hodgkin's Lymphoma cell line L540cy. The IC₅₀ value for hAC10ec-MC1 (38.5) was 313 ng/mL (circles), the IC₅₀ value for hAC10ec-MC1 (20) was 501 ng/mL (squares), and the IC50 value for hAC10ec-MC1 (10) was >10 k (triangles).

### Example 14: Rat Pharmacokinetic Data for IgG1-MC6(8)-MC7(16)/-MC8(16)/-MC9(16)/-MC10(16) and IgG1-MC2(8)-MC8(16)

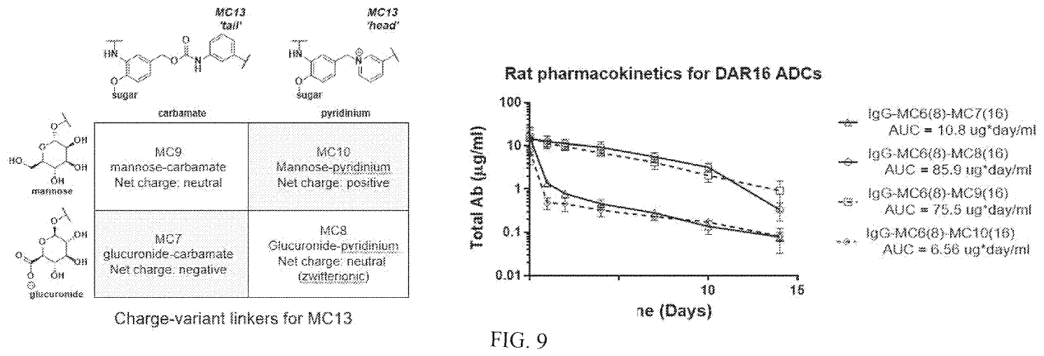

FIG. 9 shows the rat pharmacokinetic data of DAR16 conjugates of antibody IgG1 with an NAMPT inhibitor, having different charges at the L²-D units. Constructs with neutral or zwitterionic L²-D units showed extended half-lives compared to those with net negative or positive charge (which were rapidly cleared). Results can be seen by comparing ADCs with L²-D=MC9 (neutral, dashed line with squares) or MC8 (zwitterionic, solid line with circles) with those having L²-D=MC7 (negatively charged, solid line with triangles) and MC10 (positively charged, dashed line with diamonds).

### Example 15: Xenograft Efficacy Data for cAC10-MC6(8)-(L²-D)(16)

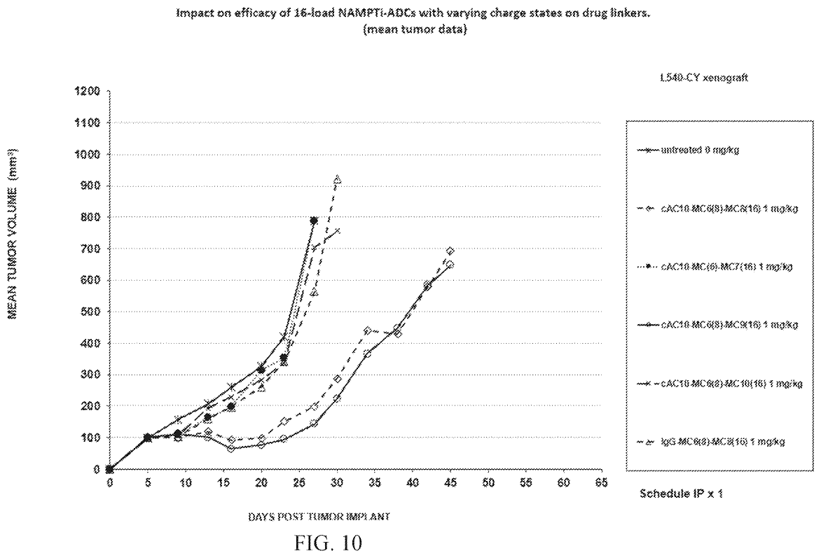

FIG. 10 shows the xenograft efficacy of cAC10 and IgG1 conjugates with an NAMPT inhibitor having the general formula of cAC10-MC6(8)-(L²-D)(16) on L540cy-161 cells, wherein L²-D is MC7, MC8, MC9, or MC10. Post-implant mean tumor volume absent treatment (i.e., 0 mg/kg (* markers, solid line))) is compared with the mean tumor volume following treatment with cAC10-MC6(8)-MC8(16) 1 mg/kg (open diamonds, short dash)), cAC10-MC6(8)-MC7(16) 1 mg/kg (filled circles, dotted line), cAC10-MC6(8)-MC9(16) 1 mg/kg (open circles, solid line), cAC10-MC6(8)-MC10(16) 1 mg/kg (X markers, long dash), and IgG-MC6(8)-MC8(16) 1 mg/kg (open triangle, short dash).

### Example 16: Xenograft Efficacy Data for Ab3(ec)-MC6(10)-MC9(20) Versus Ab3(ec)-MC7(10) (KG-1 Xenograft Model)

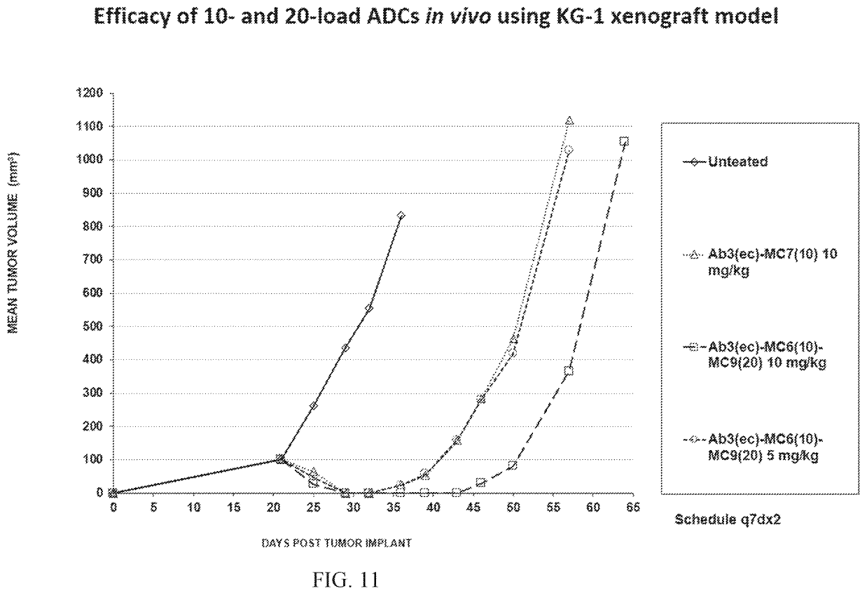

FIG. 11 shows the xenograft efficacy of Ab3(ec)-MC6(10)-MC9(20) and Ab3(ec)-MC7(10) ADCs on KG-1 cells. 10- and 20-load ADCs are compared in vivo using both Ab- and drug normalized dosing (mean tumor data). Mean tumor volume with untreated KG-1 cells 0 mg/kg (open diamonds, solid line) is compared with the mean tumor volume following treatment with Ab3(ec)-MC7(10) 10 mg/kg (open triangles, dotted line), Ab3(ec)-MC6(10)-MC9-(20) 10 mg/kg (open squares, long-dash line), and Ab3(ec)-MC6(10)-MC9(20) 5 mg/kg (open circles, short-dash line). Dosing schedule is 27dx2.

### Example 17: Experimental Data of NAD-Glo Assay of High Load ADCs

Experimental data from Nad-Glo (Promega) Assays according to manufactures instructions.

TABLE 1A
In vitro data for cAC10 high load ADCs

| | | | Cell lines; x50 (ng/ml) | | |
|---|---|---|---|---|---|
| ADC | Antigen | Assay | L540cy | L428 | Karpas-299 |
| cAC10-MC6(8)- MC7(16) | CD30 | NAD-Glo | 8.4 | 74 | 44 |
| cAC10-MC6(8)- MC8(16) | CD30 | NAD-Glo | 6.8 | 35 | 27 |
| cAC10-MC6(8)- MC9(16) | CD30 | NAD-Glo | 2.7 | 24 | 10 |
| cAC10-MC6(8)- | CD30 | NAD-Glo | 6.5 | 430 | 78 |
| MC10(16) | | | | | |

### Example 18: Experimental Data of CTG Assays of High Load ADCs

Experimental data from CTG Assays (Promega) according to manufactures instructions.

TABLE 1B

| | | | Cell lines; x50 (ng/ml) | | |
|---|---|---|---|---|---|
| ADC | Antigen | Assay | L540cy | L428 | Karpas-299 |
| cAC10-MC6(8)-MC7(16) | CD30 | CTG | 100 | >2000 | 1230 |
| cAC10-MC6(8)-MC8(16) | CD30 | CTG | 55 | >2000 | >2000 |
| cAC10-MC6(8)-MC9(16) | CD30 | CTG | 35 | >2000 | >2000 |
| cAC10-MC6(8)- | CD30 | CTG | 170 | >2000 | >2000 |
| MC10(16) | | | | | |

### Example 19: Experimental Data of Nad-Glo Assays of High Load ADCs Against Acute Myeloid Leukemia (AML) Cell Lines

TABLE 2
In vitro data for various ADCs against AML cell lines

| | | | Cell lines; x50 (ng/ml) | | | |
|---|---|---|---|---|---|---|
| | | | HL- | HNT- | | MOLM- |
| ADC | Antigen | Assay | 60 | 34 | KG-1 | 13 |
| Ab1ec-MC6-MC9 (20) | Ag1 | NAD-Glo | 90 | 29 | 19 | 49 |
| Ab2(ec)-MC6-MC9 (20) | Ag2 | NAD-Glo | 782 | 432 | 183 | 3 |
| Ab3(ec)-MC6-MC9 (20) | Ag3 | NAD-Glo | >2000 | 27 | 71 | 7 |

### Example 20: Experimental Data of Nad-Glo Assays of High Load ADCs Against Multiple Myeloma (MM) Cell Lines

TABLE 3
In vitro data for various ADCs against MM cell lines

| | | | Cell lines; x50 (ng/ml) | | |
|---|---|---|---|---|---|
| ADC | Antigen | Assay | MM.1R | MM.1S | U-266 |
| Ab4-MC6(8)-MC9(16) | Ag4 | NAD-Glo | 4 | 3 | 20 |
| Ab5-MC6(8)-MC9(16) | Ag5 | NAD-Glo | 25 | 28 | 180 |
| Ab6-MC6(8)-MC9(16) | Ag6 | NAD-Glo | 2 | 3 | 62 |

The chemical entities recited in the foregoing examples have the following structures:

| Com- | |
|---|---|
| pound | Structure |
| MC1 | 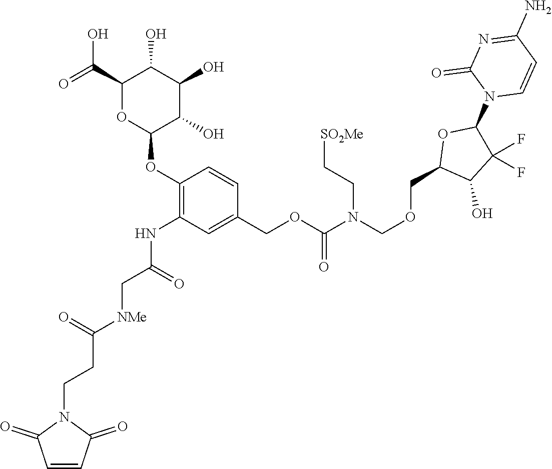 [SMILES: CN(CC(=O)Nc1cc(COC(=O)N(CCS(C)(=O)=O)COC[C@H]2O[C@@H](n3ccc(N)nc3=O)C(F)(F)[C@@H]2O)ccc1O[C@H]1O[C@@H](C(=O)O)[C@H](O)[C@@H](O)[C@@H]1O)C(=O)CCN1C(=O)C=CC1=O] |
| MC2 diacet- amide | 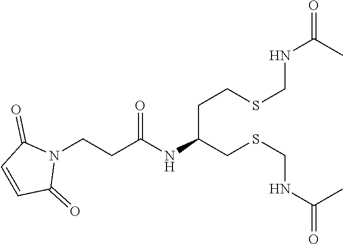 [SMILES: CC(=O)NCSCC[C@@H](CSCNC(C)=O)NC(=O)CCN1C(=O)C=CC1=O] |
| MC2 | 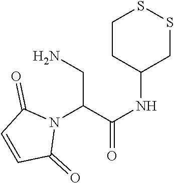 [SMILES: NCC(C(=O)NC1CCSSC1)N1C(=O)C=CC1=O] |
| MC3 | 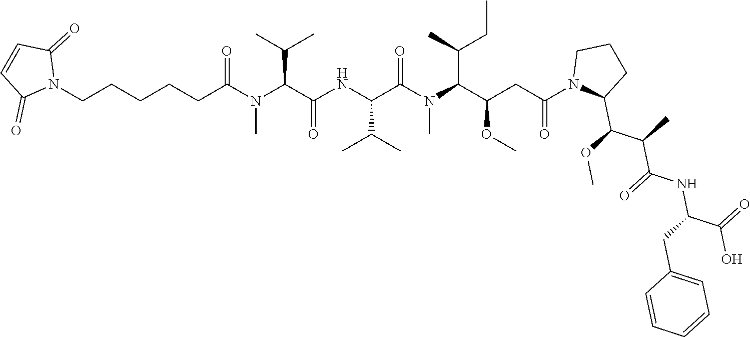 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] |
| MC4 | 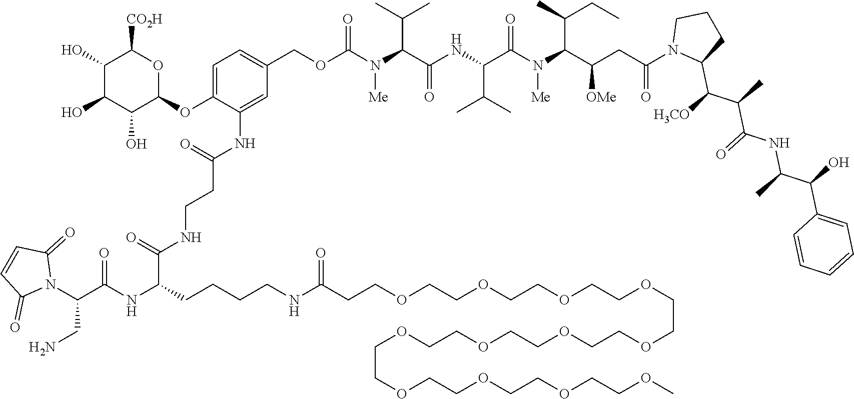 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)[C@H](CCCCNC(=O)CCOCCOCCOCCOCCOCCOCCOCCOCCOCCOCCOCCOC)NC(=O)[C@H](CN)N2C(=O)C=CC2=O)c1)C(C)C] |
| MC5 | 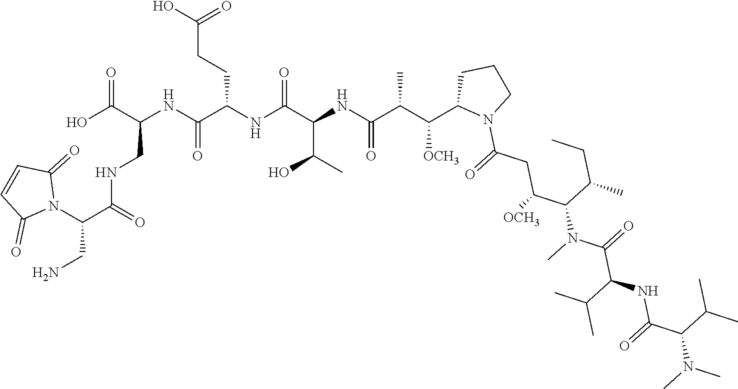 |
| MC6 | 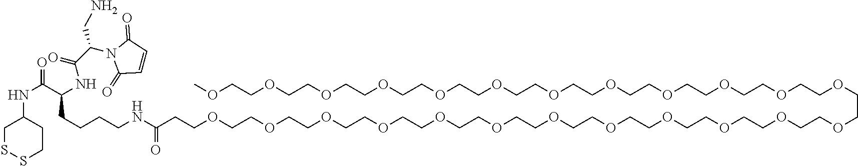 [SMILES: COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@H](CN)N1C(=O)C=CC1=O)C(=O)NC1CCSSC1] |
| MC7 | 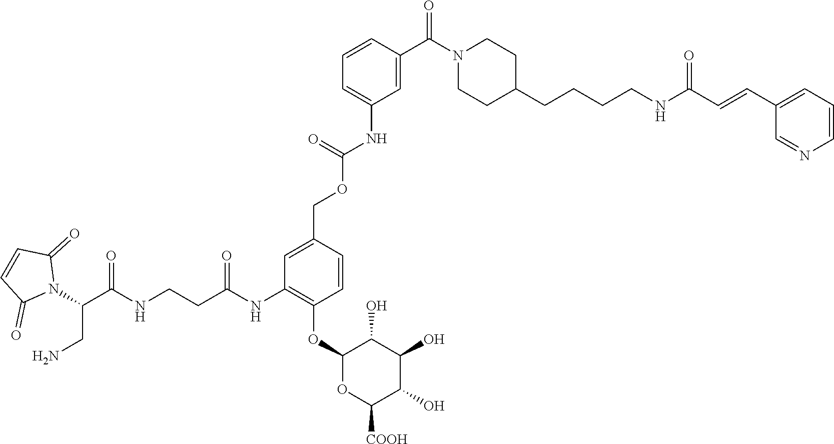 [SMILES: NC[C@@H](C(=O)NCCC(=O)Nc1cc(COC(=O)Nc2cccc(C(=O)N3CCC(CCCCNC(=O)/C=C/c4cccnc4)CC3)c2)ccc1O[C@@H]1O[C@H](C(=O)O)[C@@H](O)[C@H](O)[C@H]1O)N1C(=O)C=CC1=O] |
| MC8 | 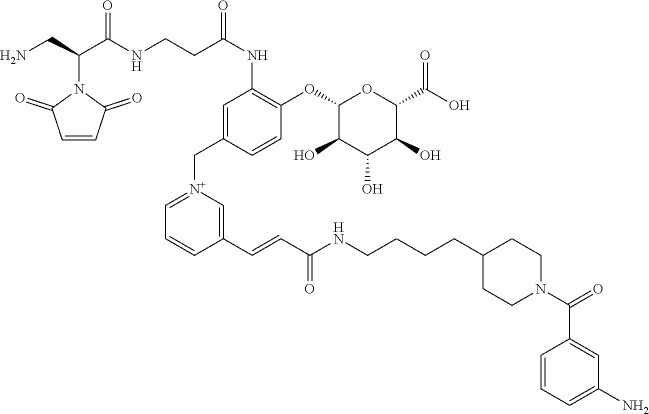 [SMILES: NC[C@@H](C(=O)NCCC(=O)Nc1cc(C[n+]2cccc(/C=C/C(=O)NCCCCC3CCN(C(=O)c4cccc(N)c4)CC3)c2)ccc1O[C@@H]1O[C@H](C(=O)O)[C@@H](O)[C@H](O)[C@H]1O)N1C(=O)C=CC1=O] |
| MC9 | 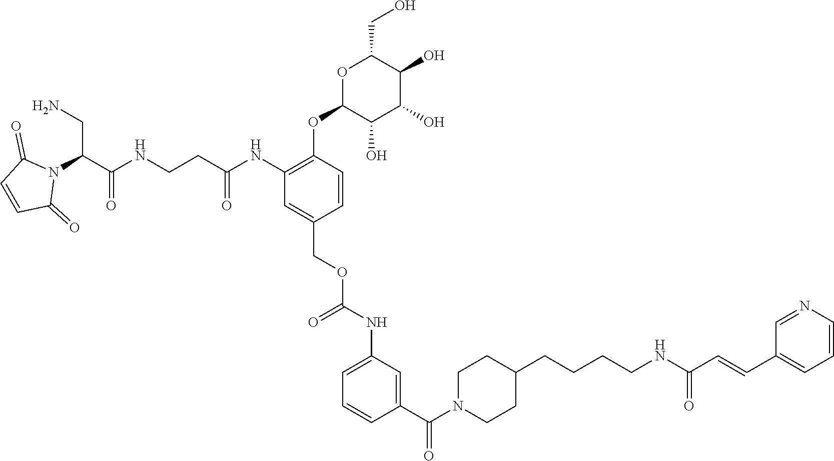 [SMILES: NC[C@@H](C(=O)NCCC(=O)Nc1cc(COC(=O)Nc2cccc(C(=O)N3CCC(CCCCNC(=O)/C=C/c4cccnc4)CC3)c2)ccc1O[C@H]1O[C@H](CO)[C@@H](O)[C@H](O)[C@@H]1O)N1C(=O)C=CC1=O] |
| MC10 | 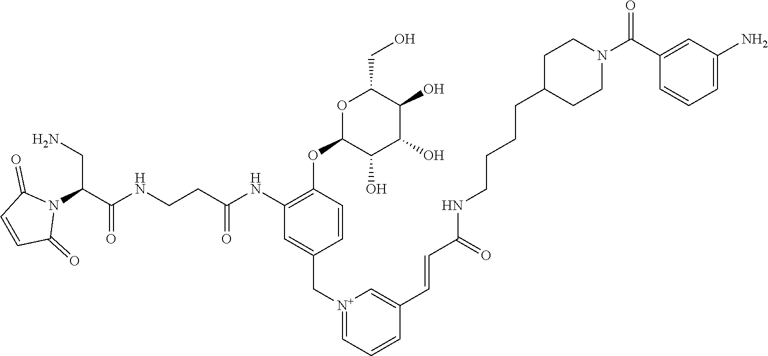 [SMILES: NC[C@@H](C(=O)NCCC(=O)Nc1cc(C[n+]2cccc(/C=C/C(=O)NCCCCC3CCN(C(=O)c4cccc(N)c4)CC3)c2)ccc1O[C@H]1O[C@H](CO)[C@@H](O)[C@H](O)[C@@H]1O)N1C(=O)C=CC1=O] |

What is claimed is:

1. An antibody-drug conjugate (ADC) compound of Formula (I):
Ab-{(S*-L¹)-[(M)ₓ-(L²-D)_{y}]}ₚ (I)
wherein:
Ab is an antibody;
each S* is a sulfur atom from a cysteine residue of the antibody, an ϵ-nitrogen atom from a lysine residue of the antibody, or a triazole moiety, and
each L¹ is a first linker optionally substituted with a PEG Unit ranging from PEG2 to PEG72;
wherein S*-L¹ is selected from the group consisting of formulae A-K: 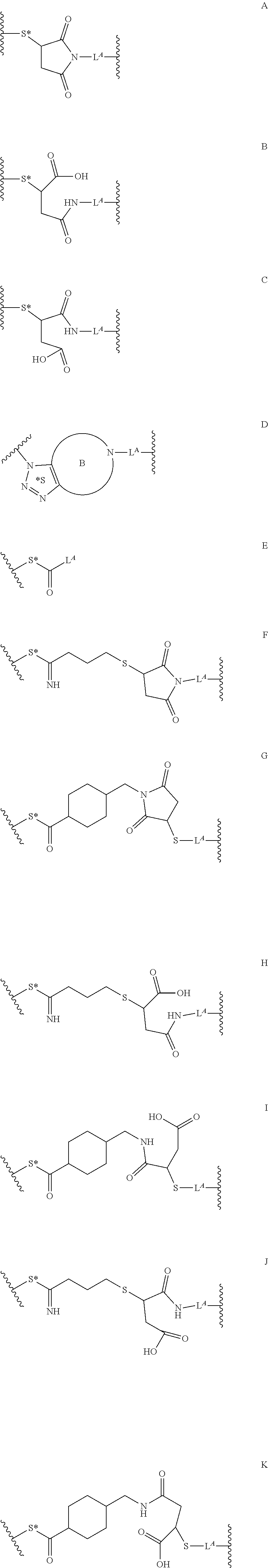
wherein:
each L^{A} is a C₁₋₁₀ alkylene optionally substituted with 1-3 independently selected R^{a}, or a 2-24 membered heteroalkylene optionally substituted with 1-3 independently selected R^{b};
each Ring B is an 8-12 membered heterocyclyl optionally substituted with 1-3 independently selected R^{c}, and further optionally fused to 1-2 rings each independently selected from the group consisting of C₆₋₁₀ aryl and 5-6 membered heteroaryl;
each R^{a}, R^{b}, and R^{c} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d}R^{e}, —C(O)NR^{d}R^{e}, —C(O)(C₁₋₆ alkyl), —(C₁₋₆ alkylene)-NR^{d}R^{e}, and —C(O)O(C₁₋₆ alkyl);
each R^{d} and R^{e} are independently hydrogen or C₁₋₃ alkyl; or R^{d} and R^{e} together with the nitrogen atom to which both are attached form a 5-6 membered heterocyclyl;
L² is an optional second linker optionally substituted with a PEG Unit selected from PEG2 to PEG20;
each M is a multiplexer;
subscript x is 0, 1, 2, 3, or 4;
subscript y is 2^{x};
each D is a Drug Unit;
wherein L¹ and each (M)ₓ-(D)_{y} when L² is absent, or each (M)ₓ-(L²-D)_{y} when L² is present, have a net zero charge at physiological pH;
subscript p is an integer ranging from 2 to 10; and
the ratio of D to Ab is 8:1 to 64:1.

2. The ADC compound of claim 1, wherein each S* is a sulfur atom from a cysteine residue of the antibody.

3. The ADC compound of claim 1 or 2, wherein the cysteine residues are native cysteine residues.

4. The ADC compound of claim 1 or 2, wherein the cysteine residues are from reduced interchain disulfide bonds, or are from engineered cysteine residues, or a combination thereof.

5. The ADC compound of claim 1 or 2, wherein the cysteine residues are engineered cysteine residues.

6. The ADC compound of claim 1 or 2, wherein one or more S* is a sulfur atom from an engineered cysteine residue(s); and each remaining S* is a sulfur atom from a native cysteine residue.

7. The ADC compound of claim 1, wherein each S* is an ϵ-nitrogen atom from a lysine residue of the antibody.

8. The ADC compound of claim 1 or 7, wherein the lysine residues are native lysine residues.

9. The ADC compound of claim 1 or 7, wherein the lysine residues are engineered lysine residues.

10. The ADC compound of claim 1 or 7, wherein one or more S* is an ϵ-nitrogen atom from an engineered lysine residue(s) of the antibody; and each remaining S* is an ϵ-nitrogen atom from a native lysine residue of the antibody.

11. The ADC compound of claim 1, wherein each S* of formula D is a triazole moiety.

12. The ADC compound of any one of claims 1-11, wherein L^{A} is substituted with a PEG Unit ranging from PEG2 to PEG36.

13. The ADC compound of any one of claims 1-6, wherein S*-L¹ is: 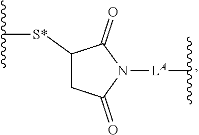
wherein L^{A} is a C₁₋₁₀ alkylene or a 2-10-membered heteroalkylene optionally substituted with 1 R^{a} or 1 R^{b}, respectively, and optionally substituted with a PEG Unit ranging from PEG8 to PEG24 or PEG12 to PEG32.

14. The ADC compound of any one of claims 1-6, wherein S*-L¹ is: 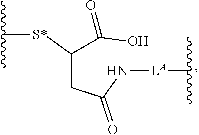
wherein L^{A} is a C₂₋₁₀ alkylene or 2-10-membered heteroalkylene either of which is unsubstituted or substituted with 1 R^{a}, wherein R^{a} is —NR^{d}R^{e}.

15. The ADC compound of any one of claims 1-6, wherein S*-L¹ is: 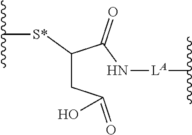
wherein L^{A} is a C₂₋₁₀ alkylene or 2-10-membered heteroalkylene; each optionally substituted with 1 R^{a} or 1 R^{b}, respectively.

16. The ADC compound of claim 1 or 11, wherein S*-L¹ is: 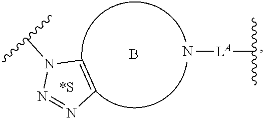
wherein L^{A} is C₁₋₁₀ alkylene or a 2-10 membered heteroalkylene; each optionally substituted with 1-2 R^{a} or 1-2 R^{b}, respectively, provided that one R^{b} is ═O and the carbon atom of the 2-10 membered heteroalkylene so substituted is covalently attached to the nitrogen atom of Ring B;
wherein Ring B is unsubstituted or substituted with 1-2 R^{c}, and is optionally fused to 1-2 rings each independently selected from the group consisting of C₆₋₁₀ aryl and 5-6 membered heteroaryl.

17. The ADC compound of any one of claims 1-16, wherein L^{A} is 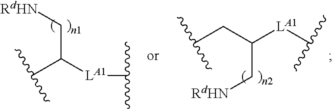
wherein L^{A1} is a bond or a C₁₋₄ alkylene optionally substituted with 1 R^{a}; subscript n1 is 1-4; and subscript n2 is 0-4.

18. The ADC compound of any one of claims 1-17, wherein R^{a} and R^{b} are —(C₁₋₆ alkylene)-NR^{d}R^{e}.

19. The ADC compound of any one of claims 1-18, wherein R^{d} and R^{e} are each hydrogen or are each methyl.

20. The ADC compound of claim 19, wherein L^{A} is 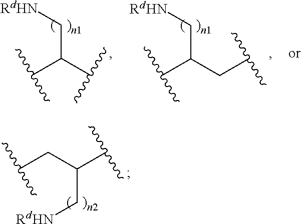
wherein subscript n1 is 1 or 2; and subscript n2 is 0, 1, or 2.

21. The ADC compound of any one of claims 1-20, wherein L^{A} is 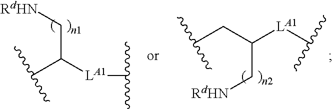
wherein L^{A2} is a C₂₋₁₀ alkylene; subscript n1 is 1 or 2; subscript n2 is 0 or 1; and L^{A2} is further optionally substituted with a PEG Unit ranging from PEG12 to PEG32.

22. The ADC compound of any one of claims 1-21, wherein L^{A} is further optionally substituted with a PEG Unit ranging from PEG8 to PEG32.

23. The ADC compound of any one of claims 1-16 and 22, wherein L^{A} is 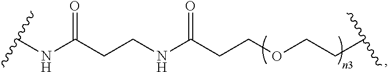
wherein subscript n3 is 1-5.

24. The ADC compound of any one of claims 1, 7, and 16-23, wherein Ring B is an unsubstituted, unfused 8-12 membered heterocyclyl ring.

25. The ADC compound of any one of claims 1, 7, and 16-23, wherein Ring B is an unsubstituted 8-12 membered heterocyclyl fused to a C₆₋₁₀ aryl or 5-6 membered heteroaryl ring.

26. The ADC compound of any one of claims 1, 7, and 16-23, wherein Ring B is an unsubstituted 8-12 membered heterocyclyl fused to two C₆₋₁₀ aryl rings or two 5-6 membered heteroaryl ring rings.

27. The ADC compound of any one of claims 1, 7, and 16-23, wherein Ring B is an unfused 8-12 membered heterocyclyl substituted with 1 R^{c}.

28. The ADC compound of any one of claims 1, 7, and 16-23, wherein Ring B is an 8-12 membered heterocyclyl substituted with 1 R¹, and fused to a C₆₋₁₀ aryl or 5-6 membered heteroaryl ring.

29. The ADC compound of any one of claims 1, 7, and 16-23, wherein Ring B is an unsubstituted 8-12 membered heterocyclyl and fused to two C₆₋₁₀ aryl rings or two 5-6 membered heteroaryl ring rings.

30. The ADC compound of any one of claims 1, 7, and 16-23, wherein Ring B is:

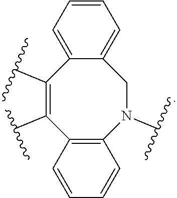 [SMILES: */C1=C(\*)c2ccccc2N(*)Cc2ccccc21]

31. The ADC compound of any one of claim 1-6, wherein S*-L¹ is selected from the group consisting of: 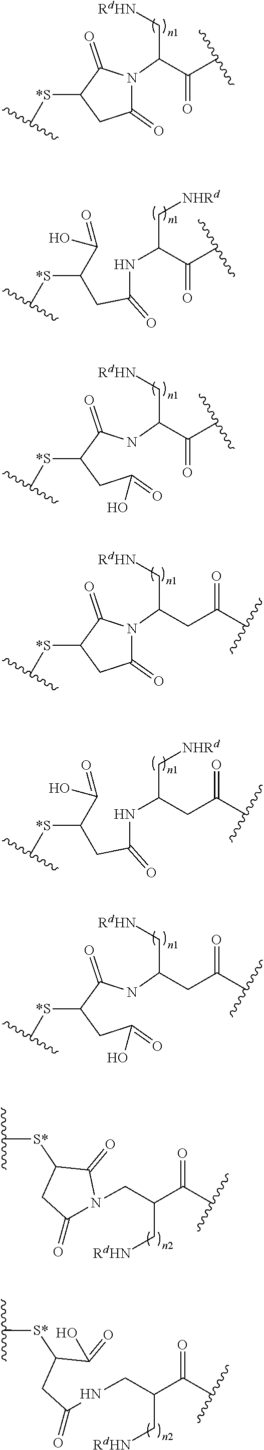 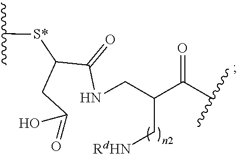
wherein subscript n1 is 1 or 2; and subscript n2 is 0, 1, or 2; and S* is a sulfur atom from a cysteine residue of the antibody.

32. The ADC compound of claim 31, wherein *S-L is selected from the group consisting of: 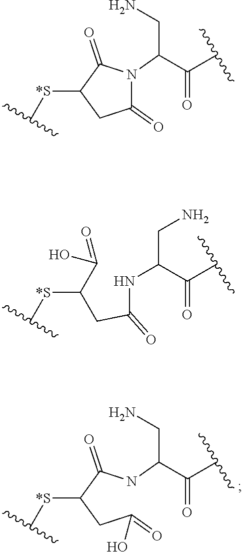
wherein S* is a sulfur atom from a cysteine residue of the antibody.

33. The ADC compound of any one of claims 1-6, wherein S*-L: 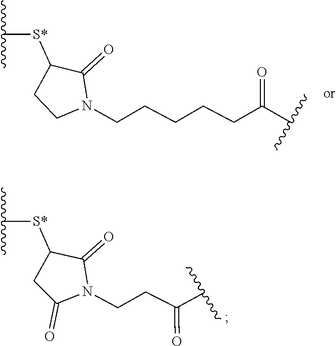
wherein S* is a sulfur atom from a cysteine residue of the antibody.

34. The ADC compound of any one of claims 1-6, wherein *S-L¹ is selected from the group consisting of: 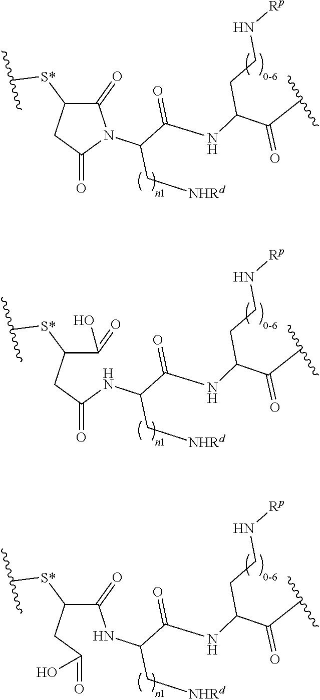
wherein R^{p} is a PEG Unit ranging from PEG8-PEG24, wherein the PEG Unit comprises a —(C₁₋₃ alkylene)C(═O)— group, the carbonyl carbon atom of which provides covalent attachment of R^{p} to the nitrogen atom; and S* is a sulfur atom from a cysteine residue of the antibody.

35. The ADC compound of claim 34, wherein *S-L1 is selected from the group consisting of:

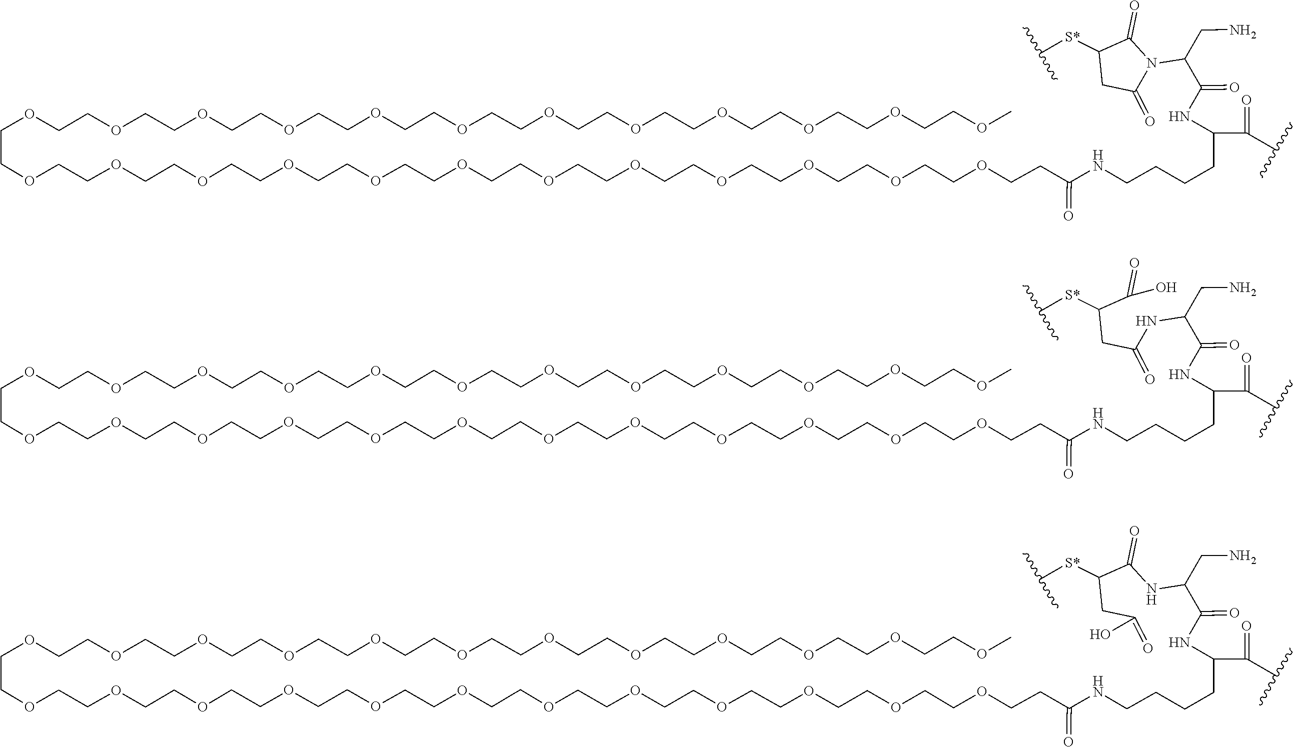

36. The ADC compound of claim 1 or 7, wherein *S-L¹ is:

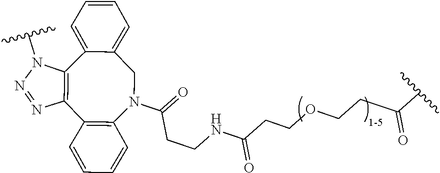

37. The ADC compound of any one of claims 1-36, wherein subscript x is 1.

38. The ADC compound of claim 1 or 37, wherein M is: 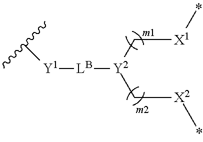
wherein the wavy line represents the covalent attachment of M to L¹;
each * represents the covalent attachment of M to -L²-D;
Y¹ is selected from the group consisting of: a bond, —S—, —O—, and —NH—;
Y² is selected from the group consisting of: CH and N;
L^{B} is absent or a C₁₋₆ alkylene that is optionally interrupted with a group selected from the group consisting of: —O—, —NH—, —N(C₁₋₃ alkyl)-, —C(═O)NH—, —NHC(═O)—, —C(═O)O—, and —O(C═O)—;
X¹ and X² are each independently —S—, —O—, or —NH—; and
subscripts m1 and m2 are each independently 1-4.

39. The ADC compound of any one of claim 1 or 37-38, wherein Y¹ is —NH—; L^{B} is present; Y² is CH; and X¹ and X² are each —S—.

40. The ADC compound of any one of claim 1 or 37-38, wherein Y¹ is a bond; L^{B} is absent; Y² is N; and X¹ and X² are each —S—.

41. The ADC compound of any one of claim 1 or 37-38, wherein M is selected from the group consisting of: 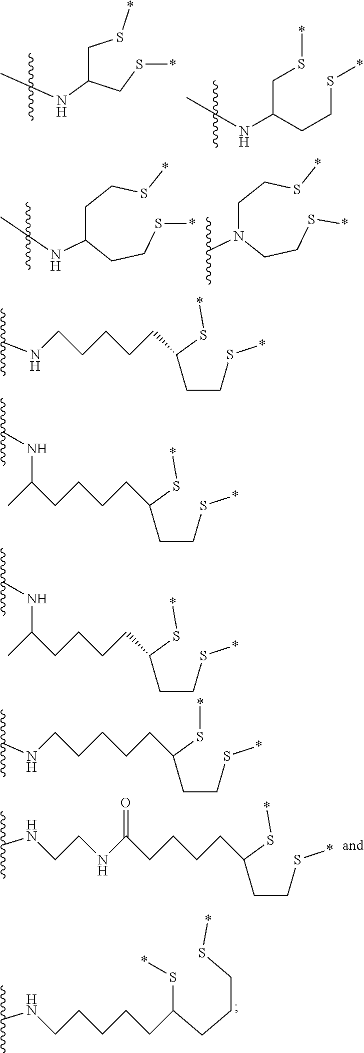
wherein the wavy line represents the covalent attachment of M to L¹; and
wherein each * represents the covalent attachment of M to -(L²-D).

42. The ADC compound of any one of claims 1-36, wherein M is

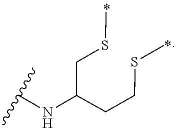 [SMILES: *NC(CCSC)CSC]

43. The ADC compound of any one of claims 1-36, wherein subscript x is 2-4; and (M)ₓ is -M¹-(M²)ₓ₋₁, wherein M¹ and each M² are independently selected multiplexers.

44. The ADC compound of claim 43, wherein subscript x is 2; and (M)ₓ is -M¹-M².

45. The ADC compound of claim 43, wherein subscript x is 3; and (M)ₓ is -M¹-(M²)₂.

46. The ADC compound of any one of claims 3-45, wherein M¹ is: 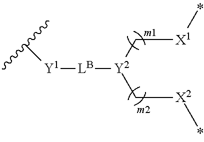
wherein the wavy line represents the covalent attachment of M to L¹;
each * represents the covalent attachment of M¹ to M²;
Y¹ is selected from the group consisting of: a bond, —S—, —O—, and —NH—;
Y² is selected from the group consisting of: CH and N;
L^{B} is absent or a C₁₋₆ alkylene that is optionally interrupted with a group selected from the group consisting of: —O—, —NH—, —N(C₁₋₃ alkyl)-, —C(═O)NH—, —NHC(═O)—, —C(═O)O—, and —O(C═O)—;
X¹ and X² are each independently —S—, —O—, or —NH—; and
subscripts m1 and m2 are each independently 1-4.

47. The ADC compound of claim 46, wherein Y¹ is —NH—; L^{B} is present; Y² is CH; and X¹ and X² are each —S—.

48. The ADC compound of claim 46, wherein Y¹ is a bond; L^{B} is absent; Y² is N; and X¹ and X² are each —S—.

49. The ADC compound of claim 46, wherein Y¹ is a bond; L^{B} is absent; Y² is N; and X¹ and X² are each —NH.

50. The ADC compound of claim 46, wherein M¹ is selected from the group consisting of: 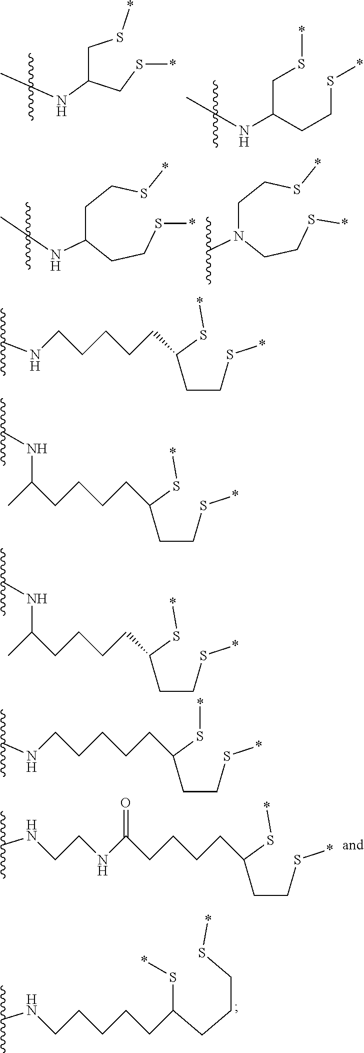
wherein the wavy line represents the covalent attachment of M to L¹; and
wherein each * represents the covalent attachment of M to -(L²-D).

51. The ADC compound of claim 46, wherein M¹ is

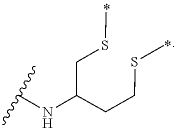 [SMILES: *NC(CCSC)CSC]

52. The ADC compound claim 46, wherein M¹ is

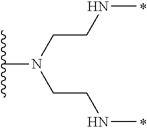 [SMILES: *N(CCNC)CCNC]

53. The ADC compound of any one of claims 43-52, wherein each M² is independently: 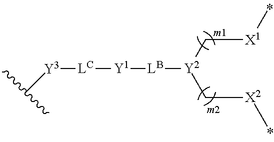
wherein the wavy line represents the covalent attachment of M² to M¹ or to another M²;
each * represents the covalent attachment of M² to L²-D or another M²;
Y¹ is a bond, —S—, —O—, or —NH—;
Y² is CH or N;
Y³ is an optional group that provides covalent attachment of M¹ to the L^{C} (when present) or to Y¹ (when L^{C} is absent) of M²;
L^{B} is absent or a C₁₋₆ alkylene that is optionally interrupted with a group selected from the group consisting of: —O—, —NH—, —N(C₁₋₃ alkyl)-, —C(═O)NH—, —NHC(═O)—, —C(═O)O—, and —O(C═O)—;
X¹ and X² are each independently —S—, —O—, or —NH—;
L^{C} is a C₁₋₁₀ alkylene optionally substituted with 1-3 substituents each independently selected from —(C₁₋₆ alkylene)-NR^{d}R^{e}, NRdRe, and oxo; and
subscripts m1 and m2 are each independently 1-4.

54. The ADC compound of claim 53, wherein Y³ is —C(═O)—.

55. The ADC compound of claim 53, wherein Y³ is selected from the group consisting of: 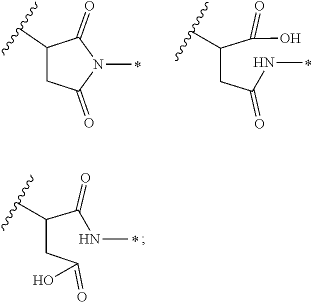
wherein * represents the covalent attachment to L^{C}; and the wavy line represents the covalent attachment to M¹ or another M².

56. The ADC compound of claim 53, wherein Y³-L^{C} is selected from the group consisting of: 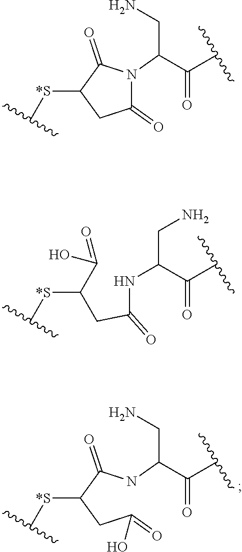
wherein * represents covalent attachment to Y¹; and the wavy line represents the covalent attachment to M¹ or another M².

57. The ADC compound of any one of claims 53-56, wherein Y¹ is —NH—; L^{B} is present; Y² is CH; and X¹ and X² are each —S—.

58. The ADC compound of any one of claims 53-56, wherein Y¹ is a bond; L^{B} is absent; Y² is N; and X¹ and X² are each —NH.

59. The ADC compound of any one of claims 43-52, wherein M² is selected from the group consisting of: 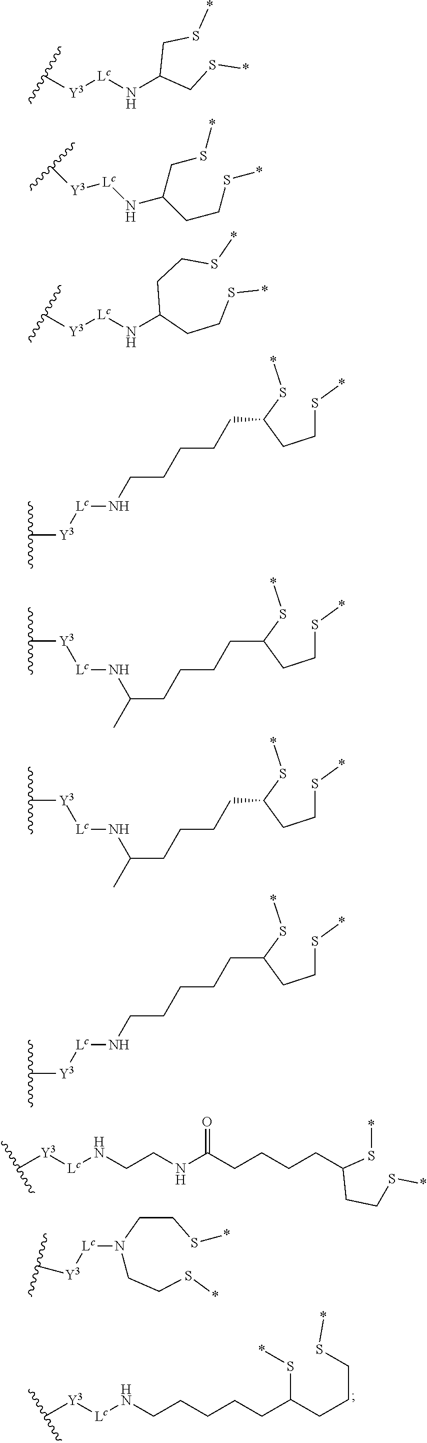
wherein each * represents the covalent attachment to L²-D or another M²; and the wavy bond presents the covalent attachment to M¹ or another M².

60. The ADC compound of any one of claims 43-52, wherein M² is selected from the group consisting of: 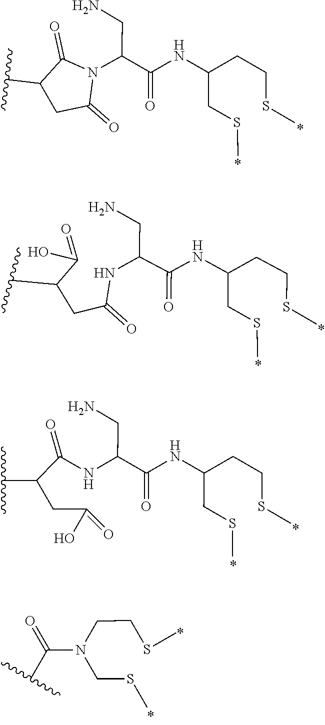 [SMILES: *C1CC(=O)N(C(CN)C(=O)NC(CCSC)CSC)C1=O ; *C(CC(=O)NC(CN)C(=O)NC(CCSC)CSC)C(=O)O ; *C(CC(=O)O)C(=O)NC(CN)C(=O)NC(CCSC)CSC ; *C(=O)N(CCSC)CSC]
wherein each * represents the covalent attachment to L²-D or another M²; and the wavy bond presents the covalent attachment to M¹ or another M².

61. The ADC compound of any one of claims 43-52, wherein subscript x is 2; and (M)ₓ is: 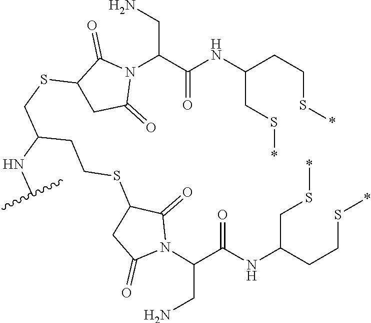 [SMILES: *NC(CCSC1CC(=O)N(C(CN)C(=O)NC(CCS*)CS*)C1=O)CSC1CC(=O)N(C(CN)C(=O)NC(CCS*)CS*)C1=O]
wherein each * represents the covalent attachment to L²-D; the wavy line represents the covalent attachment to L¹; and each succinimide ring is in hydrolyzed form.

62. The ADC compound of any one of claims 1-36, wherein subscript x is 3; and (M)ₓ is: 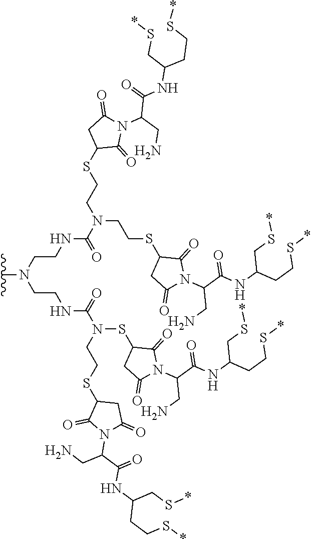 [SMILES: *SCCC(CS*)NC(=O)C(CN)N1C(=O)CC(SCCN(CCSC2CC(=O)N(C(CN)C(=O)NC(CCS*)CS*)C2=O)C(=O)NCCN(*)CCNC(=O)N(CCSC2CC(=O)N(C(CN)C(=O)NC(CCS*)CS*)C2=O)SC2CC(=O)N(C(CN)C(=O)NC(CCS*)CS*)C2=O)C1=O]
wherein each * represents the covalent attachment to L²-D; and each succinimide ring is in hydrolyzed form.

63. The ADC compound of any one of claims 1-36, wherein subscript x is 0.

64. The ADC compound of any one of claims 1-63, wherein L² is substituted with a PEG Unit ranging from PEG2 to PEG36.

65. The ADC compound of any one of claims 1-63, wherein L² is not substituted with a PEG Unit.

66. The ADC compound of any one of claims 1-63, wherein L² has the formula -(Q)_{q}-(A)ₐ-(W)_{w}—(Y)_{y}, wherein:
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
Q is a succinimide or hydrolyzed succinimide;
subscript q is 0 or 1;
subscript a is 0 or 1;
subscript w is 0 or 1;
wherein when subscript w is 1 then W is from 1-12 amino acids or has the structure: 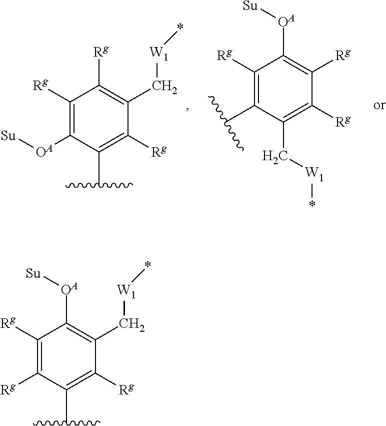
wherein Su is a Sugar moiety;
—O^{A}— represents the oxygen atom of a glycosidic bond;
each R⁹ is independently hydrogen, halogen, —CN, or —NO₂;
W¹ is selected from the group consisting of: a bond, —O—, —NH—, —N(C₁₋₆ alkyl)-, —[N(C₁₋₆ alkyl)₂]⁺-, and —OC(═O)—;
the wavy line represents the covalent attachment to A, Q, or L¹; and
the * represents the covalent attachment to Y or D;
y is 0 or 1; and
Y is a self-immolative or non-self-immolative moiety; and y is 0 or 1.

67. The ADC compound of any one of claims 1-66, wherein each L²-D is uncharged.

68. The ADC compound of any one of claims 1-66, wherein each L²-D has a net zero charge.

69. The ADC compound of any one of claims 66-68, wherein Q-A is selected from the group consisting of: 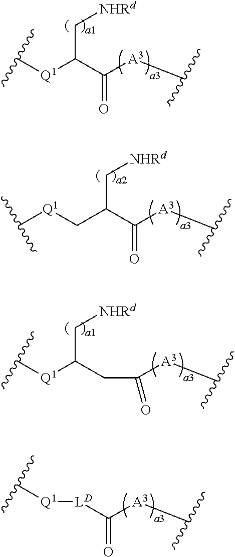
wherein Q¹ is selected from the group consisting of: PGP 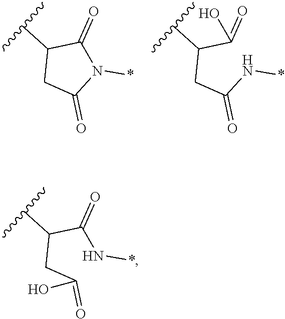
wherein the wavy line adjacent to
Q¹ represents covalent attachment to (M)ₓ;
subscript a1 is 1-4;
subscript a2 is 0-3;
subscript a3 is 0 or 1;
L^{D} is a C₁₋₆ alkylene;
A³ is —NH—(C₁₋₁₀ alkylene)-C(═O)— or —NH-(2-20 membered heteroalkylene)-C(═O)—, wherein the C₁₋₆ alkylene is optionally substituted with 1-3 independently selected R^{a}, and the 2-20 membered heteroalkylene is optionally substituted with 1-3 independently selected R^{b}; and
wherein A³ is further optionally substituted with a PEG Unit selected from PEG8 to PEG24.

70. The ADC compound of claim 69, wherein subscript a3 is 1.

71. The ADC compound of any one of claims 68-70, wherein A³ is —NH—(C₁₋₁₀ alkylene)-C(═O)—.

72. The ADC compound of any one of claims 68-70, wherein A³ is —NH—(CH₂CH₂)—C(═O)—.

73. The ADC compound of any one of claims 68-70, wherein A³ is —NH-(2-20 membered heteroalkylene)-C(═O)—, wherein the 2-20 membered heteroalkylene is optionally substituted with 1-3 independently selected R^{b}; and
wherein A³ is further optionally substituted with a PEG Unit selected from PEG8 to PEG24.

74. The ADC compound of claim 69, wherein A³ is 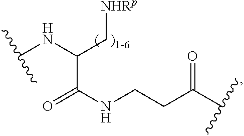
wherein R^{p} is selected from PEG2 to PEG24.

75. The ADC compound of claim 74, wherein R^{p} is PEG12.

76. The ADC compound of claim 74, wherein the PEG Unit R^{p} comprises a —(C₁₋₆ alkylene)C(═O)— group, the carbonyl carbon atom of which provides covalent attachment of R^{p} to the nitrogen atom.

77. The ADC compound of any one of claims 66-76, wherein W is from 2 to 12 amino acids independently selected from natural and unnatural amino acids.

78. The ADC compound of claim 77, wherein W is a dipeptide.

79. The ADC compound of any one of claims 66-78, wherein the bond between W, and D or Y, is enzymatically cleavable by a tumor-associated protease.

80. The ADC compound of claim 79, wherein the tumor-associate protease is a cathepsin.

81. The ADC compound of any one of claims 66-76, wherein W has the structure of: 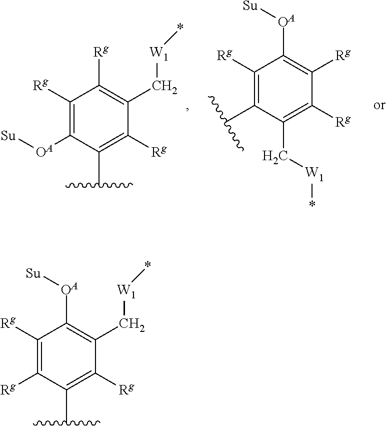
wherein Su is a Sugar moiety;
—O^{A}— represents the oxygen atom of a glycosidic bond;
each R^{g} is independently hydrogen, halogen, —CN, or —NO₂;
W¹ is selected from the group consisting of: a bond, —O—, —C(═O)—, —S(O)₀₋₂—, —NH—, —N(C₁₋₆ alkyl)-, —[N(C₁₋₆ alkyl)₂]⁺-, —OC(═O)—, —NHC(═O)—, —C(═O)O—, and —C(═O)NH—;
the wavy line represents the covalent attachment to A, Q, or L¹; and
the * represents the covalent attachment to Y or D.

82. The ADC compound of any one of claims 66-75 and 81, wherein O^{A}-Su is charge neutral at physiological pH.

83. The ADC compound of any one of claims 66-75 and 81-82, wherein Su of O^{A}-Su is mannose.

84. The ADC compound of any one of claims 66-75 and 81, wherein O^{A}-Su is

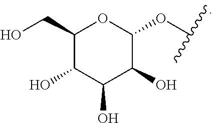 [SMILES: *O[C@H]1O[C@H](CO)[C@@H](O)[C@H](O)[C@@H]1O]

85. The ADC compound of any one of claims 66-75 and 81, wherein Su of O^{A}-Su comprises a carboxylate moiety.

86. The ADC compound of any one of claims 66-75, 81, and 85, wherein Su of O^{A}-Su is glucuronic acid.

87. The ADC compound of claim 77, wherein O^{A}-Su is

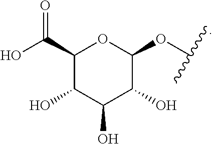 [SMILES: *O[C@@H]1O[C@H](C(=O)O)[C@@H](O)[C@H](O)[C@H]1O]

88. The ADC compound of any one of claims 66-75 and 81, wherein W is

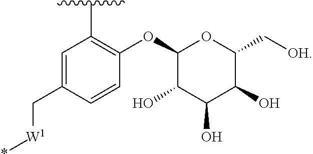

89. The ADC compound of any one of claims 66-75 and 81, wherein W is

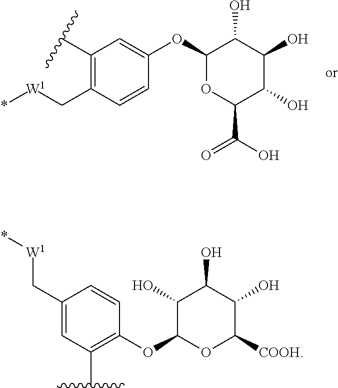

90. The ADC compound of any one of claims 66-89, wherein W¹ is a bond.

91. The ADC compound of any one of claims 66-89, wherein W¹ is —O(C═O)—.

92. The ADC compound of any one of claims 66-91, wherein subscript y is 0.

93. The ADC compound of claims 66-91, wherein subscript y is 1; and Y is 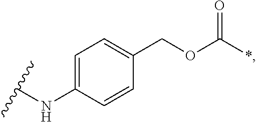
wherein the wavy line represents covalent attachment to W or A; and
the * represents covalent attachment to D.

94. The ADC compound of any one of claims 66-68, wherein Q-A is 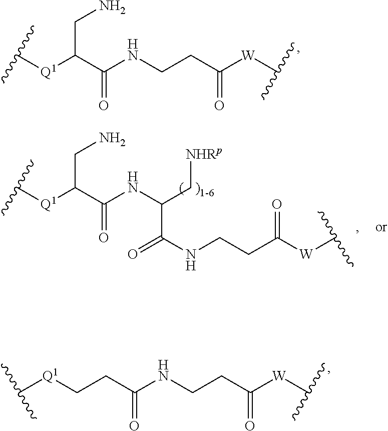
wherein R^{p} is PEG8 to PEG24,

95. The ADC compound of claim 94, wherein R^{p} is PEG12.

96. The ADC compound of claim 94 or 95, wherein the PEG Unit R^{p} comprises a —(C₁₋₆ alkylene)C(═O)— group, the carbonyl carbon atom of which provides covalent attachment of R^{P} to the nitrogen atom.

97. The ADC compound of any one of claims 66-76, 81, and 92-96, wherein W has the structure of: 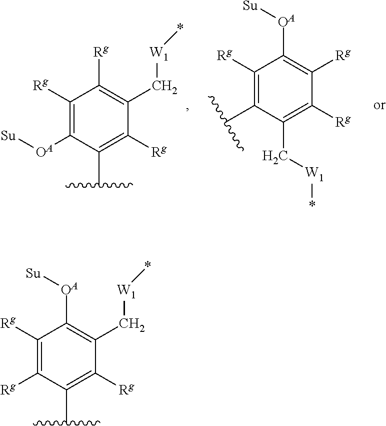
wherein Su is a Sugar moiety;
—O^{A}— represents the oxygen atom of a glycosidic bond;
each R^{g} is independently hydrogen, halogen, —CN, or —NO₂;
W¹ is selected from the group consisting of: a bond, —O—, —C(═O)—, —S(O)₀₋₂—, —NH—, —N(C₁₋₆ alkyl)-, and —[N(C₁₋₆ alkyl)₂]⁺-;
the wavy line represents the covalent attachment to A, Q, or L¹; and
the * represents the covalent attachment to Y or D.

98. The ADC compound of any one of claims 66, 81, and 96, wherein each R^{g} is hydrogen or one R^{g} is halogen, —CN, or —NO₂ and each remaining R^{g} is hydrogen.

99. The ADC compound of claim 97, wherein W¹ is —OC(═O)—; and O^{A}-Su is charged neutral.

100. The ADC compound of claim 97, wherein W¹ is a bond; D is conjugated to W through a nitrogen atom which forms an ammonium cation at physiological pH; and O^{A}-Su comprises a carboxylate.

101. The ADC compound of any one of claims 1-100 wherein D is a hydrophilic Drug Unit.

102. The ADC compound of any one of claims 1-101, wherein D is from a cytotoxic agent.

103. The ADC compound of any one of claims 1-100 wherein D is from gemcitabine, MMAE, or MMAF.

104. The ADC compound of any one of claims 1-100 wherein D is a from a NAMPT inhibitor.

105. The ADC compound of any one of claims 1-100 and 104, wherein D has the following formula: 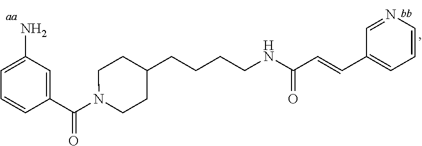
wherein D is covalently attached to L² at the aa or bb position.

106. The ADC compound of any one of claims 1-105, wherein each L²-D has zero net charge at physiological pH.

107. The ADC compound of any one of claims 1-106, wherein each L²-D has no charged species at physiological pH.

108. The ADC compound of any one of claims 1-105, wherein each L²-D is zwitterionic at physiological pH.

109. The ADC compound of claims 1-106 and 108, wherein each L²-D comprises a carboxylate and an ammonium.

110. The ADC compound of claim 109, wherein the ammonium is a quaternary ammonium.

111. The ADC compound of claim 110, wherein the quaternary ammonium is pyridinium.

112. The ADC compound of any one of claims 1-106, wherein L² is anionic; and D is cationic.

113. The ADC compound of any one of claims 1-106 and 108-109, wherein L² comprises a carboxylate; and D comprises an ammonium.

114. The ADC compound of any one of claims 1-113, wherein the ratio of D to Ab is 8:1.

115. The ADC compound of any one of claims 1-113, wherein the ratio of D to Ab is 16:1 to 64:1

116. The ADC compound of any one of claims 1-113, wherein the ratio of D to Ab is 16:1 to 32:1.

117. The ADC compound of any one of claims 1-113, wherein the ratio of D to Ab is 16:1.

118. The ADC of any one of claims 1-113, wherein the ratio of D to Ab is 8:1; subscript y of (L²-D)_{y} is 4; and subscript p is 2.

119. The ADC of any one of claims 1-113, wherein the ratio of D to Ab is 8:1; y of (L²-D)_{y} is 2; and subscript p is 4.

120. The ADC of any one of claims 1-113, wherein the ratio of D to Ab is 16:1; y of (L²-D)_{y} is 8; and subscript p is 2.

121. The ADC of any one of claims 1-113, wherein the ratio of D to Ab is 16:1; y of (L²-D)_{y} is 4; and subscript p is 4.

122. The ADC of any one of claims 1-113, wherein the ratio of D to Ab is 16:1; y of (L²-D)_{y} is 2; and subscript p is 8.

123. The ADC of any one of claims 1-122, wherein the total number of charges for each instance of (M)ₓ-(L²-D)_{y} is an even number at physiological pH.

124. The ADC of any one of claims 1-123, wherein the total number of charges for each instance of (M)ₓ-(L²-D)_{y}≥2(x+2y) at physiological pH.

125. The ADC of any one of claims 1-124, wherein the total number of charges for each instance of (M)ₓ-(L²-D)_{y} is 2(x+2y) at physiological pH.

126. A composition comprising the ADC of any one of claims 1-125, or a pharmaceutically acceptable salt thereof.

127. A method of treating cancer in a subject in need thereof, comprising administering to the subject a therapeutically effective amount of the ADC of any one of claims 1-125, or a pharmaceutically acceptable salt thereof, or the composition of claim 126.

128. A method of treating an autoimmune disorder in a subject in need thereof, comprising administering to the subject a therapeutically effective amount of the ADC of any one of claims 1-125, or a pharmaceutically acceptable salt thereof, or the composition of claim 126.
